SEQUENCE LISTING (511 sequences)

SEQ ID NO: 1 - protein, 387 aa, Homo sapiens
MPVPASWPHPPGPFLLLTLLLGLTEVAGEEELQMIQPEKLLLVTVGKTATLHCTVTSLLPVGPVLWFRGVGPGRELIYNQKEGHFPRVTTVSDLTKRNNMDFSIRISSITPADVGTYYCVKFRKGSPENVEFKSGPGTEMALGAKPSAPVVLGPAARTTPEHTVSFTCESHGFSPRDITLKWFKNGNELSDFQTNVDPTGQSVAYSIRSTARVVLDPWDVRSQVICEVAHVTLQGDPLRGTANLSEAIRVPPTLEVTQQPMRVGNQVNVTCQVRKFYPQSLQLTWSENGNVCQRETASTLTENKDGTYNWTSWFLVNISDQRDDVVLTCQVKHDGQLAVSKRLALEVTVHQKDQSSDATPGPASSLTALLLIAVLLGPIYVPWKQKT

SEQ ID NO: 2 - protein, 170 aa, Homo sapiens
MPVPASWPHPPGPFLLLTLLLGLTEVAGEEELQMIQPEKLLLVTVGKTATLHCTVTSLLPVGPVLWFRGVGPGRELIYNQKEGHFPRVTTVSDLTKRNNMDFSIRISSITPADVGTYYCVKFRKGSPENVEFKSGPGTEMALGGPASSLTALLLIAVLLGPIYVPWKQKT

SEQ ID NO: 3 - protein, 276 aa, Homo sapiens
MPVPASWPHPPGPFLLLTLLLGLTEVAGEEELQMIQPEKLLLVTVGKTATLHCTVTSLLPVGPVLWFRGVGPGRELIYNQKEGHFPRVTTVSDLTKRNNMDFSIRISSITPADVGTYYCVKFRKGSPENVEFKSGPGTEMALGAKPSAPVVLGPAARTTPEHTVSFTCESHGFSPRDITLKWFKNGNELSDFQTNVDPTGQSVAYSIRSTARVVLDPWDVRSQVICEVAHVTLQGDPLRGTANLSEAIRGPASSLTALLLIAVLLGPIYVPWKQKT

SEQ ID NO: 4 - protein, 387 aa, Macaca fascicularis
MPVPASWSHPPGPFLLLTLLLGFTEVAGEEELQMIQPEKLLLVAVGESATLNCTVTSLLPVGPVLWFRGVGPGRELIYNQKEGHFPRVTTVSDLTKRNNMDFSIRISSITPADAGTYYCVKFRKGSPENVEFKSGPGTEMALRAKPSAPVVSGPAARATPEHTVSFTCKSHGFSPRDITLKWFKNGNELSDFQTNVDPAGQSVSYSIRSTARVVLAPWDVRSQVTCEVAHVTLQGDPLRGTANLSEAIRVPPTLEVTQQPMRAGNQVNITYQVRNFYPQNLQLTWLENGNVCRTETASTLTENKDGTYNWTSWLLVNTSDQRDDVVLTCQVKHDGQLAVNKSLVLEVSAHQKDQSSDATHGPASSLTVLLLIAVLLGPIYVPWKQKS

SEQ ID NO: 5 - protein, 330 aa, Homo sapiens [sequence omitted]

SEQ ID NO: 6 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 330 aa, Homo sapiens; identical to SEQ ID NO: 6

SEQ ID NO: 13 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 14 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 15 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 16 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 17 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKXEPKSCDKTHTCPPCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKXEPKSCDKTHTCPPCPAPELLXGPXVFLFPPKPKDTLMISRTPEVTCVVVDVSHEDPEVKFNWYVDGVEVHNAKTKPREEQYNSTYRVVSVLTVLHQDWLNGKEYKCKVSNKALPAPXEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKXEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 26 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKXEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVXHEALHXHYTQKSLSLSPGK

SEQ ID NO: 27 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKXVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 29 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 30 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 31 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 32 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEALGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 33 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 34 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 35 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPXCPAPEFXGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 36 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 354 aa, Homo sapiens
MIQPEKLLLVTVGKTATLHCTVTSLLPVGPVLWFRGVGPGRELIYNQKEGHFPRVTTVSDLTKRNNMDFSIRISSITPADVGTYYCVKFRKGSPENVEFKSGPGTEMALGAKPSAPVVLGPAARTTPEHTVSFTCESHGFSPRDITLKWFKNGNELSDFQTNVDPTGQSVAYSIRSTARVVLDPWDVRSQVICEVAHVTLQGDPLRGTANLSEAIRVPPTLEVTQQPMRVGNQVNVTCQVRKFYPQSLQLTWSENGNVCQRETASTLTENKDGTYNWTSWFLVNISDQRDDVVLTCQVKHDGQLAVSKRLALEVTVHQKDQSSDATPGPASSLTALLLIAVLLGPIYVPWKQKT

SEQ ID NO: 38 - protein, 107 aa, Homo sapiens [sequence omitted]

SEQ ID NO: 39 - protein, 106 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 40 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 41 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 42 - protein, 106 aa, Homo sapiens
GQPKAAPSVTLFPPSSEELQANKATLVCLVSDFNPGAVTVAWKADGSPVKVGVETTKPSKQSNNKYAASSYLSLTPEQWKSHRSYSCRVTHEGSTVEKTVAPAECS

SEQ ID NO: 43-99 - skipped (57 entries)

SEQ ID NO: 100 - protein, 6 aa, synthetic construct
QSVSSN

SEQ ID NO: 101 - protein, 6 aa, synthetic construct
QSISSW

SEQ ID NO: 102 - protein, 12 aa, synthetic construct
RASQSLGSTYLA

SEQ ID NO: 103 - protein, 11 aa, synthetic construct
QASQDIHNYLN

SEQ ID NO: 104 - protein, 11 aa, synthetic construct
RASQTINNYLN

SEQ ID NO: 105 - protein, 11 aa, synthetic construct
RASQNIRNYLN

SEQ ID NO: 106 - protein, 16 aa, synthetic construct
RSSQSLLHSNGYNYLD

SEQ ID NO: 107 - protein, 11 aa, synthetic construct
RASQGVGDRSA

SEQ ID NO: 108 - protein, 11 aa, synthetic construct
RASQGISTYLA

SEQ ID NO: 109 - protein, 11 aa, synthetic construct
RASQSISSYLN

SEQ ID NO: 110 - protein, 11 aa, synthetic construct
RASQGIGNDLA

SEQ ID NO: 111 - protein, 11 aa, synthetic construct
RASESISHWLA

SEQ ID NO: 112 - protein, 17 aa, synthetic construct
KSSQSLLYSSNNKNYLA

SEQ ID NO: 113 - protein, 11 aa, synthetic construct
RASQDIRRALA

SEQ ID NO: 114 - protein, 11 aa, synthetic construct
QASQGISSYLN

SEQ ID NO: 115 - protein, 11 aa, synthetic construct
RASQGIGNDLG

SEQ ID NO: 116 - protein, 11 aa, synthetic construct
RTSQSIGTYLN

SEQ ID NO: 117 - protein, 11 aa, synthetic construct
RASQSVDTWLA

SEQ ID NO: 118 - protein, 11 aa, synthetic construct
RASQSISNLLA

SEQ ID NO: 119 - protein, 11 aa, synthetic construct
RASQGISNWLA

SEQ ID NO: 120 - protein, 12 aa, synthetic construct
RASQSISGSYLA

SEQ ID NO: 121 - protein, 11 aa, synthetic construct
RASQDIRNDLA

SEQ ID NO: 122 - protein, 17 aa, synthetic construct
RSSQSLFHSSTNANNLA

SEQ ID NO: 123 - protein, 11 aa, synthetic construct
RASQSISNWLA

SEQ ID NO: 124 - protein, 11 aa, synthetic construct
RAGQSIGDYLN

SEQ ID NO: 125 - protein, 11 aa, synthetic construct
QASQGISNYLN

SEQ ID NO: 126 - protein, 11 aa, synthetic construct
RASQSISRWLA

SEQ ID NO: 127 - protein, 17 aa, synthetic construct
RSSQSVLYSSNNKNYLA

SEQ ID NO: 128 - protein, 11 aa, synthetic construct
RASQSVSSYLA

SEQ ID NO: 129 - protein, 17 aa, synthetic construct
KSSQSVLYSSNNKNYLA

SEQ ID NO: 130 - protein, 17 aa, synthetic construct
KSSQSLLYTSNDRNYLA

SEQ ID NO: 131 - protein, 11 aa, synthetic construct
RASQSISSWLA

SEQ ID NO: 132 - protein, 11 aa, synthetic construct
RASQSVSSNLA

SEQ ID NO: 133 - protein, 11 aa, synthetic construct
QASQDISNYLN

SEQ ID NO: 134 - protein, 11 aa, synthetic construct
RASQRIGSYLN

SEQ ID NO: 135 - protein, 11 aa, synthetic construct
RASESITNWLA

SEQ ID NO: 136 - protein, 11 aa, synthetic construct
RASQGIRNDLG

SEQ ID NO: 137 - protein, 11 aa, synthetic construct
RASQSVSKYLA

SEQ ID NO: 138-139 - skipped (2 entries)

SEQ ID NO: 140 - protein, 7 aa, synthetic construct
GASTRAT

SEQ ID NO: 141 - protein, 7 aa, synthetic construct
DASNLET

SEQ ID NO: 142 - protein, 7 aa, synthetic construct
AASSLQG

SEQ ID NO: 143 - protein, 7 aa, synthetic construct
AASSLQS

SEQ ID NO: 144 - protein, 7 aa, synthetic construct
AASTLQS

SEQ ID NO: 145 - protein, 7 aa, synthetic construct
DASSLES

SEQ ID NO: 146 - protein, 7 aa, synthetic construct
DAKDLHP

SEQ ID NO: 147 - protein, 7 aa, synthetic construct
GASTLQS

SEQ ID NO: 148 - protein, 7 aa, synthetic construct
WASIRES

SEQ ID NO: 149 - protein, 7 aa, synthetic construct
LGSSRAS

SEQ ID NO: 150 - protein, 7 aa, synthetic construct
AASSLQN

SEQ ID NO: 151 - protein, 7 aa, synthetic construct
AASNLET

SEQ ID NO: 152 - protein, 7 aa, synthetic construct
DASNLPT

SEQ ID NO: 153 - protein, 7 aa, synthetic construct
EASTLES

SEQ ID NO: 154 - protein, 7 aa, synthetic construct
DASNLDA

SEQ ID NO: 155 - protein, 7 aa, synthetic construct
WASTRES

SEQ ID NO: 156 - protein, 7 aa, synthetic construct
LASTRAP

SEQ ID NO: 157 - protein, 7 aa, synthetic construct
AASSLES

SEQ ID NO: 158 - protein, 7 aa, synthetic construct
GTSTRAT

SEQ ID NO: 159 - protein, 7 aa, synthetic construct
WASTRAS

SEQ ID NO: 160 - protein, 7 aa, synthetic construct
AASTLQT

SEQ ID NO: 161 - protein, 7 aa, synthetic construct
GASNLQS

SEQ ID NO: 162 - protein, 7 aa, synthetic construct
KASSLES

SEQ ID NO: 163 - protein, 7 aa, synthetic construct
DTSNLQG

SEQ ID NO: 164 - protein, 7 aa, synthetic construct
SASNLQS

SEQ ID NO: 165 - protein, 7 aa, synthetic construct
DATTLES

SEQ ID NO: 166 - protein, 7 aa, synthetic construct
AISARAA

SEQ ID NO: 167 - protein, 9 aa, synthetic construct
QQYNNWPLT

SEQ ID NO: 168 - protein, 8 aa, synthetic construct
QQYDSYYT

SEQ ID NO: 169 - protein, 11 aa, synthetic construct [sequence omitted]

SEQ ID NO: 170 - protein, 11 aa, synthetic construct [sequence omitted]

SEQ ID NO: 171 - protein, 11 aa, synthetic construct [sequence omitted]

SEQ ID NO: 172 - protein, 11 aa, synthetic construct [sequence omitted]

SEQ ID NO: 173 - protein, 11 aa, synthetic construct [sequence omitted]

SEQ ID NO: 174 - protein, 11 aa, synthetic construct
CMQGLQTLYSF

SEQ ID NO: 175 - protein, 11 aa, synthetic construct [sequence omitted]

SEQ ID NO: 176 - protein, 11 aa, synthetic construct [sequence omitted]

SEQ ID NO: 177 - protein, 11 aa, synthetic construct [sequence omitted]

SEQ ID NO: 178 - protein, 11 aa, synthetic construct [sequence omitted]

SEQ ID NO: 179 - protein, 11 aa, synthetic construct
CQQYLSPPLTF

SEQ ID NO: 180 - protein, 11 aa, synthetic construct [sequence omitted]

SEQ ID NO: 181 - protein, 10 aa, synthetic construct [sequence omitted]

SEQ ID NO: 182 - protein, 11 aa, synthetic construct [sequence omitted]

SEQ ID NO: 183 - protein, 11 aa, synthetic construct [sequence omitted]

SEQ ID NO: 184 - protein, 9 aa, synthetic construct
CQNYNSALF

SEQ ID NO: 185 - protein, 11 aa, synthetic construct [sequence omitted]

SEQ ID NO: 186 - protein, 11 aa, synthetic construct [sequence omitted]

SEQ ID NO: 187 - protein, 11 aa, synthetic construct [sequence omitted]

SEQ ID NO: 188 - protein, 11 aa, synthetic construct [sequence omitted]

SEQ ID NO: 189 - protein, 11 aa, synthetic construct [sequence omitted]

SEQ ID NO: 190 - protein, 11 aa, synthetic construct [sequence omitted]

SEQ ID NO: 191 - protein, 11 aa, synthetic construct [sequence omitted]

SEQ ID NO: 192 - protein, 11 aa, synthetic construct
CMQGSHWPPSF

SEQ ID NO: 193 - protein, 11 aa, synthetic construct [sequence omitted]

SEQ ID NO: 194 - protein, 11 aa, synthetic construct [sequence omitted]

SEQ ID NO: 195 - protein, 11 aa, synthetic construct [sequence omitted]

SEQ ID NO: 196 - protein, 11 aa, synthetic construct [sequence omitted]

SEQ ID NO: 197 - protein, 11 aa, synthetic construct [sequence omitted]

SEQ ID NO: 198 - protein, 11 aa, synthetic construct [sequence omitted]

SEQ ID NO: 199 - protein, 11 aa, synthetic construct [sequence omitted]

SEQ ID NO: 200 - protein, 11 aa, synthetic construct [sequence omitted]

SEQ ID NO: 201 - protein, 11 aa, synthetic construct [sequence omitted]

SEQ ID NO: 202 - protein, 11 aa, synthetic construct [sequence omitted]

SEQ ID NO: 203 - protein, 11 aa, synthetic construct [sequence omitted]

SEQ ID NO: 204 - protein, 11 aa, synthetic construct [sequence omitted]

SEQ ID NO: 205 - protein, 11 aa, synthetic construct [sequence omitted]

SEQ ID NO: 206 - protein, 11 aa, synthetic construct [sequence omitted]

SEQ ID NO: 207 - protein, 11 aa, synthetic construct
CQQSYDVPLTF

SEQ ID NO: 208 - protein, 11 aa, synthetic construct [sequence omitted]

SEQ ID NO: 209 - protein, 8 aa, synthetic construct
GFTFSNYG

SEQ ID NO: 210 - protein, 8 aa, synthetic construct
GYTFTGYH

SEQ ID NO: 211 - protein, 9 aa, synthetic construct
YSFSDYYMH

SEQ ID NO: 212 - protein, 9 aa, synthetic construct
GTFSNYDIS

SEQ ID NO: 213 - protein, 9 aa, synthetic construct
YTFTSYDIN

SEQ ID NO: 214 - protein, 9 aa, synthetic construct
GTFGSYGIN

SEQ ID NO: 215 - protein, 9 aa, synthetic construct
FTFSNSDMN

SEQ ID NO: 216 - protein, 9 aa, synthetic construct
YTFTSYYMH

SEQ ID NO: 217 - protein, 9 aa, synthetic construct
YSFTSYGIS

SEQ ID NO: 218 - protein, 9 aa, synthetic construct
YTFTRNYVH

SEQ ID NO: 219 - protein, 9 aa, synthetic construct
YTFTGYYMH

SEQ ID NO: 220 - protein, 9 aa, synthetic construct
YTFTAYYMH

SEQ ID NO: 221 - protein, 9 aa, synthetic construct
FTLGSYWMH

SEQ ID NO: 222 - protein, 9 aa, synthetic construct
YTFTSHDIN

SEQ ID NO: 223 - protein, 9 aa, synthetic construct
GTFSNYDIN

SEQ ID NO: 224 - protein, 9 aa, synthetic construct
FTVGSWYMS

SEQ ID NO: 225 - protein, 9 aa, synthetic construct
YTFTSYNIH

SEQ ID NO: 226 - protein, 9 aa, synthetic construct
GTFSSYAIS

SEQ ID NO: 227 - protein, 9 aa, synthetic construct
YTFTNFDIN

SEQ ID NO: 228 - protein, 9 aa, synthetic construct
GTFRSYAIS

SEQ ID NO: 229 - protein, 9 aa, synthetic construct
YTFTSYYMY

SEQ ID NO: 230 - protein, 9 aa, synthetic construct
YTFSNYYMH

SEQ ID NO: 231 - protein, 9 aa, synthetic construct
YTFTSYYIH

SEQ ID NO: 232 - protein, 9 aa, synthetic construct
YTFTDNYLH

SEQ ID NO: 233 - protein, 9 aa, synthetic construct
YTLNNYDIN

SEQ ID NO: 234 - protein, 9 aa, synthetic construct
GTFSSYDIN

SEQ ID NO: 235 - protein, 9 aa, synthetic construct
GTFSNYGFS

SEQ ID NO: 236 - protein, 9 aa, synthetic construct
GTFSNYAIS

SEQ ID NO: 237 - protein, 9 aa, synthetic construct
YTFTSYHMH

SEQ ID NO: 238 - protein, 9 aa, synthetic construct
GTFTSYDIN

SEQ ID NO: 239 - protein, 9 aa, synthetic construct
GTFSKIAVS

SEQ ID NO: 240 - protein, 9 aa, synthetic construct
YTFTSYGIS

SEQ ID NO: 241 - protein, 9 aa, synthetic construct
YTFSSYAIS

SEQ ID NO: 242 - protein, 9 aa, synthetic construct
YTFTTYGIS

SEQ ID NO: 243 - protein, 9 aa, synthetic construct
YTFTRYDIN

SEQ ID NO: 244 - protein, 9 aa, synthetic construct
HTFNNNYIH

SEQ ID NO: 245 - protein, 8 aa, synthetic construct
IWYDGSNK

SEQ ID NO: 246 - protein, 8 aa, synthetic construct
INPNSGGT

SEQ ID NO: 247 - protein, 13 aa, synthetic construct
GWMSPNSGNTGYA

SEQ ID NO: 248 - protein, 13 aa, synthetic construct
GMIDPSGGSTSYA

SEQ ID NO: 249 - protein, 13 aa, synthetic construct
GWMNPNSGNTGYA

SEQ ID NO: 250 - protein, 13 aa, synthetic construct
GWISAYNGNTNYA

SEQ ID NO: 251 - protein, 13 aa, synthetic construct
SYISSSGNTIYYA

SEQ ID NO: 252 - protein, 13 aa, synthetic construct
GWVDPNNGDTGYA

SEQ ID NO: 253 - protein, 13 aa, synthetic construct
GWISPYNGNTNYA

SEQ ID NO: 254 - protein, 13 aa, synthetic construct
GWINPNSGNAGFA

SEQ ID NO: 255 - protein, 13 aa, synthetic construct
GRINPNSGGTNTA

SEQ ID NO: 256 - protein, 13 aa, synthetic construct
GIINPSGGSTSYA

SEQ ID NO: 257 - protein, 13 aa, synthetic construct
SLISWDGGSTYYA

SEQ ID NO: 258 - protein, 13 aa, synthetic construct
GISNPSGGSTYYA

SEQ ID NO: 259 - protein, 13 aa, synthetic construct
GIINPSGGSTNYA

SEQ ID NO: 260 - protein, 13 aa, synthetic construct
AHVSDDGSNEFYA

SEQ ID NO: 261 - protein, 13 aa, synthetic construct
GRINPNSGGTKYA

SEQ ID NO: 262 - protein, 13 aa, synthetic construct
GWMNANNGNAGYA

SEQ ID NO: 263 - protein, 13 aa, synthetic construct
GIINPSSSSTGYA

SEQ ID NO: 264 - protein, 13 aa, synthetic construct
GWISAYNGHTNYA

SEQ ID NO: 265 - protein, 13 aa, synthetic construct
GWINPNSGNTGYA

SEQ ID NO: 266 - protein, 13 aa, synthetic construct
GWMNPNSDNTGYA

SEQ ID NO: 267 - protein, 13 aa, synthetic construct
GTINPSGGSTSYA

SEQ ID NO: 268 - protein, 13 aa, synthetic construct
GIIETSGGSTDYA

SEQ ID NO: 269 - protein, 13 aa, synthetic construct
GWISGYNGNTNYA

SEQ ID NO: 270 - protein, 13 aa, synthetic construct
GWMSPSSGNTGYA

SEQ ID NO: 271 - protein, 13 aa, synthetic construct
GRIHPRDGSTDYA

SEQ ID NO: 272 - protein, 14 aa, synthetic construct
GGIIPIFGSTASYA

SEQ ID NO: 273 - protein, 13 aa, synthetic construct
GLINPNSGGTDYA

SEQ ID NO: 274 - protein, 13 aa, synthetic construct
GWMDPNSGNTGYA

SEQ ID NO: 275 - protein, 13 aa, synthetic construct
GWMDPNSGSTGYA

SEQ ID NO: 276 - protein, 13 aa, synthetic construct
GVINPSGGSTSYA

SEQ ID NO: 277 - protein, 9 aa, synthetic construct
AREEQWLDY

SEQ ID NO: 278 - protein, 19 aa, synthetic construct
ARERVGYCSRPSCLDDLNI

SEQ ID NO: 279 - protein, 18 aa, synthetic construct
CARQREEYGHYYYGMDVW

SEQ ID NO: 280 - protein, 15 aa, synthetic construct [sequence omitted]

SEQ ID NO: 281 - protein, 18 aa, synthetic construct
CAKGAVAGTYYYYGMDVW

SEQ ID NO: 282 - protein, 17 aa, synthetic construct
CARDGIDYGGNSGYDYW

SEQ ID NO: 283 - protein, 18 aa, synthetic construct
CARSNWNYPLDYYYMDVW

SEQ ID NO: 284 - protein, 12 aa, synthetic construct
CARDNWGSLDVW

SEQ ID NO: 285 - protein, 16 aa, synthetic construct
CAREAYGDYGDYFDYW

SEQ ID NO: 286 - protein, 15 aa, synthetic construct
CARGPVYSYGPFDYW

SEQ ID NO: 287 - protein, 16 aa, synthetic construct
CARLGDVAVAASFDYW

SEQ ID NO: 288 - protein, 15 aa, synthetic construct
CARGSGYDAGVFDYW

SEQ ID NO: 289 - protein, 19 aa, synthetic construct
CARDSGWYAPGYYYGMDVW

SEQ ID NO: 290 - protein, 21 aa, synthetic construct
CARESYAYCSSTSCYDAFDIW

SEQ ID NO: 291 - protein, 24 aa, synthetic construct
CAKGPWVGYSGYDLNYYYYGMDVW

SEQ ID NO: 292 - protein, 15 aa, synthetic construct
CASSRRYWNGAFDYW

SEQ ID NO: 293 - protein, 14 aa, synthetic construct
CAAEAYGDYSLDPW

SEQ ID NO: 294 - protein, 13 aa, synthetic construct
CARGPIGYGMDVW

SEQ ID NO: 295 - protein, 22 aa, synthetic construct
CARSPGYCSSTSCYRYYGMDVW

SEQ ID NO: 296 - protein, 12 aa, synthetic construct
CARLGSAYFDLW

SEQ ID NO: 297 - protein, 23 aa, synthetic construct
CAKDLSPSSSWSTYYYYYGMDVW

SEQ ID NO: 298 - protein, 15 aa, synthetic construct
CALDGGQLGVAFDIW

SEQ ID NO: 299 - protein, 23 aa, synthetic construct
CARAGYYDSSGYYYSPYYGMDVW

SEQ ID NO: 300 - protein, 13 aa, synthetic construct
CAQEAAAGYFDYW

SEQ ID NO: 301 - protein, 14 aa, synthetic construct
CARESVLKGGMDVW

SEQ ID NO: 302 - protein, 22 aa, synthetic construct
CAKDRRRGVGLAAAGTSAFDIW

SEQ ID NO: 303 - protein, 18 aa, synthetic construct
CARVGVGYGYYYYGMDVW

SEQ ID NO: 304 - protein, 15 aa, synthetic construct
CARASMYSSSGFDYW

SEQ ID NO: 305 - protein, 19 aa, synthetic construct
CARETSRWLRDYYYGMDVW

SEQ ID NO: 306 - protein, 17 aa, synthetic construct
CARSFGGYSYDDAFDIW

SEQ ID NO: 307 - protein, 14 aa, synthetic construct
CARVGSSWEENDQW

SEQ ID NO: 308 - protein, 21 aa, synthetic construct
CARDLTLYCSSTSCLDAFDYW

SEQ ID NO: 309 - protein, 13 aa, synthetic construct [sequence omitted]

SEQ ID NO: 310 - protein, 17 aa, synthetic construct
CARVSLRFLEDYGMDVW

SEQ ID NO: 311 - protein, 13 aa, synthetic construct
CARGVGDDTLTDW

SEQ ID NO: 312 - protein, 19 aa, synthetic construct
CARGGRVVRGVIKGWFDPW

SEQ ID NO: 313 - protein, 13 aa, synthetic construct
CAREDSSWYFDLW

SEQ ID NO: 314 - protein, 18 aa, synthetic construct
CAKDFRGGDYYYYGMDVW

SEQ ID NO: 315 - protein, 22 aa, synthetic construct
CARGTYYDYIWGSYRYSWFDYW

SEQ ID NO: 316 - protein, 23 aa, synthetic construct
CARESSYYYDSSGYYPGWYFDLW

SEQ ID NO: 317 - protein, 17 aa, synthetic construct
CARGRGNSYYYYYMDVW

SEQ ID NO: 318 - protein, 15 aa, synthetic construct
CARDQMYSSYGMDVW

SEQ ID NO: 319 - protein, 14 aa, synthetic construct
CARESRGYYGMDVW

SEQ ID NO: 320 - protein, 11 aa, synthetic construct
CVKGNGYYDLW

SEQ ID NO: 321 - protein, 21 aa, synthetic construct
CARERVGYCSSTSCLHPLDYW

SEQ ID NO: 322 - protein, 14 aa, synthetic construct
CARSFDWLLLLDYW

SEQ ID NO: 323 - protein, 18 aa, synthetic construct
CARQIQDYGYYYYGMDVW

SEQ ID NO: 324 - protein, 116 aa, synthetic construct
QVQLVESGGGVVQPGRSLRLSCAASGFTFSNYGMHWVRQAPGKGLEWVAFIWYDGSNKNYADSVKGRFTISRDNPTNTLYLQMNSLRAEDTAVYYCAREEQWLDYWGQGTLVTVSS

SEQ ID NO: 325 - protein, 126 aa, synthetic construct
QVQLVQSGAEVKKPGASVKVSCKASGYTFTGYHIHWVRQAPGQGLEWMGWINPNSGGTNYAQKFQGRVTMTRDTSINTAYMELNRLRSDDTAVFYCARERVGYCSRPSCLDDLNIWGQGTMVTVSS

SEQ ID NO: 326 - protein, 123 aa, synthetic construct
QVQLVQSGAEVKKPGASVKVSCKASGYSFSDYYMHWVRQAPGQGLEWMGWMSPNSGNTGYAQKFQGRVTMTRDTSTSTVYMELSSLRSEDTAVYYCARQREEYGHYYYGMDVWGQGTMVTVSS

SEQ ID NO: 327 - protein, 120 aa, synthetic construct
QVQLVQSGAEVKKPGASVKVSCKASGGTFSNYDISWVRQAPGQGLEWMGMIDPSGGSTSYAQKFQGRVTMTRDTSTSTVYMELSSLRSEDTAVYYCARPYSSGWYYFDYWGQGTLVTVSS

SEQ ID NO: 328 - protein, 123 aa, synthetic construct
QVQLVQSGAEVKKPGASVKVSCKASGYTFTSYDINWVRQAPGQGLEWMGWMNPNSGNTGYAQKFQGRVTMTRDTSTSTVYMELSSLRSEDTAVYYCAKGAVAGTYYYYGMDVWGQGTTVTVSS

SEQ ID NO: 329 - protein, 122 aa, synthetic construct
QVQLVQSGAEVKKPGASVKVSCKASGGTFGSYGINWVRQAPGQGLEWMGWISAYNGNTNYAQKLQGRVTMTRDTSTSTVYMELSSLRSEDTAVYYCARDGIDYGGNSGYDYWGQGTLVTVSS

SEQ ID NO: 330 - protein, 123 aa, synthetic construct
EVQLLESGGGLVQPGGSLRLSCAASGFTFSNSDMNWVRQAPGKGLEWVSYISSSGNTIYYADSVKGRFTISRDNSKNTLYLQMNSLRAEDTAVYYCARSNWNYPLDYYYMDVWGKGTTVTVSS

SEQ ID NO: 331 - protein, 117 aa, synthetic construct
QVQLVQSGAEVKKPGASVKVSCKASGYTFTSYYMHWVRQAPGQGLEWMGWVDPNNGDTGYAQKFQGRVTMTRDTSTSTVYMELSSLRSEDTAVYYCARDNWGSLDVWGKGTTVTVSS

SEQ ID NO: 332 - protein, 121 aa, synthetic construct
QVQLVQSGAEVKKPGASVKVSCKASGYSFTSYGISWVRQAPGQGLEWMGWISPYNGNTNYAQKLQGRVTMTRDTSTSTVYMELSSLRSEDTAVYYCAREAYGDYGDYFDYWGQGTLVTVSS

SEQ ID NO: 333 - protein, 120 aa, synthetic construct
QVQLVQSGAEVKKPGASVKVSCKASGYTFTSYYMHWVRQAPGQGLEWMGWINPNSGNAGFAQKFQGRVTMTRDTSTSTVYMELSSLRSEDTAVYYCARGPVYSYGPFDYWGQGTLVTVSS

SEQ ID NO: 334 - protein, 121 aa, synthetic construct
QVQLVQSGAEVKKPGASVKVSCKASGYTFTRNYVHWVRQAPGQGLEWMGRINPNSGGTNTAQKFQGRVTMTRDTSTSTVYMELSSLRSEDTAVYYCARLGDVAVAASFDYWGQGTLVTVSS

SEQ ID NO: 335 - protein, 120 aa, synthetic construct
QVQLVQSGAEVKKPGASVKVSCKASGYTFTGYYMHWVRQAPGQGLEWMGWMNPNSGNTGYAQKFQGRVTMTRDTSTSTVYMELSSLRSEDTAVYYCARGSGYDAGVFDYWGQGTLVTVSS

SEQ ID NO: 336 - protein, 124 aa, synthetic construct
QVQLVQSGAEVKKPGASVKVSCKASGYTFTAYYMHWVRQAPGQGLEWMGIINPSGGSTSYAQKFQGRVTMTRDTSTSTVYMELSSLRSEDTAVYYCARDSGWYAPGYYYGMDVWGQGTTVTVSS

SEQ ID NO: 337 - protein, 126 aa, synthetic construct
QVQLVQSGAEVKKPGASVKVSCKASGYTFTSYDINWVRQAPGQGLEWMGWMNPNSGNTGYAQKFQGRVTMTRDTSTSTVYMELSSLRSEDTAVYYCARESYAYCSSTSCYDAFDIWGQGTMVTVSS

SEQ ID NO: 338 - protein, 129 aa, synthetic construct
EVQLLESGGGLVQPGGSLRLSCAASGFTLGSYWMHWVRQAPGKGLEWVSLISWDGGSTYYADSVKGRFTISRDNSKNTLYLQMNSLRAEDTAVYYCAKGPWVGYSGYDLNYYYYGMDVWGQGTTVTVSS

SEQ ID NO: 339 - protein, 120 aa, synthetic construct
QVQLVQSGAEVKKPGASVKVSCKASGYTFTSHDINWVRQAPGQGLEWMGISNPSGGSTYYAQKFQGRVTMTRDTSTSTVYMELSSLRSEDTAVYYCASSRRYWNGAFDYWGQGTLVTVSS

SEQ ID NO: 340 - protein, 119 aa, synthetic construct
QVQLVQSGAEVKKPGASVKVSCKASGGTFSNYDINWVRQAPGQGLEWMGWISAYNGNTNYAQKLQGRVTMTRDTSTSTVYMELSSLRSEDTAVYYCAAEAYGDYSLDPWGQGTLVTVSS

SEQ ID NO: 341 - protein, 118 aa, synthetic construct
QVQLVQSGAEVKKPGASVKVSCKASGYTFTGYYMHWVRQAPGQGLEWMGIINPSGGSTNYAQKFQGRVTMTRDTSTSTVYMELSSLRSEDTAVYYCARGPIGYGMDVWGQGTLVTVSS

SEQ ID NO: 342 - protein, 127 aa, synthetic construct
EVQLLESGGGLVQPGGSLRLSCAASGFTVGSWYMSWVRQAPGKGLEWVAHVSDDGSNEFYADSVKGRFTISRDNSKNTLYLQMNSLRAEDTAVYYCARSPGYCSSTSCYRYYGMDVWGQGTTVTVSS

SEQ ID NO: 343 - protein, 117 aa, synthetic construct
QVQLVQSGAEVKKPGASVKVSCKASGYTFTSYYMHWVRQAPGQGLEWMGRINPNSGGTKYAQKFQGRVTMTRDTSTSTVYMELSSLRSEDTAVYYCARLGSAYFDLWGRGTLVTVSS

SEQ ID NO: 344 - protein, 128 aa, synthetic construct
QVQLVQSGAEVKKPGASVKVSCKASGYTFTSHDINWVRQAPGQGLEWMGWMNANNGNAGYAQKFQGRVTMTRDTSTSTVYMELSSLRSEDTAVYYCAKDLSPSSSWSTYYYYYGMDVWGQGTMVTVSS

SEQ ID NO: 345 - protein, 120 aa, synthetic construct
QVQLVQSGAEVKKPGASVKVSCKASGYTFTSYNIHWVRQAPGQGLEWMGWISAYNGNTNYAQKLQGRVTMTRDTSTSTVYMELSSLRSEDTAVYYCALDGGQLGVAFDIWGQGTMVTVSS

SEQ ID NO: 346 - protein, 128 aa, synthetic construct
QVQLVQSGAEVKKPGASVKVSCKASGGTFSSYAISWVRQAPGQGLEWMGIINPSSSSTGYAQKFQGRVTMTRDTSTSTVYMELSSLRSEDTAVYYCARAGYYDSSGYYYSPYYGMDVWGQGTTVTVSS

SEQ ID NO: 347 - protein, 118 aa, synthetic construct
QVQLVQSGAEVKKPGASVKVSCKASGYTFTNFDINWVRQAPGQGLEWMGWISAYNGHTNYAQNLQGRVTMTRDTSTSTVYMELSSLRSEDTAVYYCAQEAAAGYFDYWGQGTLVTVSS

SEQ ID NO: 348 - protein, 119 aa, synthetic construct
QVQLVQSGAEVKKPGASVKVSCKASGGTFRSYAISWVRQAPGQGLEWMGWMNPNSGNTGYAQKFQGRVTMTRDTSTSTVYMELSSLRSEDTAVYYCARESVLKGGMDVWGQGTTVTVSS

SEQ ID NO: 349 - protein, 127 aa, synthetic construct
QVQLVQSGAEVKKPGASVKVSCKASGYTFTSYYMYWVRQAPGQGLEWMGWINPNSGNTGYAQKFQGRVTMTRDTSTSTVYMELSSLRSEDTAVYYCAKDRRRGVGLAAAGTSAFDIWGQGTMVTVSS

SEQ ID NO: 350 - protein, 123 aa, synthetic construct
QVQLVQSGAEVKKPGASVKVSCKASGYTFTSYYMHWVRQAPGQGLEWMGWMNPNSDNTGYAQKFQGRVTMTRDTSTSTVYMELSSLRSEDTAVYYCARVGVGYGYYYYGMDVWGQGTTVTVSS

SEQ ID NO: 351 - protein, 120 aa, synthetic construct
QVQLVQSGAEVKKPGASVKVSCKASGYTFSNYYMHWVRQAPGQGLEWMGWMNPNSGNTGYAQKFQGRVTMTRDTSTSTVYMELSSLRSEDTAVYYCARASMYSSSGFDYWGQGTLVTVSS

SEQ ID NO: 352 - protein, 124 aa, synthetic construct
QVQLVQSGAEVKKPGASVKVSCKASGYTFTSYYIHWVRQAPGQGLEWMGTINPSGGSTSYAQKFQGRVTMTRDTSTSTVYMELSSLRSEDTAVYYCARETSRWLRDYYYGMDVWGQGTTVTVSS

SEQ ID NO: 353 - protein, 122 aa, synthetic construct
QVQLVQSGAEVKKPGASVKVSCKASGYTFTDNYLHWVRQAPGQGLEWMGIIETSGGSTDYAQKFQGRVTMTRDTSTSTVYMELSSLRSEDTAVYYCARSFGGYSYDDAFDIWGQGTTVTVSS

SEQ ID NO: 354 - protein, 119 aa, synthetic construct
QVQLVQSGAEVKKPGASVKVSCKASGYTLNNYDINWVRQAPGQGLEWMGWISGYNGNTNYAQKLQGRVTMTRDTSTSTVYMELSSLRSEDTAVYYCARVGSSWEENDQWGQGTLVTVSS

SEQ ID NO: 355 - protein, 126 aa, synthetic construct
QVQLVQSGAEVKKPGASVKVSCKASGGTFSSYDINWVRQAPGQGLEWMGWMNPNSGNTGYAQKFQGRVTMTRDTSTSTVYMELSSLRSEDTAVYYCARDLTLYCSSTSCLDAFDYWGQGTLVTVSS

SEQ ID NO: 356 - protein, 118 aa, synthetic construct
QVQLVQSGAEVKKPGASVKVSCKASGGTFSNYGFSWVRQAPGQGLEWMGWMSPSSGNTGYAQKFQGRVTMTRDTSTSTVYMELSSLRSEDTAVYYCARERSGSYFDYWGQGTLVTVSS

SEQ ID NO: 357 - protein, 122 aa, synthetic construct
QVQLVQSGAEVKKPGSSVKVSCKASGGTFSSYAISWVRQAPGQGLEWVGRIHPRDGSTDYAQKFQGRVTITADESTSTAYMELSSLRSEDTAVYYCARVSLRFLEDYGMDVWGQGTTVTVSS

SEQ ID NO: 358 - protein, 119 aa, synthetic construct
QVQLVQSGAEVKKPGSSVKVSCKASGGTFSNYAISWVRQAPGQGLEWMGGIIPIFGSTASYAQKFQGRVTITADESTSTAYMELSSLRSEDTAVYYCARGVGDDTLTDWGQGTLVTVSS

SEQ ID NO: 359 - protein, 124 aa, synthetic construct
QVQLVQSGAEVKKPGASVKVSCKASGYTFTSYHMHWVRQAPGQGLEWMGWMNPNSGNTGYAQKFQGRVTMTRDTSTSTVYMELSSLRSEDTAVYYCARGGRVVRGVIKGWFDPWGQGTLVTVSS

SEQ ID NO: 360 - protein, 118 aa, synthetic construct
QVQLVQSGAEVKKPGASVKVSCKASGGTFTSYDINWVRQAPGQGLEWMGWISAYNGHTNYAQNLQGRVTMTRDTSTSTVYMELSSLRSEDTAVYYCAREDSSWYFDLWGRGTLVTVSS

SEQ ID NO: 361 - protein, 123 aa, synthetic construct
QVQLVQSGAEVKKPGSSVKVSCKASGGTFSKIAVSWVRQAPGQGLEWMGLINPNSGGTDYAQKFQGRVTITADESTSTAYMELSSLRSEDTAVYYCAKDFRGGDYYYYGMDVWGQGTTVTVSS

SEQ ID NO: 362 - protein, 127 aa, synthetic construct
QVQLVQSGAEVKKPGASVKVSCKASGYTFTSYGISWVRQAPGQGLEWMGWISAYNGNTNYAQKFQGRVTMTRDTSTSTVYMELSSLRSEDTAVYYCARGTYYDYIWGSYRYSWFDYWGQGTLVTVSS

SEQ ID NO: 363 - protein, 128 aa, synthetic construct
QVQLVQSGAEVKKPGASVKVSCKASGYTFSSYAISWVRQAPGQGLEWMGWMDPNSGNTGYAQKFQGRVTMTRDTSTSTVYMELSSLRSEDTAVYYCARESSYYYDSSGYYPGWYFDLWGRGTLVTVSS

SEQ ID NO: 364 - protein, 122 aa, synthetic construct
QVQLVQSGAEVKKPGASVKVSCKASGYTFTTYGISWVRQAPGQGLEWMGWMDPNSGSTGYAQKFQGRVTMTRDTSTSTVYMELSSLRSEDTAVYYCARGRGNSYYYYYMDVWGKGTTVTVSS

SEQ ID NO: 365 - protein, 120 aa, synthetic construct
QVQLVQSGAEVKKPGASVKVSCKASGYTFTSYYMHWVRQAPGQGLEWMGWMNPNSGNTGYAQKFQGRVTMTRDTSTSTVYMELSSLRSEDTAVYYCARDQMYSSYGMDVWGQGTTVTVSS

SEQ ID NO: 366 - protein, 119 aa, synthetic construct
QVQLVQSGAEVKKPGSSVKVSCKASGGTFSSYAISWVRQAPGQGLEWMGVINPSGGSTSYAQKFQGRVTITADESTSTAYMELSSLRSEDTAVYYCARESRGYYGMDVWGQGTTVTVSS

SEQ ID NO: 367 - protein, 116 aa, synthetic construct
QVQLVQSGAEVKKPGASVKVSCKASGYTFTGYYMHWVRQAPGQGLEWMGTINPSGGSTSYAQKFQGRVTMTRDTSTSTVYMELSSLRSEDTAVYYCVKGNGYYDLWGQGTLVTVSS

SEQ ID NO: 368 - protein, 126 aa, synthetic construct
QVQLVQSGAEVKKPGASVKVSCKASGYTFTRYDINWVRQAPGQGLEWMGWMNPNSGNTGYAQKLQGRVTMTRDTSTSTVYMELSSLRSEDTAVYYCARERVGYCSSTSCLHPLDYWGQGTLVTVSS

SEQ ID NO: 369 - protein, 119 aa, synthetic construct
QVQLVQSGAEVKKPGASVKVSCKASGYTFTSYDINWVRQAPGQGLEWMGWISGYNGNTNYAQKFQGRVTMTRDTSTSTVYMELSSLRSEDTAVYYCARSFDWLLLLDYWGQGTLVTVSS

SEQ ID NO: 370 - protein, 123 aa, synthetic construct
QVQLVQSGAEVKKPGASVKVSCKASGHTFNNNYIHWVRQAPGQGLEWMGIINPSGGSTSYAQKFQGRVTMTRDTSTSTVYMELSSLRSEDTAVYYCARQIQDYGYYYYGMDVWGQGTTVTVSS

SEQ ID NO: 371 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPLTFGGGTKVEIK

SEQ ID NO: 372 - protein, 106 aa, synthetic construct
DIQMTQSPSTLSASVGDRVTITCRASQSISSWLAWYQQKPGKAPKLLIYKASSLETGVPSRFSGSGSGTEFTLTISSLQPDDFATYYCQQYDSYYTFGQGTKLEIK

SEQ ID NO: 373 - protein, 108 aa, synthetic construct
EIVMTQSPATLSVSPGERATLSCRASQSLGSTYLAWYQQKPGQAPRLLIYGASTRATGIPARFSGSGSGTEFTLTISSLQSEDFAVYYCQQYYSSPPTFGQGTKVEIK

SEQ ID NO: 374 - protein, 107 aa, synthetic construct
DIQMTQSPSSLSASVGDRVTITCQASQDIHNYLNWYQQKPGKAPKLLIYDASNLETGVPSRFSGSGSGTDFTLTISSLQPEDFATYYCQQSYNTPPTFGQGTRLEIK

SEQ ID NO: 375 - protein, 107 aa, synthetic construct
DIQMTQSPSSLSASVGDRVTITCRASQTINNYLNWYQQKPGKAPKLLIYDASNLETGVPSRFSGSGSGTDFTLTISSLQPEDFATYYCQQANSFPLTFGQGTKVEIK

SEQ ID NO: 376 - protein, 107 aa, synthetic construct
DIQMTQSPSSLSASVGDRVTITCRASQNIRNYLNWYQQKPGKAPKLLIYAASSLQGGVPSRFSGSGSGTDFTLTISSLQPEDFATYYCQQSYNGPYTFGQGTKVEIK

SEQ ID NO: 377 - protein, 112 aa, synthetic construct
DIVMTQSPLSLPVTPGEPASISCRSSQSLLHSNGYNYLDWYLQKPGQSPQLLIYAASSLQSGVPDRFSGSGSGTDFTLKISRVEAEDVGVYYCMQGTHWPPTFGQGTKVEIK

SEQ ID NO: 378 - protein, 112 aa, synthetic construct
DIVMTQSPLSLPVTPGEPASISCRSSQSLLHSNGYNYLDWYLQKPGQSPQLLIYAASTLQSGVPDRFSGSGSGTDFTLKISRVEAEDVGVYYCMQGLQTLYSFGQGTKLEIK

SEQ ID NO: 379 - protein, 107 aa, synthetic construct
DIQMTQSPSSLSASVGDRVTITCRASQGVGDRSAWYQQKPGKAPKLLIYDASNLETGVPSRFSGSGSGTDFTLTISSLQPEDFATYYCQQADTFPYTFGQGTKLEIK

SEQ ID NO: 380 - protein, 107 aa, synthetic construct
DIQMTQSPSSLSASVGDRVTITCRASQGISTYLAWYQQKPGKAPKLLIYAASTLQSGVPSRFSGSGSGTDFTLTISSLQPEDFATYYCQQSHSAPITFGQGTKVEIK

SEQ ID NO: 381 - protein, 107 aa, synthetic construct
DIQMTQSPSSLSASVGDRVTITCRASQSISSYLNWYQQKPGKAPKLLIYDASSLESGVPSRFSGSGSGTDFTLTISSLQPEDFATYYCQQANSFPLTFGPGTKVDIK

SEQ ID NO: 382 - protein, 107 aa, synthetic construct
DIQMTQSPSSLSASVGDRVTITCRASQGIGNDLAWYQQKPGKAPKLLIYDAKDLHPGVPSRFSGSGSGTDFTLTISSLQPEDFATYYCQQSYSFPRTFGQGTRLEIK

SEQ ID NO: 383 - protein, 107 aa, synthetic construct
DIQMTQSPSSLSASVGDRVTITCRASESISHWLAWYQQKPGKAPKLLIYGASTLQSGVPSRFSGSGSGTDFTLTISSLQPEDFATYYCQQSYTPPLTFGGGTKVEIK

SEQ ID NO: 384 - protein, 113 aa, synthetic construct
DIVMTQSPDSLAVSLGERATINCKSSQSLLYSSNNKNYLAWYQQKPGQPPKLLIYWASIRESGVPDRFSGSGSGTDFTLTISSLQAEDVAVYYCQQYLSPPLTFGQGTKVEIK

SEQ ID NO: 385 - protein, 107 aa, synthetic construct
DIQMTQSPSSLSASVGDRVTITCRASQDIRRALAWYQQKPGKAPKLLIYAASSLQSGVPSRFSGSGSGTDFTLTISSLQPEDFATYYCLQHNSYPPTFGQGTKLEIK

SEQ ID NO: 386 - protein, 111 aa, synthetic construct
DIVMTQSPLSLPVTPGEPASISCRSSQSLLHSNGYNYLDWYLQKPGQSPQLLIYLGSSRASGVPDRFSGSGSGTDFTLKISRVEAEDVGVYYCMQALQTPTFGQGTRLEIK

SEQ ID NO: 387 - protein, 107 aa, synthetic construct
DIQMTQSPSSLSASVGDRVTITCQASQGISSYLNWYQQKPGKAPKLLIYAASSLQNGVPSRFSGSGSGTDFTLTISSLQPEDFATYYCQQSYSTPITFGQGTRLEIK

SEQ ID NO: 388 - protein, 107 aa, synthetic construct
DIQMTQSPSSLSASVGDRVTITCRASQGIGNDLGWYQQKPGKAPKLLIYAASNLETGVPSRFSGSGSGTDFTLTISSLQPEDFATYYCLQHHSYPFTFGQGTKLEIK

SEQ ID NO: 389 - protein, 105 aa, synthetic construct
DIQMTQSPSSLSASVGDRVTITCRTSQSIGTYLNWYQQKPGKAPKLLIYDASNLPTGVPSRFSGSGSGTDFTLTISSLQPEDFATYYCQNYNSALFGPGTKVDIK

SEQ ID NO: 390 - protein, 107 aa, synthetic construct
DIQMTQSPSSLSASVGDRVTITCRASQSVDTWLAWYQQKPGKAPKLLIYEASTLESGVPSRFSGSGSGTDFTLTISSLQPEDFATYYCQQANSFPITFGQGTRLEIK

SEQ ID NO: 391 - protein, 107 aa, synthetic construct
DIQMTQSPSSLSASVGDRVTITCRASQSISNLLAWYQQKPGKAPKLLIYAASSLQSGVPSRFSGSGSGTDFTLTISSLQPEDFATYYCQQSYSTPYTFGQGTKLEIK

SEQ ID NO: 392 - protein, 107 aa, synthetic construct
DIQMTQSPSSLSASVGDRVTITCRASQGISNWLAWYQQKPGKAPKLLIYDASNLDAGVPSRFSGSGSGTDFTLTISSLQPEDFATYYCQQANSLPLTFGGGTKVEIK

SEQ ID NO: 393 - protein, 108 aa, synthetic construct
EIVMTQSPATLSVSPGERATLSCRASQSISGSYLAWYQQKPGQAPRLLIYGASTRATGIPARFSGSGSGTEFTLTISSLQSEDFAVYYCQQYGSFPFTFGPGTKVDIK

SEQ ID NO: 394 - protein, 107 aa, synthetic construct
DIQMTQSPSSLSASVGDRVTITCRASQDIRNDLAWYQQKPGKAPKLLIYDASNLETGVPSRFSGSGSGTDFTLTISSLQPEDFATYYCQQSYGTPYTFGQGTKLEIK

SEQ ID NO: 395 - protein, 113 aa, synthetic construct
DIVMTQSPDSLAVSLGERATINCRSSQSLFHSSTNANNLAWYQQKPGQPPKLLIYWASTRESGVPDRFSGSGSGTDFTLTISSLQAEDVAVYYCQQYYITPLTFGQGTRLEIK

SEQ ID NO: 396 - protein, 107 aa, synthetic construct
DIQMTQSPSSLSASVGDRVTITCRASQSISNWLAWYQQKPGKAPKLLIYAASSLQSGVPSRFSGSGSGTDFTLTISSLQPEDFATYYCQQSYSTPYTFGQGTKLEIK

SEQ ID NO: 397 - protein, 107 aa, synthetic construct
DIQMTQSPSSLSASVGDRVTITCRAGQSIGDYLNWYQQKPGKAPKLLIYAASSLQSGVPSRFSGSGSGTDFTLTISSLQPEDFATYYCQQSYSTPWTFGQGTKVEIK

SEQ ID NO: 398 - protein, 112 aa, synthetic construct
DIVMTQSPLSLPVTPGEPASISCRSSQSLLHSNGYNYLDWYLQKPGQSPQLLIYLASTRAPGVPDRFSGSGSGTDFTLKISRVEAEDVGVYYCMQGSHWPPSFGQGTRLEIK

SEQ ID NO: 399 - protein, 112 aa, synthetic construct
DIVMTQSPLSLPVTPGEPASISCRSSQSLLHSNGYNYLDWYLQKPGQSPQLLIYDASSLESGVPDRFSGSGSGTDFTLKISRVEAEDVGVYYCMQGTHWPLTFGGGTKVEIK

SEQ ID NO: 400 - protein, 107 aa, synthetic construct
DIQMTQSPSSLSASVGDRVTITCQASQGISNYLNWYQQKPGKAPKLLIYAASSLQSGVPSRFSGSGSGTDFTLTISSLQPEDFATYYCQQSYSSPYTFGQGTKVEIK

SEQ ID NO: 401 - protein, 107 aa, synthetic construct
DIQMTQSPSSLSASVGDRVTITCRASQSISRWLAWYQQKPGKAPKLLIYAASSLESGVPSRFSGSGSGTDFTLTISSLQPEDFATYYCQQTHSFPLTFGGGTKVEIK

SEQ ID NO: 402 - protein, 113 aa, synthetic construct
DIVMTQSPDSLAVSLGERATINCRSSQSVLYSSNNKNYLAWYQQKPGQPPKLLIYWASTRESGVPDRFSGSGSGTDFTLTISSLQAEDVAVYYCQQYYSTPPTFGQGTKLEIK

SEQ ID NO: 403 - protein, 107 aa, synthetic construct
EIVMTQSPATLSVSPGERATLSCRASQSVSSYLAWYQQKPGQAPRLLIYGTSTRATGIPARFSGSGSGTEFTLTISSLQSEDFAVYYCQQYGSLPFTFGQGTRLEIK

SEQ ID NO: 404 - protein, 113 aa, synthetic construct
DIVMTQSPDSLAVSLGERATINCKSSQSVLYSSNNKNYLAWYQQKPGQPPKLLIYWASTRASGVPDRFSGSGSGTDFTLTISSLQAEDVAVYYCQQYYTTPPTFGQGTRLEIK

SEQ ID NO: 405 - protein, 112 aa, synthetic construct
DIVMTQSPLSLPVTPGEPASISCRSSQSLLHSNGYNYLDWYLQKPGQSPQLLIYAASSLQSGVPDRFSGSGSGTDFTLKISRVEAEDVGVYYCMQALQTPLTFGGGTKVEIK

SEQ ID NO: 406 - protein, 113 aa, synthetic construct
DIVMTQSPDSLAVSLGERATINCKSSQSLLYTSNDRNYLAWYQQKPGQPPKLLIYWASTRESGVPDRFSGSGSGTDFTLTISSLQAEDVAVYYCQQYYSSPYTFGQGTKVEIK

SEQ ID NO: 407 - protein, 107 aa, synthetic construct
DIQMTQSPSSLSASVGDRVTITCRASQSISSWLAWYQQKPGKAPKLLIYDASNLETGVPSRFSGSGSGTDFTLTISSLQPEDFATYYCQQSFSTPPTFGQGTKVEIK

SEQ ID NO: 408 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNTYPITFGQGTKVEIK

SEQ ID NO: 409 - protein, 107 aa, synthetic construct [sequence omitted]

SEQ ID NO: 410 - protein, 107 aa, synthetic construct
DIQMTQSPSSLSASVGDRVTITCQASQDISNYLNWYQQKPGKAPKLLIYAASTLQTGVPSRFSGSGSGTDFTLTISSLQPEDFATYYCQQSYSTPWTFGPGTKVDIK

SEQ ID NO: 411 - protein, 107 aa, synthetic construct
DIQMTQSPSSLSASVGDRVTITCRASQRIGSYLNWYQQKPGKAPKLLIYGASNLQSGVPSRFSGSGSGTDFTLTISSLQPEDFATYYCQQANSFPFTFGPGTKVDIK

SEQ ID NO: 412 - protein, 107 aa, synthetic construct
DIQMTQSPSSLSASVGDRVTITCRASESITNWLAWYQQKPGKAPKLLIYKASSLESGVPSRFSGSGSGTDFTLTISSLQPEDFATYYCQQSYGTPYTFGQGTKVEIK

SEQ ID NO: 413 - protein, 113 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPYTFGQGTKVEIK

SEQ ID NO: 414 - protein, 107 aa, synthetic construct
DIQMTQSPSSLSASVGDRVTITCRASQSISSWLAWYQQKPGKAPKLLIYDTSNLQGGVPSRFSGSGSGTDFTLTISSLQPEDFATYYCQQYDSFPWTFGQGTKVEIK

SEQ ID NO: 415 - protein, 107 aa, synthetic construct
DIQMTQSPSSLSASVGDRVTITCRASQGIRNDLGWYQQKPGKAPKLLIYSASNLQSGVPSRFSGSGSGTDFTLTISSLQPEDFATYYCQQSYDVPLTFGGGTKVEIK

SEQ ID NO: 416 - protein, 107 aa, synthetic construct
DIQMTQSPSSLSASVGDRVTITCRASQSISSWLAWYQQKPGKAPKLLIYDATTLESGVPSRFSGSGSGTDFTLTISSLQPEDFATYYCQQYDSYPWTFGQGTKLEIK

SEQ ID NO: 417 - protein, 107 aa, synthetic construct
EIVMTQSPATLSVSPGERATLSCRASQSVSKYLAWYQQKPGQAPRLLIYAISARAAGIPARFSGSGSGTEFTLTISSLQSEDFAVYYCQQYYSSPPTFGQGTKVEIK

SEQ ID NO: 418 - DNA, 348 bp, synthetic construct
  source 1..348: mol_type = other DNA
caggtgcagctggtggagtctgggggaggcgtggtccagcctgggaggtccctgagactctcctgtgcagcgtctggattcaccttcagtaactatggcatgcactgggtccgccaggctccaggcaaggggctggagtgggtggcatttatatggtatgatggaagtaataaaaactatgcagactccgtgaagggccgattcaccatctccagagacaatcccacgaacacgctgtatctgcaaatgaacagcctgagagccgaggacacggctgtgtattactgtgcgagagaggagcagtggcttgactactggggccagggaaccctggtcaccgtctcctca

SEQ ID NO: 419 - DNA, 378 bp, synthetic construct
  source 1..378: mol_type = other DNA
caggtgcagctggtgcagtctggggctgaggtgaagaagcctggggcctcagtgaaggtctcctgcaaggcttctggatacaccttcaccggctatcatatacactgggtgcgacaggcccctggacaagggcttgagtggatgggatggatcaaccctaacagtggtggcacaaactatgcacagaagtttcagggcagggtcaccatgaccagggacacgtccatcaacacggcctacatggagctgaacaggctgagatctgacgacacggccgtgttttactgtgcgagagagagagtaggatattgtagtcgtcccagctgccttgatgatcttaatatctggggccaggggacaatggtcaccgtctcttca

SEQ ID NO: 420 - DNA, 369 bp, synthetic construct
  source 1..369: mol_type = other DNA
caggtgcagctggtgcagtctggggctgaggtgaagaagcctggggcctcagtgaaggtttcctgcaaggcatctggatacagcttctctgactactatatgcactgggtgcgacaggcccctggacaagggcttgagtggatgggatggatgtcccctaatagtggtaacacaggctacgcacagaagttccagggcagagtcaccatgaccagggacacgtccacgagcacagtctacatggagctgagcagcctgagatctgaggacacggccgtgtattactgtgctagacaacgcgaggaatacggccactactactacgggatggatgtctggggccaagggacaatggtcaccgtctcttca

SEQ ID NO: 421 - DNA, 360 bp, synthetic construct
  source 1..360: mol_type = other DNA
caggtgcagctggtgcagtctggggctgaggtgaagaagcctggggcctcagtgaaggtttcctgcaaggcatctggagggaccttctctaactacgacatctcgtgggtgcgacaggcccctggacaagggcttgagtggatgggaatgatcgatcctagtggtggcagcacaagctacgcacagaagttccagggcagagtcaccatgaccagggacacgtccacgagcacagtctacatggagctgagcagcctgagatctgaggacacggccgtgtattactgtgctagaccttattcaagcgggtggtactactttgactactggggccaagggaccctggtcaccgtctcctca

SEQ ID NO: 422 - DNA, 369 bp, synthetic construct
  source 1..369: mol_type = other DNA
caggtgcagctggtgcagtctggggctgaggtgaagaagcctggggcctcagtgaaggtctcctgcaaggcttctggatacaccttcaccagttatgatatcaactgggtgcgacaggcccctggacaagggcttgagtggatgggatggatgaaccccaacagtggtaacacaggctatgcacagaagttccagggcagagtcaccatgaccagggacacctccacgagcacagtctacatggagctgagcagcctgagatctgaggacacggccgtgtattactgtgcgaaaggcgctgtggccggaacttactactattacggaatggacgtgtggggccaagggaccacggtcaccgtctcctca

SEQ ID NO: 423 - DNA, 366 bp, synthetic construct
  source 1..366: mol_type = other DNA
caggttcagctggtgcagtctggagctgaggtgaagaagcctggggcctcagtgaaggtctcctgcaaggcttctggtggcacctttggcagctatggtatcaactgggtgcgacaggcccctggacaagggcttgagtggatgggatggatcagcgcctacaatggtaacacaaactatgcacagaagctccagggcagagtcaccatgacccgggacacatccacgagcacagtctacatggagctgagcagcctgagatctgaagacacggccgtgtattactgtgcgagagatggaatcgattacggcgggaactctggatatgactactggggccaagggaccctggtcaccgtctcctca

SEQ ID NO: 424 - DNA, 369 bp, synthetic construct
  source 1..369: mol_type = other DNA
gaggtgcagctgctcgagtctgggggaggcttggtacagcctggagggtccctgagactctcctgtgcagcctctggattcaccttcagtaacagtgacatgaactgggtccgccaggctccagggaaggggctggagtgggtttcatacattagtagtagtggtaatactatatactacgcagactctgtgaagggccgattcaccatctccagagacaactcaaagaacacgctgtatctgcaaatgaacagcctgagagccgaggacacggctgtttattactgtgcgagaagcaactggaattaccctcttgactactactacatggacgtctggggcaaagggaccacggtcaccgtctcctca

SEQ ID NO: 425 - DNA, 351 bp, synthetic construct
  source 1..351: mol_type = other DNA
caggtgcagctggtgcagtctggggctgaggtgaagaagcctggggcctcagtgaaggtttcctgcaaggcatctggatacaccttcaccagctactatatgcactgggtgcgacaggcccctggacaagggcttgagtggatgggatgggtagatcccaataacggtgacacaggatacgcacagaagttccagggcagagtcaccatgaccagggacacgtccacgagcacagtctacatggagctgagcagcctgagatctgaggacacggccgtgtattactgtgctagagataactggggctccctggatgtctggggcaaagggacaaccgtcaccgtctcttca

SEQ ID NO: 426 - DNA, 363 bp, synthetic construct
  source 1..363: mol_type = other DNA
caggttcagctggtgcagtctggagctgaggtgaagaagcctggggcctcagtgaaggtctcctgcaaggcttctggttactcctttaccagctatggtatcagctgggtgcgacaggcccctggacaagggcttgagtggatgggatggatcagcccgtacaatggtaacacaaactatgcacagaagctccagggcagagtcaccatgaccagagacacatccacgagcacagtttacatggagctgtccagcctgagatctgaagacacggccgtgtattactgtgcgagagaggcttacggcgactacggcgactactttgactactggggccaagggaccctggtcaccgtctcctca

SEQ ID NO: 427 - DNA, 360 bp, synthetic construct
  source 1..360: mol_type = other DNA
caggtgcagctggtgcagtctggggctgaggtgaagaagcctggggcctcagtgaaggtttcctgcaaggcatctggatacaccttcaccagctactatatgcactgggtgcgacaggcccctggacaagggcttgagtggatgggatggatcaacccaaacagtggtaacgccgggtttgcacagaagttccagggcagagtcaccatgaccagggacacgtccacgagcacagtctacatggagctgagcagcctgagatctgaggacacggccgtgtattactgtgctagaggacccgtctattcctacggtccgtttgactactggggccaagggaccctggtcaccgtctcctca

SEQ ID NO: 428 - DNA, 363 bp, synthetic construct
  source 1..363: mol_type = other DNA
caggtgcagctggtgcagtctggggctgaggtgaagaagcctggggcctcagtgaaggtttcctgcaaggcatctggatacaccttcaccaggaattatgtccactgggtgcgacaggcccctggacaagggcttgagtggatgggacggatcaaccccaatagtggtgggacaaacaccgcacagaagttccagggcagagtcaccatgaccagggacacgtccacgagcacagtctacatggagctgagcagcctgagatctgaggacacggccgtgtattactgtgctagactgggcgacgtggccgtcgctgcgtcctttgactactggggccaagggaccctggtcaccgtctcctca

SEQ ID NO: 429 - DNA, 360 bp, synthetic construct
  source 1..360: mol_type = other DNA
caggtgcagctggtgcagtctggggctgaggtgaagaagcctggggcctcagtgaaggtttcctgcaaggcatctggatacaccttcaccggctactatatgcactgggtgcgacaggcccctggacaagggcttgagtggatgggatggatgaacccaaatagtggtaatacaggttacgcacagaagttccagggcagagtcaccatgaccagggacacgtccacgagcacagtctacatggagctgagcagcctgagatctgaggacacggccgtgtattactgtgctagaggatctggctacgatgctggggtgtttgactactggggccaagggaccctggtcaccgtctcctca

SEQ ID NO: 430 - DNA, 372 bp, synthetic construct
  source 1..372: mol_type = other DNA
caggtgcagctggtgcagtctggggctgaggtgaagaagcctggggcctcagtgaaggtttcctgcaaggcatctggatacaccttcaccgcatactatatgcactgggtgcgacaggcccctggacaagggcttgagtggatgggaataatcaaccctagtggtggcagcacaagctacgcacagaagttccagggcagagtcaccatgaccagggacacgtccacgagcacagtctacatggagctgagcagcctgagatctgaggacacggccgtgtattactgtgctagagatagcgggtggtacgcacccggatactattatgggatggatgtctggggccaagggacaacagtcaccgtctcttca

SEQ ID NO: 431 - DNA, 378 bp, synthetic construct
  source 1..378: mol_type = other DNA
caggtgcagctggtgcagtctggggctgaggtgaagaagcctggggcctcagtgaaggtctcctgcaaggcttctggatacaccttcaccagttatgatatcaactgggtgcgacaggccccaggacaagggcttgagtggatgggatggatgaaccccaacagtggtaacacaggctatgcacagaagttccagggcagagtcaccatgaccagggacacctccacaagcacagtttacatggagctgagcagcctgagatctgaggacacggccgtgtattactgtgcgagagagtcatacgcatattgctcaagtacatcctgttatgatgcttttgatatctggggccaagggacaatggtcaccgtctcttca

SEQ ID NO: 432 - DNA, 387 bp, synthetic construct
  source 1..387: mol_type = other DNA
gaggtgcagctgttggagtctgggggaggcttggtacagcctggggggtccctgagactctcctgtgcagcctctggattcaccttggggagctattggatgcattgggtccgccaggctccagggaaggggctggagtgggtctcattaattagttgggatggtggctctacatactacgcagactccgtgaagggccggttcaccatctccagagacaattccaagaacacgctgtatctgcaaatgaacagcctgagagccgaggacacggccgtatattactgtgcgaaaggcccatgggtcggttactcaggctacgatctgaattactactactacggcatggacgtttggggccaagggaccacagtcaccgtctcctca

SEQ ID NO: 433 - DNA, 360 bp, synthetic construct
  source 1..360: mol_type = other DNA
caggtgcagctggtgcagtctggggctgaggtgaagaagcctggggcctcagtgaaggtttcctgcaaggcatctggatacaccttcaccagccacgacattaattgggtgcgacaggcccctggacaagggcttgagtggatgggaatatcaaaccctagtggaggtagcacatactacgcacagaagttccagggcagagtcaccatgaccagggacacgtccacgagcacagtctacatggagctgagcagcctgagatctgaggacacggccgtgtattactgtgcttcatcccgacggtactggaatggcgcctttgactactggggccaagggaccctggtcaccgtctcctca

SEQ ID NO: 434 - DNA, 357 bp, synthetic construct
  source 1..357: mol_type = other DNA
caggttcagctggtgcagtctggagctgaggtgaagaagcctggggcctcagtgaaggtctcctgcaaggcttctggtgggacctttagtaactatgatatcaattgggtgcgacaggcccctggacaagggcttgagtggatgggatggatcagcgcgtacaatggtaacacaaactatgcacagaagctccagggcagagtcaccatgacccgggacacatccacgagcacagtgtacatggagctgagcagcctgagatctgaagacacggccgtgtattactgtgcggcagaagcctacggagattactcactggacccctggggccagggaaccctggtcaccgtctcctca

SEQ ID NO: 435 - DNA, 354 bp, synthetic construct
  source 1..354: mol_type = other DNA
caggtgcagctggtgcagtctggggctgaggtgaagaagcctggggcctcagtgaaggtttcctgcaaggcatctggatacaccttcaccggatactatatgcactgggtgcgacaggcccctggacaagggcttgagtggatgggaataatcaaccctagtggtggcagcacaaactacgcacagaagttccagggcagagtcaccatgaccagggacacgtccacgagcacagtctacatggagctgagcagcctgagatctgaggacacggccgtgtattactgtgctagaggcccgattggctacggtatggacgtgtggggccaagggaccctggtcaccgtctcctca

SEQ ID NO: 436 - DNA, 381 bp, synthetic construct
  source 1..381: mol_type = other DNA
gaggtgcagctgctggagtctgggggaggcttggtccagcctggggggtccctgagactctcctgtgcagcctctggattcaccgtcggcagctggtacatgagctgggtccgccaggctccagggaaggggctggagtgggtcgctcatgtctcggacgacggtagcaatgaattttacgcagactccgtgaagggccgattcaccatctccagagacaattccaagaacacgctgtatcttcaaatgaacagcctgagagctgaggacacggctgtgtattactgtgcgagaagccccggctattgtagctccaccagctgttaccgatactatggtatggacgtctggggccaagggaccacagtcaccgtctcctca

SEQ ID NO: 437 - DNA, 351 bp, synthetic construct
  source 1..351: mol_type = other DNA
caggtgcagctggtgcagtctggggctgaggtgaagaagcctggggcctcagtgaaggtttcctgcaaggcatctggatacaccttcaccagctactatatgcactgggtgcgacaggcccctggacaagggcttgagtggatgggacgtatcaacccgaatagtggtggaacaaagtacgcacagaagttccagggcagagtcaccatgaccagggacacgtccacgagcacagtctacatggagctgagcagcctgagatctgaggacacggccgtgtattactgtgctagactaggctctgcctacttcgatctctggggccgtggcaccctggtcactgtctcctca

SEQ ID NO: 438 - DNA, 384 bp, synthetic construct
  source 1..384: mol_type = other DNA
caggtgcagctggtgcagtctggggctgaggtgaagaagcctggggcctcagtgaaggtctcctgcaaggcttctggatacaccttcaccagtcatgatatcaactgggtgcgacaggccccaggacaagggcttgagtggatgggatggatgaacgctaacaacggtaacgctggctatgcacagaagttccagggcagagtcaccatgaccagggatacctccaccagcacagtttacatggagctgagcagcctgagatctgaggacacggccgtgtattactgtgcgaaggacttgtctccatcttcttcctggtccacctactattactattacgggatggatgtctggggccaagggacaatggtcaccgtctcttca

SEQ ID NO: 439 - DNA, 360 bp, synthetic construct
  source 1..360: mol_type = other DNA
caggttcagctggtgcagtctggagctgaggtgaagaagcctggggcctcagtgaaggtctcctgcaaggcttctggttacacctttaccagctataatatccactgggtgcgacaggcccctggacaagggcttgagtggatgggatggatcagcgcctacaatggtaacacaaactatgcacagaagctccagggcagagtcaccatgaccagagacacatccacgagcacagtctacatggagctgagcagcctgagatctgaggacacggccgtgtattactgtgcgctggatggagggcagctaggcgtggcttttgatatctggggccaagggacaatggtcaccgtctcttca

SEQ ID NO: 440 - DNA, 384 bp, synthetic construct
  source 1..384: mol_type = other DNA
caggtgcagctggtgcagtctggggctgaggtgaagaagcctggggcctcagtgaaggtttcctgcaaggcatctggaggaaccttcagcagctacgctatcagctgggtgcgacaggcccctggacaagggcttgagtggatgggaataatcaaccctagttccagcagcacaggctacgcacagaagttccagggcagagtcaccatgaccagggacacgtccacgagcacagtctacatggagctgagcagcctgagatctgaggacacggccgtgtattactgtgctagagccggttactacgactcttcgggctattactattccccttactatggcatggacgtgtggggccaagggaccacagtcaccgtctcctca

SEQ ID NO: 441 - DNA, 354 bp, synthetic construct
  source 1..354: mol_type = other DNA
caggttcagctggtgcagtctggagctgaggtgaagaagcctggggcctcagtgaaggtctcctgcaaggcttctggttacacctttaccaacttcgacatcaattgggtgcgacaggcccctggacaagggcttgagtggatgggatggatcagcgcctacaatggtcatacaaactatgcacagaacctccagggcagagtcaccatgaccagggacacatccacgagcacagtgtacatggagctgtctagcctgagatctgaagacacggccgtgtattactgtgcgcaggaagccgcggctgggtactttgactactggggccaagggaccctggtcaccgtctcctca

SEQ ID NO: 442 - DNA, 357 bp, synthetic construct
  source 1..357: mol_type = other DNA
caggtgcagctggtgcagtctggggctgaggtgaagaagcctggggcctcagtgaaggtctcctgcaaggcttctggaggaaccttccgcagttatgccatcagctgggtgcgacaggccccaggacaagggcttgagtggatgggatggatgaaccctaacagtggtaacacaggctatgcacagaagttccagggcagagtcaccatgaccagggatacctccacaagcacagtgtacatggagctgagcagcctgagatctgaggacacggccgtgtattactgtgcgagagagagcgtgctcaaaggaggtatggatgtctggggccaagggacaacggtcaccgtctcttca

SEQ ID NO: 443 - DNA, 381 bp, synthetic construct
  source 1..381: mol_type = other DNA
caggtgcagctggtgcagtctggggctgaggtgaagaagcctggggcctcagtgaaggtttcctgcaaggcatctggatacaccttcaccagctactatatgtattgggtgcgacaggcccctggacaagggcttgagtggatgggatggatcaacccaaatagtggtaacacaggatacgcacagaagttccagggcagagtcaccatgaccagggacacgtccacgagcacagtctacatggagctgagcagcctgagatctgaggacacggccgtgtattactgtgctaaggataggcgccgtggagtgggcctagccgctgccggcaccagcgcttttgatatctggggccaagggacaatggtcaccgtctcttca

SEQ ID NO: 444 - DNA, 369 bp, synthetic construct
  source 1..369: mol_type = other DNA
caggtgcagctggtgcagtctggggctgaggtgaagaagcctggggcctcagtgaaggtttcctgcaaggcatctggatacaccttcaccagctactatatgcactgggtgcgacaggcccctggacaagggcttgagtggatgggatggatgaaccctaactctgacaacacaggttacgcacagaagttccagggcagagtcaccatgaccagggacacgtccacgagcacagtctacatggagctgagcagcctgagatctgaggacacggccgtgtattactgtgctagagtgggggtcggttatggctactactactacggaatggacgtgtggggccaagggaccaccgtcaccgtctcctca

SEQ ID NO: 445 - DNA, 360 bp, synthetic construct
  source 1..360: mol_type = other DNA
caggtgcagctggtgcagtctggggctgaggtgaagaagcctggggcctcagtgaaggtttcctgcaaggcatctggatacaccttctcaaattactatatgcactgggtgcgacaggcccctggacaagggcttgagtggatgggatggatgaaccccaatagtggtaacacagggtacgcacagaagttccagggcagagtcaccatgaccagggacacgtccacgagcacagtctacatggagctgagcagcctgagatctgaggacacggccgtgtattactgtgctagagcctcaatgtactcttcaagtgggtttgactactggggccaagggaccctggtcaccgtctcctca

SEQ ID NO: 446 - DNA, 372 bp, synthetic construct
  source 1..372: mol_type = other DNA
caggtgcagctggtgcagtctggggctgaggtgaagaagcctggggcctcagtgaaggtttcctgcaaggcatctggatacaccttcaccagctactatatccactgggtgcgacaggcccctggacaagggcttgagtggatgggaacgatcaaccctagtggtggcagcacaagctacgcacagaagttccagggcagagtcaccatgaccagggacacgtccacgagcacagtctacatggagctgagcagcctgagatctgaggacacggccgtgtattactgtgctagagagactagccggtggctcagagattattattacggaatggatgtctggggccaagggacaactgtcaccgtctcttca

SEQ ID NO: 447 - DNA, 366 bp, synthetic construct
  source 1..366: mol_type = other DNA
caggtgcagctggtgcagtctggggctgaggtgaagaagcctggggcctcagtgaaggtttcctgcaaggcatctggatacaccttcaccgacaattatctacactgggtgcgacaggcccctggacaagggcttgagtggatgggaataatcgagacgagtggtggcagcacagactacgcacagaagttccagggcagagtcaccatgaccagggacacgtccacgagcacagtctacatggagctgagcagcctgagatctgaggacacggccgtgtattactgtgctagaagctttggtggatattcgtatgacgatgcttttgatatctggggccaagggacaacggtcaccgtctcttca

SEQ ID NO: 448 - DNA, 357 bp, synthetic construct
  source 1..357: mol_type = other DNA
caggttcagctggtgcagtctggagctgaggtgaagaagcctggggcctcagtgaaggtctcctgcaaggcttctggttacaccctcaataattatgatatcaattgggtgcgacaggcccctggacaagggcttgagtggatgggatggatcagcggctacaatggtaacacaaactatgcacagaagctccagggcagagtcaccatgacccgcgacacatccacgagcacagtgtacatggagctgtcgagcctgagatctgaggacacggccgtgtattactgtgcgagagtgggctcctcttgggaagaaaacgaccagtggggccaagggaccctggtcaccgtctcctca

SEQ ID NO: 449 - DNA, 378 bp, synthetic construct
  source 1..378: mol_type = other DNA
caggtgcagctggtgcagtctggggctgaggtgaagaagcctggggcctcagtgaaggtctcctgcaaggcttctggaggcaccttctcgagttatgatatcaactgggtgcgacaggccccaggacaagggcttgagtggatgggatggatgaaccccaacagtggtaacacaggctatgcacagaagttccagggcagagtcaccatgaccagggacacctccacaagcacagtgtacatggagctgagcagcctgagatctgaggacacggccgtgtattactgtgcgagagatctcacgctgtattgtagcagtaccagctgtcttgacgcctttgactactggggccaagggaccctggtcaccgtctcctca

SEQ ID NO: 450 - DNA, 354 bp, synthetic construct
  source 1..354: mol_type = other DNA
caggtgcagctggtgcagtctggggctgaggtgaagaagcctggggcctcagtgaaggtctcctgcaaggcttctggaggaaccttctctaattatggttttagctgggtgcgacaggccccaggacaagggcttgagtggatgggatggatgtcaccttctagtggtaacacaggctatgcacagaagttccagggcagagtcaccatgaccagggatacctccacaagcacagtgtacatggagctgagcagcctgagatctgaggacacggccgtgtattactgtgcgagagaacgctccggttcctactttgactactggggccaagggaccctggtcaccgtctcctca

SEQ ID NO: 451 - DNA, 366 bp, synthetic construct
  source 1..366: mol_type = other DNA
caggtccagctggtgcagtctggggctgaggtgaagaagcctgggtcctcggtgaaggtctcctgcaaggcttctggaggcaccttcagcagctatgctatcagctgggtgcgacaggcccctggacaagggcttgagtgggtgggaaggatccatccacgcgacggtagcacagactacgcacagaagttccagggcagagtcacgattaccgcggacgaatccacgagcacagcctacatggagctgagcagcctgagatctgaggacacggccgtgtattactgtgcgagagtttccctgcgcttcctagaggactacggtatggacgtctggggccaagggaccacggtcaccgtctcctca

SEQ ID NO: 452 - DNA, 357 bp, synthetic construct
  source 1..357: mol_type = other DNA
caggtgcagctggtgcagtctggggctgaggtgaagaagcctgggtcctcggtgaaggtctcctgcaaggcttctggaggcaccttcagcaactatgctatcagctgggtgcgacaggcccctggacaagggcttgagtggatgggagggatcattcccatctttggttcgacagcaagctacgcacagaagttccagggcagagtcacgattaccgcggacgaatccacgagcacagcctacatggagctgagcagcctgagatctgaggacacggccgtgtattactgtgcgagaggtgtcggagacgacaccctgactgattggggccaagggaccctggtcaccgtctcctca

SEQ ID NO: 453 - DNA, 372 bp, synthetic construct
  source 1..372: mol_type = other DNA
caggtgcagctggtgcagtctggggctgaggtgaagaagcctggggcctcagtgaaggtttcctgcaaggcatctggatacaccttcaccagctaccacatgcactgggtgcgacaggcccctggacaagggcttgagtggatgggatggatgaacccgaacagtggtaacacagggtacgcacagaagttccagggcagagtcaccatgaccagggacacgtccacgagcacagtctacatggagctgagcagcctgagatctgaggacacggccgtgtattactgtgctagaggtggacgcgtagtgaggggggtgatcaaagggtggttcgacccctggggccagggaaccctggtcaccgtctcctca

SEQ ID NO: 454 - DNA, 354 bp, synthetic construct
  source 1..354: mol_type = other DNA
caggttcagctggtgcagtctggagctgaggtgaagaagcctggggcctcagtgaaggtctcctgcaaggcttctggtggaacctttaccagctatgacatcaattgggtgcgacaggcccctggacaagggcttgagtggatgggatggatcagcgcatacaatggtcatacaaactatgcacagaatctccagggcagagtcaccatgacccgcgacacatccacgagcacagtgtacatggagctgtcgagcctgagatctgaagacacggccgtgtattactgtgcgagagaagacagttcttggtacttcgatctctggggccgtggcaccctggtcactgtctcctca

SEQ ID NO: 455 - DNA, 369 bp, synthetic construct
  source 1..369: mol_type = other DNA
caggtgcagctggtgcagtctggggctgaggtgaagaagcctgggtcctcggtgaaggtctcctgcaaggcttctggaggcaccttcagcaagattgctgtgagctgggtgcgacaggcccctggacaagggcttgagtggatgggacttatcaacccaaactccggtgggactgactacgcacagaagttccagggcagagtcacgattaccgcggacgaatccacgagcacagcctacatggagctgagcagcctgagatctgaggacacggccgtgtattactgtgcgaaggactttagaggcggggattattactattatggaatggacgtatggggccaagggaccacggtcaccgtctcctca

SEQ ID NO: 456 - DNA, 381 bp, synthetic construct
  source 1..381: mol_type = other DNA
caggttcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcctacaatggtaacacaaactatgcacagaagttccagggcagagtcaccatgacccgagacacatccacgagcacagtgtacatggagctgagcagcctgagatctgaggacacggccgtgtattactgtgcgagaggaacttactacgattatatttggggatcttacaggtacagctggtttgactactggggccaagggaccctggtcaccgtctcctca

SEQ ID NO: 457 - DNA, 384 bp, synthetic construct
  source 1..384: mol_type = other DNA
caggtgcagctggtgcagtctggggctgaggtgaagaagcctggggcctcagtgaaggtctcctgcaaggcttctggatacaccttctccagttatgctatctcttgggtgcgacaggcccccggacaagggcttgagtggatgggatggatggaccctaacagtggtaacacaggctatgcacagaagttccagggcagagtcaccatgaccagggatacctccaccagcacagtctacatggagctgagcagcctgagatctgaggacacggccgtgtattactgtgcgagagagtcgtcctactattatgatagcagcggctactatcctggatggtacttcgatctctggggccgtggcaccctggtcactgtctcctca

SEQ ID NO: 458 - DNA, 366 bp, synthetic construct
  source 1..366: mol_type = other DNA
caggtgcagctggtgcagtctggggctgaggtgaagaagcctggggcctcagtgaaggtctcctgcaaggcttctggatacaccttcaccacctatgggatctcctgggtgcgacaggccccaggacaagggcttgagtggatgggatggatggacccaaacagtggttctacaggctatgcacagaagttccagggcagagtcaccatgaccagggatacctccaccagcacagtgtacatggagctgagcagcctgagatctgaggacacggccgtgtattactgtgcgagaggccggggcaacagctactactactactacatggacgtctggggcaaagggaccacggtcaccgtctcctca

SEQ ID NO: 459 - DNA, 360 bp, synthetic construct
  source 1..360: mol_type = other DNA
caggtgcagctggtgcagtctggggctgaggtgaagaagcctggggcctcagtgaaggtttcctgcaaggcatctggatacaccttcaccagctactatatgcactgggtgcgacaggcccctggacaagggcttgagtggatgggatggatgaaccccaatagtggtaatacaggctacgcacagaagttccagggcagagtcaccatgaccagggacacgtccacgagcacagtctacatggagctgagcagcctgagatctgaggacacggccgtgtattactgtgctagagatcagatgtatagttcatacggcatggatgtctggggccaagggacaactgtcaccgtctcttca

SEQ ID NO: 460 - DNA, 357 bp, synthetic construct
  source 1..357: mol_type = other DNA
caggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagtcatcaacccttcaggtgggtccacatcatacgcacagaagttccagggcagagtcacgattaccgcggacgaatccacgagcacagcctacatggagctgagcagcctgagatctgaggacacggccgtgtattactgtgcgagagaatctagagggtactatgggatggacgtgtggggccaagggaccactgtcaccgtctcctca

SEQ ID NO: 461 - DNA, 348 bp, synthetic construct
  source 1..348: mol_type = other DNA
caggtgcagctggtgcagtctggggctgaggtgaagaagcctggggcctcagtgaaggtttcctgcaaggcatctggatacaccttcaccggctactatatgcactgggtgcgacaggcccctggacaagggcttgagtggatgggaaccatcaaccctagtggtggcagcacaagctacgcacagaagttccagggcagagtcaccatgaccagggacacgtccacgagcacagtctacatggagctgagcagcctgagatctgaggacacggccgtgtattactgtgtcaaaggtaacggctactacgacctgtggggccaagggaccctggtcaccgtctcctca

SEQ ID NO: 462 - DNA, 378 bp, synthetic construct
  source 1..378: mol_type = other DNA
caggtgcagctggtgcagtctggggctgaggtgaagaagcctggggcctcagtgaaggtctcctgcaaggcttctggatacaccttcacccgatatgatatcaactgggtgcgacaggcccccggacaagggcttgagtggatgggatggatgaaccctaacagtggtaacacaggctatgcacagaagttacagggcagagtcaccatgaccagggacacctccacaagcacagtctacatggagctgagcagcctgagatctgaggacacggccgtgtattactgtgcgagagagcgtgtggggtactgctcgagcacctcgtgtcttcatccactggactactggggccaagggaccctggtcaccgtctcctca

SEQ ID NO: 463 - DNA, 357 bp, synthetic construct
  source 1..357: mol_type = other DNA
caggttcagctggtgcagtctggagctgaggtgaagaagcctggggcctcagtgaaggtctcctgcaaggcttctggttacacctttaccagctatgacatcaactgggtgcgacaggcccctggacaagggcttgagtggatgggatggatcagcgggtacaatggtaacacaaactatgcacagaagttccagggcagagtcaccatgaccagggacacatccacgagcacagtttacatggagctgagcagcctgagatctgaggacacggccgtgtattactgtgcgagatcttttgattggttactgctacttgactactggggccaagggaccctggtcaccgtctcctca

SEQ ID NO: 464 - DNA, 369 bp, synthetic construct
  source 1..369: mol_type = other DNA
caggtgcagctggtgcagtctggggctgaggtgaagaagcctggggcctcagtgaaggtttcctgcaaggcatctggacacaccttcaacaataattatattcactgggtgcgacaggcccctggacaagggcttgagtggatgggaataatcaaccctagtggtgggagcacaagctacgcacagaagttccagggcagagtcaccatgaccagggacacgtccacgagcacagtctacatggagctgagcagcctgagatctgaggacacggccgtgtattactgtgcgagacaaatacaggattacggatactactattacggcatggacgtgtggggccaagggaccaccgtcaccgtctcctca

SEQ ID NO: 465 - DNA, 321 bp, synthetic construct
  source 1..321: mol_type = other DNA
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggcctctcactttcggcggagggaccaaggtggagatcaaa

SEQ ID NO: 466 - DNA, 318 bp, synthetic construct
  source 1..318: mol_type = other DNA
gacatccagatgacccagtctccttccaccctgtctgcatctgttggagacagagtcaccatcacttgccgggccagtcagagtattagtagctggttggcctggtatcagcagaaaccagggaaagcccctaagctcctgatctataaggcgtctagtttagaaactggggtcccatcaaggttcagcggcagtggatctgggacagaattcactctcaccatcagcagcctgcagcctgatgattttgccacttattactgccaacagtatgatagttactacacttttggccaggggaccaagctggagatcaaa

SEQ ID NO: 467 - DNA, 324 bp, synthetic construct
  source 1..324: mol_type = other DNA
gaaatagtgatgacgcagtctccagccaccctgtctgtgtctccaggggaaagagccaccctctcctgcagggccagtcagagtctgggcagcacttatttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtattatagctcccctcccacgttcggccaagggaccaaggtggaaatcaaa

SEQ ID NO: 468 - DNA, 321 bp, synthetic construct
  source 1..321: mol_type = other DNA
gacatccagatgacccagtctccatcctccctgtctgcatctgtaggagacagagtcaccatcacttgccaggcgagtcaggacattcacaactatttaaattggtatcagcagaaaccagggaaagcccctaagctcctgatctacgatgcatccaatttggaaacaggggtcccatcaaggttcagtggaagtggatctgggacagattttactctcaccatcagcagcctgcagcctgaagattttgcaacatattactgtcaacagagttataatacccctcccaccttcggccaagggacacgactggagattaaa

SEQ ID NO: 469 - DNA, 321 bp, synthetic construct
  source 1..321: mol_type = other DNA
gacatccagatgacccagtctccatcctccctgtctgcatctgtaggagacagagtcaccatcacttgccgcgcgagtcagaccattaataactatttaaattggtatcagcagaaaccagggaaagcccctaagctcctgatctacgatgcatccaatttggaaacaggggtcccatcaaggttcagtggaagtggatctgggacagattttactctgaccatcagcagcctgcagcctgaagattttgcaacatattactgtcaacaggctaatagtttccctctcactttcggccaggggaccaaggtggagatcaaa

SEQ ID NO: 470 - DNA, 321 bp, synthetic construct
  source 1..321: mol_type = other DNA
gacatccagatgacccagtctccatcctccctgtctgcatctgtaggagacagagtcaccatcacttgccgggcaagtcagaacattaggaactatttaaattggtatcagcagaaaccagggaaagcccctaagctcctgatctatgctgcatccagtttgcaaggaggggtcccatcaaggttcagtggcagtggatctgggacagatttcactctcaccatcagcagtctgcaacctgaagattttgcaacttactactgtcaacagagttacaacgggccttacacgttcggccaagggaccaaggtggaaatcaaa

SEQ ID NO: 471 - DNA, 336 bp, synthetic construct
  source 1..336: mol_type = other DNA
gatattgtgatgactcagtctccactctccctgcccgtcacccctggagagccggcctccatctcctgcaggtctagtcagagcctcctgcactctaacgggtacaactatttggattggtacctgcagaagccagggcagtctccacagctcctgatctatgcagcgtctagtctgcagtccggggtccctgacaggttcagtggcagtggatcaggcacagattttacactgaaaatcagcagagtggaggctgaggatgttggggtttattactgcatgcaagggacccactggcctccaacgttcggccaagggaccaaggtggaaatcaaa

SEQ ID NO: 472 - DNA, 336 bp, synthetic construct
  source 1..336: mol_type = other DNA
gatattgtgatgactcagtctccactctccctgcccgtcacccctggagagccggcctccatctcctgcaggtctagtcagagcctcctgcattcgaatgggtacaactatttggattggtacctgcagaagccagggcagtctccacagctcctgatctatgcagcttctaccctccagtccggggtccctgacaggttcagtggcagtggatcaggcacagattttacactgaaaatcagcagagtggaggctgaggatgttggggtttattactgcatgcaaggtctacaaactttatacagttttggccaggggaccaagctggagatcaaa

SEQ ID NO: 473 - DNA, 321 bp, synthetic construct
  source 1..321: mol_type = other DNA
gacatccagatgacccagtctccatcttctctgtctgcatctgtaggagacagagtcaccatcacttgtcgggcgagtcagggtgtgggcgatagaagtgcctggtatcagcagaaaccagggaaagcccctaagctcctgatctatgacgcatccaacttggagacaggggtcccatcaaggttcagcggcagtggatctgggacagatttcactctcactatcagcagcctgcagcctgaagattttgcaacttactattgtcaacaggctgatactttcccttacacttttggccaggggaccaagctggagatcaaa

SEQ ID NO: 474 - DNA, 321 bp, synthetic construct
  source 1..321: mol_type = other DNA
gacatccagatgacccagtctccatcctccctgtctgcatctgtaggagacagagtcaccatcacttgccgggcaagtcagggcattagcacttatttagcctggtatcagcagaaaccagggaaagcccctaagctcctgatctatgctgcatccacattgcaaagtggggtcccatcaaggttcagtggcagtggatctgggacagatttcactctcaccatcagcagtctgcaacctgaagattttgcaacttactactgtcaacagagtcacagtgcacctatcacgttcggccaagggaccaaggtggaaatcaaa

SEQ ID NO: 475 - DNA, 321 bp, synthetic construct
  source 1..321: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgacgcatccagtttggagagtggggtcccatcaaggttcagtggcagtggatctgggacagatttcactctcaccatcagcagtctgcaacctgaagattttgcaacttactactgtcaacaggcaaacagtttccctttaactttcggccctgggaccaaagtggatatcaaa

SEQ ID NO: 476 - DNA, 321 bp, synthetic construct
  source 1..321: mol_type = other DNA
gacatccagatgacccagtctccatcttctctttctgcatctgtaggagacagagtcaccatcacttgtcgggcgagtcagggtattggaaatgacttagcctggtatcagcagaaaccagggaaagcccctaagctcctgatctatgacgcaaaggatttgcaccccggggtcccatcaaggttcagcggcagtggatctgggacagatttcactctcactatcagcagcctgcagcctgaagattttgcaacttactattgtcaacagagctacagtttccctaggaccttcggccaagggacacgactggagattaaa

SEQ ID NO: 477 - DNA, 321 bp, synthetic construct
  source 1..321: mol_type = other DNA
gacatccagatgacccagtctccatcctccctgtctgcatctgtaggagacagagtcaccatcacttgccgggcaagtgagagcattagccattggttagcctggtatcagcagaaaccagggaaagcccctaagctcctgatctatggtgcatccactttgcaaagtggggtcccatcaaggttcagtggcagtggatctgggacagatttcactctcaccatcagcagtctgcaacctgaagattttgcaacttactactgtcaacagagttacacccctcccctcactttcggcggagggaccaaggtggagatcaaa

SEQ ID NO: 478 - DNA, 339 bp, synthetic construct
  source 1..339: mol_type = other DNA
gacatcgtgatgacccagtctccagactccctggctgtgtctctgggcgagagggccaccatcaactgcaagtccagccagagtctcctctacagctctaataacaagaactacttagcttggtaccagcagaaaccaggacagcctcctaagctgctcatttactgggcatctatccgggaatccggggtccctgaccgattcagtggcagcgggtctgggacagatttcactctcaccatcagcagcctgcaggctgaagatgtggcagtttattactgtcagcaatatctcagtcctccactcactttcggccaggggaccaaggtggagatcaaa

SEQ ID NO: 479 - DNA, 321 bp, synthetic construct
  source 1..321: mol_type = other DNA
gacatccagatgacccagtctccatcctccctgtctgcatctgtaggagacagagtcaccatcacttgccgggcaagtcaggatattagacgagcattagcctggtatcagcagaaaccagggaaagcccctaagctgctgatctatgctgcatccagtttgcaaagtggggtcccatcaaggttcagcggcagtggatctgggacagatttcactctcacaatcagcagcctgcagcctgaagattttgcaacttattactgtctacagcataatagttaccctcccacttttggccaggggaccaagctggagatcaaa

SEQ ID NO: 480 - DNA, 333 bp, synthetic construct
  source 1..333: mol_type = other DNA
gatattgtgatgactcagtctccactctccctgcccgtcacccctggagagccggcctccatctcctgcaggtctagtcagagcctcctccacagtaatggatacaactatttggattggtacctgcagaagccagggcagtctccacagctcctgatctatttgggttctagtcgggcctccggggtccctgacaggttcagtggcagtggatcaggcacagattttacactgaaaatcagcagagtggaggctgaggatgttggggtttattactgcatgcaagctctacaaactcctaccttcggccaagggacacgactggagattaaa

SEQ ID NO: 481 - DNA, 321 bp, synthetic construct
  source 1..321: mol_type = other DNA
gacatccagatgacccagtctccatcctccctgtctgcatctgtaggagacagagtcaccatcacttgccaggcaagtcagggtattagcagctatttaaattggtatcagcagaaaccagggaaagcccctaagctcctgatctatgctgcatccagtttgcaaaatggggtcccatcaaggttcagtggcagtggatctgggacagatttcactctcaccatcagcagtctgcaacctgaagattttgcaacttactactgtcaacagagttacagtacccctatcaccttcggccaagggacacgactggagattaaa

SEQ ID NO: 482 - DNA, 321 bp, synthetic construct
  source 1..321: mol_type = other DNA
gacatccagatgacccagtctccatcctccctgtctgcatctgtaggagacagagtcaccatcacttgccgggcaagtcagggcattggcaatgatttaggctggtatcagcagaaaccagggaaagcccctaagcttctgatctatgctgcatccaacttggaaacgggggtcccatcaaggttcagcggcagtggatctgggacagatttcactctcacaatcagcagcctgcagcctgaagattttgcaacttattactgtctacagcatcatagttacccttttacttttggccaggggaccaagctggagatcaaa

SEQ ID NO: 483 - DNA, 315 bp, synthetic construct
  source 1..315: mol_type = other DNA
gacatccagatgacccagtctccatcctccctgtctgcatctgtaggagacagagtcaccatcacttgccggaccagtcagagcattggcacctatttaaattggtatcagcagaaaccagggaaagcccctaagctcctgatctatgacgcatccaatttgccgactggggtcccatcaaggttcagtggcagtggatctgggacagatttcactctcaccatcagcagtctgcaacctgaagattttgcaacttactactgtcaaaactataacagtgctctgttcggccctgggaccaaagtggatatcaaa

SEQ ID NO: 484 - DNA, 321 bp, synthetic construct
  source 1..321: mol_type = other DNA
gacatccagatgacccagtctccatcttctctctctgcatctgtaggagacagagtcaccatcacttgtcgggcgagtcagtctgtggatacctggttagcctggtatcagcagaaaccagggaaagcccctaagctcctgatctatgaagcatccacattggaaagtggggtcccatcaaggttcagcggcagtggatctgggacagatttcactctcactatcagcagcctgcagcctgaagattttgcaacttactattgtcaacaggctaacagtttccctatcaccttcggccaagggacacgactggagattaaa

SEQ ID NO: 485 - DNA, 321 bp, synthetic construct
  source 1..321: mol_type = other DNA
gacatccagatgacccagtctccatcctccctgtctgcatctgtaggagacagagtcaccatcacttgccgggcaagtcagagcattagcaatctcttagcgtggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaccccttacacttttggccaggggaccaagctggagatcaaa

SEQ ID NO: 486 - DNA, 321 bp, synthetic construct
  source 1..321: mol_type = other DNA
gacatccagatgacccagtctccatcttctctttctgcatctgtaggagacagagtcaccatcacttgtcgggcgagtcagggtattagcaattggttagcctggtatcagcagaaaccagggaaagcccctaagctcctgatctatgatgcatccaatttggacgccggggtcccatcaaggttcagcggcagtggatctgggacagatttcactctcactatcagcagcctgcagcctgaagattttgcaacttactattgtcaacaggctaacagtttacctctcactttcggcggagggaccaaggtggagatcaaa

SEQ ID NO: 487 - DNA, 324 bp, synthetic construct
  source 1..324: mol_type = other DNA
gaaatagtgatgacgcagtctccagccaccctgtctgtgtctccaggggaaagagccaccctctcctgcagggccagtcagagtatatctgggagctatttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtatgggagctttcctttcactttcggccctgggaccaaagtggatatcaaa

SEQ ID NO: 488 - DNA, 321 bp, synthetic construct
  source 1..321: mol_type = other DNA
gacatccagatgacccagtctccatcctccctgtctgcatctgtaggagacagagtcaccatcacttgccgggcaagtcaggatattcgtaatgatttagcctggtatcagcagaaaccagggaaagcccctaagctcctgatctatgacgcatccaatttggagacgggggtcccatcaaggttcagtggcagtggatctgggacagatttcactctcaccatcagcagtctgcaacctgaagattttgcaacttactactgtcaacagagttacggtaccccttacacttttggccaggggaccaagctggagatcaaa

SEQ ID NO: 489 - DNA, 339 bp, synthetic construct
  source 1..339: mol_type = other DNA
gacatcgtgatgacccagtctccagactccctggctgtgtctctgggcgagagggccaccatcaactgccggtccagccagagtctctttcacagcagcaccaacgccaacaacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatattactcctctgaccttcggccaagggacacgactggagattaaa

SEQ ID NO: 490 - DNA, 321 bp, synthetic construct
  source 1..321: mol_type = other DNA
gacatccagatgacccagtctccatcctccctgtctgcatctgtaggagacagagtcaccatcacttgccgggcaagtcagagcattagcaactggttagcctggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaccccttacacttttggccaggggaccaagctggagatcaaa

SEQ ID NO: 491 - DNA, 321 bp, synthetic construct
  source 1..321: mol_type = other DNA
gacatccagatgacccagtctccatcctccctgtctgcatctgtaggagacagagtcaccatcacttgccgggcaggtcagagcattggcgactatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaccccttggacgttcggccaagggaccaaggtggaaatcaaa

SEQ ID NO: 492 - DNA, 336 bp, synthetic construct
  source 1..336: mol_type = other DNA
gatattgtgatgactcagtctccactctccctgcccgtcacccctggagagccggcctccatctcctgcaggtctagtcagagcctcctgcacagcaatgggtacaactatttggattggtacctgcagaagccagggcagtctccacagctcctgatctatttggcgtctactcgggcccctggggtccctgacaggttcagtggcagtggatcaggcacagattttacactgaaaatcagcagagtggaggctgaggatgttggggtttattactgcatgcaaggatcccattggcctcctagttttggccaggggacccggctggagatcaaa

SEQ ID NO: 493 - DNA, 336 bp, synthetic construct
  source 1..336: mol_type = other DNA
gatattgtgatgactcagtctccactctccctgcccgtcacccctggagagccggcctccatctcctgcaggtctagtcagagcctcctacactctaatggttacaactatttggattggtacctgcagaagccagggcagtctccacagctcctgatctatgacgcctctagcctggagtccggggtccctgacaggttcagtggcagtggatcaggcacagattttacactgaaaatcagcagagtggaggctgaggatgttggggtttattactgcatgcaagggactcactggcctctcactttcggcggagggaccaaggtggagatcaaa

SEQ ID NO: 494 - DNA, 321 bp, synthetic construct
  source 1..321: mol_type = other DNA
gacatccagatgacccagtctccatcctccctgtctgcatctgtaggagacagagtcaccatcacttgccaggcaagtcagggtattagcaattatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtagcccttacacgttcggccaagggaccaaggtggaaatcaaa

SEQ ID NO: 495 - DNA, 321 bp, synthetic construct
  source 1..321: mol_type = other DNA
gacatccagatgacccagtctccatcttctctgtctgcatctgtaggagacagagtcaccatcacttgtcgggcgagtcagtccattagccggtggttagcctggtatcagcagaaaccagggaaagcccctaagctcctgatctatgctgcatccagtttggagagtggggtcccatcaaggttcagcggcagtggatctgggacagatttcactctcactatcagcagcctgcagcctgaagattttgcaacttactattgtcaacagacacacagtttccctctcactttcggcggagggaccaaggtggagatcaaa

SEQ ID NO: 496 - DNA, 339 bp, synthetic construct
  source 1..339: mol_type = other DNA
gacatcgtgatgacccagtctccagactccctggctgtgtctctgggcgagagggccaccatcaactgccgatccagccagagtgttttgtactcatcaaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtactcctccaacttttggccaggggaccaagctggagatcaaa

SEQ ID NO: 497 - DNA, 321 bp, synthetic construct
  source 1..321: mol_type = other DNA
gaaatagtgatgacgcagtctccagccaccctgtctgtgtctccaggggaaagagccaccctctcctgcagggccagtcagagtgttagcagctatttagcctggtaccagcagaaacctggccaggctcccaggctcctcatctatggtacctccaccagggccactggtatcccagccaggttcagtggcagtgggtctgggacagagttcactctcaccatcagcagcctgcagtctgaagattttgcagtttattactgtcagcagtatggctcgctacctttcaccttcggccaagggacacgactggagattaaa

SEQ ID NO: 498 - DNA, 339 bp, synthetic construct
  source 1..339: mol_type = other DNA
gacatcgtgatgacccagtctccagactccctggctgtgtctctgggcgagagggccaccatcaactgcaagtccagccagagtgttttgtacagttcaaataacaagaactacttagcttggtaccagcagaaaccaggacagcctcctaagctgctcatttactgggcatctacccgggcatccggggtccctgaccgattcagtggcagcgggtctgggacagatttcactctcaccatcagcagcctgcaggctgaagatgtggcagtttattactgtcagcaatattatactactcctccaaccttcggccaagggacacgactggagattaaa

SEQ ID NO: 499 - DNA, 336 bp, synthetic construct
  source 1..336: mol_type = other DNA
gatattgtgatgactcagtctccactctccctgcccgtcacccctggagagccggcctccatctcctgcaggtctagtcagagcctcctgcattcaaacgggtacaactatttggattggtacctgcagaagccagggcagtctccacagctcctgatctatgctgcttctagcctgcagtccggggtccctgacaggttcagtggcagtggatcaggcacagattttacactgaaaatcagcagagtggaggctgaggatgttggggtttattactgcatgcaagctctacaaactcctctcactttcggcggagggaccaaggtggagatcaaa

SEQ ID NO: 500 - DNA, 339 bp, synthetic construct
  source 1..339: mol_type = other DNA
gacatcgtgatgacccagtctccagactccctggctgtgtctctgggcgagagggccaccatcaactgcaagtccagccagagtcttctgtatacttccaacgatcgcaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtagcccttacacgttcggccaagggaccaaggtggaaatcaaa

SEQ ID NO: 501 - DNA, 321 bp, synthetic construct
  source 1..321: mol_type = other DNA
gacatccagatgacccagtctccatcctccctgtctgcatctgtaggagacagagtcaccatcacttgccgggcaagtcagagcattagcagctggttagcctggtatcagcagaaaccagggaaagcccctaagctcctgatctatgacgcatccaatttggagactggggtcccatcaaggttcagtggcagtggatctgggacagatttcactctcaccatcagcagtctgcaacctgaagattttgcaacttactactgtcaacagagtttcagtacccctccaacgttcggccaagggaccaaggtggaaatcaaa

SEQ ID NO: 502 - DNA, 321 bp, synthetic construct
  source 1..321: mol_type = other DNA
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataatacataccctatcacgttcggccaagggaccaaggtggaaatcaaa

SEQ ID NO: 503 - DNA, 321 bp, synthetic construct
  source 1..321: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggagggaccaaggtggagatcaaa

SEQ ID NO: 504 - DNA, 321 bp, synthetic construct
  source 1..321: mol_type = other DNA
gacatccagatgacccagtctccatcctccctgtctgcatctgtaggagacagagtcaccatcacttgccaggcaagtcaggacattagcaactatttaaattggtatcagcagaaaccagggaaagcccctaagctcctgatctatgctgcatccaccttgcaaacaggggtcccatcaaggttcagtggcagtggatctgggacagatttcactctcaccatcagcagtctgcaacctgaagattttgcaacttactactgtcaacagagttacagtaccccttggactttcggccctgggaccaaagtggatatcaaa

SEQ ID NO: 505 - DNA, 321 bp, synthetic construct
  source 1..321: mol_type = other DNA
gacatccagatgacccagtctccatcttctctttctgcatctgtaggagacagagtcaccatcacttgtcgggcgagtcagcgcattggtagctacttaaactggtatcagcagaaaccagggaaagcccctaagctcctgatctatggagcatccaatttgcaaagtggggtcccatcaaggttcagcggcagtggatctgggacagatttcactctcactatcagcagcctgcagcctgaagattttgcaacttactattgtcaacaggctaacagtttccctttcactttcggccctgggaccaaagtggatatcaaa

SEQ ID NO: 506 - DNA, 321 bp, synthetic construct
  source 1..321: mol_type = other DNA
gacatccagatgacccagtctccatcctccctgtctgcatctgtaggagacagagtcaccatcacttgccgggcaagtgaaagcattacaaactggttagcttggtatcagcagaaaccagggaaagcccctaagctcctgatctataaagcatccagtttggaaagtggggtcccatcaaggttcagtggcagtggatctgggacagatttcactctcaccatcagcagtctgcaacctgaagattttgcaacttactactgtcaacagagttacggtaccccttacacgttcggccaagggaccaaggtggaaatcaaa

SEQ ID NO: 507 - DNA, 339 bp, synthetic construct
  source 1..339: mol_type = other DNA
gacatcgtgatgacccagtctccagactccctggctgtgtctctgggcgagagggccaccatcaactgcaagtccagccagagtgttctctattcgtccaataacaagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtactccttacacgttcggccaagggaccaaggtggaaatcaaa

SEQ ID NO: 508 - DNA, 321 bp, synthetic construct
  source 1..321: mol_type = other DNA
gacatccagatgacccagtctccatcttctctgtctgcatctgtaggagacagagtcaccatcacttgtcgggcgagtcagtcaattagcagctggttagcctggtatcagcagaaaccagggaaagcccctaagctcctgatctatgatacatccaacttgcaaggtggggtcccatcaaggttcagcggcagtggatctgggacagatttcactctcactatcagcagcctgcagcctgaagattttgcaacttactattgtcaacagtatgatagtttcccttggacgttcggccaagggaccaaggtggaaatcaaa

SEQ ID NO: 509 - DNA, 321 bp, synthetic construct
  source 1..321: mol_type = other DNA
gacatccagatgacccagtctccatcctccctgtctgcatctgtaggagacagagtcaccatcacttgccgggcaagtcagggcattagaaatgatttaggctggtatcagcagaaaccagggaaagcccctaagctcctgatctatagtgcatccaatttacaaagtggggtcccatcaaggttcagcggcagtggatctggcacagatttcactctcaccatcagcagcctgcagcctgaagattttgcaacttattactgtcagcaaagctacgatgtccctctcactttcggcggagggaccaaggtggagatcaaa

SEQ ID NO: 510 - DNA, 321 bp, synthetic construct
  source 1..321: mol_type = other DNA
gacatccagatgacccagtctccttccagtctgtctgcatctgtaggagacagagtcaccatcacttgccgggccagtcagagtattagtagctggttggcctggtatcagcagaaaccagggaaagcccctaagctcctgatctatgatgccactaccttggaaagtggggtcccatcaaggttcagcggcagtggatctgggacagatttcactctcaccatcagcagcctgcagcctgaagattttgcaacttattactgccaacagtatgacagttatccatggacgttcggccaagggaccaagctcgaaatcaaa

SEQ ID NO: 511 - DNA, 321 bp, synthetic construct
  source 1..321: mol_type = other DNA
gaaatagtgatgacgcagtctccagccaccctgtctgtgtctccaggggaaagagccaccctctcctgcagggccagtcagagtgttagcaagtatttagcctggtaccagcagaaacctggccaggctcccaggctcctcatctatgccatctccgcgagggccgctggtatcccagccaggttcagtggcagtgggtctgggacagagttcactctcaccatcagcagcctgcagtctgaagattttgcagtttattactgtcagcagtattattccagtcctcctacgttcggccaagggaccaaggtggaaatcaaa

(19) United States
(12) Patent Application Publication
(10) Pub. No.: US 2024/0400677 A1
(43) Pub. Date: Dec. 5, 2024

(54) SIRP GAMMA ANTIBODIES AND USES THEREOF

(71) Applicant: Electra Therapeutics, Inc., South San Francisco, CA (US)

(72) Inventors: Sandip PANICKER, Pacifica, CA (US); Adam David ROSENTHAL, San Francisco, CA (US); Eileen Lingshu ROSE, San Bruno, CA (US); Allen Guo Yang CAI, Millbrae, CA (US)

(21) Appl. No.: 18/660,086

(22) Filed: May 9, 2024

Related U.S. Application Data
(63) Continuation of application No. PCT/US2022/079668, filed on Nov. 10, 2022.
(60) Provisional application No. 63/277,966, filed on Nov. 10, 2021.

(51) Int. Cl. C07K 16/28; A61K 39/00

(52) U.S. Cl. CPC C07K 16/2803; A61K 2039/505; C07K 2317/622; C07K 2317/732; C07K 2317/92

(57) ABSTRACT

Provided herein are antibodies that bind signal regulatory protein SIRPγ and methods of making and using such antibodies. In some embodiments, the antibodies comprise an Fc, and are useful for preferential depletion of certain SIRPγ expressing cells. In some embodiments, the antibodies provided herein are useful for treating a disease or condition associated with the dysregulation of cells (e.g. overactivation or aberrant proliferation of T-cells).

### BACKGROUND

This application claims priority to U.S. Provisional Patent Application No. 63/277,966, filed on Nov. 10, 2021. The contents of this application are incorporated by reference herein in its entirety.

### INCORPORATION BY REFERENCE OF SEQUENCE LISTING

The contents of the electronic sequence listing (ELTH_003_01WO_SegListST26.xml; Size: 459,345 bytes; and Date of Creation: Nov. 8, 2022) are herein incorporated by reference in its entirety.

### BACKGROUND

Signal regulatory proteins (SIRPs) are a family of cell-surface immune receptors with Ig-like extracellular domains and include three members, SIRPα, SIRPβ1, and SIRPγ. SIRPα and SIRPβ1 are found on myeloid cells including monocytes, macrophages, dendritic cells, and on granulocytes such as neutrophils, eosinophils, and basophils. Unlike SIRPα and SIRPβ1, SIRPγ (also called CD172-antigen-like family member B, CD172γ, SIRPβ2, and SIRP-beta-2) is not expressed by myeloid cells but is instead primarily expressed by T-cells and has also been found on activated NK-cells and a subset of B-cells. Like SIRPα, SIRPγ has been found to bind to CD47, a ubiquitously expressed cell surface protein, albeit with a lower affinity when compared to SIRPα. Unlike SIRPα, SIRPγ does not have a known signaling mechanism. Rather, SIRPγ has been shown to mediate cell-cell adhesion where it has been posited to promote T-cell-antigen presenting cell interactions for immune synapse stabilization and immune activation (Blood (2005) 105 (6): 2421-2427).

There is a need for agents that bind to specific populations of cells, while sparing other cells, useful for the targeted treatment of a variety of diseases and conditions. Such agents would provide a safe and effective approach to treat oncology, autoimmune and inflammatory disorders driven by the pathological activity of certain populations of cells. Provided herein are such agents that target specific populations of SIRPγ-expressing cells, useful for the targeted treatment of a variety of diseases and conditions.

### SUMMARY

Provided herein are antibodies specific for SIRPγ, useful for the targeted depletion of certain cell populations. In some embodiments, SIRPγ may be upregulated upon certain cell states (e.g. stimulation or exhaustion), and the use of the antibodies of the disclosure induces a preferential depletion of such cells in a particular state. In some embodiments, different SIRPγ isoforms may be expressed on different subsets of cells, and the use of the antibodies of the disclosure induces a preferential depletion of such different subsets. Also provided herein are methods of making, and methods of use of the SIRPγ antibodies.

Exemplary antibody amino acid sequences are provided in Tables 1 and 2.

More specifically, in one aspect, provided herein is an Fc-containing antibody that is specific for SIRPγ, wherein the antibody has low or no affinity for binding SIRPα and SIRPβ1, and wherein binding of the antibody to a SIRPγ-expressing cell induces effector-mediated depletion of the SIRPγ-expressing cell.

In another aspect, provided herein is a SIRPγ antibody that has low or no affinity for binding SIRPα and SIRPβ1, and wherein the antibody exhibits preferential binding to and depletion of an activated (stimulated) T-cell, as compared to an unstimulated T-cell. In related aspects, provided herein is: (a) a SIRPγ antibody that has low or no affinity for binding SIRPα and SIRPβ1, and wherein the antibody exhibits preferential binding to and depletion of a CD8+ T-cell; (b) a SIRPγ antibody that has low or no affinity for binding SIRPα and SIRPβ1, and wherein the antibody exhibits preferential binding to and depletion of a CD4+ T-cell; (c) a SIRPγ antibody that has low or no affinity for binding SIRPα and SIRPβ1, and wherein the antibody exhibits preferential binding to and depletion of a CD8+/CD69+ T-cell; (d) a SIRPγ antibody that has low or no affinity for binding SIRPα and SIRPβ1, and wherein the antibody exhibits preferential binding to and depletion of a CD8+/CD25+ T-cell; (e) a SIRPγ antibody that has low or no affinity for binding SIRPα and SIRPβ1, and wherein the antibody exhibits preferential binding to and depletion of a CD8+ T-cell, when compared to a SIRPγ-expressing CD4+ T-cell; (f) a SIRPγ antibody that has low or no affinity for binding SIRPα and SIRPβ1, and wherein the antibody exhibits preferential binding to and depletion of a CD4+ T-cell, when compared to a SIRPγ-expressing CD8+ T-cell; (g) a SIRPγ antibody that has low or no affinity for binding SIRPα and SIRPβ1, and wherein the antibody exhibits preferential binding to and depletion of a CD8+/CD69+ T-cell, when compared to a SIRPγ-expressing CD8+/CD69− T-cell; (h) a SIRPγ antibody that has low or no affinity for binding SIRPα and SIRPβ1, and wherein the antibody exhibits preferential binding to and depletion of a CD8+/CD25+ T-cell, when compared to a SIRPγ-expressing CD8+/CD25− T-cell; or (i) a SIRPγ antibody that has low or no affinity for binding SIRPα and SIRPβ1, and wherein the antibody exhibits preferential binding to and depletion of a PD1+ T-cell, when compared to a SIRPγ-expressing PD1− T-cell.

In another aspect, provided herein is a method of inducing the preferential depletion of a population of SIRPγ-expressing cells, the method comprising contacting the population with any of the SIRPγ antibodies of the disclosure.

In another aspect provided herein is a method of treating a disease or condition in a subject in need thereof, comprising administering to the subject a therapeutically effective amount of any of the SIRPγ antibodies of the disclosure. In some embodiments, the Fc-containing SIRPγ antibodies provided herein are useful for the treatment of an autoimmune, inflammatory, or oncological disease or condition.

Also provided herein are pharmaceutical compositions comprising any of the SIRPγ antibodies of the disclosure, and optionally a pharmaceutically acceptable carrier. Also provided herein are nucleic acids encoding for the SIRPγ antibodies of the disclosure, and methods of making the same. Exemplary nucleic acids are provided for in Table 3.

### BRIEF DESCRIPTION OF THE DRAWINGS

FIG. 1 shows the binding of Antibody 1 and 2 to human and cynomolgus monkey (cyno) SIRPγ, SIRPα, and SIRPβ1 by enzyme-linked immunosorbent assay (ELISA).

FIG. 2A shows binding curves of selected antibodies to human and cynomolgus monkey SIRPγ by ELISA.

FIG. 2B shows binding curves of selected antibodies to human SIRPαV1, SIRPαV2, and SIRPβ1 by ELISA.

FIGS. 2C-2G show binding curves of selected antibodies to human and cynomolgus monkey SIRPγ, human SIRPαV1, and human SIRPβ1 by ELISA.

FIG. 3A shows the binding curves of selected antibodies to human CD3+ T-cells, CD20+ B-cells, and CD56+ NK-cells in human whole blood by flow cytometry.

FIGS. 3B and 3C show the binding curves of selected antibodies to human CD14+ monocytes and granulocytes in human whole blood by flow cytometry.

FIG. 3D shows the binding curves of selected antibodies to human CD4+ T-cells, CD8+ T-cells, CD20+ B-cells, and CD56+ NK-cells in human whole blood by flow cytometry.

FIGS. 4A-4C show that T-cell activation leads to differential regulation of cell surface SIRPγ expression, and that SIRPγ-specific antibodies are capable of increased binding to such activated T-cells. Ex vivo activation of healthy donor T-cells, using anti-CD3 and anti-CD28 antibodies, resulted in the upregulation of surface SIRPγ expression, compared to their unstimulated counterparts.

FIG. 4D is a model—upon activation of T-cells, SIRPγ expression is increased, and such increased target expression allows for preferential depletion of activated T-cells.

FIGS. 5A-5F show the binding curves of selected SIRPγ antibodies to human SIRPγ-expressing CHO cells and cynomolgus monkey SIRPγ-expressing CHO cells by flow cytometry.

FIGS. 5G-5L show the binding curves of selected SIRPγ antibodies to human and cynomolgus monkey SIRPα-expressing CHO cells, and human and cynomolgus monkey SIRPβ1-expressing CHO cells by flow cytometry.

FIGS. 6A-6C show the effect of selected antibodies on ADCC of human CD3+ resting (left) and stimulated (right) T-cells in vitro.

FIG. 7 shows the effect of selected antibodies on ADCP of human CD3+ resting (left panel) and stimulated (right panel) T-cells in vitro.

FIGS. 8A-8B show the results of an ELISA experiment assessing the ability of selected antibodies to disrupt CD47 binding to human SIRPγ.

FIG. 9 shows the results of a mixed lymphocyte reaction (MLR) experiment, assessing the effect of selected antibodies on T-cell proliferation.

FIG. 10 shows the effect of selected antibodies on human CD3+ T-cell depletion in vivo.

### DETAILED DESCRIPTION

Provided herein are antibodies that bind to SIRPγ. Also provided are methods of making and using such antibodies. Given the restricted and unique patterns of SIRPγ expression, these antibodies may be useful for targeting particular cell types, and treating diseases or conditions involving cells, or cell states expressing SIRPγ, e.g. activated cells. For example, in some embodiments, the antibodies may be used for treating diseases or conditions involving dysfunction, dysregulation, overactivation and/or hyperproliferation of SIRPγ-expressing cells as a part of their pathology.

Where elements are presented in a list format (e.g., in a Markush group), it should be understood that each possible subgroup of the elements is also disclosed, and that any one or more elements can be removed from the list or group.

It should be understood that, unless clearly indicated, in any method described or disclosed herein that includes more than one act, the order of the acts is not necessarily limited to the order in which the acts of the method are recited, but the disclosure encompasses exemplary embodiments in which the order of the acts is so limited.

The terms used throughout the specification are defined as follows unless otherwise limited in specific instances. As used in the specification and the claims, the singular forms “a,” “an,” and “the” include plural referents unless the context clearly dictates otherwise. All technical and scientific terms, acronyms, and abbreviates used in the specification and claims have the same meaning as commonly understood by one of ordinary skill in the art to which the disclosure pertains, unless defined or stated otherwise. All numerical ranges are inclusive of the values defining the range as well as all integer values in between, unless indicated or defined otherwise.

The term “antibody” as used herein throughout is used in the broadest sense and includes a monoclonal antibody, polyclonal antibody, human antibody, humanized antibody, non-human antibody, chimeric antibody, a monovalent antibody, and an antibody fragment. The term may refer to an intact tetrameric antibody containing two light chains and two heavy chains, each with a variable region, and a constant region (“full-length”). Alternatively, it may refer to antibody fragments.

Antibody fragments of the disclosure retain SIRPγ antigen binding specificity. Antibody fragments include antigen-binding fragments (Fab), variable fragments (Fv) containing VH and VL sequences, single chain variable fragments (scFv) containing VH and VL sequences linked together in one chain, single chain antibody fragments (scAb) or other antibody variable region fragments, such as Fab′, F(ab′)2, dsFv diabody, and Fd polypeptide fragments.

The term “depletion” as used herein throughout refers to cell death as a Fc-mediated effector function. Without being bound to theory or mechanism, the Fc-containing SIRPγ antibodies of the disclosure are capable of depleting SIRPγ-expressing target cells, and involve effector functions for their mechanism of action. Without being bound to theory or mechanism, it is thought that Fc-containing antibodies of the disclosure bind to SIRPγ-expressing cells via their complementarity determining regions (CDR), the Fc-regions of the antibodies interact with Fc-receptors on the surfaces of effector immune cells or circulating complement proteins, thus resulting in the depletion (cell death) of the SIRPγ-expressing target cells. The depletion can be effectuated via immune cell effector processes like antibody-dependent cell-mediated cytotoxicity (ADCC) or antibody-dependent cellular phagocytosis (ADCP); additionally or alternatively, the Fc-region can bind to a complement component and cause complement-dependent cytotoxicity (CDC). The Fc-mediated cell death (depletion) describe herein is independent of any CDR-mediated signal transduction that leads to cell death. However, it is acknowledged that the depletion that occurs with the use of any of the Fc-containing SIRPγ antibodies of the disclosure may also include a CDR-mediated cell death component, but it is not required.

The terms “individual,” “subject,” and “patient” are used interchangeably herein and refer to any subject for whom treatment or therapy is desired. The subject may be a mammalian subject. Mammalian subjects include, e.g., humans, non-human primates, rodents, (e.g., rats, mice), lagomorphs (e.g., rabbits), ungulates (e.g., cows, sheep, pigs, horses, goats, and the like), etc. In some embodiments, the subject is a human. In some embodiments, the subject is a non human primate, for example a cynomolgus monkey. In some embodiments, the subject is a companion animal (e.g. cats, dogs).

All publications, patents, and patent applications mentioned in this specification are herein incorporated by reference to the same extent as if each individual publication, patent, or patent application was specifically and individually indicated to be incorporated by reference.

### I. Antibodies

### A. SIRPγ Antibodies

Provided herein are antibodies that bind to SIRPγ, and exhibit little or no binding to SIRPα and SIRPβ1.

As used herein, “SIRPγ” includes all isoforms, from any species. In the human, there are multiple SIRPγ transcripts, including a full length, two alternatively spliced forms that lack significant portions of their extracellular domains, and one alternatively spliced form that lacks the signal peptide sequence.

The amino acid sequence of hSIRPγ isoform 1 (full length) is provided as SEQ ID NO: 1 (referencing UniProtKB ID Q9P1W8 Isoform 1).

| (SEQ ID NO: 1) | |
|---|---|
| 1 | MPVPASWPHP PGPFLLLTLL LGLTEVAGEE ELQMIQPEKL LLVTVGKTAT LHCTVTSLLP |
| 61 | VGPVLWFRGV GPGRELIYNQ KEGHFPRVTT VSDLTKRNNM DFSIRISSIT PADVGTYYCV |
| 121 | KFRKGSPENV EFKSGPGTEM ALGAKPSAPV VLGPAARTTP EHTVSFTCES HGFSPRDITL |
| 181 | KWFKNGNELS DFQTNVDPTG QSVAYSIRST ARVVLDPWDV RSQVICEVAH VTLQGDPLRG |
| 241 | TANLSEAIRV PPTLEVTQQP MRVGNQVNVT COVRKFYPQS LOLTWSENGN VCORETASTL |
| 301 | TENKDGTYNW TSWFLVNISD QRDDVVLTCQ VKHDGQLAVS KRLALEVTVH QKDOSSDATP |
| 361 | GPASSLTALL LIAVLLGPIY VPWKOKT |

The amino acid sequence of a splice variant hSIRPγ (isoform 2) is provided as SEQ ID NO: 37. (referencing UniProtKB ID Q9P1W8 Isoform 2).

(SEQ ID NO: 37)
MIQPEKLLLVTVGKTATLHCTVTSLLPVGPVLWFRGVGPGRELIYNQKEG
HFPRVTTVSDLTKRNNMDFSIRISSITPADVGTYYCVKFRKGSPENVEFK
SGPGTEMALGAKPSAPVVLGPAARTTPEHTVSFTCESHGFSPRDITLKWF
KNGNELSDFQTNVDPTGQSVAYSIRSTARVVLDPWDVRSQVICEVAHVTL
QGDPLRGTANLSEAIRVPPTLEVTQQPMRVGNQVNVTCQVRKFYPQSLQL
TWSENGNVCQRETASTLTENKDGTYNWTSWFLVNISDQRDDVVLTCQVKH
DGQLAVSKRLALEVTVHQKDQSSDATPGPASSLTALLLIAVLLGPIYVPW
KQKT

The amino acid sequence of a splice variant hSIRPγ (isoform 3) is provided as SEQ ID NO: 2. (referencing UniProtKB ID Q9P1W8 Isoform 3).

(SEQ ID NO: 2)
MPVPASWPHPPGPFLLLTLLLGLTEVAGEEELQMIQPEKLLLVTVGKTAT
LHCTVTSLLPVGPVLWERGVGPGRELIYNQKEGHFPRVTTVSDLTKRNNM
DFSIRISSITPADVGTYYCVKFRKGSPENVEFKSGPGTEMALGGPASSLT
ALLLIAVLLGPIYVPWKQKT

The amino acid sequence of another splice variant hSIRPγ (isoform 4) is provided as SEQ ID NO: 3 (referencing UniProtKB ID Q9P1W8 Isoform 4).

(SEQ ID NO: 3)
MPVPASWPHPPGPFLLLTLLLGLTEVAGEEELQMIQPEKLLLVTVGKTAT
LHCTVTSLLPVGPVLWFRGVGPGRELIYNQKEGHFPRVTTVSDLTKRNNM
DFSIRISSITPADVGTYYCVKFRKGSPENVEFKSGPGTEMALGAKPSAPV
VLGPAARTTPEHTVSFTCESHGFSPRDITLKWFKNGNELSDFQTNVDPTG
QSVAYSIRSTARVVLDPWDVRSQVICEVAHVTLQGDPLRGTANLSEAIRG
PASSLTALLLIAVLLGPIYVPWKQKT

The amino acid sequence of cynomolgus monkey SIRPγ is provided as SEQ ID NO: 4. (referencing GenBank: EHH65481.1).

(SEQ ID NO: 4)
MPVPASWSHPPGPFLLLTLLLGFTEVAGEEELQMIQPEKLLLVAVGESA
TLNCTVTSLLPVGPVLWFRGVGPGRELIYNQKEGHFPRVTTVSDLTKRN
NMDFSIRISSITPADAGTYYCVKFRKGSPENVEFKSGPGTEMALRAKPS
APVVSGPAARATPEHTVSFTCKSHGFSPRDITLKWFKNGNELSDFQTNV
DPAGQSVSYSIRSTARVVLAPWDVRSQVTCEVAHVTLQGDPLRGTANLS
EAIRVPPTLEVTQQPMRAGNQVNITYQVRNFYPQNLQLTWLENGNVCRT
ETASTLTENKDGTYNWTSWLLVNTSDQRDDVVLTCQVKHDGQLAVNKSL
VLEVSAHQKDQSSDATHGPASSLTVLLLIAVLLGPIYVPWKQKS

Accordingly, the SIRPγ antibodies of the disclosure may bind to one or more isoforms of a SIRPγ of a single species. In some embodiments, the SIRPγ antibodies also bind to one or more isoforms of a SIRPγ of more than one species. In some embodiments, the SIRPγ antibodies bind to one or more isoforms of human SIRPγ. In some embodiments, the SIRPγ antibodies also bind to one or more isoforms of a non-human primate SIRPγ, e.g. a cynomolgus monkey SIRPγ.

In some embodiments, the SIRPγ antibodies bind to a plurality of SIRPγ variants or isoforms found in a particular species, e.g. the SIRPγ antibodies bind to more than one of SIRPγ human isoforms 1-3. In some embodiments, the SIRPγ antibodies bind the extracellular domain of SIRPγ (e.g. amino acids 1-360 of SEQ ID NO: 1).

In some embodiments, a SIRPγ antibody of the disclosure binds to a plurality of SIRPγ isoforms found in a particular species, e.g. the SIRPγ antibody binds to more than one SIRPγ human isoform (e.g. the antibody binds to the full length and one or more splice variants). In other embodiments, the SIRPγ antibody binds to a some but not all SIRPγ isoforms found in a particular species (e.g. the SIRPγ antibody binds to one splice variant, but not all).

The skilled artisan will recognize that antibodies that exhibit little or no binding to a target antigen can be described as having a low affinity, and a high equilibrium dissociation constant (KD) for the target antigen, for example a KD of about 10 μM or greater, about 100 μM or greater, about 1 mM or greater, or about 10 mM or greater. The skilled artisan will also recognize that antibodies that exhibit little or no binding to a target antigen can be described as having a low affinity, and a high equilibrium dissociation constant (KD) for the target antigen, for example a KD of about 10 μM or greater, about 100 μM or greater, about 1 mM or greater, or about 10 mM or greater. For example, a SIRPγ antibody that binds to SIRPγ may bind to SIRPβ1 and/or SIRPα with low affinity. A SIRPγ antibody of the disclosure with low affinity for SIRPβ1 and/or SIRPα may bind to SIRPβ1 and/or SIRPα with a KD of about 10 μM or greater, about 100 μM or greater, about 1 mM or greater, or about 10 mM or greater but retain higher binding affinity for SIRPγ.

In some embodiments, provided herein are SIRPγ antibodies comprising a binding affinity (KD) to SIRPγ of about 0.0001 nM, about 0.0005 nM, about 0.001 nM, about 0.005 nM, about 01M, about 0.05 nM, about 0.1 nM, about 0.5 nM, about 1 nM, about 5 nM, about 10 nM, about 50 nM, about 100 nM, about 500 nM, about 1 μM, about 2 μM or about 3 μM.

In some embodiments, provided herein are SIRPγ antibodies comprising a binding affinity (KD) to SIRPγ of between about 0.0001 nM and 5 μM, between about 0.0005 nM and 5 μM, between about 0.05 nM and 5 μM, between about 0.5 nM and 5 μM, between about mM and 5 μM, between about 5 nM and 5 μM, 0.0001 nM and 2 μM, between about 0.0005 nM and 2 μM, between about 0.05 nM and 2 μM, between about 0.5 nM and 2 μM, between about mM and 2 μM, between about 5 nM and 2 μM, 0.0001 nM and 1 μM, between about 0.0005 nM and 1 μM, between about 0.05 nM and 1 μM, between about 0.5 nM and 1 μM, between about mM and 1 μM, between about 5 nM and 1 μM, between about 0.0001 nM and 500 nM, between about 0.0005 nM and 500 nM, between about 0.05 nM and 500 nM, between about 0.5 nM and 500 nM, between about 1 nM and 500 nM, between about 5 nM and 500 nM, between about 0.0001 nM and 50 nM, between about 0.0005 nM and 50 nM, between about 0.05 nM and 50 nM, between about 0.5 nM and 50 nM, between about 1 nM and 50 nM, or between about 5 nM and 50 nM.

In some embodiments, a SIRPγ antibody of the disclosure does not disrupt CD47 binding to SIRPγ on a cell or other surface. Exemplary antibodies that do not disrupt CD47 binding to SIRPγ include an antibody comprising the following sequences, making reference to Table 1 below:
a. the CDR-H1 amino acid sequence of SEQ ID NO: 210; the CDR-H2 amino acid sequence of SEQ ID NO: 246; the CDR-H3 amino acid sequence of SEQ ID NO: 278; the CDR-L1 amino acid sequence of SEQ ID NO: 101; the CDR-L2 amino acid sequence of SEQ ID NO: 139; and the CDR-L3 amino acid sequence of SEQ ID NO: 168.
b. the CDR-H1 amino acid sequence of SEQ ID NO: 213; the CDR-H2 amino acid sequence of SEQ ID NO: 249; the CDR-H3 amino acid sequence of SEQ ID NO: 290; the CDR-L1 amino acid sequence of SEQ ID NO: 112; the CDR-L2 amino acid sequence of SEQ ID NO: 148; and the CDR-L3 amino acid sequence of SEQ ID NO: 179.
c. the CDR-H1 amino acid sequence of SEQ ID NO: 234; the CDR-H2 amino acid sequence of SEQ ID NO: 249; the CDR-H3 amino acid sequence of SEQ ID NO: 308; the CDR-L1 amino acid sequence of SEQ ID NO: 127; the CDR-L2 amino acid sequence of SEQ ID NO: 155; and the CDR-L3 amino acid sequence of SEQ ID NO: 196.
d. the CDR-H1 amino acid sequence of SEQ ID NO: 243; the CDR-H2 amino acid sequence of SEQ ID NO: 249; the CDR-H3 amino acid sequence of SEQ ID NO: 321; the CDR-L1 amino acid sequence of SEQ ID NO: 136; the CDR-L2 amino acid sequence of SEQ ID NO: 164; and the CDR-L3 amino acid sequence of SEQ ID NO: 207.

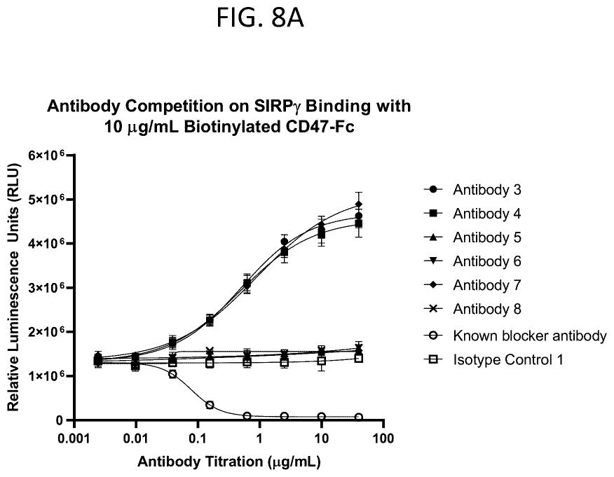

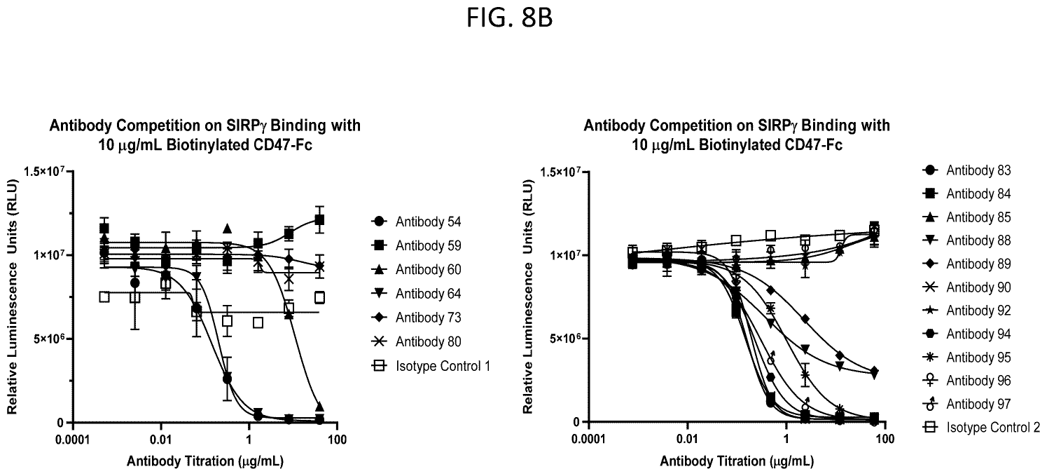

Exemplary antibodies of the disclosure that do not disrupt CD47 binding to SIRPγ include Antibodies 5, 6, 8, 59, 73, 80, 85, 92, and 96 (depicted in FIGS. 8A and 8B). The CDR and VH/VL sequences for these antibodies are provided in Tables 1 and 2.

In other embodiments, a SIRPγ antibody of the disclosure disrupts the binding of CD47 to SIRPγ on a cell or other surface. In other embodiments, a SIRPγ antibody of the disclosure enhances or promotes the binding of CD47 to SIRPγ on a cell or other surface. Exemplary antibodies that do not disrupt CD47 binding to SIRPγ include an antibody comprising the following sequences, making reference to Table 2: an antibody that comprises the CDR-H1 amino acid sequence of SEQ ID NO: 209; the CDR-H2 amino acid sequence of SEQ ID NO: 245; the CDR-H3 amino acid sequence of SEQ ID NO: 277; the CDR-L1 amino acid sequence of SEQ ID NO: 100; the CDR-L2 amino acid sequence of SEQ ID NO: 138; and the CDR-L3 amino acid sequence of SEQ ID NO: 167. Exemplary antibodies of the disclosure that enhance or promote CD47 binding to SIRPγ include Antibodies 3, 4, and 7. The CDR and VH/VL sequences for these antibodies are provided in Tables 1 and 2.

Also provided herein are Fc-containing SIRPγ antibodies. In some embodiments, Fc domain of (interchangeably referred to as a Fc sequence, Fc region, or simply Fc) of the SIRPγ antibody is a human Fc domain. In some embodiments, the Fc domain of a SIRPγ antibody is human IgG1, human IgG2, human IgG3, or human IgG4. In some embodiments, the Fc domain of a SIRPγ antibody is that of a mouse. In some embodiments, the Fc domain of a SIRPγ antibody is mouse IgG1 or mouse IgG2a. In some embodiments, the Fc domain of a SIRPγ antibody is that of a rat. In some embodiments, the Fc domain of a SIRPγ antibody is rat IgG1 or rat IgG2b. In embodiments, the Fc domain of a SIRPγ antibody is that of a non-human primate, e.g. it is a cynomolgus monkey Fc domain.

In some embodiments, the SIRPγ antibodies provided herein are full-length antibodies (comprising an intact tetrameric antibody containing two light chains and two heavy chains, each with a variable region, and a constant region). In some embodiments, the constant region of the full-length SIRPγ antibodies comprises a human Fc domain. In some embodiments, the Fc domain of a full-length SIRPγ antibody is from a human IgG1, human IgG2, human IgG3, or human IgG4. In some embodiments, the Fc domain of a full-length SIRPγ antibody is that of a mouse immunoglobulin. In some embodiments, the Fc domain of a full-length SIRPγ antibody is that of a mouse IgG1 or mouse IgG2a. In some embodiments, the Fc domain of a full-length SIRPγ antibody is that of a rat. In some embodiments, the Fc domain of a full-length SIRPγ antibody is from a rat IgG1 or rat IgG2b. In embodiments, the Fc domain of a full-length SIRPγ antibody is that of a non-human primate, e.g. it is a cynomolgus monkey Fc domain.

In some embodiments, the SIRPγ antibody contains an Fc domain (referred to as an “Fc-containing antibody”), wherein binding of the Fc-containing antibody to a SIRPγ-expressing cell can mediate effector cell-mediated depletion of the SIRPγ-expressing cell. In some embodiments, the Fc domain of a SIRPγ antibody is a human IgG1 Fc. Exemplary, but non-limiting, sequences of heavy chain constant regions (CH) of human IgG1 encompassing Fc domains of interest are provided as SEQ ID NO: 5-27, and SEQ ID NO: 36. SEQ ID NO: 5 provides the canonical human IgG1 heavy chain constant region (CH) sequence.

| (SEQ ID NO: 5) | |
|---|---|
| 1 | ASTKGPSVFP LAPSSKSTSG GTAALGCLVK DYFPEPVTVS WNSGALTSGV HTFPAVLQSS |
| 61 | GLYSLSSVVT VPSSSLGTQT YICNVNHKPS NTKVDKKVEP KSCDKTHTCP PCPAPELLGG |
| 121 | PSVFLFPPKP KDTLMISRTP EVTCVVVDVS HEDPEVKFNW YVDGVEVHNA KTKPREEQYN |
| 181 | STYRVVSVLT VLHQDWLNGK EYKCKVSNKA LPAPIEKTIS KAKGQPREPQ VYTLPPSRDE |
| 241 | LTKNQVSLTC LVKGFYPSDI AVEWESNGQP ENNYKTTPPV LDSDGSFFLY SKLTVDKSRW |
| 301 | QQGNVFSCSV MHEALHNHYT QKSLSLSPGK |
| (SEQ ID NO: 6) | |
| 1 | ASTKGPSVFP LAPSSKSTSG GTAALGCLVK DYFPEPVTVS WNSGALTSGV HTFPAVLQSS |
| 61 | GLYSLSSVVT VPSSSLGTQT YICNVNHKPS NTKVDKKAEP KSCDKTHTCP PCPAPELLGG |
| 121 | PSVFLFPPKP KDTLMISRTP EVTCVVVDVS HEDPEVKFNW YVDGVEVHNA KTKPREEQYN |
| 181 | STYRVVSVLT VLHQDWLNGK EYKCKVSNKA LPAPIEKTIS KAKGQPREPQ VYTLPPSRDE |
| 241 | LTKNQVSLTC LVKGFYPSDI AVEWESNGQP ENNYKTTPPV LDSDGSFFLY SKLTVDKSRW |
| 301 | QQGNVFSCSV MHEALHNHYT QKSLSLSPGK |
| (SEQ ID NO: 7) | |
| 1 | ASTKGPSVFP LAPSSKSTSG GTAALGCLVK DYFPEPVTVS WNSGALTSGV HTFPAVLQSS |
| 61 | GLYSLSSVVT VPSSSLGTQT YICNVNHKPS NTKVDKKAEP KSCDKTHTCP PCPAPELLAG |
| 121 | PDVFLFPPKP KDTLMISRTP EVTCVVVDVS HEDPEVKFNW YVDGVEVHNA KTKPREEQYN |
| 181 | STYRVVSVLT VLHQDWLNGK EYKCKVSNKA LPAPEEKTIS KAKGQPREPQ VYTLPPSRDE |
| 241 | LTKNQVSLTC LVKGFYPSDI AVEWESNGQP ENNYKTTPPV LDSDGSFFLY SKLTVDKSRW |
| 301 | QQGNVFSCSV MHEALHNHYT QKSLSLSPGK |
| (SEQ ID NO: 8) | |
| 1 | ASTKGPSVFP LAPSSKSTSG GTAALGCLVK DYFPEPVTVS WNSGALTSGV HTFPAVLQSS |
| 61 | GLYSLSSVVT VPSSSLGTQT YICNVNHKPS NTKVDKKVEP KSCDKTHTCP PCPAPELLAG |
| 121 | PDVFLFPPKP KDTLMISRTP EVTCVVVDVS HEDPEVKFNW YVDGVEVHNA KTKPREEQYN |
| 181 | STYRVVSVLT VLHQDWLNGK EYKCKVSNKA LPAPEEKTIS KAKGQPREPQ VYTLPPSRDE |
| 241 | LTKNQVSLTC LVKGFYPSDI AVEWESNGQP ENNYKTTPPV LDSDGSFFLY SKLTVDKSRW |
| 301 | QQGNVFSCSV MHEALHNHYT QKSLSLSPGK |
| (SEQ ID NO: 9) | |
| 1 | ASTKGPSVFP LAPSSKSTSG GTAALGCLVK DYFPEPVTVS WNSGALTSGV HTFPAVLQSS |
| 61 | GLYSLSSVVT VPSSSLGTQT YICNVNHKPS NTKVDKKVEP KSCDKTHTCP PCPAPELLGG |
| 121 | PDVFLFPPKP KDTLMISRTP EVTCVVVDVS HEDPEVKFNW YVDGVEVHNA KTKPREEQYN |
| 181 | STYRVVSVLT VLHQDWLNGK EYKCKVSNKA LPAPEEKTIS KAKGQPREPQ VYTLPPSRDE |
| 241 | LTKNQVSLTC LVKGFYPSDI AVEWESNGQP ENNYKTTPPV LDSDGSFFLY SKLTVDKSRW |
| 301 | QQGNVFSCSV MHEALHNHYT QKSLSLSPGK |
| (SEQ ID NO: 10) | |
| 1 | ASTKGPSVFP LAPSSKSTSG GTAALGCLVK DYFPEPVTVS WNSGALTSGV HTFPAVLQSS |
| 61 | GLYSLSSVVT VPSSSLGTQT YICNVNHKPS NTKVDKKVEP KSCDKTHTCP PCPAPELLGG |
| 121 | PSVFLFPPKP KDTLMISRTP EVTCVVVDVS HEDPEVKFNW YVDGVEVHNA KTKPREEQYN |
| 181 | STYRVVSVLT VLHQDWLNGK EYKCKVSNKA LPAPEEKTIS KAKGQPREPQ VYTLPPSRDE |
| 241 | LTKNQVSLTC LVKGFYPSDI AVEWESNGQP ENNYKTTPPV LDSDGSFFLY SKLTVDKSRW |
| 301 | QQGNVFSCSV MHEALHNHYT QKSLSLSPGK |
| (SEQ ID NO: 11) | |
| 1 | ASTKGPSVFP LAPSSKSTSG GTAALGCLVK DYFPEPVTVS WNSGALTSGV HTFPAVLQSS |
| 61 | GLYSLSSVVT VPSSSLGTQT YICNVNHKPS NTKVDKKVEP KSCDKTHTCP PCPAPELLGG |
| 121 | PDVFLFPPKP KDTLMISRTP EVTCVVVDVS HEDPEVKFNW YVDGVEVHNA KTKPREEQYN |
| 181 | STYRVVSVLT VLHQDWLNGK EYKCKVSNKA LPAPIEKTIS KAKGQPREPQ VYTLPPSRDE |
| 241 | LTKNQVSLTC LVKGFYPSDI AVEWESNGQP ENNYKTTPPV LDSDGSFFLY SKLTVDKSRW |
| 301 | QQGNVFSCSV MHEALHNHYT QKSLSLSPGK |
| (SEQ ID NO: 12) | |
| 1 | ASTKGPSVFP LAPSSKSTSG GTAALGCLVK DYFPEPVTVS WNSGALTSGV HTFPAVLQSS |
| 61 | GLYSLSSVVT VPSSSLGTQT YICNVNHKPS NTKVDKKAEP KSCDKTHTCP PCPAPELLGG |
| 121 | PSVFLFPPKP KDTLMISRTP EVTCVVVDVS HEDPEVKFNW YVDGVEVHNA KTKPREEQYN |
| 181 | STYRVVSVLT VLHQDWLNGK EYKCKVSNKA LPAPIEKTIS KAKGQPREPQ VYTLPPSRDE |
| 241 | LTKNQVSLTC LVKGFYPSDI AVEWESNGQP ENNYKTTPPV LDSDGSFFLY SKLTVDKSRW |
| 301 | QQGNVFSCSV MHEALHNHYT QKSLSLSPGK |
| (SEQ ID NO: 13) | |
| 1 | ASTKGPSVFP LAPSSKSTSG GTAALGCLVK DYFPEPVTVS WNSGALTSGV HTFPAVLQSS |
| 61 | GLYSLSSVVT VPSSSLGTQT YICNVNHKPS NTKVDKKVEP KSCDKTHTCP PCPAPELLGG |
| 121 | PSVFLFPPKP KDTLMISRTP EVTCVVVDVS HEDPEVKFNW YVDGVEVHNA KTKPREEQYN |
| 181 | STYRVVSVLT VLHQDWLNGK EYKCKVSNKA LPAPIEKTIS KAKGQPREPQ VYTLPPSRDE |
| 241 | LTKNQVSLTC LVKGFYPSDI AVEWESNGQP ENNYKTTPPV LDSDGSFFLY SKLTVDKSRW |
| 301 | QQGNVFSCSV LHEALHNHYT QKSLSLSPGK |
| (SEQ ID NO: 14) | |
| 1 | ASTKGPSVFP LAPSSKSTSG GTAALGCLVK DYFPEPVTVS WNSGALTSGV HTFPAVLQSS |
| 61 | GLYSLSSVVT VPSSSLGTQT YICNVNHKPS NTKVDKKVEP KSCDKTHTCP PCPAPELLGG |
| 121 | PSVFLFPPKP KDTLMISRTP EVTCVVVDVS HEDPEVKFNW YVDGVEVHNA KTKPREEQYN |
| 181 | STYRVVSVLT VLHQDWLNGK EYKCKVSNKA LPAPIEKTIS KAKGQPREPQ VYTLPPSRDE |
| 241 | LTKNQVSLTC LVKGFYPSDI AVEWESNGQP ENNYKTTPPV LDSDGSFFLY SKLTVDKSRW |
| 301 | QQGNVFSCSV MHEALHSHYT QKSLSLSPGK |
| (SEQ ID NO: 15) | |
| 1 | ASTKGPSVFP LAPSSKSTSG GTAALGCLVK DYFPEPVTVS WNSGALTSGV HTFPAVLQSS |
| 61 | GLYSLSSVVT VPSSSLGTQT YICNVNHKPS NTKVDKKAEP KSCDKTHTCP PCPAPELLGG |
| 121 | PSVFLFPPKP KDTLMISRTP EVTCVVVDVS HEDPEVKFNW YVDGVEVHNA KTKPREEQYN |
| 181 | STYRVVSVLT VLHQDWLNGK EYKCKVSNKA LPAPIEKTIS KAKGQPREPQ VYTLPPSRDE |
| 241 | LTKNQVSLTC LVKGFYPSDI AVEWESNGQP ENNYKTTPPV LDSDGSFFLY SKLTVDKSRW |
| 301 | QQGNVFSCSV LHEALHNHYT QKSLSLSPGK |
| (SEQ ID NO: 16) | |
| 1 | ASTKGPSVFP LAPSSKSTSG GTAALGCLVK DYFPEPVTVS WNSGALTSGV HTFPAVLQSS |
| 61 | GLYSLSSVVT VPSSSLGTQT YICNVNHKPS NTKVDKKAEP KSCDKTHTCP PCPAPELLGG |
| 121 | PSVFLFPPKP KDTLMISRTP EVTCVVVDVS HEDPEVKFNW YVDGVEVHNA KTKPREEQYN |
| 181 | STYRVVSVLT VLHQDWLNGK EYKCKVSNKA LPAPIEKTIS KAKGQPREPQ VYTLPPSRDE |
| 241 | LTKNQVSLTC LVKGFYPSDI AVEWESNGQP ENNYKTTPPV LDSDGSFFLY SKLTVDKSRW |
| 301 | QQGNVFSCSV MHEALHSHYT QKSLSLSPGK |
| (SEQ ID NO: 17) | |
| 1 | ASTKGPSVFP LAPSSKSTSG GTAALGCLVK DYFPEPVTVS WNSGALTSGV HTFPAVLQSS |
| 61 | GLYSLSSVVT VPSSSLGTQT YICNVNHKPS NTKVDKRVEP KSCDKTHTCP PCPAPELLGG |
| 121 | PSVFLFPPKP KDTLMISRTP EVTCVVVDVS HEDPEVKFNW YVDGVEVHNA KTKPREEQYN |
| 181 | STYRVVSVLT VLHQDWLNGK EYKCKVSNKA LPAPIEKTIS KAKGQPREPQ VYTLPPSRDE |
| 241 | LTKNQVSLTC LVKGFYPSDI AVEWESNGQP ENNYKTTPPV LDSDGSFFLY SKLTVDKSRW |
| 301 | QQGNVFSCSV MHEALHNHYT QKSLSLSPGK |
| (SEQ ID NO: 18) | |
| 1 | ASTKGPSVFP LAPSSKSTSG GTAALGCLVK DYFPEPVTVS WNSGALTSGV HTFPAVLQSS |
| 61 | GLYSLSSVVT VPSSSLGTQT YICNVNHKPS NTKVDKRVEP KSCDKTHTCP PCPAPELLGG |
| 121 | PSVFLFPPKP KDTLMISRTP EVTCVVVDVS HEDPEVKFNW YVDGVEVHNA KTKPREEQYN |
| 181 | STYRVVSVLT VLHQDWLNGK EYKCKVSNKA LPAPIEKTIS KAKGQPREPQ VYTLPPSREE |
| 241 | LTKNQVSLTC LVKGFYPSDI AVEWESNGQP ENNYKTTPPV LDSDGSFFLY SKLTVDKSRW |
| 301 | QQGNVFSCSV MHEALHNHYT QKSLSLSPGK |
| (SEQ ID NO: 19) | |
| 1 | ASTKGPSVFP LAPSSKSTSG GTAALGCLVK DYFPEPVTVS WNSGALTSGV HTFPAVLQSS |
| 61 | GLYSLSSVVT VPSSSLGTQT YICNVNHKPS NTKVDKKVEP KSCDKTHTCP PCPAPELLGG |
| 121 | PSVFLFPPKP KDTLMISRTP EVTCVVVDVS HEDPEVKFNW YVDGVEVHNA KTKPREEQYN |
| 181 | STYRVVSVLT VLHQDWLNGK EYKCKVSNKA LPAPIEKTIS KAKGQPREPQ VYTLPPSREE |
| 241 | LTKNQVSLTC LVKGFYPSDI AVEWESNGQP ENNYKTTPPV LDSDGSFFLY SKLTVDKSRW |
| 301 | QQGNVFSCSV MHEALHNHYT QKSLSLSPGK |
| (SEQ ID NO: 20) | |
| 1 | ASTKGPSVFP LAPSSKSTSG GTAALGCLVK DYFPEPVTVS WNSGALTSGV HTFPAVLQSS |
| 61 | GLYSLSSVVT VPSSSLGTQT YICNVNHKPS NTKVDKRVEP KSCDKTHTCP PCPAPELLGG |
| 121 | PSVFLFPPKP KDTLMISRTP EVTCVVVDVS HEDPEVKFNW YVDGVEVHNA KTKPREEQYN |
| 181 | STYRVVSVLT VLHQDWLNGK EYKCKVSNKA LPAPIEKTIS KAKGQPREPQ VYTLPPSRDE |
| 241 | MTKNQVSLTC LVKGFYPSDI AVEWESNGQP ENNYKTTPPV LDSDGSFFLY SKLTVDKSRW |
| 301 | QQGNVFSCSV MHEALHNHYT QKSLSLSPGK |
| (SEQ ID NO: 21) | |
| 1 | ASTKGPSVFP LAPSSKSTSG GTAALGCLVK DYFPEPVTVS WNSGALTSGV HTFPAVLQSS |
| 61 | GLYSLSSVVT VPSSSLGTQT YICNVNHKPS NTKVDKKAEP KSCDKTHTCP PCPAPELLGG |
| 121 | PSVFLFPPKP KDTLMISRTP EVTCVVVDVS HEDPEVKFNW YVDGVEVHNA KTKPREEQYN |
| 181 | STYRVVSVLT VLHQDWLNGK EYKCKVSNKA LPAPIEKTIS KAKGQPREPQ VYTLPPSRDE |
| 241 | MTKNQVSLTC LVKGFYPSDI AVEWESNGQP ENNYKTTPPV LDSDGSFFLY SKLTVDKSRW |
| 301 | QQGNVFSCSV MHEALHNHYT QKSLSLSPGK |
| (SEQ ID NO: 22) | |
| 1 | ASTKGPSVFP LAPSSKSTSG GTAALGCLVK DYFPEPVTVS WNSGALTSGV HTFPAVLQSS |
| 61 | GLYSLSSVVT VPSSSLGTQT YICNVNHKPS NTKVDKKAEP KSCDKTHTCP PCPAPELLGG |
| 121 | PSVFLFPPKP KDTLMISRTP EVTCVVVDVS HEDPEVKFNW YVDGVEVHNA KTKPREEQYN |
| 181 | STYRVVSVLT VLHQDWLNGK EYKCKVSNKA LPAPEEKTIS KAKGQPREPQ VYTLPPSRDE |
| 241 | LTKNQVSLTC LVKGFYPSDI AVEWESNGQP ENNYKTTPPV LDSDGSFFLY SKLTVDKSRW |
| 301 | QQGNVFSCSV MHEALHNHYT QKSLSLSPGK |

In some embodiments, the constant region of human IgG1 heavy chain sequence encompassing a Fc domain of interest is SEQ ID NO: 23, wherein X1 is V or A.

| (SEQ ID NO: 23) | |
|---|---|
| 1 | ASTKGPSVFP LAPSSKSTSG GTAALGCLVK DYFPEPVTVS WNSGALTSGV HTFPAVLQSS |
| 61 | GLYSLSSVVT VPSSSLGTQT YICNVNHKPS NTKVDKKX₁EP KSCDKTHTCP PCPAPELLAG |
| 121 | PDVFLFPPKP KDTLMISRTP EVTCVVVDVS HEDPEVKFNW YVDGVEVHNA KTKPREEQYN |
| 181 | STYRVVSVLT VLHQDWLNGK EYKCKVSNKA LPAPEEKTIS KAKGQPREPQ VYTLPPSRDE |
| 241 | LTKNQVSLTC LVKGFYPSDI AVEWESNGQP ENNYKTTPPV LDSDGSFFLY SKLTVDKSRW |
| 301 | QQGNVFSCSV MHEALHNHYT QKSLSLSPGK |

In some embodiments, the constant region of human IgG1 heavy chain sequence encompassing a Fc domain of interest is SEQ ID NO: 24, wherein X1 is V or A; X2 is G or A; X3 is S or D; and X4 is I or E.

| (SEQ ID NO: 24) | |
|---|---|
| 1 | ASTKGPSVFP LAPSSKSTSG GTAALGCLVK DYFPEPVTVS WNSGALTSGV HTFPAVLQSS |
| 61 | GLYSLSSVVT VPSSSLGTQT YICNVNHKPS NTKVDKKX₁EP KSCDKTHTCP PCPAPELLX₂G |
| 121 | PX₃VFLFPPKP KDTLMISRTP EVTCVVVDVS HEDPEVKFNW YVDGVEVHNA KTKPREEQYN |
| 181 | STYRVVSVLT VLHQDWLNGK EYKCKVSNKA LPAPX₄EKTIS KAKGQPREPQ VYTLPPSRDE |
| 241 | LTKNQVSLTC LVKGFYPSDI AVEWESNGQP ENNYKTTPPV LDSDGSFFLY SKLTVDKSRW |
| 301 | QQGNVFSCSV MHEALHNHYT QKSLSLSPGK |

In some embodiments, the constant region of human IgG1 heavy chain sequence encompassing a Fc domain of interest is SEQ ID NO: 25, wherein X1 is V or A.

| (SEQ ID NO: 25) | |
|---|---|
| 1 | ASTKGPSVFP LAPSSKSTSG GTAALGCLVK DYFPEPVTVS WNSGALTSGV HTFPAVLQSS |
| 61 | GLYSLSSVVT VPSSSLGTQT YICNVNHKPS NTKVDKKX₁EP KSCDKTHTCP PCPAPELLGG |
| 121 | PSVFLFPPKP KDTLMISRTP EVTCVVVDVS HEDPEVKFNW YVDGVEVHNA KTKPREEQYN |
| 181 | STYRVVSVLT VLHQDWLNGK EYKCKVSNKA LPAPIEKTIS KAKGQPREPQ VYTLPPSRDE |
| 241 | LTKNQVSLTC LVKGFYPSDI AVEWESNGQP ENNYKTTPPV LDSDGSFFLY SKLTVDKSRW |
| 301 | QQGNVFSCSV LHEALHSHYT QKSLSLSPGK |

In some embodiments, the constant region of human IgG1 heavy chain sequence encompassing a Fc domain of interest is SEQ ID NO: 26, wherein X1 is V or A; X2 is M or L; and X3 is N or S.

| (SEQ ID NO: 26) | |
|---|---|
| 1 | ASTKGPSVFP LAPSSKSTSG GTAALGCLVK DYFPEPVTVS WNSGALTSGV HTFPAVLQSS |
| 61 | GLYSLSSVVT VPSSSLGTQT YICNVNHKPS NTKVDKKX₁EP KSCDKTHTCP PCPAPELLGG |
| 121 | PSVFLFPPKP KDTLMISRTP EVTCVVVDVS HEDPEVKFNW YVDGVEVHNA KTKPREEQYN |
| 181 | STYRVVSVLT VLHQDWLNGK EYKCKVSNKA LPAPIEKTIS KAKGQPREPQ VYTLPPSRDE |
| 241 | LTKNQVSLTC LVKGFYPSDI AVEWESNGQP ENNYKTTPPV LDSDGSFFLY SKLTVDKSRW |
| 301 | QQGNVFSCSV X₂HEALHX₃HYT QKSLSLSPGK |

In some embodiments, the constant region of human IgG1 heavy chain sequence encompassing a Fc domain of interest is SEQ ID NO: 27, wherein X1 is K or R; X2 is D or E; and X3 is L or M.

| (SEQ ID NO: 27) | |
|---|---|
| 1 | ASTKGPSVFP LAPSSKSTSG GTAALGCLVK DYFPEPVTVS WNSGALTSGV HTFPAVLQSS |
| 61 | GLYSLSSVVT VPSSSLGTQT YICNVNHKPS NTKVDKX₁VEP KSCDKTHTCP PCPAPELLGG |
| 121 | PSVFLFPPKP KDTLMISRTP EVTCVVVDVS HEDPEVKFNW YVDGVEVHNA KTKPREEQYN |
| 181 | STYRVVSVLT VLHQDWLNGK EYKCKVSNKA LPAPIEKTIS KAKGQPREPQ VYTLPPSRX₂E |
| 241 | X₃TKNQVSLTC LVKGFYPSDI AVEWESNGQP ENNYKTTPPV LDSDGSFFLY SKLTVDKSRW |
| 301 | QQGNVFSCSV MHEALHNHYT QKSLSLSPGK |

In some embodiments, the constant region of human IgG1 heavy chain sequence encompassing a Fc domain of interest is SEQ ID NO: 36, and comprises L234A, L235A, P329G substitutions (referred to as LALA-PG substitutions).

(SEQ ID NO: 36)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPEAAGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALGAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGK

In some embodiments, the Fc domain of a Fc-containing SIRPγ antibody is a human IgG4 Fc. Exemplary, but non-limiting, sequences of heavy chain constant regions (CH) of human IgG4 encompassing Fc domains of interest are provided as SEQ ID NO: 28-35. SEQ ID NO: 28 provides the canonical human IgG4 heavy chain constant region (CH) sequence.

(SEQ ID NO: 28)
ASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRV
ESKYGPPCPSCPAPEFLGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK
(SEQ ID NO: 29)
ASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRV
ESKYGPPCPPCPAPEFEGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK
(SEQ ID NO: 30)
ASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRV
ESKYGPPCPSCPAPEFEGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK
(SEQ ID NO: 31)
ASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRV
ESKYGPPCPPCPAPEFLGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK
(SEQ ID NO: 32)
ASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRV
ESKYGPPCPSCPAPEALGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK
(SEQ ID NO: 33)
ASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRV
ESKYGPPCPSCPAPEAAGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK
(SEQ ID NO: 34)
ASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRV
ESKYGPPCPSCPAPEFAGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK

In some embodiments, the constant region of human IgG4 heavy chain sequence encompassing a Fc domain of interest is SEQ ID NO: 35, wherein X1 is S or P; AND X2 is L or E.

(SEQ ID NO: 35)
ASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRV
ESKYGPPCPX₁CPAPEFX₂GGPSVFLFPPKPKDTLMISRTPEVTCVVVD
VSQEDPEVQFNWYVDGVEVHNAKTKPREEQFNSTYRVVSVLTVLHQDWL
NGKEYKCKVSNKGLPSSIEKTISKAKGQPREPQVYTLPPSQEEMTKNQV
SLTCLVKGFYPSDIAVEWESNGQPENNYKTTPPVLDSDGSFFLYSRLTV
DKSRWQEGNVFSCSVMHEALHNHYTQKSLSLSLGK

In some embodiments, the SIRPγ antibodies provided herein are chimeric and comprise a variable region from one species, and a constant region from another species, e.g. comprise a human variable region and a rat constant region. In some embodiments, the rat constant region comprises sequences from a rat IgG1 or rat IgG2b. In some embodiments, the antibodies comprise a human variable region and a mouse constant region. In some embodiments, the mouse constant region is mouse IgG1, or mouse IgG2a. In some embodiments, the antibodies comprise a human variable region and a human constant region. In exemplary embodiments, the human constant region comprises sequences from human IgG1, human IgG2, human IgG3, or human IgG4.

The EU numbering scheme is one of many available antibody numbering schemes based on the residue numbers assigned to a canonical antibody sequence. Accordingly, a skilled artisan would understand that reference to a particular residue using the EU numbering scheme may or may not be exactly the residue in one of the SIRPγ antibodies of the disclosure. For example, if a SIRPγ antibody of the disclosure comprises a V215A substitution in the Fc region of the heavy chain, wherein the position number of the amino acid residue is of the EU numbering scheme, the residue may not be the actual residue 215 in that particular SIRPγ antibody. It may be actual residue number 213, or 214, or 215, or 216 or others. Accordingly, a skilled artisan will understand how to correspond the recited residue using the EU numbering scheme, to the actual residue in a SIRPγ antibody of the disclosure. The EU numbering system for antibodies is known in the art and is described, for example, at imgt.org/IMGTScientificChart/Numbering/Hu_IGHGnber.html.

In some embodiments, the Fc domain of a SIRPγ antibody is from a human IgG1 constant heavy chain (e.g. SEQ ID NO: 5), and heavy chain Fc substitutions are introduced to increase effector function (e.g. those that exhibit increased affinity to FcγR or promote complement protein binding).

In some embodiments, the Fc domain of a SIRPγ antibody is from a human IgG1 constant heavy chain (e.g. SEQ ID NO: 5), and heavy chain Fc substitutions are introduced to decrease effector function (e.g. silence).

In some embodiments, the Fc domain of a SIRPγ antibody is from a human IgG1 constant heavy chain (e.g. SEQ ID NO: 5), and heavy chain Fc substitutions are introduced to increase antibody half-life.

In some embodiments, the Fc domain of a SIRPγ antibody is from a human IgG4 constant heavy chain (e.g. SEQ ID NO: 28), and heavy chain Fc substitutions are introduced to increase effector function (e.g. those that exhibit increased affinity to FcγR or promote complement protein binding).

In some embodiments, the Fc domain of a SIRPγ antibody is from a human IgG4 constant heavy chain (e.g. SEQ ID NO: 28), and heavy chain Fc substitutions are introduced to decrease effector function (e.g. silence).

In some embodiments, the Fc domain of a SIRPγ antibody is from a human IgG4 constant heavy chain (e.g. SEQ ID NO: 28), and heavy chain Fc substitutions are introduced to increase antibody half-life.

In some embodiments, the Fc domain of a SIRPγ antibody is an IgG1 Fc domain (e.g. the Fc domain from any one of the IgG1 constant heavy chain sequences of SEQ ID NOS: 5-27, 36) or is an IgG4 human Fc domain (e.g. the Fc domain from any one of the IgG4 constant heavy chain sequences of SEQ ID NOS: 28-35).

In some embodiments, the Fc domain of a SIRPγ antibody is an IgG1 Fc domain (e.g. the Fc domain from any one of the IgG1 constant heavy chain sequences of SEQ ID NOS: 5-27, or 36) or is an IgG4 human Fc domain (e.g. the Fc domain from any one of the IgG4 constant heavy chain sequences of SEQ ID NOS: 28, 29 or 35), and comprises at least one amino acid substitution in the heavy chain at a position selected from the group consisting of: 214, 215, 221, 222, 228, 234, 235, 236, 239, 240, 241, 243, 244, 245, 247, 250, 252, 254, 256, 262, 263, 264, 265, 266, 267, 268, 269, 270, 292, 296, 297, 298, 299, 300, 305, 313, 324, 325, 326, 327, 328, 329, 330, 332, 333, 334, 345, 356, 358, 396, 428, 430, 433, 434, and 440 wherein the position numbers of the amino acid residues are of the EU numbering scheme.

In some embodiments, the Fc domain of a SIRPγ antibody is from heavy chain SEQ ID NOS: 5-27, or 36, optionally with one or more heavy chain Fc amino acid substitutions, for example at least one amino acid substitution at a position selected from the group consisting of: 214, 215, 221, 222, 228, 234, 235, 236, 239, 240, 241, 243, 244, 245, 247, 250, 252, 254, 256, 262, 263, 264, 265, 266, 267, 268, 269, 270, 292, 296, 297, 298, 299, 300, 305, 313, 324, 325, 326, 327, 328, 329, 330, 332, 333, 334, 345, 356, 358, 396, 428, 430, 433, 434, and 440 wherein the position numbers of the amino acid residues are of the EU numbering scheme. Exemplary substitutions include one or more of K214R, V215A, G236A, S239D, I332E, D356E, L358M, M428L, N434S, wherein the position numbers of the amino acid residues are of the EU numbering scheme.

In some embodiments, the Fc domain of a SIRPγ antibody is from a human IgG1 constant heavy chain (e.g. SEQ ID NO: 5-27, or 36), and heavy chain Fc substitutions are introduced to, among other effects, increase effector function (e.g. one or more of FcR binding on an immune effector cell, and binding to complement C1q), selected from the group consisting of V215A, G236A, S239D, I332E, G236A/S239D, G236A/I332E, S239D/I332E, V215A/G236A/S239D/I332E, G236A/S239D/I332E, V215A/G236A/S239D/I332E, K326W/E333 S, S267E/H268F/S324T, E345R, E430G, E345K, S440Y, K326W, E333S, S267E, H268F, S324T, and E345R/E430G/S440Y, F243L/R292P/Y300L/V305I/P396L, S239D/I332E, S298A/E333A/K334A, L234Y/L235Q/G236W/S239M/H268D/D270E/S298A, and D270E/K326D/A330M/K334E wherein the position numbers of the amino acid residues are of the EU numbering scheme.

In some embodiments, the Fc domain of a SIRPγ antibody is from a human IgG1 constant heavy chain (e.g. SEQ ID NO: 5-27, or 36), and heavy chain Fc substitutions are introduced to reduce (e.g. silence) effector function, including one or more of N297A, N297Q, N297G, L235E, L234A, L235A, K214R, P329G, D356E, and L358M, wherein the position numbers of the amino acid residues are of the EU numbering scheme.

In some embodiments, the Fc domain of a SIRPγ antibody is from a human IgG1 constant heavy chain (e.g. SEQ ID NO: 5, SEQ ID NO: 6, SEQ ID NO: 36), and heavy chain Fc substitutions are introduced to reduce effector function (e.g. silence), including L234A, L235A, and P329G, wherein the position numbers of the amino acid residues are of the EU numbering scheme.

In some embodiments, the Fc domain of a SIRPγ antibody is from a human IgG4 constant heavy chain (e.g. SEQ ID NOS: 28, 29 or 35), and heavy chain Fc substitutions are introduced to reduce effector function, including one or more of L235E, and F234A/L235A, wherein the position numbers of the amino acid residues are of the EU numbering scheme.

In some embodiments, the Fc domain of a SIRPγ antibody is from a human IgG2 constant heavy chain, and heavy chain Fc substitutions are introduced to reduce effector function, including H268Q/V309L/A330S/P331S and V234A/G237A/P238S/H268A/V309L/A330S/P331S, wherein the position numbers of the amino acid residues are of the EU numbering scheme.

In some embodiments, the Fc domain of a SIRPγ antibody is from a human IgG4 constant heavy chain (e.g. SEQ ID NO: 28), and the antibody is prone to the dynamic process of Fab-arm exchange. Accordingly, in some embodiments the IgG4 heavy chain Fc domain comprises a S228P substitution, resulting in the reduction of Fab-arm exchange, wherein the position number of the amino acid residues are of the EU numbering scheme.

In some embodiments, the Fc domain of a SIRPγ antibody is from a human IgG4 constant heavy chain (e.g. SEQ ID NO: 28, 29 or 35), and one or more of the following heavy chain Fc substitution are introduced to reduce effector function: L235A, L235E, S228P, L235E/S228P, S228P/F234A, S228P/F234A/L235A, wherein the position numbers of the amino acid residues are of the EU numbering scheme.

In other embodiments, the Fc domain of a SIRPγ antibody is altered to increase its serum half-life. Such alterations include heavy chain Fc substitutions of a human IgG1, IgG2, IgG3 or IgG4 such as M428L, N343S, T250Q/M428L, M252Y/S254T/T256E, M428L/N434S, S267E/L328F, N325S/L328F, and H433K/N434F, wherein the position number of the amino acid residues are of the EU numbering scheme.

In some embodiments the SIRPγ antibody comprises a light chain constant region, in addition to the SIRPγ antigen-binding light chain variable region, for example the exemplary CDR-containing light chain variable regions provided in Table 2. Exemplary light chain constant region amino acid sequences are provided in SEQ ID NOS: 38-42.

In some embodiments, the SIRPγ antibody contains a kappa light chain constant region. An exemplary kappa light chain constant region is provided as SEQ ID NO: 38.

(SEQ ID NO: 38)
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSENRGEC

In some embodiments, the SIRPγ antibody contains a lambda light chain constant region. Exemplary lambda light chain constant regions are provided as SEQ ID NO: 39-41.

(SEQ ID NO: 39)
GQPKANPTVTLFPPSSEELQANKATLVCLISDFYPGAVTVAWKADGSPV
KAGVETTKPSKQSNNKYAASSYLSLTPEQWKSHRSYSCQVTHEGSTVEK
TVAPTECS
(SEQ ID NO: 40)
GQPKAAPSVTLFPPSSEELQANKATLVCLISDFYPGAVTVAWKADSSPV
KAGVETTTPSKQSNNKYAASSYLSLTPEQWKSHRSYSCQVTHEGSTVEK
TVAPTECS
(SEQ ID NO: 41)
GQPKAAPSVTLFPPSSEELQANKATLVCLISDFYPGAVTVAWKADSSPV
KAGVETTTPSKQSNNKYAASSYLSLTPEQWKSHKSYSCQVTHEGSTVEK
TVAPTECS
(SEQ ID NO: 42)
GQPKAAPSVTLFPPSSEELQANKATLVCLVSDENPGAVTVAWKADGSPV
KVGVETTKPSKQSNNKYAASSYLSLTPEQWKSHRSYSCRVTHEGSTVEK
TVAPAECS

In some embodiments the SIRPγ antibody comprises a light chain constant region and heavy chain constant region, in addition to the SIRPγ antigen-binding light and heavy chain variable regions, for example the exemplary CDR-containing light chain and heavy chain variable regions provided in Table 2. Exemplary light chain constant region amino acid sequences of the disclosure are provided in SEQ ID NOS: 38-42; and exemplary heavy chain constant region amino acid sequences of the disclosure are provided in SEQ ID NOS: 5-36.

In exemplary embodiments, the SIRPγ antibodies provided herein are monoclonal antibodies (mAbs). In exemplary embodiments, the SIRPγ antibodies provided herein are human antibodies. In exemplary embodiments, the SIRPγ antibodies provided herein are humanized antibodies. In exemplary embodiments, the SIRPγ antibodies provided herein are monoclonal human antibodies, or monoclonal humanized antibodies. In exemplary embodiments, the SIRPγ antibodies provided herein are chimeric antibodies. In exemplary embodiments, the SIRPγ antibodies provided herein are monoclonal chimeric antibodies. In some embodiments, the SIRPγ antibody is provided as an antibody fragment.

Also provided herein are SIRPγ antibody-drug conjugates, bispecific antibodies comprising at least one arm specific for SIRPγ, and multispecific antibodies that exhibit binding for SIRPγ.

i. Exemplary SIRPγ Antibodies—CDR Sequences

Provided herein are sequences for exemplary SIRPγ antibodies of the disclosure. Included are complementarity determining region (CDR) sequences and the variable heavy and light domain sequences (VH, VL) that constitute the SIRPγ antigen binding domains of the disclosure.

As referred below, a light chain variable (VL) domain CDR1 region is referred to as CDR-L1; a VL CDR2 region is referred to as CDR-L2; a VL CDR3 region is referred to as CDR-L3; a heavy chain variable (VH) domain CDR1 region is referred to as CDR-H1; a VH CDR2 region is referred to as CDR-H2; and a VH CDR3 region is referred to as CDR-H3. Table 1 provides 47 exemplary CDR combinations of antibodies of the disclosure.

TABLE 1
Exemplary SIRPγ Antibody CDR Combinations

| Comb- | | | | | | | |
|---|---|---|---|---|---|---|---|
| ination | Antibody | | | | | | |
| No. | No. | CDR-L1 | CDR-L2 | CDR-L3 | CDR-H1 | CDR-H2 | CDR-H3 |
| 1 | Antibody | QSVSSN | GAS | QQYNNWPLT | GFTFSNYG | IWYDGSNK | AREEQWLDY |
| | 1, 3, 4, 7, | (SEQ ID NO: | (SEQ ID NO: | (SEQ ID NO: | (SEQ ID NO: | (SEQ ID NO: | (SEQ ID NO: |
| | 110 | 100) | 138) | 167) | 209) | 245) | 277) |
| 2 | Antibody | QSISSW | KAS | QQYDSYYT | GYTFTGYH | INPNSGGT | ARERVGYCSRP |
| | 2, 5, 6, 8, | (SEQ ID NO: | (SEQ ID NO: | (SEQ ID NO: | (SEQ ID NO: | (SEQ ID NO: | SCLDDLNI |
| | 111 | 101) | 139) | 168) | 210) | 246) | (SEQ ID NO: |
| | | | | | | | 278) |
| 3 | Antibody | RASQSLGSTYL | GASTRAT | CQQYYSSPPTF | YSFSDYYMH | GWMSPNSGN | CARQREEYGH |
| | 9, 54, 83, | A | (SEQ ID NO: | (SEQ ID NO: | (SEQ ID NO: | TGYA | YYYGMDVW |
| | 98 | (SEQ ID NO: | 140) | 169) | 211) | (SEQ ID NO: | (SEQ ID NO: |
| | | 102) | | | | 247) | 279) |
| 4 | Antibody | QASQDIHNYL | DASNLET | CQQSYNTPPTF | GTFSNYDIS | GMIDPSGGST | CARPYSSGWY |
| | 10 | N | (SEQ ID NO: | (SEQ ID NO: | (SEQ ID NO: | SYA | YFDYW |
| | | (SEQ ID NO: | 141) | 170) | 212) | (SEQ ID NO: | (SEQ ID NO: |
| | | 103) | | | | 248) | 280) |
| 5 | Antibody | RASQTINNYLN | DASNLET | CQQANSFPLTF | YTFTSYDIN | GWMNPNSGN | CAKGAVAGTY |
| | 11 | (SEQ ID NO: | (SEQ ID NO: | (SEQ ID NO: | (SEQ ID NO: | TGYA | YYYGMDVW |
| | | 104) | 141) | 171) | 213) | (SEQ ID NO: | (SEQ ID NO: |
| | | | | | | 249) | 281) |
| 6 | Antibody | RASQNIRNYLN | AASSLQG | CQQSYNGPYT | GTFGSYGIN | GWISAYNGNT | CARDGIDYGG |
| | 12 | (SEQ ID NO: | (SEQ ID NO: | F | (SEQ ID NO: | NYA | NSGYDYW |
| | | 105) | 142) | (SEQ ID NO: | 214) | (SEQ ID NO: | (SEQ ID NO: |
| | | | | 172) | | 250) | 282) |
| 7 | Antibody | RSSQSLLHSNG | AASSLQS | CMQGTHWPP | FTFSNSDMN | SYISSSGNTIYY | CARSNWNYPL |
| | 13, 55 | YNYLD | (SEQ ID NO: | TF | (SEQ ID NO: | A | DYYYMDVW |
| | | (SEQ ID NO: | 143) | (SEQ ID NO: | 215) | (SEQ ID NO: | (SEQ ID NO: |
| | | 106) | | 173) | | 251) | 283) |
| 8 | Antibody | RSSQSLLHSNG | AASTLQS | CMQGLQTLYS | YTFTSYYMH | GWVDPNNGD | CARDNWGSLD |
| | 14, 56 | YNYLD | (SEQ ID NO: | F | (SEQ ID NO: | TGYA | VW |
| | | (SEQ ID NO: | 144) | (SEQ ID NO: | 216) | (SEQ ID NO: | (SEQ ID NO: |
| | | 106) | | 174) | | 252) | 284) |
| 9 | Antibody | RASQGVGDRS | DASNLET | CQQADTFPYT | YSFTSYGIS | GWISPYNGNT | CAREAYGDYG |
| | 15 | A | (SEQ ID NO: | F | (SEQ ID NO: | NYA | DYFDYW |
| | | (SEQ ID NO: | 141) | (SEQ ID NO: | 217) | (SEQ ID NO: | (SEQ ID NO: |
| | | 107) | | 175) | | 253) | 285) |
| 10 | Antibody | RASQGISTYLA | AASTLQS | CQQSHSAPITF | YTFTSYYMH | GWINPNSGNA | CARGPVYSYG |
| | 16, 57, 84, | (SEQ ID NO: | (SEQ ID NO: | (SEQ ID NO: | (SEQ ID NO: | GFA | PFDYW |
| | 99 | 108) | 144) | 176) | 216) | (SEQ ID NO: | (SEQ ID NO: |
| | | | | | | 254) | 286) |
| 11 | Antibody | RASQSISSYLN | DASSLES | CQQANSFPLTF | YTFTRNYVH | GRINPNSGGT | CARLGDVAVA |
| | 17 | (SEQ ID NO: | (SEQ ID NO: | (SEQ ID NO: | (SEQ ID NO: | NTA | ASFDYW |
| | | 109) | 145) | 171) | 218) | (SEQ ID NO: | (SEQ ID NO: |
| | | | | | | 255) | 287) |
| 12 | Antibody | RASQGIGNDL | DAKDLHP | CQQSYSFPRTF | YTFTGYYMH | GWMNPNSGN | CARGSGYDAG |
| | 18 | A | (SEQ ID NO: | (SEQ ID NO: | (SEQ ID NO: | TGYA | VFDYW |
| | | (SEQ ID NO: | 146) | 177) | 219) | (SEQ ID NO: | (SEQ ID NO: |
| | | 110) | | | | 249) | 288) |
| 13 | Antibody | RASESISHWLA | GASTLQS | CQQSYTPPLTF | YTFTAYYMH | GIINPSGGSTSY | CARDSGWYAP |
| | 19, 58 | (SEQ ID NO: | (SEQ ID NO: | (SEQ ID NO: | (SEQ ID NO: | A | GYYYGMDVW |
| | | 111) | 147) | 178) | 220) | (SEQ ID NO: | (SEQ ID NO: |
| | | | | | | 256) | 289) |
| 14 | Antibody | KSSQSLLYSSN | WASIRES | CQQYLSPPLTF | YTFTSYDIN | GWMNPNSGN | CARESYAYCSS |
| | 20, 59, 85, | NKNYLA | (SEQ ID NO: | (SEQ ID NO: | (SEQ ID NO: | TGYA | TSCYDAFDIW |
| | 100 | (SEQ ID NO: | 148) | 179) | 213) | (SEQ ID NO: | (SEQ ID NO: |
| | | 112) | | | | 249) | 290) |
| 15 | Antibody | RASQDIRRALA | AASSLQS | CLQHNSYPPTF | FTLGSYWMH | SLISWDGGSTY | CAKGPWVGYS |
| | 21, 60, 86, | (SEQ ID NO: | (SEQ ID NO: | (SEQ ID NO: | (SEQ ID NO: | YA | GYDLNYYYYG |
| | 101 | 113) | 143) | 180) | 221) | (SEQ ID NO: | MDVW |
| | | | | | | 257) | (SEQ ID NO: |
| | | | | | | | 291) |
| 16 | Antibody | RSSQSLLHSNG | LGSSRAS | CMQALQTPTF | YTFTSHDIN | GISNPSGGSTY | CASSRRYWNG |
| | 22, 61, 87 | YNYLD | (SEQ ID NO: | (SEQ ID NO: | (SEQ ID NO: | YA | AFDYW |
| | | (SEQ ID NO: | 149) | 181) | 222) | (SEQ ID NO: | (SEQ ID NO: |
| | | 106) | | | | 258) | 292) |
| 17 | Antibody | QASQGISSYLN | AASSLQN | CQQSYSTPITF | GTFSNYDIN | GWISAYNGNT | CAAEAYGDYSL |
| | 23, 62 | (SEQ ID NO: | (SEQ ID NO: | (SEQ ID NO: | (SEQ ID NO: | NYA | DPW |
| | | 114) | 150) | 182) | 223) | (SEQ ID NO: | (SEQ ID NO: |
| | | | | | | 250) | 293) |
| 18 | Antibody | RASQGIGNDL | AASNLET | CLQHHSYPFTF | YTFTGYYMH | GIINPSGGSTN | CARGPIGYGM |
| | 24, 63 | G | (SEQ ID NO: | (SEQ ID NO: | (SEQ ID NO: | YA | DVW |
| | | (SEQ ID NO: | 151) | 183) | 219) | (SEQ ID NO: | (SEQ ID NO: |
| | | 115) | | | | 259) | 294) |
| 19 | Antibody | RTSQSIGTYLN | DASNLPT | CQNYNSALF | FTVGSWYMS | AHVSDDGSNE | CARSPGYCSST |
| | 25 | (SEQ ID NO: | (SEQ ID NO: | (SEQ ID NO: | (SEQ ID NO: | FYA | SCYRYYGMDV |
| | | 116) | 152) | 184) | 224) | (SEQ ID NO: | W |
| | | | | | | 260) | (SEQ ID NO: |
| | | | | | | | 295) |
| 20 | Antibody | RASQSVDTWL | EASTLES | CQQANSFPITF | YTFTSYYMH | GRINPNSGGT | CARLGSAYFDL |
| | 26, 64 | A | (SEQ ID NO: | (SEQ ID NO: | (SEQ ID NO: | KYA | W |
| | | (SEQ ID NO: | 153) | 185) | 216) | (SEQ ID NO: | (SEQ ID NO: |
| | | 117) | | | | 261) | 296) |
| 21 | Antibody | RASQSISNLLA | AASSLQS | CQQSYSTPYTF | YTFTSHDIN | GWMNANNG | CAKDLSPSSSW |
| | 27, 65, 88, | (SEQ ID NO: | (SEQ ID NO: | (SEQ ID NO: | (SEQ ID NO: | NAGYA | STYYYYYGMD |
| | 102 | 118) | 143) | 186) | 222) | (SEQ ID NO: | VW |
| | | | | | | 262) | (SEQ ID NO: |
| | | | | | | | 297) |
| 22 | Antibody | RASQGISNWL | DASNLDA | CQQANSLPLTF | YTFTSYNIH | GWISAYNGNT | CALDGGQLGV |
| | 28 | A | (SEQ ID NO: | (SEQ ID NO: | (SEQ ID NO: | NYA | AFDIW |
| | | (SEQ ID NO: | 154) | 187) | 225) | (SEQ ID NO: | (SEQ ID NO: |
| | | 119) | | | | 250) | 298) |
| 23 | Antibody | RASQSISGSYL | GASTRAT | CQQYGSFPFTF | GTFSSYAIS | GIINPSSSSTGY | CARAGYYDSS |
| | 29, 66, 89, | A | (SEQ ID NO: | (SEQ ID NO: | (SEQ ID NO: | A | GYYYSPYYGM |
| | 103 | (SEQ ID NO: | 140) | 188) | 226) | (SEQ ID NO: | DVW |
| | | 120) | | | | 263) | (SEQ ID NO: |
| | | | | | | | 299) |
| 24 | Antibody | RASQDIRNDLA | DASNLET | CQQSYGTPYTF | YTFTNFDIN | GWISAYNGHT | CAQEAAAGYF |
| | 30, 67 | (SEQ ID NO: | (SEQ ID NO: | (SEQ ID NO: | (SEQ ID NO: | NYA | DYW |
| | | 121) | 141) | 189) | 227) | (SEQ ID NO: | (SEQ ID NO: |
| | | | | | | 264) | 300) |
| 25 | Antibody | RSSQSLFHSST | WASTRES | CQQYYITPLTF | GTFRSYAIS | GWMNPNSGN | CARESVLKGG |
| | 31 | NANNLA | (SEQ ID NO: | (SEQ ID NO: | (SEQ ID NO: | TGYA | MDVW |
| | | (SEQ ID NO: | 155) | 190) | 228) | (SEQ ID NO: | (SEQ ID NO: |
| | | 122) | | | | 249) | 301) |
| 26 | Antibody | RASQSISNWL | AASSLQS | CQQSYSTPYTF | YTFTSYYMY | GWINPNSGNT | CAKDRRRGVG |
| | 32, 68 | A | (SEQ ID NO: | (SEQ ID NO: | (SEQ ID NO: | GYA | LAAAGTSAFDI |
| | | (SEQ ID NO: | 143) | 186) | 229) | (SEQ ID NO: | W |
| | | 123) | | | | 265) | (SEQ ID NO: |
| | | | | | | | 302) |
| 27 | Antibody | RAGQSIGDYLN | AASSLQS | CQQSYSTPWT | YTFTSYYMH | GWMNPNSDN | CARVGVGYGY |
| | 33, 69, 90, | (SEQ ID NO: | (SEQ ID NO: | F | (SEQ ID NO: | TGYA | YYYGMDVW |
| | 104 | 124) | 143) | (SEQ ID NO: | 216) | (SEQ ID NO: | (SEQ ID NO: |
| | | | | 191) | | 266) | 303) |
| 28 | Antibody | RSSQSLLHSNG | LASTRAP | CMQGSHWPP | YTFSNYYMH | GWMNPNSGN | CARASMYSSS |
| | 34, 70, 91 | YNYLD | (SEQ ID NO: | SF | (SEQ ID NO: | TGYA | GFDYW |
| | | (SEQ ID NO: | 156) | (SEQ ID NO: | 230) | (SEQ ID NO: | (SEQ ID NO: |
| | | 106) | | 192) | | 249) | 304) |
| 29 | Antibody | RSSQSLLHSNG | DASSLES | CMQGTHWPL | YTFTSYYIH | GTINPSGGSTS | CARETSRWLR |
| | 35, 71 | YNYLD | (SEQ ID NO: | TF | (SEQ ID NO: | YA | DYYYGMDVW |
| | | (SEQ ID NO: | 145) | (SEQ ID NO: | 231) | (SEQ ID NO: | (SEQ ID NO: |
| | | 106) | | 193) | | 267) | 305) |
| 30 | Antibody | QASQGISNYLN | AASSLQS | CQQSYSSPYTF | YTFTDNYLH | GIIETSGGSTDY | CARSFGGYSYD |
| | 36, 72 | (SEQ ID NO: | (SEQ ID NO: | (SEQ ID NO: | (SEQ ID NO: | A | DAFDIW |
| | | 125) | 143) | 194) | 232) | (SEQ ID NO: | (SEQ ID NO: |
| | | | | | | 268) | 306) |
| 31 | Antibody | RASQSISRWLA | AASSLES | CQQTHSFPLTF | YTLNNYDIN | GWISGYNGNT | CARVGSSWEE |
| | 37 | (SEQ ID NO: | (SEQ ID NO: | (SEQ ID NO: | (SEQ ID NO: | NYA | NDQW |
| | | 126) | 157) | 195) | 233) | (SEQ ID NO: | (SEQ ID NO: |
| | | | | | | 269) | 307) |
| 32 | Antibody | RSSQSVLYSSN | WASTRES | CQQYYSTPPTF | GTFSSYDIN | GWMNPNSGN | CARDLTLYCSS |
| | 38, 73, 92, | NKNYLA | (SEQ ID NO: | (SEQ ID NO: | (SEQ ID NO: | TGYA | TSCLDAFDYW |
| | 105 | (SEQ ID NO: | 155) | 196) | 234) | (SEQ ID NO: | (SEQ ID NO: |
| | | 127) | | | | 249) | 308) |
| 33 | Antibody | RASQSVSSYLA | GTSTRAT | CQQYGSLPFTF | GTFSNYGFS | GWMSPSSGN | CARERSGSYFD |
| | 39, 74 | (SEQ ID NO: | (SEQ ID NO: | (SEQ ID NO: | (SEQ ID NO: | TGYA | YW |
| | | 128) | 158) | 197) | 235) | (SEQ ID NO: | (SEQ ID NO: |
| | | | | | | 270) | 309) |
| 34 | Antibody | KSSQSVLYSSN | WASTRAS | CQQYYTTPPTF | GTFSSYAIS | GRIHPRDGSTD | CARVSLRFLED |
| | 40 | NKNYLA | (SEQ ID NO: | (SEQ ID NO: | (SEQ ID NO: | YA | YGMDVW |
| | | (SEQ ID NO: | 159) | 198) | 226) | (SEQ ID NO: | (SEQ ID NO: |
| | | 129) | | | | 271) | 310) |
| 35 | Antibody | RSSQSLLHSNG | AASSLQS | CMQALQTPLT | GTFSNYAIS | GGIIPIFGSTAS | CARGVGDDTL |
| | 41, 75 | YNYLD | (SEQ ID NO: | F | (SEQ ID NO: | YA | TDW |
| | | (SEQ ID NO: | 143) | (SEQ ID NO: | 236) | (SEQ ID NO: | (SEQ ID NO: |
| | | 106) | | 199) | | 272) | 311) |
| 36 | Antibody | KSSQSLLYTSN | WASTRES | CQQYYSSPYTF | YTFTSYHMH | GWMNPNSGN | CARGGRVVRG |
| | 42 | DRNYLA | (SEQ ID NO: | (SEQ ID NO: | (SEQ ID NO: | TGYA | VIKGWFDPW |
| | | (SEQ ID NO: | 155) | 200) | 237) | (SEQ ID NO: | (SEQ ID NO: |
| | | 130) | | | | 249) | 312) |
| 37 | Antibody | RASQSISSWLA | DASNLET | CQQSFSTPPTF | GTFTSYDIN | GWISAYNGHT | CAREDSSWYF |
| | 43 | (SEQ ID NO: | (SEQ ID NO: | (SEQ ID NO: | (SEQ ID NO: | NYA | DLW |
| | | 131) | 141) | 201) | 238) | (SEQ ID NO: | (SEQ ID NO: |
| | | | | | | 264) | 313) |
| 38 | Antibody | RASQSVSSNLA | GASTRAT | CQQYNTYPITF | GTFSKIAVS | GLINPNSGGTD | CAKDFRGGDY |
| | 44, 76, 93 | (SEQ ID NO: | (SEQ ID NO: | (SEQ ID NO: | (SEQ ID NO: | YA | YYYGMDVW |
| | | 132) | 140) | 202) | 239) | (SEQ ID NO: | (SEQ ID NO: |
| | | | | | | 273) | 314) |
| 39 | Antibody | RASQSISSYLN | AASSLQS | CQQSYSTPLTF | YTFTSYGIS | GWISAYNGNT | CARGTYYDYI |
| | 45, 77, 94, | (SEQ ID NO: | (SEQ ID NO: | (SEQ ID NO: | (SEQ ID NO: | NYA | WGSYRYSWFD |
| | 106 | 109) | 143) | 203) | 240) | (SEQ ID NO: | YW |
| | | | | | | 250) | (SEQ ID NO: |
| | | | | | | | 315) |
| 40 | Antibody | QASQDISNYLN | AASTLQT | CQQSYSTPWT | YTFSSYAIS | GWMDPNSGN | CARESSYYYDS |
| | 46 | (SEQ ID NO: | (SEQ ID NO: | F | (SEQ ID NO: | TGYA | SGYYPGWYFD |
| | | 133) | 160) | (SEQ ID NO: | 241) | (SEQ ID NO: | LW |
| | | | | 191) | | 274) | (SEQ ID NO: |
| | | | | | | | 316) |
| 41 | Antibody | RASQRIGSYLN | GASNLQS | CQQANSFPFTF | YTFTTYGIS | GWMDPNSGS | CARGRGNSYY |
| | 47, 78 | (SEQ ID NO: | (SEQ ID NO: | (SEQ ID NO: | (SEQ ID NO: | TGYA | YYYMDVW |
| | | 134) | 161) | 204) | 242) | (SEQ ID NO: | (SEQ ID NO: |
| | | | | | | 275) | 317) |
| 42 | Antibody | RASESITNWLA | KASSLES | CQQSYGTPYTF | YTFTSYYMH | GWMNPNSGN | CARDQMYSSY |
| | 48, 79, 95, | (SEQ ID NO: | (SEQ ID NO: | (SEQ ID NO: | (SEQ ID NO: | TGYA | GMDVW |
| | 107 | 135) | 162) | 189) | 216) | (SEQ ID NO: | (SEQ ID NO: |
| | | | | | | 249) | 318) |
| 43 | Antibody | KSSQSVLYSSN | WASTRES | CQQYYSTPYTF | GTFSSYAIS | GVINPSGGSTS | CARESRGYYG |
| | 49 | NKNYLA | (SEQ ID NO: | (SEQ ID NO: | (SEQ ID NO: | YA | MDVW |
| | | (SEQ ID NO: | 155) | 205) | 226) | (SEQ ID NO: | (SEQ ID NO: |
| | | 129) | | | | 276) | 319) |
| 44 | Antibody | RASQSISSWLA | DTSNLQG | CQQYDSFPWT | YTFTGYYMH | GTINPSGGSTS | CVKGNGYYDL |
| | 50 | (SEQ ID NO: | (SEQ ID NO: | F | (SEQ ID NO: | YA | W |
| | | 131) | 163) | (SEQ ID NO: | 219) | (SEQ ID NO: | (SEQ ID NO: |
| | | | | 206) | | 267) | 320) |
| 45 | Antibody | RASQGIRNDL | SASNLQS | CQQSYDVPLTF | YTFTRYDIN | GWMNPNSGN | CARERVGYCSS |
| | 51, 80, 96, | G | (SEQ ID NO: | (SEQ ID NO: | (SEQ ID NO: | TGYA | TSCLHPLDYW |
| | 108 | (SEQ ID NO: | 164) | 207) | 243) | (SEQ ID NO: | (SEQ ID NO: |
| | | 136) | | | | 249) | 321) |
| 46 | Antibody | RASQSISSWLA | DATTLES | CQQYDSYPWT | YTFTSYDIN | GWISGYNGNT | CARSFDWLLLL |
| | 52,81 | (SEQ ID NO: | (SEQ ID NO: | F | (SEQ ID NO: | NYA | DYW |
| | | 131) | 165) | (SEQ ID NO: | 213) | (SEQ ID NO: | (SEQ ID NO: |
| | | | | 208) | | 269) | 322) |
| 47 | Antibody | RASQSVSKYLA | AISARAA | CQQYYSSPPTF | HTFNNNYIH | GIINPSGGSTSY | CARQIQDYGY |
| | 53, 82, 97, | (SEQ ID NO: | (SEQ ID NO: | (SEQ ID NO: | (SEQ ID NO: | A | YYYGMDVW |
| | 109 | 137) | 166) | 169) | 244) | (SEQ ID NO: | (SEQ ID NO: |
| | | | | | | 256) | 323) |

In some embodiments, provided herein is a SIRPγ antibody, wherein the antibody comprises the amino acid sequences of the following three VL CDRs: SEQ ID NO: 100, SEQ ID NO: 138, SEQ ID NO: 167; and/or comprises the amino acid sequences of the following three VH CDRs SEQ ID NO: 209, SEQ ID NO: 245, and SEQ ID NO: 277.

In some embodiments, provided herein is a SIRPγ antibody, wherein the antibody comprises the amino acid sequences of the following three VL CDRs: SEQ ID NO: 101, SEQ ID NO: 139, SEQ ID NO: 168; and/or comprises the amino acid sequences of the following three VH CDRs SEQ ID NO: 210, SEQ ID NO: 246, and SEQ ID NO: 278.

In some embodiments, provided herein is a SIRPγ antibody, wherein the antibody comprises the amino acid sequences of the following three VL CDRs: SEQ ID NO: 102, SEQ ID NO: 140, SEQ ID NO: 169; and/or comprises the amino acid sequences of the following three VH CDRs SEQ ID NO: 211, SEQ ID NO: 247, and SEQ ID NO: 279.

In some embodiments, provided herein is a SIRPγ antibody, wherein the antibody comprises the amino acid sequences of the following three VL CDRs: SEQ ID NO: 103, SEQ ID NO: 141, SEQ ID NO: 170; and/or comprises the amino acid sequences of the following three VH CDRs SEQ ID NO: 212, SEQ ID NO: 248, and SEQ ID NO: 280.

In some embodiments, provided herein is a SIRPγ antibody, wherein the antibody comprises the amino acid sequences of the following three VL CDRs: SEQ ID NO: 104, SEQ ID NO: 141, SEQ ID NO: 171; and/or comprises the amino acid sequences of the following three VH CDRs SEQ ID NO: 213, SEQ ID NO: 249, and SEQ ID NO: 281.

In some embodiments, provided herein is a SIRPγ antibody, wherein the antibody comprises the amino acid sequences of the following three VL CDRs: SEQ ID NO: 105, SEQ ID NO: 142, SEQ ID NO: 172; and/or comprises the amino acid sequences of the following three VH CDRs SEQ ID NO: 214, SEQ ID NO: 250, and SEQ ID NO: 282.

In some embodiments, provided herein is a SIRPγ antibody, wherein the antibody comprises the amino acid sequences of the following three VL CDRs: SEQ ID NO: 106, SEQ ID NO: 143, SEQ ID NO: 173; and/or comprises the amino acid sequences of the following three VH CDRs SEQ ID NO: 215, SEQ ID NO: 251, and SEQ ID NO: 283.

In some embodiments, provided herein is a SIRPγ antibody, wherein the antibody comprises the amino acid sequences of the following three VL CDRs: SEQ ID NO: 106, SEQ ID NO: 144, SEQ ID NO: 174; and/or comprises the amino acid sequences of the following three VH CDRs SEQ ID NO: 216, SEQ ID NO: 252, and SEQ ID NO: 284.

In some embodiments, provided herein is a SIRPγ antibody, wherein the antibody comprises the amino acid sequences of the following three VL CDRs: SEQ ID NO: 107, SEQ ID NO: 141, SEQ ID NO: 175; and/or comprises the amino acid sequences of the following three VH CDRs SEQ ID NO: 217, SEQ ID NO: 253, and SEQ ID NO: 285.

In some embodiments, provided herein is a SIRPγ antibody, wherein the antibody comprises the amino acid sequences of the following three VL CDRs: SEQ ID NO: 108, SEQ ID NO: 144, SEQ ID NO: 176; and/or comprises the amino acid sequences of the following three VH CDRs SEQ ID NO: 216, SEQ ID NO: 254, and SEQ ID NO: 286.

In some embodiments, provided herein is a SIRPγ antibody, wherein the antibody comprises the amino acid sequences of the following three VL CDRs: SEQ ID NO: 109, SEQ ID NO: 145, SEQ ID NO: 171; and/or comprises the amino acid sequences of the following three VH CDRs SEQ ID NO: 218, SEQ ID NO: 255, and SEQ ID NO: 287.

In some embodiments, provided herein is a SIRPγ antibody, wherein the antibody comprises the amino acid sequences of the following three VL CDRs: SEQ ID NO: 110, SEQ ID NO: 146, SEQ ID NO: 177; and/or comprises the amino acid sequences of the following three VH CDRs SEQ ID NO: 219, SEQ ID NO: 249, and SEQ ID NO: 288.

In some embodiments, provided herein is a SIRPγ antibody, wherein the antibody comprises the amino acid sequences of the following three VL CDRs: SEQ ID NO: 111, SEQ ID NO: 147, SEQ ID NO: 178; and/or comprises the amino acid sequences of the following three VH CDRs SEQ ID NO: 220, SEQ ID NO: 256, and SEQ ID NO: 289.

In some embodiments, provided herein is a SIRPγ antibody, wherein the antibody comprises the amino acid sequences of the following three VL CDRs: SEQ ID NO: 112, SEQ ID NO: 148, SEQ ID NO: 179; and/or comprises the amino acid sequences of the following three VH CDRs SEQ ID NO: 213, SEQ ID NO: 249, and SEQ ID NO: 290.

In some embodiments, provided herein is a SIRPγ antibody, wherein the antibody comprises the amino acid sequences of the following three VL CDRs: SEQ ID NO: 113, SEQ ID NO: 143, SEQ ID NO: 180; and/or comprises the amino acid sequences of the following three VH CDRs SEQ ID NO: 221, SEQ ID NO: 257, and SEQ ID NO: 291.

In some embodiments, provided herein is a SIRPγ antibody, wherein the antibody comprises the amino acid sequences of the following three VL CDRs: SEQ ID NO: 106, SEQ ID NO: 149, SEQ ID NO: 181; and/or comprises the amino acid sequences of the following three VH CDRs SEQ ID NO: 222, SEQ ID NO: 258, and SEQ ID NO: 292.

In some embodiments, provided herein is a SIRPγ antibody, wherein the antibody comprises the amino acid sequences of the following three VL CDRs: SEQ ID NO: 114, SEQ ID NO: 150, SEQ ID NO: 182; and/or comprises the amino acid sequences of the following three VH CDRs SEQ ID NO: 223, SEQ ID NO: 250, and SEQ ID NO: 293.

In some embodiments, provided herein is a SIRPγ antibody, wherein the antibody comprises the amino acid sequences of the following three VL CDRs: SEQ ID NO: 115, SEQ ID NO: 151, SEQ ID NO: 183; and/or comprises the amino acid sequences of the following three VH CDRs SEQ ID NO: 219, SEQ ID NO: 259, and SEQ ID NO: 294.

In some embodiments, provided herein is a SIRPγ antibody, wherein the antibody comprises the amino acid sequences of the following three VL CDRs: SEQ ID NO: 116, SEQ ID NO: 152, SEQ ID NO: 184; and/or comprises the amino acid sequences of the following three VH CDRs SEQ ID NO: 224, SEQ ID NO: 260, and SEQ ID NO: 295.

In some embodiments, provided herein is a SIRPγ antibody, wherein the antibody comprises the amino acid sequences of the following three VL CDRs: SEQ ID NO: 117, SEQ ID NO: 153, SEQ ID NO: 185; and/or comprises the amino acid sequences of the following three VH CDRs SEQ ID NO: 216, SEQ ID NO: 261, and SEQ ID NO: 296.

In some embodiments, provided herein is a SIRPγ antibody, wherein the antibody comprises the amino acid sequences of the following three VL CDRs: SEQ ID NO: 118, SEQ ID NO: 143, SEQ ID NO: 186; and/or comprises the amino acid sequences of the following three VH CDRs SEQ ID NO: 222, SEQ ID NO: 262, and SEQ ID NO: 297.

In some embodiments, provided herein is a SIRPγ antibody, wherein the antibody comprises the amino acid sequences of the following three VL CDRs: SEQ ID NO: 119, SEQ ID NO: 154, SEQ ID NO: 187; and/or comprises the amino acid sequences of the following three VH CDRs SEQ ID NO: 225, SEQ ID NO: 250, and SEQ ID NO: 298.

In some embodiments, provided herein is a SIRPγ antibody, wherein the antibody comprises the amino acid sequences of the following three VL CDRs: SEQ ID NO: 120, SEQ ID NO: 140, SEQ ID NO: 188; and/or comprises the amino acid sequences of the following three VH CDRs SEQ ID NO: 226, SEQ ID NO: 263, and SEQ ID NO: 299.

In some embodiments, provided herein is a SIRPγ antibody, wherein the antibody comprises the amino acid sequences of the following three VL CDRs: SEQ ID NO: 121, SEQ ID NO: 141, SEQ ID NO: 189; and/or comprises the amino acid sequences of the following three VH CDRs SEQ ID NO: 227, SEQ ID NO: 264, and SEQ ID NO: 300.

In some embodiments, provided herein is a SIRPγ antibody, wherein the antibody comprises the amino acid sequences of the following three VL CDRs: SEQ ID NO: 122, SEQ ID NO: 155, SEQ ID NO: 190; and/or comprises the amino acid sequences of the following three VH CDRs SEQ ID NO: 228, SEQ ID NO: 249, and SEQ ID NO: 301.

In some embodiments, provided herein is a SIRPγ antibody, wherein the antibody comprises the amino acid sequences of the following three VL CDRs: SEQ ID NO: 123, SEQ ID NO: 143, SEQ ID NO: 186; and/or comprises the amino acid sequences of the following three VH CDRs SEQ ID NO: 229, SEQ ID NO: 265, and SEQ ID NO: 302.

In some embodiments, provided herein is a SIRPγ antibody, wherein the antibody comprises the amino acid sequences of the following three VL CDRs: SEQ ID NO: 124, SEQ ID NO: 143, SEQ ID NO: 191; and/or comprises the amino acid sequences of the following three VH CDRs SEQ ID NO: 216, SEQ ID NO: 266, and SEQ ID NO: 303.

In some embodiments, provided herein is a SIRPγ antibody, wherein the antibody comprises the amino acid sequences of the following three VL CDRs: SEQ ID NO: 106, SEQ ID NO: 156, SEQ ID NO: 192; and/or comprises the amino acid sequences of the following three VH CDRs SEQ ID NO: 230, SEQ ID NO: 249, and SEQ ID NO: 304.

In some embodiments, provided herein is a SIRPγ antibody, wherein the antibody comprises the amino acid sequences of the following three VL CDRs: SEQ ID NO: 106, SEQ ID NO: 145, SEQ ID NO: 193; and/or comprises the amino acid sequences of the following three VH CDRs SEQ ID NO: 231, SEQ ID NO: 267, and SEQ ID NO: 305.

In some embodiments, provided herein is a SIRPγ antibody, wherein the antibody comprises the amino acid sequences of the following three VL CDRs: SEQ ID NO: 125, SEQ ID NO: 143, SEQ ID NO: 194; and/or comprises the amino acid sequences of the following three VH CDRs SEQ ID NO: 232, SEQ ID NO: 268, and SEQ ID NO: 306.

In some embodiments, provided herein is a SIRPγ antibody, wherein the antibody comprises the amino acid sequences of the following three VL CDRs: SEQ ID NO: 126, SEQ ID NO: 157, SEQ ID NO: 195; and/or comprises the amino acid sequences of the following three VH CDRs SEQ ID NO: 233, SEQ ID NO: 269, and SEQ ID NO: 307.

In some embodiments, provided herein is a SIRPγ antibody, wherein the antibody comprises the amino acid sequences of the following three VL CDRs: SEQ ID NO: 127, SEQ ID NO: 155, SEQ ID NO: 196; and/or comprises the amino acid sequences of the following three VH CDRs SEQ ID NO: 234, SEQ ID NO: 249, and SEQ ID NO: 308.

In some embodiments, provided herein is a SIRPγ antibody, wherein the antibody comprises the amino acid sequences of the following three VL CDRs: SEQ ID NO: 128, SEQ ID NO: 158, SEQ ID NO: 197; and/or comprises the amino acid sequences of the following three VH CDRs SEQ ID NO: 235, SEQ ID NO: 270, and SEQ ID NO: 309.

In some embodiments, provided herein is a SIRPγ antibody, wherein the antibody comprises the amino acid sequences of the following three VL CDRs: SEQ ID NO: 129, SEQ ID NO: 159, SEQ ID NO: 198; and/or comprises the amino acid sequences of the following three VH CDRs SEQ ID NO: 226, SEQ ID NO: 271, and SEQ ID NO: 310.

In some embodiments, provided herein is a SIRPγ antibody, wherein the antibody comprises the amino acid sequences of the following three VL CDRs: SEQ ID NO: 106, SEQ ID NO: 143, SEQ ID NO: 199; and/or comprises the amino acid sequences of the following three VH CDRs SEQ ID NO: 236, SEQ ID NO: 272, and SEQ ID NO: 311.

In some embodiments, provided herein is a SIRPγ antibody, wherein the antibody comprises the amino acid sequences of the following three VL CDRs: SEQ ID NO: 130, SEQ ID NO: 155, SEQ ID NO: 200; and/or comprises the amino acid sequences of the following three VH CDRs SEQ ID NO: 237, SEQ ID NO: 249, and SEQ ID NO: 312.

In some embodiments, provided herein is a SIRPγ antibody, wherein the antibody comprises the amino acid sequences of the following three VL CDRs: SEQ ID NO: 131, SEQ ID NO: 141, SEQ ID NO: 201; and/or comprises the amino acid sequences of the following three VH CDRs SEQ ID NO: 238, SEQ ID NO: 264, and SEQ ID NO: 313.

In some embodiments, provided herein is a SIRPγ antibody, wherein the antibody comprises the amino acid sequences of the following three VL CDRs: SEQ ID NO: 132, SEQ ID NO: 140, SEQ ID NO: 202; and/or comprises the amino acid sequences of the following three VH CDRs SEQ ID NO: 239, SEQ ID NO: 273, and SEQ ID NO: 314.

In some embodiments, provided herein is a SIRPγ antibody, wherein the antibody comprises the amino acid sequences of the following three VL CDRs: SEQ ID NO: 109, SEQ ID NO: 143, SEQ ID NO: 203; and/or comprises the amino acid sequences of the following three VH CDRs SEQ ID NO: 240, SEQ ID NO: 250, and SEQ ID NO: 315.

In some embodiments, provided herein is a SIRPγ antibody, wherein the antibody comprises the amino acid sequences of the following three VL CDRs: SEQ ID NO: 133, SEQ ID NO: 160, SEQ ID NO: 191; and/or comprises the amino acid sequences of the following three VH CDRs SEQ ID NO: 241, SEQ ID NO: 274, and SEQ ID NO: 316.

In some embodiments, provided herein is a SIRPγ antibody, wherein the antibody comprises the amino acid sequences of the following three VL CDRs: SEQ ID NO: 134, SEQ ID NO: 161, SEQ ID NO: 204; and/or comprises the amino acid sequences of the following three VH CDRs SEQ ID NO: 242, SEQ ID NO: 275, and SEQ ID NO: 317.

In some embodiments, provided herein is a SIRPγ antibody, wherein the antibody comprises the amino acid sequences of the following three VL CDRs: SEQ ID NO: 135, SEQ ID NO: 162, SEQ ID NO: 189; and/or comprises the amino acid sequences of the following three VH CDRs SEQ ID NO: 216, SEQ ID NO: 249, and SEQ ID NO: 318.

In some embodiments, provided herein is a SIRPγ antibody, wherein the antibody comprises the amino acid sequences of the following three VL CDRs: SEQ ID NO: 129, SEQ ID NO: 155, SEQ ID NO: 205; and/or comprises the amino acid sequences of the following three VH CDRs SEQ ID NO: 226, SEQ ID NO: 276, and SEQ ID NO: 319.

In some embodiments, provided herein is a SIRPγ antibody, wherein the antibody comprises the amino acid sequences of the following three VL CDRs: SEQ ID NO: 131, SEQ ID NO: 163, SEQ ID NO: 206; and/or comprises the amino acid sequences of the following three VH CDRs SEQ ID NO: 219, SEQ ID NO: 267, and SEQ ID NO: 320.

In some embodiments, provided herein is a SIRPγ antibody, wherein the antibody comprises the amino acid sequences of the following three VL CDRs: SEQ ID NO: 136, SEQ ID NO: 164, SEQ ID NO: 207; and/or comprises the amino acid sequences of the following three VH CDRs SEQ ID NO: 243, SEQ ID NO: 249, and SEQ ID NO: 321.

In some embodiments, provided herein is a SIRPγ antibody, wherein the antibody comprises the amino acid sequences of the following three VL CDRs: SEQ ID NO: 131, SEQ ID NO: 165, SEQ ID NO: 208; and/or comprises the amino acid sequences of the following three VH CDRs SEQ ID NO: 213, SEQ ID NO: 269, and SEQ ID NO: 322.

In some embodiments, provided herein is a SIRPγ antibody, wherein the antibody comprises the amino acid sequences of the following three VL CDRs: SEQ ID NO: 137, SEQ ID NO: 166, SEQ ID NO: 169; and/or comprises the amino acid sequences of the following three VH CDRs SEQ ID NO: 244, SEQ ID NO: 256, and SEQ ID NO: 323.

ii. Exemplary SIRPγ Antibodies—Variable Region Sequences

The term variable domain and variable region are used interchangeably and refer to the portions of the light and heavy chains of an antibody that include the complementarity determining regions and framework regions (FRs).

Table 2 provides amino acid sequences for the variable domains of exemplary SIRPγ antibodies of the disclosure. Accordingly, in some embodiments a SIRPγ antibody of the disclosure comprises a variable heavy chain comprising an amino acid sequence selected from SEQ ID NOS: 324-370, or at least 80% sequence identity thereto; and/or in some embodiments a SIRPγ antibody of the disclosure comprises a variable light chain comprising an amino acid sequence selected from SEQ ID NOS: 371-417, or at least 80% sequence identity thereto.

In some embodiments, a SIRPγ antibody of the disclosure comprises the combination of VH/VL variable chain amino acid sequences of any one of the 47 combinations presented in Table 2.

TABLE 2
Exemplary Variable Heavy Chain and Variable Light Chain Amino Acid Sequence
Combinations of SIRPγ Antibodies

| Comb- | | | |
|---|---|---|---|
| ination | Antibody | Variable Heavy Chain Amino Acid | Variable Light Chain Amino Acid |
| No. | No. | Sequence | Sequence |
| 1 | Antibody | QVQLVESGGGVVQPGRSLRLSCAASGFTFS | EIVMTQSPATLSVSPGERATLSCRASQSVSS |
| | 1, 3, 4, 7, | NYGMHWVRQAPGKGLEWVAFIWYDGSN | NLAWYQQKPGQAPRLLIYGASTRATGIPARF |
| | 110 | KNYADSVKGRFTISRDNPTNTLYLQMNSLRA | SGSGSGTEFTLTISSLQSEDFAVYYCQQYNN |
| | | EDTAVYYCAREEQWLDYWGQGTLVTVSS | WPLTFGGGTKVEIK (SEQ ID NO: 371) |
| | | (SEQ ID NO: 324) | |
| 2 | Antibody | QVQLVQSGAEVKKPGASVKVSCKASGYTFT | DIQMTQSPSTLSASVGDRVTITCRASQSISS |
| | 2, 5, 6, 8, | GYHIHWVRQAPGQGLEWMGWINPNSGG | WLAWYQQKPGKAPKLLIYKASSLETGVPSRF |
| | 111 | TNYAQKFQGRVTMTRDTSINTAYMELNRLR | SGSGSGTEFTLTISSLQPDDFATYYCQQYDSY |
| | | SDDTAVFYCARERVGYCSRPSCLDDLNIWG | YTFGQGTKLEIK (SEQ ID NO: 372) |
| | | QGTMVTVSS (SEQ ID NO: 325) | |
| 3 | Antibody 9, | QVQLVQSGAEVKKPGASVKVSCKASGYSFS | EIVMTQSPATLSVSPGERATLSCRASQSLGST |
| | 54, 83, 98 | DYYMHWVRQAPGQGLEWMGWMSPNSG | YLAWYQQKPGQAPRLLIYGASTRATGIPARF |
| | | NTGYAQKFQGRVTMTRDTSTSTVYMELSSL | SGSGSGTEFTLTISSLQSEDFAVYYCQQYYSS |
| | | RSEDTAVYYCARQREEYGHYYYGMDVWGQ | PPTFGQGTKVEIK (SEQ ID NO: 373) |
| | | GTMVTVSS (SEQ ID NO: 326) | |
| 4 | Antibody 10 | QVQLVQSGAEVKKPGASVKVSCKASGGTFS | DIQMTQSPSSLSASVGDRVTITCQASQDIHN |
| | | NYDISWVRQAPGQGLEWMGMIDPSGGST | YLNWYQQKPGKAPKLLIYDASNLETGVPSRF |
| | | SYAQKFQGRVTMTRDTSTSTVYMELSSLRSE | SGSGSGTDFTLTISSLQPEDFATYYCQQSYNT |
| | | DTAVYYCARPYSSGWYYFDYWGQGTLVTVS | PPTFGQGTRLEIK (SEQ ID NO: 374) |
| | | S (SEQ ID NO: 327) | |
| 5 | Antibody 11 | QVQLVQSGAEVKKPGASVKVSCKASGYTFT | DIQMTQSPSSLSASVGDRVTITCRASQTINN |
| | | SYDINWVRQAPGQGLEWMGWMNPNSG | YLNWYQQKPGKAPKLLIYDASNLETGVPSRF |
| | | NTGYAQKFQGRVTMTRDTSTSTVYMELSSL | SGSGSGTDFTLTISSLOPEDFATYYCQQANSF |
| | | RSEDTAVYYCAKGAVAGTYYYYGMDVWGQ | PLTFGQGTKVEIK (SEQ ID NO: 375) |
| | | GTTVTVSS (SEQ ID NO: 328) | |
| 6 | Antibody 12 | QVQLVQSGAEVKKPGASVKVSCKASGGTFG | DIQMTQSPSSLSASVGDRVTITCRASQNIRN |
| | | SYGINWVRQAPGQGLEWMGWISAYNGNT | YLNWYQQKPGKAPKLLIYAASSLOGGVPSRF |
| | | NYAQKLQGRVTMTRDTSTSTVYMELSSLRS | SGSGSGTDFTLTISSLQPEDFATYYCQQSYN |
| | | EDTAVYYCARDGIDYGGNSGYDYWGQGTL | GPYTFGQGTKVEIK (SEQ ID NO: 376) |
| | | VTVSS (SEQ ID NO: 329) | |
| 7 | Antibody 13, | EVQLLESGGGLVQPGGSLRLSCAASGFTFSN | DIVMTQSPLSLPVTPGEPASISCRSSQSLLHS |
| | 55 | SDMNWVRQAPGKGLEWVSYISSSGNTIYYA | NGYNYLDWYLQKPGQSPQLLIYAASSLQSG |
| | | DSVKGRFTISRDNSKNTLYLQMNSLRAEDTA | VPDRFSGSGSGTDFTLKISRVEAEDVGVYYC |
| | | VYYCARSNWNYPLDYYYMDVWGKGTTVTV | MQGTHWPPTFGQGTKVEIK (SEQ ID NO: |
| | | SS (SEQ ID NO: 330) | 377) |
| 8 | Antibody 14, | QVQLVQSGAEVKKPGASVKVSCKASGYTFT | DIVMTQSPLSLPVTPGEPASISCRSSQSLLHS |
| | 56 | SYYMHWVRQAPGQGLEWMGWVDPNNG | NGYNYLDWYLQKPGQSPQLLIYAASTLQSG |
| | | DTGYAQKFQGRVTMTRDTSTSTVYMELSSL | VPDRFSGSGSGTDFTLKISRVEAEDVGVYYC |
| | | RSEDTAVYYCARDNWGSLDVWGKGTTVTV | MQGLQTLYSFGQGTKLEIK (SEQ ID NO: |
| | | SS (SEQ ID NO: 331) | 378) |
| 9 | Antibody 15 | QVQLVQSGAEVKKPGASVKVSCKASGYSFT | DIQMTQSPSSLSASVGDRVTITCRASQGVG |
| | | SYGISWVRQAPGQGLEWMGWISPYNGNT | DRSAWYQQKPGKAPKLLIYDASNLETGVPS |
| | | NYAQKLQGRVTMTRDTSTSTVYMELSSLRS | RFSGSGSGTDFTLTISSLQPEDFATYYCQQA |
| | | EDTAVYYCAREAYGDYGDYFDYWGQGTLV | DTFPYTFGQGTKLEIK (SEQ ID NO: 379) |
| | | TVSS (SEQ ID NO: 332) | |
| 10 | Antibody 16, | QVQLVQSGAEVKKPGASVKVSCKASGYTFT | DIQMTQSPSSLSASVGDRVTITCRASQGISTY |
| | 57, 84, 99 | SYYMHWVRQAPGQGLEWMGWINPNSGN | LAWYQQKPGKAPKLLIYAASTLQSGVPSRFS |
| | | AGFAQKFQGRVTMTRDTSTSTVYMELSSLR | GSGSGTDFTLTISSLQPEDFATYYCQQSHSAP |
| | | SEDTAVYYCARGPVYSYGPFDYWGQGTLVT | ITFGQGTKVEIK (SEQ ID NO: 380) |
| | | VSS (SEQ ID NO: 333) | |
| 11 | Antibody 17 | QVQLVQSGAEVKKPGASVKVSCKASGYTFT | DIQMTQSPSSLSASVGDRVTITCRASQSISSY |
| | | RNYVHWVRQAPGQGLEWMGRINPNSGGT | LNWYQQKPGKAPKLLIYDASSLESGVPSRFS |
| | | NTAQKFQGRVTMTRDTSTSTVYMELSSLRS | GSGSGTDFTLTISSLQPEDFATYYCQQANSF |
| | | EDTAVYYCARLGDVAVAASFDYWGQGTLV | PLTFGPGTKVDIK (SEQ ID NO: 381) |
| | | TVSS (SEQ ID NO: 334) | |
| 12 | Antibody 18 | QVQLVQSGAEVKKPGASVKVSCKASGYTFT | DIQMTQSPSSLSASVGDRVTITCRASQGIGN |
| | | GYYMHWVRQAPGQGLEWMGWMNPNS | DLAWYQQKPGKAPKLLIYDAKDLHPGVPSR |
| | | GNTGYAQKFQGRVTMTRDTSTSTVYMELSS | FSGSGSGTDFTLTISSLQPEDFATYYCQQSYS |
| | | LRSEDTAVYYCARGSGYDAGVFDYWGQGT | FPRTFGQGTRLEIK (SEQ ID NO: 382) |
| | | LVTVSS (SEQ ID NO: 335) | |
| 13 | Antibody 19, | QVQLVQSGAEVKKPGASVKVSCKASGYTFT | DIQMTQSPSSLSASVGDRVTITCRASESISH |
| | 58 | AYYMHWVRQAPGQGLEWMGIINPSGGST | WLAWYQQKPGKAPKLLIYGASTLQSGVPSR |
| | | SYAQKFQGRVTMTRDTSTSTVYMELSSLRSE | FSGSGSGTDFTLTISSLOPEDFATYYCQQSYT |
| | | DTAVYYCARDSGWYAPGYYYGMDVWGQG | PPLTFGGGTKVEIK (SEQ ID NO: 383) |
| | | TTVTVSS (SEQ ID NO: 336) | |
| 14 | Antibody 20, | QVQLVQSGAEVKKPGASVKVSCKASGYTFT | DIVMTQSPDSLAVSLGERATINCKSSQSLLYS |
| | 59, 85, 100 | SYDINWVRQAPGQGLEWMGWMNPNSG | SNNKNYLAWYQQKPGQPPKLLIYWASIRES |
| | | NTGYAQKFQGRVTMTRDTSTSTVYMELSSL | GVPDRFSGSGSGTDFTLTISSLQAEDVAVYY |
| | | RSEDTAVYYCARESYAYCSSTSCYDAFDIWG | CQQYLSPPLTFGQGTKVEIK (SEQ ID NO: |
| | | QGTMVTVSS (SEQ ID NO: 337) | 384) |
| 15 | Antibody 21, | EVQLLESGGGLVQPGGSLRLSCAASGFTLGS | DIQMTQSPSSLSASVGDRVTITCRASQDIRR |
| | 60, 86, 101 | YWMHWVRQAPGKGLEWVSLISWDGGSTY | ALAWYQQKPGKAPKLLIYAASSLQSGVPSRF |
| | | YADSVKGRFTISRDNSKNTLYLQMNSLRAED | SGSGSGTDFTLTISSLQPEDFATYYCLQHNSY |
| | | TAVYYCAKGPWVGYSGYDLNYYYYGMDV | PPTFGQGTKLEIK (SEQ ID NO: 385) |
| | | WGQGTTVTVSS (SEQ ID NO: 338) | |
| 16 | Antibody 22, | QVQLVQSGAEVKKPGASVKVSCKASGYTFT | DIVMTQSPLSLPVTPGEPASISCRSSQSLLHS |
| | 61, 87 | SHDINWVRQAPGQGLEWMGISNPSGGSTY | NGYNYLDWYLQKPGQSPQLLIYLGSSRASG |
| | | YAQKFQGRVTMTRDTSTSTVYMELSSLRSE | VPDRFSGSGSGTDFTLKISRVEAEDVGVYYC |
| | | DTAVYYCASSRRYWNGAFDYWGQGTLVTV | MQALQTPTFGQGTRLEIK (SEQ ID NO: 386) |
| | | SS (SEQ ID NO: 339) | |
| 17 | Antibody 23, | QVQLVQSGAEVKKPGASVKVSCKASGGTFS | DIQMTQSPSSLSASVGDRVTITCQASQGISS |
| | 62 | NYDINWVRQAPGQGLEWMGWISAYNGN | YLNWYQQKPGKAPKLLIYAASSLQNGVPSRF |
| | | TNYAQKLQGRVTMTRDTSTSTVYMELSSLR | SGSGSGTDFTLTISSLQPEDFATYYCQQSYST |
| | | SEDTAVYYCAAEAYGDYSLDPWGQGTLVTV | PITFGQGTRLEIK (SEQ ID NO: 387) |
| | | SS (SEQ ID NO: 340) | |
| 18 | Antibody 24, | QVQLVQSGAEVKKPGASVKVSCKASGYTFT | DIQMTQSPSSLSASVGDRVTITCRASQGIGN |
| | 63 | GYYMHWVRQAPGQGLEWMGIINPSGGST | DLGWYQQKPGKAPKLLIYAASNLETGVPSRF |
| | | NYAQKFQGRVTMTRDTSTSTVYMELSSLRS | SGSGSGTDFTLTISSLQPEDFATYYCLQHHSY |
| | | EDTAVYYCARGPIGYGMDVWGQGTLVTVS | PFTFGQGTKLEIK (SEQ ID NO: 388) |
| | | S (SEQ ID NO: 341) | |
| 19 | Antibody 25 | EVQLLESGGGLVQPGGSLRLSCAASGFTVGS | DIQMTQSPSSLSASVGDRVTITCRTSQSIGTY |
| | | WYMSWVRQAPGKGLEWVAHVSDDGSNE | LNWYQQKPGKAPKLLIYDASNLPTGVPSRFS |
| | | FYADSVKGRFTISRDNSKNTLYLQMNSLRAE | GSGSGTDFTLTISSLQPEDFATYYCQNYNSAL |
| | | DTAVYYCARSPGYCSSTSCYRYYGMDVWG | FGPGTKVDIK (SEQ ID NO: 389) |
| | | QGTTVTVSS (SEQ ID NO: 342) | |
| 20 | Antibody 26, | QVQLVQSGAEVKKPGASVKVSCKASGYTFT | DIQMTQSPSSLSASVGDRVTITCRASQSVDT |
| | 64 | SYYMHWVRQAPGQGLEWMGRINPNSGG | WLAWYQQKPGKAPKLLIYEASTLESGVPSRF |
| | | TKYAQKFQGRVTMTRDTSTSTVYMELSSLRS | SGSGSGTDFTLTISSLOPEDFATYYCQQANSF |
| | | EDTAVYYCARLGSAYFDLWGRGTLVTVSS | PITFGQGTRLEIK (SEQ ID NO: 390) |
| | | (SEQ ID NO: 343) | |
| 21 | Antibody 27, | QVQLVQSGAEVKKPGASVKVSCKASGYTFT | DIQMTQSPSSLSASVGDRVTITCRASQSISNL |
| | 65, 88, 102 | SHDINWVRQAPGQGLEWMGWMNANNG | LAWYQQKPGKAPKLLIYAASSLQSGVPSRFS |
| | | NAGYAQKFQGRVTMTRDTSTSTVYMELSSL | GSGSGTDFTLTISSLQPEDFATYYCQQSYSTP |
| | | RSEDTAVYYCAKDLSPSSSWSTYYYYYGMDV | YTFGQGTKLEIK (SEQ ID NO: 391) |
| | | WGQGTMVTVSS (SEQ ID NO: 344) | |
| 22 | Antibody 28 | QVQLVQSGAEVKKPGASVKVSCKASGYTFT | DIQMTQSPSSLSASVGDRVTITCRASQGISN |
| | | SYNIHWVRQAPGQGLEWMGWISAYNGNT | WLAWYQQKPGKAPKLLIYDASNLDAGVPSR |
| | | NYAQKLQGRVTMTRDTSTSTVYMELSSLRS | FSGSGSGTDFTLTISSLQPEDFATYYCQQANS |
| | | EDTAVYYCALDGGQLGVAFDIWGQGTMVT | LPLTFGGGTKVEIK (SEQ ID NO: 392) |
| | | VSS (SEQ ID NO: 345) | |
| 23 | Antibody 29, | QVQLVQSGAEVKKPGASVKVSCKASGGTFS | EIVMTQSPATLSVSPGERATLSCRASQSISGS |
| | 66, 89, 103 | SYAISWVRQAPGQGLEWMGIINPSSSSTGY | YLAWYQQKPGQAPRLLIYGASTRATGIPARF |
| | | AQKFQGRVTMTRDTSTSTVYMELSSLRSED | SGSGSGTEFTLTISSLQSEDFAVYYCQQYGSF |
| | | TAVYYCARAGYYDSSGYYYSPYYGMDVWG | PFTFGPGTKVDIK (SEQ ID NO: 393) |
| | | QGTTVTVSS (SEQ ID NO: 346) | |
| 24 | Antibody 30, | QVQLVQSGAEVKKPGASVKVSCKASGYTFT | DIQMTQSPSSLSASVGDRVTITCRASQDIRN |
| | 67 | NFDINWVRQAPGQGLEWMGWISAYNGHT | DLAWYQQKPGKAPKLLIYDASNLETGVPSRF |
| | | NYAQNLQGRVTMTRDTSTSTVYMELSSLRS | SGSGSGTDFTLTISSLQPEDFATYYCQQSYGT |
| | | EDTAVYYCAQEAAAGYFDYWGQGTLVTVSS | PYTFGQGTKLEIK (SEQ ID NO: 394) |
| | | (SEQ ID NO: 347) | |
| 25 | Antibody 31 | QVQLVQSGAEVKKPGASVKVSCKASGGTFR | DIVMTQSPDSLAVSLGERATINCRSSQSLFHS |
| | | SYAISWVRQAPGQGLEWMGWMNPNSGN | STNANNLAWYQQKPGQPPKLLIYWASTRES |
| | | TGYAQKFQGRVTMTRDTSTSTVYMELSSLR | GVPDRFSGSGSGTDFTLTISSLQAEDVAVYY |
| | | SEDTAVYYCARESVLKGGMDVWGQGTTVT | CQQYYITPLTFGQGTRLEIK (SEQ ID NO: |
| | | VSS (SEQ ID NO: 348) | 395) |
| 26 | Antibody 32, | QVQLVQSGAEVKKPGASVKVSCKASGYTFT | DIQMTQSPSSLSASVGDRVTITCRASQSISN |
| | 68 | SYYMYWVRQAPGQGLEWMGWINPNSGN | WLAWYQQKPGKAPKLLIYAASSLQSGVPSR |
| | | TGYAQKFQGRVTMTRDTSTSTVYMELSSLR | FSGSGSGTDFTLTISSLQPEDFATYYCQQSYS |
| | | SEDTAVYYCAKDRRRGVGLAAAGTSAFDIW | TPYTFGQGTKLEIK (SEQ ID NO: 396) |
| | | GQGTMVTVSS (SEQ ID NO: 349) | |
| 27 | Antibody 33, | QVQLVQSGAEVKKPGASVKVSCKASGYTFT | DIQMTQSPSSLSASVGDRVTITCRAGQSIGD |
| | 69, 90, 104 | SYYMHWVRQAPGQGLEWMGWMNPNSD | YLNWYQQKPGKAPKLLIYAASSLQSGVPSRF |
| | | NTGYAQKFQGRVTMTRDTSTSTVYMELSSL | SGSGSGTDFTLTISSLOPEDFATYYCQQSYST |
| | | RSEDTAVYYCARVGVGYGYYYYGMDVWG | PWTFGQGTKVEIK (SEQ ID NO: 397) |
| | | QGTTVTVSS (SEQ ID NO: 350) | |
| 28 | Antibody 34, | QVQLVQSGAEVKKPGASVKVSCKASGYTFS | DIVMTQSPLSLPVTPGEPASISCRSSQSLLHS |
| | 70, 91 | NYYMHWVRQAPGQGLEWMGWMNPNS | NGYNYLDWYLQKPGQSPQLLIYLASTRAPG |
| | | GNTGYAQKFQGRVTMTRDTSTSTVYMELSS | VPDRFSGSGSGTDFTLKISRVEAEDVGVYYC |
| | | LRSEDTAVYYCARASMYSSSGFDYWGQGTL | MQGSHWPPSFGQGTRLEIK (SEQ ID NO: |
| | | VTVSS (SEQ ID NO: 351) | 398) |
| 29 | Antibody 35, | QVQLVQSGAEVKKPGASVKVSCKASGYTFT | DIVMTQSPLSLPVTPGEPASISCRSSQSLLHS |
| | 71 | SYYIHWVRQAPGQGLEWMGTINPSGGSTS | NGYNYLDWYLQKPGQSPQLLIYDASSLESGV |
| | | YAQKFQGRVTMTRDTSTSTVYMELSSLRSE | PDRFSGSGSGTDFTLKISRVEAEDVGVYYCM |
| | | DTAVYYCARETSRWLRDYYYGMDVWGQG | QGTHWPLTFGGGTKVEIK (SEQ ID NO: |
| | | TTVTVSS (SEQ ID NO: 352) | 399) |
| 30 | Antibody 36, | QVQLVQSGAEVKKPGASVKVSCKASGYTFT | DIQMTQSPSSLSASVGDRVTITCQASQGISN |
| | 72 | DNYLHWVRQAPGQGLEWMGIIETSGGSTD | YLNWYQQKPGKAPKLLIYAASSLQSGVPSRF |
| | | YAQKFQGRVTMTRDTSTSTVYMELSSLRSE | SGSGSGTDFTLTISSLQPEDFATYYCQQSYSS |
| | | DTAVYYCARSFGGYSYDDAFDIWGQGTTVT | PYTFGQGTKVEIK (SEQ ID NO: 400) |
| | | VSS (SEQ ID NO: 353) | |
| 31 | Antibody 37 | QVQLVQSGAEVKKPGASVKVSCKASGYTLN | DIQMTQSPSSLSASVGDRVTITCRASQSISR |
| | | NYDINWVRQAPGQGLEWMGWISGYNGN | WLAWYQQKPGKAPKLLIYAASSLESGVPSRF |
| | | TNYAQKLQGRVTMTRDTSTSTVYMELSSLR | SGSGSGTDFTLTISSLQPEDFATYYCQQTHSF |
| | | SEDTAVYYCARVGSSWEENDQWGQGTLVT | PLTFGGGTKVEIK (SEQ ID NO: 401) |
| | | VSS (SEQ ID NO: 354) | |
| 32 | Antibody 38, | QVQLVQSGAEVKKPGASVKVSCKASGGTFS | DIVMTQSPDSLAVSLGERATINCRSSQSVLYS |
| | 73, 92, 105 | SYDINWVRQAPGQGLEWMGWMNPNSG | SNNKNYLAWYQQKPGQPPKLLIYWASTRES |
| | | NTGYAQKFQGRVTMTRDTSTSTVYMELSSL | GVPDRFSGSGSGTDFTLTISSLQAEDVAVYY |
| | | RSEDTAVYYCARDLTLYCSSTSCLDAFDYWG | CQQYYSTPPTFGQGTKLEIK (SEQ ID NO: |
| | | QGTLVTVSS (SEQ ID NO: 355) | 402) |
| 33 | Antibody 39, | QVQLVQSGAEVKKPGASVKVSCKASGGTFS | EIVMTQSPATLSVSPGERATLSCRASQSVSSY |
| | 74 | NYGFSWVRQAPGQGLEWMGWMSPSSGN | LAWYQQKPGQAPRLLIYGTSTRATGIPARFS |
| | | TGYAQKFQGRVTMTRDTSTSTVYMELSSLR | GSGSGTEFTLTISSLQSEDFAVYYCQQYGSLP |
| | | SEDTAVYYCARERSGSYFDYWGQGTLVTVS | FTFGQGTRLEIK (SEQ ID NO: 403) |
| | | S (SEQ ID NO: 356) | |
| 34 | Antibody 40 | QVQLVQSGAEVKKPGSSVKVSCKASGGTFS | DIVMTQSPDSLAVSLGERATINCKSSQSVLYS |
| | | SYAISWVRQAPGQGLEWVGRIHPRDGSTD | SNNKNYLAWYQQKPGQPPKLLIYWASTRAS |
| | | YAQKFQGRVTITADESTSTAYMELSSLRSED | GVPDRFSGSGSGTDFTLTISSLQAEDVAVYY |
| | | TAVYYCARVSLRFLEDYGMDVWGQGTTVT | CQQYYTTPPTFGQGTRLEIK (SEQ ID NO: |
| | | VSS (SEQ ID NO: 357) | 404) |
| 35 | Antibody 41, | QVQLVQSGAEVKKPGSSVKVSCKASGGTFS | DIVMTQSPLSLPVTPGEPASISCRSSQSLLHS |
| | 75 | NYAISWVRQAPGQGLEWMGGIIPIFGSTAS | NGYNYLDWYLQKPGQSPQLLIYAASSLQSG |
| | | YAQKFQGRVTITADESTSTAYMELSSLRSED | VPDRFSGSGSGTDFTLKISRVEAEDVGVYYC |
| | | TAVYYCARGVGDDTLTDWGQGTLVTVSS | MQALQTPLTFGGGTKVEIK (SEQ ID NO: |
| | | (SEQ ID NO: 358) | 405) |
| 36 | Antibody 42 | QVQLVQSGAEVKKPGASVKVSCKASGYTFT | DIVMTQSPDSLAVSLGERATINCKSSQSLLYT |
| | | SYHMHWVRQAPGQGLEWMGWMNPNSG | SNDRNYLAWYQQKPGQPPKLLIYWASTRES |
| | | NTGYAQKFQGRVTMTRDTSTSTVYMELSSL | GVPDRFSGSGSGTDFTLTISSLQAEDVAVYY |
| | | RSEDTAVYYCARGGRVVRGVIKGWFDPWG | CQQYYSSPYTFGQGTKVEIK (SEQ ID NO: |
| | | QGTLVTVSS (SEQ ID NO: 359) | 406 |
| 37 | Antibody 43 | QVQLVQSGAEVKKPGASVKVSCKASGGTFT | DIQMTQSPSSLSASVGDRVTITCRASQSISS |
| | | SYDINWVRQAPGQGLEWMGWISAYNGHT | WLAWYQQKPGKAPKLLIYDASNLETGVPSR |
| | | NYAQNLQGRVTMTRDTSTSTVYMELSSLRS | FSGSGSGTDFTLTISSLQPEDFATYYCQQSFS |
| | | EDTAVYYCAREDSSWYFDLWGRGTLVTVSS | TPPTFGQGTKVEIK (SEQ ID NO: 407) |
| | | (SEQ ID NO: 360) | |
| 38 | Antibody 44, | QVQLVQSGAEVKKPGSSVKVSCKASGGTFS | EIVMTQSPATLSVSPGERATLSCRASQSVSS |
| | 76, 93 | KIAVSWVRQAPGQGLEWMGLINPNSGGTD | NLAWYQQKPGQAPRLLIYGASTRATGIPARF |
| | | YAQKFQGRVTITADESTSTAYMELSSLRSED | SGSGSGTEFTLTISSLQSEDFAVYYCQQYNTY |
| | | TAVYYCAKDFRGGDYYYYGMDVWGQGTT | PITFGQGTKVEIK (SEQ ID NO: 408) |
| | | VTVSS (SEQ ID NO: 361) | |
| 39 | Antibody 45, | QVQLVQSGAEVKKPGASVKVSCKASGYTFT | DIQMTQSPSSLSASVGDRVTITCRASQSISSY |
| | 77, 94, 106 | SYGISWVRQAPGQGLEWMGWISAYNGNT | LNWYQQKPGKAPKLLIYAASSLQSGVPSRFS |
| | | NYAQKFQGRVTMTRDTSTSTVYMELSSLRS | GSGSGTDFTLTISSLQPEDFATYYCQQSYSTP |
| | | EDTAVYYCARGTYYDYIWGSYRYSWFDYW | LTFGGGTKVEIK (SEQ ID NO: 409) |
| | | GQGTLVTVSS (SEQ ID NO: 362) | |
| 40 | Antibody 46 | QVQLVQSGAEVKKPGASVKVSCKASGYTFS | DIQMTQSPSSLSASVGDRVTITCQASQDISN |
| | | SYAISWVRQAPGQGLEWMGWMDPNSGN | YLNWYQQKPGKAPKLLIYAASTLQTGVPSRF |
| | | TGYAQKFQGRVTMTRDTSTSTVYMELSSLR | SGSGSGTDFTLTISSLQPEDFATYYCQQSYST |
| | | SEDTAVYYCARESSYYYDSSGYYPGWYFDL | PWTFGPGTKVDIK (SEQ ID NO: 410) |
| | | WGRGTLVTVSS (SEQ ID NO: 363) | |
| 41 | Antibody 47, | QVQLVQSGAEVKKPGASVKVSCKASGYTFT | DIQMTQSPSSLSASVGDRVTITCRASQRIGSY |
| | 78 | TYGISWVRQAPGQGLEWMGWMDPNSGS | LNWYQQKPGKAPKLLIYGASNLQSGVPSRFS |
| | | TGYAQKFQGRVTMTRDTSTSTVYMELSSLR | GSGSGTDFTLTISSLQPEDFATYYCQQANSF |
| | | SEDTAVYYCARGRGNSYYYYYMDVWGKGT | PFTFGPGTKVDIK (SEQ ID NO: 411) |
| | | TVTVSS (SEQ ID NO: 364) | |
| 42 | Antibody 48, | QVQLVQSGAEVKKPGASVKVSCKASGYTFT | DIQMTQSPSSLSASVGDRVTITCRASESITN |
| | 79, 95, 107 | SYYMHWVRQAPGQGLEWMGWMNPNSG | WLAWYQQKPGKAPKLLIYKASSLESGVPSRF |
| | | NTGYAQKFQGRVTMTRDTSTSTVYMELSSL | SGSGSGTDFTLTISSLQPEDFATYYCQQSYGT |
| | | RSEDTAVYYCARDQMYSSYGMDVWGQGT | PYTFGQGTKVEIK (SEQ ID NO: 412) |
| | | TVTVSS (SEQ ID NO: 365) | |
| 43 | Antibody 49 | QVQLVQSGAEVKKPGSSVKVSCKASGGTFS | DIVMTQSPDSLAVSLGERATINCKSSQSVLYS |
| | | SYAISWVRQAPGQGLEWMGVINPSGGSTS | SNNKNYLAWYQQKPGQPPKLLIYWASTRES |
| | | YAQKFQGRVTITADESTSTAYMELSSLRSED | GVPDRFSGSGSGTDFTLTISSLQAEDVAVYY |
| | | TAVYYCARESRGYYGMDVWGQGTTVTVSS | CQQYYSTPYTFGQGTKVEIK (SEQ ID NO: |
| | | (SEQ ID NO: 366) | 413) |
| 44 | Antibody 50 | QVQLVQSGAEVKKPGASVKVSCKASGYTFT | DIQMTQSPSSLSASVGDRVTITCRASQSISS |
| | | GYYMHWVRQAPGQGLEWMGTINPSGGST | WLAWYQQKPGKAPKLLIYDTSNLQGGVPSR |
| | | SYAQKFQGRVTMTRDTSTSTVYMELSSLRSE | FSGSGSGTDFTLTISSLQPEDFATYYCQQYDS |
| | | DTAVYYCVKGNGYYDLWGQGTLVTVSS | FPWTFGQGTKVEIK (SEQ ID NO: 414) |
| | | (SEQ ID NO: 367) | |
| 45 | Antibody 51, | QVQLVQSGAEVKKPGASVKVSCKASGYTFT | DIQMTQSPSSLSASVGDRVTITCRASQGIRN |
| | 80, 96, 108 | RYDINWVRQAPGQGLEWMGWMNPNSG | DLGWYQQKPGKAPKLLIYSASNLQSGVPSRF |
| | | NTGYAQKLQGRVTMTRDTSTSTVYMELSSL | SGSGSGTDFTLTISSLQPEDFATYYCQQSYDV |
| | | RSEDTAVYYCARERVGYCSSTSCLHPLDYWG | PLTFGGGTKVEIK (SEQ ID NO: 415) |
| | | QGTLVTVSS (SEQ ID NO: 368) | |
| 46 | Antibody 52, | QVQLVQSGAEVKKPGASVKVSCKASGYTFT | DIQMTQSPSSLSASVGDRVTITCRASQSISS |
| | 81 | SYDINWVRQAPGQGLEWMGWISGYNGNT | WLAWYQQKPGKAPKLLIYDATTLESGVPSR |
| | | NYAQKFQGRVTMTRDTSTSTVYMELSSLRS | FSGSGSGTDFTLTISSLQPEDFATYYCQQYDS |
| | | EDTAVYYCARSFDWLLLLDYWGQGTLVTVS | YPWTFGQGTKLEIK (SEQ ID NO: 416) |
| | | S (SEQ ID NO: 369) | |
| 47 | Antibody 53, | QVQLVQSGAEVKKPGASVKVSCKASGHTFN | EIVMTQSPATLSVSPGERATLSCRASQSVSKY |
| | 82, 97, 109 | NNYIHWVRQAPGQGLEWMGIINPSGGSTS | LAWYQQKPGQAPRLLIYAISARAAGIPARFS |
| | | YAQKFQGRVTMTRDTSTSTVYMELSSLRSE | GSGSGTEFTLTISSLQSEDFAVYYCQQYYSSP |
| | | DTAVYYCARQIQDYGYYYYGMDVWGQGTT | PTFGQGTKVEIK (SEQ ID NO: 417) |
| | | VTVSS (SEQ ID NO: 370) | |

In some embodiments, provided herein is a SIRPγ antibody, wherein the heavy chain variable domain (VH) of the antibody comprises the amino acid sequence of SEQ ID NO: 324 or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or wherein the light chain variable domain (VL) of the antibody comprises the amino acid sequence of SEQ ID NO: 371, or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain (VH) of the antibody comprises the amino acid sequence of SEQ ID NO: 325 or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or wherein the light chain variable domain (VL) of the antibody comprises the amino acid sequence of SEQ ID NO: 372, or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain (VH) of the antibody comprises the amino acid sequence of SEQ ID NO: 326 or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or wherein the light chain variable domain (VL) of the antibody comprises the amino acid sequence of SEQ ID NO: 373, or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain (VH) of the antibody comprises the amino acid sequence of SEQ ID NO: 327 or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or wherein the light chain variable domain (VL) of the antibody comprises the amino acid sequence of SEQ ID NO: 374, or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain (VH) of the antibody comprises the amino acid sequence of SEQ ID NO: 328 or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or wherein the light chain variable domain (VL) of the antibody comprises the amino acid sequence of SEQ ID NO: 375, or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain (VH) of the antibody comprises the amino acid sequence of SEQ ID NO: 329 or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or wherein the light chain variable domain (VL) of the antibody comprises the amino acid sequence of SEQ ID NO: 376, or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain (VH) of the antibody comprises the amino acid sequence of SEQ ID NO: 330 or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or wherein the light chain variable domain (VL) of the antibody comprises the amino acid sequence of SEQ ID NO: 377, or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain (VH) of the antibody comprises the amino acid sequence of SEQ ID NO: 331 or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or wherein the light chain variable domain (VL) of the antibody comprises the amino acid sequence of SEQ ID NO: 378, or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain (VH) of the antibody comprises the amino acid sequence of SEQ ID NO: 332 or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or wherein the light chain variable domain (VL) of the antibody comprises the amino acid sequence of SEQ ID NO: 379, or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain (VH) of the antibody comprises the amino acid sequence of SEQ ID NO: 333 or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or wherein the light chain variable domain (VL) of the antibody comprises the amino acid sequence of SEQ ID NO: 380, or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain (VH) of the antibody comprises the amino acid sequence of SEQ ID NO: 334 or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or wherein the light chain variable domain (VL) of the antibody comprises the amino acid sequence of SEQ ID NO: 381, or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain (VH) of the antibody comprises the amino acid sequence of SEQ ID NO: 335 or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or wherein the light chain variable domain (VL) of the antibody comprises the amino acid sequence of SEQ ID NO: 382, or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain (VH) of the antibody comprises the amino acid sequence of SEQ ID NO: 336 or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or wherein the light chain variable domain (VL) of the antibody comprises the amino acid sequence of SEQ ID NO: 383, or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain (VH) of the antibody comprises the amino acid sequence of SEQ ID NO: 337 or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or wherein the light chain variable domain (VL) of the antibody comprises the amino acid sequence of SEQ ID NO: 384, or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain (VH) of the antibody comprises the amino acid sequence of SEQ ID NO: 338 or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or wherein the light chain variable domain (VL) of the antibody comprises the amino acid sequence of SEQ ID NO: 385, or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain (VH) of the antibody comprises the amino acid sequence of SEQ ID NO: 339 or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or wherein the light chain variable domain (VL) of the antibody comprises the amino acid sequence of SEQ ID NO: 386, or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain (VH) of the antibody comprises the amino acid sequence of SEQ ID NO: 340 or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or wherein the light chain variable domain (VL) of the antibody comprises the amino acid sequence of SEQ ID NO: 387, or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain (VH) of the antibody comprises the amino acid sequence of SEQ ID NO: 341 or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or wherein the light chain variable domain (VL) of the antibody comprises the amino acid sequence of SEQ ID NO: 388, or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain (VH) of the antibody comprises the amino acid sequence of SEQ ID NO: 342 or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or wherein the light chain variable domain (VL) of the antibody comprises the amino acid sequence of SEQ ID NO: 389, or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain (VH) of the antibody comprises the amino acid sequence of SEQ ID NO: 343 or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or wherein the light chain variable domain (VL) of the antibody comprises the amino acid sequence of SEQ ID NO: 390, or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain (VH) of the antibody comprises the amino acid sequence of SEQ ID NO: 344 or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or wherein the light chain variable domain (VL) of the antibody comprises the amino acid sequence of SEQ ID NO: 391, or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain (VH) of the antibody comprises the amino acid sequence of SEQ ID NO: 345 or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or wherein the light chain variable domain (VL) of the antibody comprises the amino acid sequence of SEQ ID NO: 392, or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain (VH) of the antibody comprises the amino acid sequence of SEQ ID NO: 346 or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or wherein the light chain variable domain (VL) of the antibody comprises the amino acid sequence of SEQ ID NO: 393, or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain (VH) of the antibody comprises the amino acid sequence of SEQ ID NO: 347 or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or wherein the light chain variable domain (VL) of the antibody comprises the amino acid sequence of SEQ ID NO: 394, or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain (VH) of the antibody comprises the amino acid sequence of SEQ ID NO: 348 or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or wherein the light chain variable domain (VL) of the antibody comprises the amino acid sequence of SEQ ID NO: 395, or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain (VH) of the antibody comprises the amino acid sequence of SEQ ID NO: 349 or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or wherein the light chain variable domain (VL) of the antibody comprises the amino acid sequence of SEQ ID NO: 396, or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain (VH) of the antibody comprises the amino acid sequence of SEQ ID NO: 350 or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or wherein the light chain variable domain (VL) of the antibody comprises the amino acid sequence of SEQ ID NO: 397, or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain (VH) of the antibody comprises the amino acid sequence of SEQ ID NO: 351 or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or wherein the light chain variable domain (VL) of the antibody comprises the amino acid sequence of SEQ ID NO: 398, or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain (VH) of the antibody comprises the amino acid sequence of SEQ ID NO: 352 or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or wherein the light chain variable domain (VL) of the antibody comprises the amino acid sequence of SEQ ID NO: 399, or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain (VH) of the antibody comprises the amino acid sequence of SEQ ID NO: 353 or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or wherein the light chain variable domain (VL) of the antibody comprises the amino acid sequence of SEQ ID NO: 400, or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain (VH) of the antibody comprises the amino acid sequence of SEQ ID NO: 354 or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or wherein the light chain variable domain (VL) of the antibody comprises the amino acid sequence of SEQ ID NO: 401, or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain (VH) of the antibody comprises the amino acid sequence of SEQ ID NO: 355 or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or wherein the light chain variable domain (VL) of the antibody comprises the amino acid sequence of SEQ ID NO: 402, or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain (VH) of the antibody comprises the amino acid sequence of SEQ ID NO: 356 or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or wherein the light chain variable domain (VL) of the antibody comprises the amino acid sequence of SEQ ID NO: 403, or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain (VH) of the antibody comprises the amino acid sequence of SEQ ID NO: 357 or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or wherein the light chain variable domain (VL) of the antibody comprises the amino acid sequence of SEQ ID NO: 404, or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain (VH) of the antibody comprises the amino acid sequence of SEQ ID NO: 358 or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or wherein the light chain variable domain (VL) of the antibody comprises the amino acid sequence of SEQ ID NO: 405, or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain (VH) of the antibody comprises the amino acid sequence of SEQ ID NO: 359 or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or wherein the light chain variable domain (VL) of the antibody comprises the amino acid sequence of SEQ ID NO: 406, or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain (VH) of the antibody comprises the amino acid sequence of SEQ ID NO: 360 or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or wherein the light chain variable domain (VL) of the antibody comprises the amino acid sequence of SEQ ID NO: 407, or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain (VH) of the antibody comprises the amino acid sequence of SEQ ID NO: 361 or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or wherein the light chain variable domain (VL) of the antibody comprises the amino acid sequence of SEQ ID NO: 408, or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain (VH) of the antibody comprises the amino acid sequence of SEQ ID NO: 362 or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or wherein the light chain variable domain (VL) of the antibody comprises the amino acid sequence of SEQ ID NO: 409, or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain (VH) of the antibody comprises the amino acid sequence of SEQ ID NO: 363 or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or wherein the light chain variable domain (VL) of the antibody comprises the amino acid sequence of SEQ ID NO: 410, or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain (VH) of the antibody comprises the amino acid sequence of SEQ ID NO: 364 or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or wherein the light chain variable domain (VL) of the antibody comprises the amino acid sequence of SEQ ID NO: 411, or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain (VH) of the antibody comprises the amino acid sequence of SEQ ID NO: 365 or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or wherein the light chain variable domain (VL) of the antibody comprises the amino acid sequence of SEQ ID NO: 412, or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain (VH) of the antibody comprises the amino acid sequence of SEQ ID NO: 366 or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or wherein the light chain variable domain (VL) of the antibody comprises the amino acid sequence of SEQ ID NO: 413, or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain (VH) of the antibody comprises the amino acid sequence of SEQ ID NO: 367 or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or wherein the light chain variable domain (VL) of the antibody comprises the amino acid sequence of SEQ ID NO: 414, or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain (VH) of the antibody comprises the amino acid sequence of SEQ ID NO: 368 or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or wherein the light chain variable domain (VL) of the antibody comprises the amino acid sequence of SEQ ID NO: 415, or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain (VH) of the antibody comprises the amino acid sequence of SEQ ID NO: 369 or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or wherein the light chain variable domain (VL) of the antibody comprises the amino acid sequence of SEQ ID NO: 416, or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain (VH) of the antibody comprises the amino acid sequence of SEQ ID NO: 370 or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or wherein the light chain variable domain (VL) of the antibody comprises the amino acid sequence of SEQ ID NO: 417, or an amino acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

By way of explanation, making reference to Tables 1 and 2, forty seven (47) combinations of unique antibody sequences are provided (CDR sequences in Table 1; VH/VL sequences in Table 2). By way of example, and guidance to read the tables: Antibodies 1, 3, 4, 7, and 110 share the same combination of CDR sequences and also share the same combination of VH/VL sequences, but differ in some other aspect, e.g. may have different Fc regions. Likewise, it is noted that Antibodies 2, 5, 6, 8, and 111 share the same combination of CDR sequences and also share the same combination of VH/VL sequences but differ in some other aspect, e.g. may have different Fc regions.

Antibodies 1 and 2 comprise a rat IgG2b Fc.

Antibodies 3, 5, and 83-97 comprise a human IgG1 Fc of the disclosure.

Antibodies 4, 6, and 54-82 comprise a human IgG1 Fc of the disclosure comprising certain substitutions that lead to increased effector function, exhibiting increased affinity to FcγR.

Antibodies 7 and 8 comprise a human IgG4 Fc of the disclosure comprising certain substitutions that lead to a decrease in the dynamic process of Fab-arm exchange, and further reduced effector function.

Antibodies 98-111 comprise a human IgG1 Fc of the disclosure comprising certain substitutions that lead to reduced effector function, leading to the silencing of the Fc (e.g. can comprise the LALA-PG substitutions).

iii. SIRPγ Antibody-Mediated Cell Depletion

The Fc-containing SIRPγ antibodies provided herein are capable of targeting and preferentially depleting SIRPγ-expressing cells. In some embodiments, the antibodies preferentially deplete activated (interchangeably referred to herein as stimulated) cells as a result of an increase in surface SIRPγ expression compared to naïve unactivated (unstimulated) cells.

In some embodiments, the SIRPγ antibodies provided herein are capable of inducing the depletion of T-cells, B-cells, or NK-cells. In some embodiments, the cells are in an activated state.

Without being held to any theory or mechanism, it is envisioned that the CDR-containing antigen-binding domain of the SIRPγ antibody confers binding to the SIRPγ-expressing cell, and that the Fc portion of the antibody induces depletion and Fc-mediated effector function. Accordingly, in some embodiments, the cell depletion involves antibody dependent cellular cytotoxicity (ADCC). In some embodiments, the cell depletion involves antibody dependent cellular phagocytosis (ADCP). In some embodiments, the cell depletion involves complement-dependent cytotoxicity (CDC). In some embodiments, the cell depletion involves one, two, or all three of ADCC, ADCP, and CDC. An Fc-containing SIRPγ antibody of the disclosure includes a full-length antibody, or an antibody fragment that is linked to a Fc domain, e.g. a VH-VL-Fc single chain antibody.

In some embodiments, there is differential depletion of T-cell subsets, possibly driven by differential expression of specific isoforms on different T-cell subsets. T-cell subsets include cells that are in different cell states, e.g. stimulated, exhausted; subsets also include T-cells that express different subsets of markers. Accordingly, in some embodiments, the SIRPγ antibodies provided herein are capable of inducing the preferential depletion of specific T-cell subsets expressing specific SIRPγ isoforms.

### B. Generation of SIRPγ Antibodies

Production of the antibodies provided herein may be by use of any method known to those of ordinary skill in the art. In some embodiments, the antibodies are produced by hybridomas. In some embodiments, the antibodies are encoded by a nucleic acid and are expressed, purified, and isolated.

The terms polynucleotide and nucleic acid are used interchangeably herein, and refer to a polymeric form of nucleotides of any length, which may be ribonucleotides or deoxyribonucleotides. The terms include, but are not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivative nucleotide bases. The terms encompass nucleic acids containing known analogues of natural nucleotides and having similar binding properties, and are metabolized in a manner similar to naturally-occurring nucleotides, unless specifically limited or stated otherwise.

Accordingly, provided herein are nucleic acids encoding any of the antibodies disclosed herein, vectors comprising any of the nucleic acids encoding such antibodies, and host cells comprising any such vectors. Also provided herein are exemplary nucleic acid sequences encoding for the variable heavy chains and variable light chains of the SIRPγ antibodies disclosed herein.

Table 3 provides exemplary nucleic acid sequences for the SIRPγ antibodies of the disclosure.

TABLE 3
Exemplary Variable Heavy Chain and Variable Light Chain Nucleic Acid Sequence
of SIRPγ Antibodies

| Antibody | Variable Heavy | Variable |
|---|---|---|
| No. | Chain Nucleic Acid Sequence | Light Chain Nucleic Acid Sequence |
| Antibody | CAGGTGCAGCTGGTGGAGTCTGGGGGAGGCGT | GAAATAGTGATGACGCAGTCTCCAGCCACCCTGT |
| 1, 3, 4, 7, | GGTCCAGCCTGGGAGGTCCCTGAGACTCTCCTGT | CTGTGTCTCCAGGGGAAAGAGCCACCCTCTCCTG |
| 110 | GCAGCGTCTGGATTCACCTTCAGTAACTATGGCA | CAGGGCCAGTCAGAGTGTTAGCAGCAACTTAGC |
| | TGCACTGGGTCCGCCAGGCTCCAGGCAAGGGGC | CTGGTACCAGCAGAAACCTGGCCAGGCTCCCAG |
| | TGGAGTGGGTGGCATTTATATGGTATGATGGAA | GCTCCTCATCTATGGTGCATCCACCAGGGCCACT |
| | GTAATAAAAACTATGCAGACTCCGTGAAGGGCC | GGTATCCCAGCCAGGTTCAGTGGCAGTGGGTCT |
| | GATTCACCATCTCCAGAGACAATCCCACGAACAC | GGGACAGAGTTCACTCTCACCATCAGCAGCCTGC |
| | GCTGTATCTGCAAATGAACAGCCTGAGAGCCGA | AGTCTGAAGATTTTGCAGTTTATTACTGTCAGCA |
| | GGACACGGCTGTGTATTACTGTGCGAGAGAGGA | GTATAATAACTGGCCTCTCACTTTCGGCGGAGGG |
| | GCAGTGGCTTGACTACTGGGGCCAGGGAACCCT | ACCAAGGTGGAGATCAAA (SEQ ID NO: 465) |
| | GGTCACCGTCTCCTCA (SEQ ID NO: 418) | |
| Antibody | CAGGTGCAGCTGGTGCAGTCTGGGGCTGAGGTG | GACATCCAGATGACCCAGTCTCCTTCCACCCTGTC |
| 2, 5, 6, 8, | AAGAAGCCTGGGGCCTCAGTGAAGGTCTCCTGC | TGCATCTGTTGGAGACAGAGTCACCATCACTTGC |
| 111 | AAGGCTTCTGGATACACCTTCACCGGCTATCATA | CGGGCCAGTCAGAGTATTAGTAGCTGGTTGGCC |
| | TACACTGGGTGCGACAGGCCCCTGGACAAGGGC | TGGTATCAGCAGAAACCAGGGAAAGCCCCTAAG |
| | TTGAGTGGATGGGATGGATCAACCCTAACAGTG | CTCCTGATCTATAAGGCGTCTAGTTTAGAAACTG |
| | GTGGCACAAACTATGCACAGAAGTTTCAGGGCA | GGGTCCCATCAAGGTTCAGCGGCAGTGGATCTG |
| | GGGTCACCATGACCAGGGACACGTCCATCAACA | GGACAGAATTCACTCTCACCATCAGCAGCCTGCA |
| | CGGCCTACATGGAGCTGAACAGGCTGAGATCTG | GCCTGATGATTTTGCCACTTATTACTGCCAACAGT |
| | ACGACACGGCCGTGTTTTACTGTGCGAGAGAGA | ATGATAGTTACTACACTTTTGGCCAGGGGACCAA |
| | GAGTAGGATATTGTAGTCGTCCCAGCTGCCTTGA | GCTGGAGATCAAA (SEQ ID NO: 466) |
| | TGATCTTAATATCTGGGGCCAGGGGACAATGGTC | |
| | ACCGTCTCTTCA (SEQ ID NO: 419) | |
| Antibody | CAGGTGCAGCTGGTGCAGTCTGGGGCTGAGGTG | GAAATAGTGATGACGCAGTCTCCAGCCACCCTGT |
| 9, 54, 83, | AAGAAGCCTGGGGCCTCAGTGAAGGTTTCCTGC | CTGTGTCTCCAGGGGAAAGAGCCACCCTCTCCTG |
| 98 | AAGGCATCTGGATACAGCTTCTCTGACTACTATA | CAGGGCCAGTCAGAGTCTGGGCAGCACTTATTTA |
| | TGCACTGGGTGCGACAGGCCCCTGGACAAGGGC | GCCTGGTACCAGCAGAAACCTGGCCAGGCTCCC |
| | TTGAGTGGATGGGATGGATGTCCCCTAATAGTG | AGGCTCCTCATCTATGGTGCATCCACCAGGGCCA |
| | GTAACACAGGCTACGCACAGAAGTTCCAGGGCA | CTGGTATCCCAGCCAGGTTCAGTGGCAGTGGGT |
| | GAGTCACCATGACCAGGGACACGTCCACGAGCA | CTGGGACAGAGTTCACTCTCACCATCAGCAGCCT |
| | CAGTCTACATGGAGCTGAGCAGCCTGAGATCTG | GCAGTCTGAAGATTTTGCAGTTTATTACTGTCAG |
| | AGGACACGGCCGTGTATTACTGTGCTAGACAAC | CAGTATTATAGCTCCCCTCCCACGTTCGGCCAAG |
| | GCGAGGAATACGGCCACTACTACTACGGGATGG | GGACCAAGGTGGAAATCAAA (SEQ ID NO: 467) |
| | ATGTCTGGGGCCAAGGGACAATGGTCACCGTCT | |
| | CTTCA (SEQ ID NO: 420) | |
| Antibody 10 | CAGGTGCAGCTGGTGCAGTCTGGGGCTGAGGTG | GACATCCAGATGACCCAGTCTCCATCCTCCCTGTC |
| | AAGAAGCCTGGGGCCTCAGTGAAGGTTTCCTGC | TGCATCTGTAGGAGACAGAGTCACCATCACTTGC |
| | AAGGCATCTGGAGGGACCTTCTCTAACTACGACA | CAGGCGAGTCAGGACATTCACAACTATTTAAATT |
| | TCTCGTGGGTGCGACAGGCCCCTGGACAAGGGC | GGTATCAGCAGAAACCAGGGAAAGCCCCTAAGC |
| | TTGAGTGGATGGGAATGATCGATCCTAGTGGTG | TCCTGATCTACGATGCATCCAATTTGGAAACAGG |
| | GCAGCACAAGCTACGCACAGAAGTTCCAGGGCA | GGTCCCATCAAGGTTCAGTGGAAGTGGATCTGG |
| | GAGTCACCATGACCAGGGACACGTCCACGAGCA | GACAGATTTTACTCTCACCATCAGCAGCCTGCAG |
| | CAGTCTACATGGAGCTGAGCAGCCTGAGATCTG | CCTGAAGATTTTGCAACATATTACTGTCAACAGA |
| | AGGACACGGCCGTGTATTACTGTGCTAGACCTTA | GTTATAATACCCCTCCCACCTTCGGCCAAGGGAC |
| | TTCAAGCGGGTGGTACTACTTTGACTACTGGGGC | ACGACTGGAGATTAAA (SEQ ID NO: 468) |
| | CAAGGGACCCTGGTCACCGTCTCCTCA (SEQ ID | |
| | NO: 421) | |
| Antibody 11 | CAGGTGCAGCTGGTGCAGTCTGGGGCTGAGGTG | GACATCCAGATGACCCAGTCTCCATCCTCCCTGTC |
| | AAGAAGCCTGGGGCCTCAGTGAAGGTCTCCTGC | TGCATCTGTAGGAGACAGAGTCACCATCACTTGC |
| | AAGGCTTCTGGATACACCTTCACCAGTTATGATA | CGCGCGAGTCAGACCATTAATAACTATTTAAATT |
| | TCAACTGGGTGCGACAGGCCCCTGGACAAGGGC | GGTATCAGCAGAAACCAGGGAAAGCCCCTAAGC |
| | TTGAGTGGATGGGATGGATGAACCCCAACAGTG | TCCTGATCTACGATGCATCCAATTTGGAAACAGG |
| | GTAACACAGGCTATGCACAGAAGTTCCAGGGCA | GGTCCCATCAAGGTTCAGTGGAAGTGGATCTGG |
| | GAGTCACCATGACCAGGGACACCTCCACGAGCA | GACAGATTTTACTCTGACCATCAGCAGCCTGCAG |
| | CAGTCTACATGGAGCTGAGCAGCCTGAGATCTG | CCTGAAGATTTTGCAACATATTACTGTCAACAGG |
| | AGGACACGGCCGTGTATTACTGTGCGAAAGGCG | CTAATAGTTTCCCTCTCACTTTCGGCCAGGGGACC |
| | CTGTGGCCGGAACTTACTACTATTACGGAATGGA | AAGGTGGAGATCAAA (SEQ ID NO: 469) |
| | CGTGTGGGGCCAAGGGACCACGGTCACCGTCTC | |
| | CTCA (SEQ ID NO: 422) | |
| Antibody 12 | CAGGTTCAGCTGGTGCAGTCTGGAGCTGAGGTG | GACATCCAGATGACCCAGTCTCCATCCTCCCTGTC |
| | AAGAAGCCTGGGGCCTCAGTGAAGGTCTCCTGC | TGCATCTGTAGGAGACAGAGTCACCATCACTTGC |
| | AAGGCTTCTGGTGGCACCTTTGGCAGCTATGGTA | CGGGCAAGTCAGAACATTAGGAACTATTTAAATT |
| | TCAACTGGGTGCGACAGGCCCCTGGACAAGGGC | GGTATCAGCAGAAACCAGGGAAAGCCCCTAAGC |
| | TTGAGTGGATGGGATGGATCAGCGCCTACAATG | TCCTGATCTATGCTGCATCCAGTTTGCAAGGAGG |
| | GTAACACAAACTATGCACAGAAGCTCCAGGGCA | GGTCCCATCAAGGTTCAGTGGCAGTGGATCTGG |
| | GAGTCACCATGACCCGGGACACATCCACGAGCA | GACAGATTTCACTCTCACCATCAGCAGTCTGCAA |
| | CAGTCTACATGGAGCTGAGCAGCCTGAGATCTG | CCTGAAGATTTTGCAACTTACTACTGTCAACAGA |
| | AAGACACGGCCGTGTATTACTGTGCGAGAGATG | GTTACAACGGGCCTTACACGTTCGGCCAAGGGA |
| | GAATCGATTACGGCGGGAACTCTGGATATGACT | CCAAGGTGGAAATCAAA (SEQ ID NO: 470) |
| | ACTGGGGCCAAGGGACCCTGGTCACCGTCTCCTC | |
| | A (SEQ ID NO: 423) | |
| Antibody | GAGGTGCAGCTGCTCGAGTCTGGGGGAGGCTTG | GATATTGTGATGACTCAGTCTCCACTCTCCCTGCC |
| 13, 55 | GTACAGCCTGGAGGGTCCCTGAGACTCTCCTGTG | CGTCACCCCTGGAGAGCCGGCCTCCATCTCCTGC |
| | CAGCCTCTGGATTCACCTTCAGTAACAGTGACAT | AGGTCTAGTCAGAGCCTCCTGCACTCTAACGGGT |
| | GAACTGGGTCCGCCAGGCTCCAGGGAAGGGGCT | ACAACTATTTGGATTGGTACCTGCAGAAGCCAGG |
| | GGAGTGGGTTTCATACATTAGTAGTAGTGGTAAT | GCAGTCTCCACAGCTCCTGATCTATGCAGCGTCT |
| | ACTATATACTACGCAGACTCTGTGAAGGGCCGAT | AGTCTGCAGTCCGGGGTCCCTGACAGGTTCAGT |
| | TCACCATCTCCAGAGACAACTCAAAGAACACGCT | GGCAGTGGATCAGGCACAGATTTTACACTGAAA |
| | GTATCTGCAAATGAACAGCCTGAGAGCCGAGGA | ATCAGCAGAGTGGAGGCTGAGGATGTTGGGGTT |
| | CACGGCTGTTTATTACTGTGCGAGAAGCAACTGG | TATTACTGCATGCAAGGGACCCACTGGCCTCCAA |
| | AATTACCCTCTTGACTACTACTACATGGACGTCTG | CGTTCGGCCAAGGGACCAAGGTGGAAATCAAA |
| | GGGCAAAGGGACCACGGTCACCGTCTCCTCA | (SEQ ID NO: 471) |
| | (SEQ ID NO: 424) | |
| Antibody | CAGGTGCAGCTGGTGCAGTCTGGGGCTGAGGTG | GATATTGTGATGACTCAGTCTCCACTCTCCCTGCC |
| 14,56 | AAGAAGCCTGGGGCCTCAGTGAAGGTTTCCTGC | CGTCACCCCTGGAGAGCCGGCCTCCATCTCCTGC |
| | AAGGCATCTGGATACACCTTCACCAGCTACTATA | AGGTCTAGTCAGAGCCTCCTGCATTCGAATGGGT |
| | TGCACTGGGTGCGACAGGCCCCTGGACAAGGGC | ACAACTATTTGGATTGGTACCTGCAGAAGCCAGG |
| | TTGAGTGGATGGGATGGGTAGATCCCAATAACG | GCAGTCTCCACAGCTCCTGATCTATGCAGCTTCTA |
| | GTGACACAGGATACGCACAGAAGTTCCAGGGCA | CCCTCCAGTCCGGGGTCCCTGACAGGTTCAGTGG |
| | GAGTCACCATGACCAGGGACACGTCCACGAGCA | CAGTGGATCAGGCACAGATTTTACACTGAAAATC |
| | CAGTCTACATGGAGCTGAGCAGCCTGAGATCTG | AGCAGAGTGGAGGCTGAGGATGTTGGGGTTTAT |
| | AGGACACGGCCGTGTATTACTGTGCTAGAGATA | TACTGCATGCAAGGTCTACAAACTTTATACAGTTT |
| | ACTGGGGCTCCCTGGATGTCTGGGGCAAAGGGA | TGGCCAGGGGACCAAGCTGGAGATCAAA (SEQ |
| | CAACCGTCACCGTCTCTTCA (SEQ ID NO: 425) | ID NO: 472) |
| Antibody 15 | CAGGTTCAGCTGGTGCAGTCTGGAGCTGAGGTG | GACATCCAGATGACCCAGTCTCCATCTTCTCTGTC |
| | AAGAAGCCTGGGGCCTCAGTGAAGGTCTCCTGC | TGCATCTGTAGGAGACAGAGTCACCATCACTTGT |
| | AAGGCTTCTGGTTACTCCTTTACCAGCTATGGTAT | CGGGCGAGTCAGGGTGTGGGCGATAGAAGTGC |
| | CAGCTGGGTGCGACAGGCCCCTGGACAAGGGCT | CTGGTATCAGCAGAAACCAGGGAAAGCCCCTAA |
| | TGAGTGGATGGGATGGATCAGCCCGTACAATGG | GCTCCTGATCTATGACGCATCCAACTTGGAGACA |
| | TAACACAAACTATGCACAGAAGCTCCAGGGCAG | GGGGTCCCATCAAGGTTCAGCGGCAGTGGATCT |
| | AGTCACCATGACCAGAGACACATCCACGAGCAC | GGGACAGATTTCACTCTCACTATCAGCAGCCTGC |
| | AGTTTACATGGAGCTGTCCAGCCTGAGATCTGAA | AGCCTGAAGATTTTGCAACTTACTATTGTCAACA |
| | GACACGGCCGTGTATTACTGTGCGAGAGAGGCT | GGCTGATACTTTCCCTTACACTTTTGGCCAGGGG |
| | TACGGCGACTACGGCGACTACTTTGACTACTGGG | ACCAAGCTGGAGATCAAA (SEQ ID NO: 473) |
| | GCCAAGGGACCCTGGTCACCGTCTCCTCA (SEQ | |
| | ID NO: 426) | |
| Antibody | CAGGTGCAGCTGGTGCAGTCTGGGGCTGAGGTG | GACATCCAGATGACCCAGTCTCCATCCTCCCTGTC |
| 16, 57, 84, | AAGAAGCCTGGGGCCTCAGTGAAGGTTTCCTGC | TGCATCTGTAGGAGACAGAGTCACCATCACTTGC |
| 99 | AAGGCATCTGGATACACCTTCACCAGCTACTATA | CGGGCAAGTCAGGGCATTAGCACTTATTTAGCCT |
| | TGCACTGGGTGCGACAGGCCCCTGGACAAGGGC | GGTATCAGCAGAAACCAGGGAAAGCCCCTAAGC |
| | TTGAGTGGATGGGATGGATCAACCCAAACAGTG | TCCTGATCTATGCTGCATCCACATTGCAAAGTGG |
| | GTAACGCCGGGTTTGCACAGAAGTTCCAGGGCA | GGTCCCATCAAGGTTCAGTGGCAGTGGATCTGG |
| | GAGTCACCATGACCAGGGACACGTCCACGAGCA | GACAGATTTCACTCTCACCATCAGCAGTCTGCAA |
| | CAGTCTACATGGAGCTGAGCAGCCTGAGATCTG | CCTGAAGATTTTGCAACTTACTACTGTCAACAGA |
| | AGGACACGGCCGTGTATTACTGTGCTAGAGGAC | GTCACAGTGCACCTATCACGTTCGGCCAAGGGAC |
| | CCGTCTATTCCTACGGTCCGTTTGACTACTGGGG | CAAGGTGGAAATCAAA (SEQ ID NO: 474) |
| | CCAAGGGACCCTGGTCACCGTCTCCTCA (SEQ ID | |
| | NO: 427) | |
| Antibody 17 | CAGGTGCAGCTGGTGCAGTCTGGGGCTGAGGTG | GACATCCAGATGACCCAGTCTCCATCCTCCCTGTC |
| | AAGAAGCCTGGGGCCTCAGTGAAGGTTTCCTGC | TGCATCTGTAGGAGACAGAGTCACCATCACTTGC |
| | AAGGCATCTGGATACACCTTCACCAGGAATTATG | CGGGCAAGTCAGAGCATTAGCAGCTATTTAAATT |
| | TCCACTGGGTGCGACAGGCCCCTGGACAAGGGC | GGTATCAGCAGAAACCAGGGAAAGCCCCTAAGC |
| | TTGAGTGGATGGGACGGATCAACCCCAATAGTG | TCCTGATCTATGACGCATCCAGTTTGGAGAGTGG |
| | GTGGGACAAACACCGCACAGAAGTTCCAGGGCA | GGTCCCATCAAGGTTCAGTGGCAGTGGATCTGG |
| | GAGTCACCATGACCAGGGACACGTCCACGAGCA | GACAGATTTCACTCTCACCATCAGCAGTCTGCAA |
| | CAGTCTACATGGAGCTGAGCAGCCTGAGATCTG | CCTGAAGATTTTGCAACTTACTACTGTCAACAGG |
| | AGGACACGGCCGTGTATTACTGTGCTAGACTGG | CAAACAGTTTCCCTTTAACTTTCGGCCCTGGGACC |
| | GCGACGTGGCCGTCGCTGCGTCCTTTGACTACTG | AAAGTGGATATCAAA (SEQ ID NO: 475) |
| | GGGCCAAGGGACCCTGGTCACCGTCTCCTCA | |
| | (SEQ ID NO: 428) | |
| Antibody 18 | CAGGTGCAGCTGGTGCAGTCTGGGGCTGAGGTG | GACATCCAGATGACCCAGTCTCCATCTTCTCTTTC |
| | AAGAAGCCTGGGGCCTCAGTGAAGGTTTCCTGC | TGCATCTGTAGGAGACAGAGTCACCATCACTTGT |
| | AAGGCATCTGGATACACCTTCACCGGCTACTATA | CGGGCGAGTCAGGGTATTGGAAATGACTTAGCC |
| | TGCACTGGGTGCGACAGGCCCCTGGACAAGGGC | TGGTATCAGCAGAAACCAGGGAAAGCCCCTAAG |
| | TTGAGTGGATGGGATGGATGAACCCAAATAGTG | CTCCTGATCTATGACGCAAAGGATTTGCACCCCG |
| | GTAATACAGGTTACGCACAGAAGTTCCAGGGCA | GGGTCCCATCAAGGTTCAGCGGCAGTGGATCTG |
| | GAGTCACCATGACCAGGGACACGTCCACGAGCA | GGACAGATTTCACTCTCACTATCAGCAGCCTGCA |
| | CAGTCTACATGGAGCTGAGCAGCCTGAGATCTG | GCCTGAAGATTTTGCAACTTACTATTGTCAACAG |
| | AGGACACGGCCGTGTATTACTGTGCTAGAGGAT | AGCTACAGTTTCCCTAGGACCTTCGGCCAAGGGA |
| | CTGGCTACGATGCTGGGGTGTTTGACTACTGGG | CACGACTGGAGATTAAA (SEQ ID NO: 476) |
| | GCCAAGGGACCCTGGTCACCGTCTCCTCA (SEQ | |
| | ID NO: 429) | |
| Antibody | CAGGTGCAGCTGGTGCAGTCTGGGGCTGAGGTG | GACATCCAGATGACCCAGTCTCCATCCTCCCTGTC |
| 19, 58 | AAGAAGCCTGGGGCCTCAGTGAAGGTTTCCTGC | TGCATCTGTAGGAGACAGAGTCACCATCACTTGC |
| | AAGGCATCTGGATACACCTTCACCGCATACTATA | CGGGCAAGTGAGAGCATTAGCCATTGGTTAGCC |
| | TGCACTGGGTGCGACAGGCCCCTGGACAAGGGC | TGGTATCAGCAGAAACCAGGGAAAGCCCCTAAG |
| | TTGAGTGGATGGGAATAATCAACCCTAGTGGTG | CTCCTGATCTATGGTGCATCCACTTTGCAAAGTG |
| | GCAGCACAAGCTACGCACAGAAGTTCCAGGGCA | GGGTCCCATCAAGGTTCAGTGGCAGTGGATCTG |
| | GAGTCACCATGACCAGGGACACGTCCACGAGCA | GGACAGATTTCACTCTCACCATCAGCAGTCTGCA |
| | CAGTCTACATGGAGCTGAGCAGCCTGAGATCTG | ACCTGAAGATTTTGCAACTTACTACTGTCAACAG |
| | AGGACACGGCCGTGTATTACTGTGCTAGAGATA | AGTTACACCCCTCCCCTCACTTTCGGCGGAGGGA |
| | GCGGGTGGTACGCACCCGGATACTATTATGGGA | CCAAGGTGGAGATCAAA (SEQ ID NO: 477) |
| | TGGATGTCTGGGGCCAAGGGACAACAGTCACCG | |
| | TCTCTTCA (SEQ ID NO: 430) | |
| Antibody | CAGGTGCAGCTGGTGCAGTCTGGGGCTGAGGTG | GACATCGTGATGACCCAGTCTCCAGACTCCCTGG |
| 20, 59, 85, | AAGAAGCCTGGGGCCTCAGTGAAGGTCTCCTGC | CTGTGTCTCTGGGCGAGAGGGCCACCATCAACTG |
| 100 | AAGGCTTCTGGATACACCTTCACCAGTTATGATA | CAAGTCCAGCCAGAGTCTCCTCTACAGCTCTAAT |
| | TCAACTGGGTGCGACAGGCCCCAGGACAAGGGC | AACAAGAACTACTTAGCTTGGTACCAGCAGAAAC |
| | TTGAGTGGATGGGATGGATGAACCCCAACAGTG | CAGGACAGCCTCCTAAGCTGCTCATTTACTGGGC |
| | GTAACACAGGCTATGCACAGAAGTTCCAGGGCA | ATCTATCCGGGAATCCGGGGTCCCTGACCGATTC |
| | GAGTCACCATGACCAGGGACACCTCCACAAGCA | AGTGGCAGCGGGTCTGGGACAGATTTCACTCTCA |
| | CAGTTTACATGGAGCTGAGCAGCCTGAGATCTGA | CCATCAGCAGCCTGCAGGCTGAAGATGTGGCAG |
| | GGACACGGCCGTGTATTACTGTGCGAGAGAGTC | TTTATTACTGTCAGCAATATCTCAGTCCTCCACTC |
| | ATACGCATATTGCTCAAGTACATCCTGTTATGATG | ACTTTCGGCCAGGGGACCAAGGTGGAGATCAAA |
| | CTTTTGATATCTGGGGCCAAGGGACAATGGTCAC | (SEQ ID NO: 478) |
| | CGTCTCTTCA (SEQ ID NO: 431) | |
| Antibody | GAGGTGCAGCTGTTGGAGTCTGGGGGAGGCTTG | GACATCCAGATGACCCAGTCTCCATCCTCCCTGTC |
| 21, 60, 86, | GTACAGCCTGGGGGGTCCCTGAGACTCTCCTGTG | TGCATCTGTAGGAGACAGAGTCACCATCACTTGC |
| 101 | CAGCCTCTGGATTCACCTTGGGGAGCTATTGGAT | CGGGCAAGTCAGGATATTAGACGAGCATTAGCC |
| | GCATTGGGTCCGCCAGGCTCCAGGGAAGGGGCT | TGGTATCAGCAGAAACCAGGGAAAGCCCCTAAG |
| | GGAGTGGGTCTCATTAATTAGTTGGGATGGTGG | CTGCTGATCTATGCTGCATCCAGTTTGCAAAGTG |
| | CTCTACATACTACGCAGACTCCGTGAAGGGCCGG | GGGTCCCATCAAGGTTCAGCGGCAGTGGATCTG |
| | TTCACCATCTCCAGAGACAATTCCAAGAACACGC | GGACAGATTTCACTCTCACAATCAGCAGCCTGCA |
| | TGTATCTGCAAATGAACAGCCTGAGAGCCGAGG | GCCTGAAGATTTTGCAACTTATTACTGTCTACAGC |
| | ACACGGCCGTATATTACTGTGCGAAAGGCCCATG | ATAATAGTTACCCTCCCACTTTTGGCCAGGGGAC |
| | GGTCGGTTACTCAGGCTACGATCTGAATTACTAC | CAAGCTGGAGATCAAA (SEQ ID NO: 479) |
| | TACTACGGCATGGACGTTTGGGGCCAAGGGACC | |
| | ACAGTCACCGTCTCCTCA (SEQ ID NO: 432) | |
| Antibody | CAGGTGCAGCTGGTGCAGTCTGGGGCTGAGGTG | GATATTGTGATGACTCAGTCTCCACTCTCCCTGCC |
| 22, 61, 87 | AAGAAGCCTGGGGCCTCAGTGAAGGTTTCCTGC | CGTCACCCCTGGAGAGCCGGCCTCCATCTCCTGC |
| | AAGGCATCTGGATACACCTTCACCAGCCACGACA | AGGTCTAGTCAGAGCCTCCTCCACAGTAATGGAT |
| | TTAATTGGGTGCGACAGGCCCCTGGACAAGGGC | ACAACTATTTGGATTGGTACCTGCAGAAGCCAGG |
| | TTGAGTGGATGGGAATATCAAACCCTAGTGGAG | GCAGTCTCCACAGCTCCTGATCTATTTGGGTTCTA |
| | GTAGCACATACTACGCACAGAAGTTCCAGGGCA | GTCGGGCCTCCGGGGTCCCTGACAGGTTCAGTG |
| | GAGTCACCATGACCAGGGACACGTCCACGAGCA | GCAGTGGATCAGGCACAGATTTTACACTGAAAAT |
| | CAGTCTACATGGAGCTGAGCAGCCTGAGATCTG | CAGCAGAGTGGAGGCTGAGGATGTTGGGGTTTA |
| | AGGACACGGCCGTGTATTACTGTGCTTCATCCCG | TTACTGCATGCAAGCTCTACAAACTCCTACCTTCG |
| | ACGGTACTGGAATGGCGCCTTTGACTACTGGGG | GCCAAGGGACACGACTGGAGATTAAA (SEQ ID |
| | CCAAGGGACCCTGGTCACCGTCTCCTCA (SEQ ID | NO: 480) |
| | NO: 433) | |
| Antibody | CAGGTTCAGCTGGTGCAGTCTGGAGCTGAGGTG | GACATCCAGATGACCCAGTCTCCATCCTCCCTGTC |
| 23, 62 | AAGAAGCCTGGGGCCTCAGTGAAGGTCTCCTGC | TGCATCTGTAGGAGACAGAGTCACCATCACTTGC |
| | AAGGCTTCTGGTGGGACCTTTAGTAACTATGATA | CAGGCAAGTCAGGGTATTAGCAGCTATTTAAATT |
| | TCAATTGGGTGCGACAGGCCCCTGGACAAGGGC | GGTATCAGCAGAAACCAGGGAAAGCCCCTAAGC |
| | TTGAGTGGATGGGATGGATCAGCGCGTACAATG | TCCTGATCTATGCTGCATCCAGTTTGCAAAATGG |
| | GTAACACAAACTATGCACAGAAGCTCCAGGGCA | GGTCCCATCAAGGTTCAGTGGCAGTGGATCTGG |
| | GAGTCACCATGACCCGGGACACATCCACGAGCA | GACAGATTTCACTCTCACCATCAGCAGTCTGCAA |
| | CAGTGTACATGGAGCTGAGCAGCCTGAGATCTG | CCTGAAGATTTTGCAACTTACTACTGTCAACAGA |
| | AAGACACGGCCGTGTATTACTGTGCGGCAGAAG | GTTACAGTACCCCTATCACCTTCGGCCAAGGGAC |
| | CCTACGGAGATTACTCACTGGACCCCTGGGGCCA | ACGACTGGAGATTAAA (SEQ ID NO: 481) |
| | GGGAACCCTGGTCACCGTCTCCTCA (SEQ ID NO: | |
| | 434) | |
| Antibody | CAGGTGCAGCTGGTGCAGTCTGGGGCTGAGGTG | GACATCCAGATGACCCAGTCTCCATCCTCCCTGTC |
| 24, 63 | AAGAAGCCTGGGGCCTCAGTGAAGGTTTCCTGC | TGCATCTGTAGGAGACAGAGTCACCATCACTTGC |
| | AAGGCATCTGGATACACCTTCACCGGATACTATA | CGGGCAAGTCAGGGCATTGGCAATGATTTAGGC |
| | TGCACTGGGTGCGACAGGCCCCTGGACAAGGGC | TGGTATCAGCAGAAACCAGGGAAAGCCCCTAAG |
| | TTGAGTGGATGGGAATAATCAACCCTAGTGGTG | CTTCTGATCTATGCTGCATCCAACTTGGAAACGG |
| | GCAGCACAAACTACGCACAGAAGTTCCAGGGCA | GGGTCCCATCAAGGTTCAGCGGCAGTGGATCTG |
| | GAGTCACCATGACCAGGGACACGTCCACGAGCA | GGACAGATTTCACTCTCACAATCAGCAGCCTGCA |
| | CAGTCTACATGGAGCTGAGCAGCCTGAGATCTG | GCCTGAAGATTTTGCAACTTATTACTGTCTACAGC |
| | AGGACACGGCCGTGTATTACTGTGCTAGAGGCC | ATCATAGTTACCCTTTTACTTTTGGCCAGGGGACC |
| | CGATTGGCTACGGTATGGACGTGTGGGGCCAAG | AAGCTGGAGATCAAA (SEQ ID NO: 482) |
| | GGACCCTGGTCACCGTCTCCTCA (SEQ ID NO: | |
| | 435) | |
| Antibody 25 | GAGGTGCAGCTGCTGGAGTCTGGGGGAGGCTTG | GACATCCAGATGACCCAGTCTCCATCCTCCCTGTC |
| | GTCCAGCCTGGGGGGTCCCTGAGACTCTCCTGTG | TGCATCTGTAGGAGACAGAGTCACCATCACTTGC |
| | CAGCCTCTGGATTCACCGTCGGCAGCTGGTACAT | CGGACCAGTCAGAGCATTGGCACCTATTTAAATT |
| | GAGCTGGGTCCGCCAGGCTCCAGGGAAGGGGCT | GGTATCAGCAGAAACCAGGGAAAGCCCCTAAGC |
| | GGAGTGGGTCGCTCATGTCTCGGACGACGGTAG | TCCTGATCTATGACGCATCCAATTTGCCGACTGG |
| | CAATGAATTTTACGCAGACTCCGTGAAGGGCCGA | GGTCCCATCAAGGTTCAGTGGCAGTGGATCTGG |
| | TTCACCATCTCCAGAGACAATTCCAAGAACACGC | GACAGATTTCACTCTCACCATCAGCAGTCTGCAA |
| | TGTATCTTCAAATGAACAGCCTGAGAGCTGAGGA | CCTGAAGATTTTGCAACTTACTACTGTCAAAACTA |
| | CACGGCTGTGTATTACTGTGCGAGAAGCCCCGGC | TAACAGTGCTCTGTTCGGCCCTGGGACCAAAGTG |
| | TATTGTAGCTCCACCAGCTGTTACCGATACTATG | GATATCAAA (SEQ ID NO: 483) |
| | GTATGGACGTCTGGGGCCAAGGGACCACAGTCA | |
| | CCGTCTCCTCA (SEQ ID NO: 436) | |
| Antibody | CAGGTGCAGCTGGTGCAGTCTGGGGCTGAGGTG | GACATCCAGATGACCCAGTCTCCATCTTCTCTCTC |
| 26, 64 | AAGAAGCCTGGGGCCTCAGTGAAGGTTTCCTGC | TGCATCTGTAGGAGACAGAGTCACCATCACTTGT |
| | AAGGCATCTGGATACACCTTCACCAGCTACTATA | CGGGCGAGTCAGTCTGTGGATACCTGGTTAGCCT |
| | TGCACTGGGTGCGACAGGCCCCTGGACAAGGGC | GGTATCAGCAGAAACCAGGGAAAGCCCCTAAGC |
| | TTGAGTGGATGGGACGTATCAACCCGAATAGTG | TCCTGATCTATGAAGCATCCACATTGGAAAGTGG |
| | GTGGAACAAAGTACGCACAGAAGTTCCAGGGCA | GGTCCCATCAAGGTTCAGCGGCAGTGGATCTGG |
| | GAGTCACCATGACCAGGGACACGTCCACGAGCA | GACAGATTTCACTCTCACTATCAGCAGCCTGCAG |
| | CAGTCTACATGGAGCTGAGCAGCCTGAGATCTG | CCTGAAGATTTTGCAACTTACTATTGTCAACAGG |
| | AGGACACGGCCGTGTATTACTGTGCTAGACTAG | CTAACAGTTTCCCTATCACCTTCGGCCAAGGGAC |
| | GCTCTGCCTACTTCGATCTCTGGGGCCGTGGCAC | ACGACTGGAGATTAAA (SEQ ID NO: 484) |
| | CCTGGTCACTGTCTCCTCA (SEQ ID NO: 437) | |
| Antibody | CAGGTGCAGCTGGTGCAGTCTGGGGCTGAGGTG | GACATCCAGATGACCCAGTCTCCATCCTCCCTGTC |
| 27, 65, 88, | AAGAAGCCTGGGGCCTCAGTGAAGGTCTCCTGC | TGCATCTGTAGGAGACAGAGTCACCATCACTTGC |
| 102 | AAGGCTTCTGGATACACCTTCACCAGTCATGATA | CGGGCAAGTCAGAGCATTAGCAATCTCTTAGCGT |
| | TCAACTGGGTGCGACAGGCCCCAGGACAAGGGC | GGTATCAGCAGAAACCAGGGAAAGCCCCTAAGC |
| | TTGAGTGGATGGGATGGATGAACGCTAACAACG | TCCTGATCTATGCTGCATCCAGTTTGCAAAGTGG |
| | GTAACGCTGGCTATGCACAGAAGTTCCAGGGCA | GGTCCCATCAAGGTTCAGTGGCAGTGGATCTGG |
| | GAGTCACCATGACCAGGGATACCTCCACCAGCAC | GACAGATTTCACTCTCACCATCAGCAGTCTGCAA |
| | AGTTTACATGGAGCTGAGCAGCCTGAGATCTGA | CCTGAAGATTTTGCAACTTACTACTGTCAACAGA |
| | GGACACGGCCGTGTATTACTGTGCGAAGGACTT | GTTACAGTACCCCTTACACTTTTGGCCAGGGGAC |
| | GTCTCCATCTTCTTCCTGGTCCACCTACTATTACTA | CAAGCTGGAGATCAAA (SEQ ID NO: 485) |
| | TTACGGGATGGATGTCTGGGGCCAAGGGACAAT | |
| | GGTCACCGTCTCTTCA (SEQ ID NO: 438) | |
| Antibody 28 | CAGGTTCAGCTGGTGCAGTCTGGAGCTGAGGTG | GACATCCAGATGACCCAGTCTCCATCTTCTCTTTC |
| | AAGAAGCCTGGGGCCTCAGTGAAGGTCTCCTGC | TGCATCTGTAGGAGACAGAGTCACCATCACTTGT |
| | AAGGCTTCTGGTTACACCTTTACCAGCTATAATAT | CGGGCGAGTCAGGGTATTAGCAATTGGTTAGCC |
| | CCACTGGGTGCGACAGGCCCCTGGACAAGGGCT | TGGTATCAGCAGAAACCAGGGAAAGCCCCTAAG |
| | TGAGTGGATGGGATGGATCAGCGCCTACAATGG | CTCCTGATCTATGATGCATCCAATTTGGACGCCG |
| | TAACACAAACTATGCACAGAAGCTCCAGGGCAG | GGGTCCCATCAAGGTTCAGCGGCAGTGGATCTG |
| | AGTCACCATGACCAGAGACACATCCACGAGCAC | GGACAGATTTCACTCTCACTATCAGCAGCCTGCA |
| | AGTCTACATGGAGCTGAGCAGCCTGAGATCTGA | GCCTGAAGATTTTGCAACTTACTATTGTCAACAG |
| | GGACACGGCCGTGTATTACTGTGCGCTGGATGG | GCTAACAGTTTACCTCTCACTTTCGGCGGAGGGA |
| | AGGGCAGCTAGGCGTGGCTTTTGATATCTGGGG | CCAAGGTGGAGATCAAA (SEQ ID NO: 486) |
| | CCAAGGGACAATGGTCACCGTCTCTTCA (SEQ ID | |
| | NO: 439) | |
| Antibody | CAGGTGCAGCTGGTGCAGTCTGGGGCTGAGGTG | GAAATAGTGATGACGCAGTCTCCAGCCACCCTGT |
| 29, 66, 89, | AAGAAGCCTGGGGCCTCAGTGAAGGTTTCCTGC | CTGTGTCTCCAGGGGAAAGAGCCACCCTCTCCTG |
| 103 | AAGGCATCTGGAGGAACCTTCAGCAGCTACGCT | CAGGGCCAGTCAGAGTATATCTGGGAGCTATTTA |
| | ATCAGCTGGGTGCGACAGGCCCCTGGACAAGGG | GCCTGGTACCAGCAGAAACCTGGCCAGGCTCCC |
| | CTTGAGTGGATGGGAATAATCAACCCTAGTTCCA | AGGCTCCTCATCTATGGTGCATCCACCAGGGCCA |
| | GCAGCACAGGCTACGCACAGAAGTTCCAGGGCA | CTGGTATCCCAGCCAGGTTCAGTGGCAGTGGGT |
| | GAGTCACCATGACCAGGGACACGTCCACGAGCA | CTGGGACAGAGTTCACTCTCACCATCAGCAGCCT |
| | CAGTCTACATGGAGCTGAGCAGCCTGAGATCTG | GCAGTCTGAAGATTTTGCAGTTTATTACTGTCAG |
| | AGGACACGGCCGTGTATTACTGTGCTAGAGCCG | CAGTATGGGAGCTTTCCTTTCACTTTCGGCCCTG |
| | GTTACTACGACTCTTCGGGCTATTACTATTCCCCT | GGACCAAAGTGGATATCAAA (SEQ ID NO: 487) |
| | TACTATGGCATGGACGTGTGGGGCCAAGGGACC | |
| | ACAGTCACCGTCTCCTCA (SEQ ID NO: 440) | |
| Antibody | CAGGTTCAGCTGGTGCAGTCTGGAGCTGAGGTG | GACATCCAGATGACCCAGTCTCCATCCTCCCTGTC |
| 30, 67 | AAGAAGCCTGGGGCCTCAGTGAAGGTCTCCTGC | TGCATCTGTAGGAGACAGAGTCACCATCACTTGC |
| | AAGGCTTCTGGTTACACCTTTACCAACTTCGACAT | CGGGCAAGTCAGGATATTCGTAATGATTTAGCCT |
| | CAATTGGGTGCGACAGGCCCCTGGACAAGGGCT | GGTATCAGCAGAAACCAGGGAAAGCCCCTAAGC |
| | TGAGTGGATGGGATGGATCAGCGCCTACAATGG | TCCTGATCTATGACGCATCCAATTTGGAGACGGG |
| | TCATACAAACTATGCACAGAACCTCCAGGGCAGA | GGTCCCATCAAGGTTCAGTGGCAGTGGATCTGG |
| | GTCACCATGACCAGGGACACATCCACGAGCACA | GACAGATTTCACTCTCACCATCAGCAGTCTGCAA |
| | GTGTACATGGAGCTGTCTAGCCTGAGATCTGAAG | CCTGAAGATTTTGCAACTTACTACTGTCAACAGA |
| | ACACGGCCGTGTATTACTGTGCGCAGGAAGCCG | GTTACGGTACCCCTTACACTTTTGGCCAGGGGAC |
| | CGGCTGGGTACTTTGACTACTGGGGCCAAGGGA | CAAGCTGGAGATCAAA (SEQ ID NO: 488) |
| | CCCTGGTCACCGTCTCCTCA (SEQ ID NO: 441) | |
| Antibody 31 | CAGGTGCAGCTGGTGCAGTCTGGGGCTGAGGTG | GACATCGTGATGACCCAGTCTCCAGACTCCCTGG |
| | AAGAAGCCTGGGGCCTCAGTGAAGGTCTCCTGC | CTGTGTCTCTGGGCGAGAGGGCCACCATCAACTG |
| | AAGGCTTCTGGAGGAACCTTCCGCAGTTATGCCA | CCGGTCCAGCCAGAGTCTCTTTCACAGCAGCACC |
| | TCAGCTGGGTGCGACAGGCCCCAGGACAAGGGC | AACGCCAACAACTTAGCTTGGTACCAGCAGAAAC |
| | TTGAGTGGATGGGATGGATGAACCCTAACAGTG | CAGGACAGCCTCCTAAGCTGCTCATTTACTGGGC |
| | GTAACACAGGCTATGCACAGAAGTTCCAGGGCA | ATCTACCCGGGAATCCGGGGTCCCTGACCGATTC |
| | GAGTCACCATGACCAGGGATACCTCCACAAGCAC | AGTGGCAGCGGGTCTGGGACAGATTTCACTCTCA |
| | AGTGTACATGGAGCTGAGCAGCCTGAGATCTGA | CCATCAGCAGCCTGCAGGCTGAAGATGTGGCAG |
| | GGACACGGCCGTGTATTACTGTGCGAGAGAGAG | TTTATTACTGTCAGCAATATTATATTACTCCTCTG |
| | CGTGCTCAAAGGAGGTATGGATGTCTGGGGCCA | ACCTTCGGCCAAGGGACACGACTGGAGATTAAA |
| | AGGGACAACGGTCACCGTCTCTTCA (SEQ ID NO: | (SEQ ID NO: 489) |
| | 442) | |
| Antibody | CAGGTGCAGCTGGTGCAGTCTGGGGCTGAGGTG | GACATCCAGATGACCCAGTCTCCATCCTCCCTGTC |
| 32, 68 | AAGAAGCCTGGGGCCTCAGTGAAGGTTTCCTGC | TGCATCTGTAGGAGACAGAGTCACCATCACTTGC |
| | AAGGCATCTGGATACACCTTCACCAGCTACTATA | CGGGCAAGTCAGAGCATTAGCAACTGGTTAGCC |
| | TGTATTGGGTGCGACAGGCCCCTGGACAAGGGC | TGGTATCAGCAGAAACCAGGGAAAGCCCCTAAG |
| | TTGAGTGGATGGGATGGATCAACCCAAATAGTG | CTCCTGATCTATGCTGCATCCAGTTTGCAAAGTG |
| | GTAACACAGGATACGCACAGAAGTTCCAGGGCA | GGGTCCCATCAAGGTTCAGTGGCAGTGGATCTG |
| | GAGTCACCATGACCAGGGACACGTCCACGAGCA | GGACAGATTTCACTCTCACCATCAGCAGTCTGCA |
| | CAGTCTACATGGAGCTGAGCAGCCTGAGATCTG | ACCTGAAGATTTTGCAACTTACTACTGTCAACAG |
| | AGGACACGGCCGTGTATTACTGTGCTAAGGATA | AGTTACAGTACCCCTTACACTTTTGGCCAGGGGA |
| | GGCGCCGTGGAGTGGGCCTAGCCGCTGCCGGCA | CCAAGCTGGAGATCAAA (SEQ ID NO: 490) |
| | CCAGCGCTTTTGATATCTGGGGCCAAGGGACAAT | |
| | GGTCACCGTCTCTTCA (SEQ ID NO: 443) | |
| Antibody | CAGGTGCAGCTGGTGCAGTCTGGGGCTGAGGTG | GACATCCAGATGACCCAGTCTCCATCCTCCCTGTC |
| 33, 69, 90 | AAGAAGCCTGGGGCCTCAGTGAAGGTTTCCTGC | TGCATCTGTAGGAGACAGAGTCACCATCACTTGC |
| 104 | AAGGCATCTGGATACACCTTCACCAGCTACTATA | CGGGCAGGTCAGAGCATTGGCGACTATTTAAATT |
| | TGCACTGGGTGCGACAGGCCCCTGGACAAGGGC | GGTATCAGCAGAAACCAGGGAAAGCCCCTAAGC |
| | TTGAGTGGATGGGATGGATGAACCCTAACTCTG | TCCTGATCTATGCTGCATCCAGTTTGCAAAGTGG |
| | ACAACACAGGTTACGCACAGAAGTTCCAGGGCA | GGTCCCATCAAGGTTCAGTGGCAGTGGATCTGG |
| | GAGTCACCATGACCAGGGACACGTCCACGAGCA | GACAGATTTCACTCTCACCATCAGCAGTCTGCAA |
| | CAGTCTACATGGAGCTGAGCAGCCTGAGATCTG | CCTGAAGATTTTGCAACTTACTACTGTCAACAGA |
| | AGGACACGGCCGTGTATTACTGTGCTAGAGTGG | GTTACAGTACCCCTTGGACGTTCGGCCAAGGGAC |
| | GGGTCGGTTATGGCTACTACTACTACGGAATGGA | CAAGGTGGAAATCAAA (SEQ ID NO: 491) |
| | CGTGTGGGGCCAAGGGACCACCGTCACCGTCTC | |
| | CTCA (SEQ ID NO: 444) | |
| Antibody | CAGGTGCAGCTGGTGCAGTCTGGGGCTGAGGTG | GATATTGTGATGACTCAGTCTCCACTCTCCCTGCC |
| 34, 70, 91 | AAGAAGCCTGGGGCCTCAGTGAAGGTTTCCTGC | CGTCACCCCTGGAGAGCCGGCCTCCATCTCCTGC |
| | AAGGCATCTGGATACACCTTCTCAAATTACTATAT | AGGTCTAGTCAGAGCCTCCTGCACAGCAATGGGT |
| | GCACTGGGTGCGACAGGCCCCTGGACAAGGGCT | ACAACTATTTGGATTGGTACCTGCAGAAGCCAGG |
| | TGAGTGGATGGGATGGATGAACCCCAATAGTGG | GCAGTCTCCACAGCTCCTGATCTATTTGGCGTCTA |
| | TAACACAGGGTACGCACAGAAGTTCCAGGGCAG | CTCGGGCCCCTGGGGTCCCTGACAGGTTCAGTG |
| | AGTCACCATGACCAGGGACACGTCCACGAGCAC | GCAGTGGATCAGGCACAGATTTTACACTGAAAAT |
| | AGTCTACATGGAGCTGAGCAGCCTGAGATCTGA | CAGCAGAGTGGAGGCTGAGGATGTTGGGGTTTA |
| | GGACACGGCCGTGTATTACTGTGCTAGAGCCTCA | TTACTGCATGCAAGGATCCCATTGGCCTCCTAGTT |
| | ATGTACTCTTCAAGTGGGTTTGACTACTGGGGCC | TTGGCCAGGGGACCCGGCTGGAGATCAAA (SEQ |
| | AAGGGACCCTGGTCACCGTCTCCTCA (SEQ ID | ID NO: 492) |
| | NO: 445) | |
| Antibody | CAGGTGCAGCTGGTGCAGTCTGGGGCTGAGGTG | GATATTGTGATGACTCAGTCTCCACTCTCCCTGCC |
| 35, 71 | AAGAAGCCTGGGGCCTCAGTGAAGGTTTCCTGC | CGTCACCCCTGGAGAGCCGGCCTCCATCTCCTGC |
| | AAGGCATCTGGATACACCTTCACCAGCTACTATA | AGGTCTAGTCAGAGCCTCCTACACTCTAATGGTT |
| | TCCACTGGGTGCGACAGGCCCCTGGACAAGGGC | ACAACTATTTGGATTGGTACCTGCAGAAGCCAGG |
| | TTGAGTGGATGGGAACGATCAACCCTAGTGGTG | GCAGTCTCCACAGCTCCTGATCTATGACGCCTCTA |
| | GCAGCACAAGCTACGCACAGAAGTTCCAGGGCA | GCCTGGAGTCCGGGGTCCCTGACAGGTTCAGTG |
| | GAGTCACCATGACCAGGGACACGTCCACGAGCA | GCAGTGGATCAGGCACAGATTTTACACTGAAAAT |
| | CAGTCTACATGGAGCTGAGCAGCCTGAGATCTG | CAGCAGAGTGGAGGCTGAGGATGTTGGGGTTTA |
| | AGGACACGGCCGTGTATTACTGTGCTAGAGAGA | TTACTGCATGCAAGGGACTCACTGGCCTCTCACT |
| | CTAGCCGGTGGCTCAGAGATTATTATTACGGAAT | TTCGGCGGAGGGACCAAGGTGGAGATCAAA |
| | GGATGTCTGGGGCCAAGGGACAACTGTCACCGT | (SEQ ID NO: 493) |
| | CTCTTCA (SEQ ID NO: 446) | |
| Antibody | CAGGTGCAGCTGGTGCAGTCTGGGGCTGAGGTG | GACATCCAGATGACCCAGTCTCCATCCTCCCTGTC |
| 36, 72 | AAGAAGCCTGGGGCCTCAGTGAAGGTTTCCTGC | TGCATCTGTAGGAGACAGAGTCACCATCACTTGC |
| | AAGGCATCTGGATACACCTTCACCGACAATTATC | CAGGCAAGTCAGGGTATTAGCAATTATTTAAATT |
| | TACACTGGGTGCGACAGGCCCCTGGACAAGGGC | GGTATCAGCAGAAACCAGGGAAAGCCCCTAAGC |
| | TTGAGTGGATGGGAATAATCGAGACGAGTGGTG | TCCTGATCTATGCTGCATCCAGTTTGCAAAGTGG |
| | GCAGCACAGACTACGCACAGAAGTTCCAGGGCA | GGTCCCATCAAGGTTCAGTGGCAGTGGATCTGG |
| | GAGTCACCATGACCAGGGACACGTCCACGAGCA | GACAGATTTCACTCTCACCATCAGCAGTCTGCAA |
| | CAGTCTACATGGAGCTGAGCAGCCTGAGATCTG | CCTGAAGATTTTGCAACTTACTACTGTCAACAGA |
| | AGGACACGGCCGTGTATTACTGTGCTAGAAGCTT | GTTACAGTAGCCCTTACACGTTCGGCCAAGGGAC |
| | TGGTGGATATTCGTATGACGATGCTTTTGATATC | CAAGGTGGAAATCAAA (SEQ ID NO: 494) |
| | TGGGGCCAAGGGACAACGGTCACCGTCTCTTCA | |
| | (SEQ ID NO: 447) | |
| Antibody 37 | CAGGTTCAGCTGGTGCAGTCTGGAGCTGAGGTG | GACATCCAGATGACCCAGTCTCCATCTTCTCTGTC |
| | AAGAAGCCTGGGGCCTCAGTGAAGGTCTCCTGC | TGCATCTGTAGGAGACAGAGTCACCATCACTTGT |
| | AAGGCTTCTGGTTACACCCTCAATAATTATGATAT | CGGGCGAGTCAGTCCATTAGCCGGTGGTTAGCC |
| | CAATTGGGTGCGACAGGCCCCTGGACAAGGGCT | TGGTATCAGCAGAAACCAGGGAAAGCCCCTAAG |
| | TGAGTGGATGGGATGGATCAGCGGCTACAATGG | CTCCTGATCTATGCTGCATCCAGTTTGGAGAGTG |
| | TAACACAAACTATGCACAGAAGCTCCAGGGCAG | GGGTCCCATCAAGGTTCAGCGGCAGTGGATCTG |
| | AGTCACCATGACCCGCGACACATCCACGAGCACA | GGACAGATTTCACTCTCACTATCAGCAGCCTGCA |
| | GTGTACATGGAGCTGTCGAGCCTGAGATCTGAG | GCCTGAAGATTTTGCAACTTACTATTGTCAACAG |
| | GACACGGCCGTGTATTACTGTGCGAGAGTGGGC | ACACACAGTTTCCCTCTCACTTTCGGCGGAGGGA |
| | TCCTCTTGGGAAGAAAACGACCAGTGGGGCCAA | CCAAGGTGGAGATCAAA (SEQ ID NO: 495) |
| | GGGACCCTGGTCACCGTCTCCTCA (SEQ ID NO: | |
| | 448) | |
| Antibody | CAGGTGCAGCTGGTGCAGTCTGGGGCTGAGGTG | GACATCGTGATGACCCAGTCTCCAGACTCCCTGG |
| 38, 73, 92, | AAGAAGCCTGGGGCCTCAGTGAAGGTCTCCTGC | CTGTGTCTCTGGGCGAGAGGGCCACCATCAACTG |
| 105 | AAGGCTTCTGGAGGCACCTTCTCGAGTTATGATA | CCGATCCAGCCAGAGTGTTTTGTACTCATCAAAC |
| | TCAACTGGGTGCGACAGGCCCCAGGACAAGGGC | AATAAGAACTACTTAGCTTGGTACCAGCAGAAAC |
| | TTGAGTGGATGGGATGGATGAACCCCAACAGTG | CAGGACAGCCTCCTAAGCTGCTCATTTACTGGGC |
| | GTAACACAGGCTATGCACAGAAGTTCCAGGGCA | ATCTACCCGGGAATCCGGGGTCCCTGACCGATTC |
| | GAGTCACCATGACCAGGGACACCTCCACAAGCA | AGTGGCAGCGGGTCTGGGACAGATTTCACTCTCA |
| | CAGTGTACATGGAGCTGAGCAGCCTGAGATCTG | CCATCAGCAGCCTGCAGGCTGAAGATGTGGCAG |
| | AGGACACGGCCGTGTATTACTGTGCGAGAGATC | TTTATTACTGTCAGCAATATTATAGTACTCCTCCA |
| | TCACGCTGTATTGTAGCAGTACCAGCTGTCTTGA | ACTTTTGGCCAGGGGACCAAGCTGGAGATCAAA |
| | CGCCTTTGACTACTGGGGCCAAGGGACCCTGGTC | (SEQ ID NO: 496) |
| | ACCGTCTCCTCA (SEQ ID NO: 449) | |
| Antibody | CAGGTGCAGCTGGTGCAGTCTGGGGCTGAGGTG | GAAATAGTGATGACGCAGTCTCCAGCCACCCTGT |
| 39, 74 | AAGAAGCCTGGGGCCTCAGTGAAGGTCTCCTGC | CTGTGTCTCCAGGGGAAAGAGCCACCCTCTCCTG |
| | AAGGCTTCTGGAGGAACCTTCTCTAATTATGGTT | CAGGGCCAGTCAGAGTGTTAGCAGCTATTTAGCC |
| | TTAGCTGGGTGCGACAGGCCCCAGGACAAGGGC | TGGTACCAGCAGAAACCTGGCCAGGCTCCCAGG |
| | TTGAGTGGATGGGATGGATGTCACCTTCTAGTG | CTCCTCATCTATGGTACCTCCACCAGGGCCACTG |
| | GTAACACAGGCTATGCACAGAAGTTCCAGGGCA | GTATCCCAGCCAGGTTCAGTGGCAGTGGGTCTG |
| | GAGTCACCATGACCAGGGATACCTCCACAAGCAC | GGACAGAGTTCACTCTCACCATCAGCAGCCTGCA |
| | AGTGTACATGGAGCTGAGCAGCCTGAGATCTGA | GTCTGAAGATTTTGCAGTTTATTACTGTCAGCAG |
| | GGACACGGCCGTGTATTACTGTGCGAGAGAACG | TATGGCTCGCTACCTTTCACCTTCGGCCAAGGGA |
| | CTCCGGTTCCTACTTTGACTACTGGGGCCAAGGG | CACGACTGGAGATTAAA (SEQ ID NO: 497) |
| | ACCCTGGTCACCGTCTCCTCA (SEQ ID NO: 450) | |
| Antibody 40 | CAGGTCCAGCTGGTGCAGTCTGGGGCTGAGGTG | GACATCGTGATGACCCAGTCTCCAGACTCCCTGG |
| | AAGAAGCCTGGGTCCTCGGTGAAGGTCTCCTGC | CTGTGTCTCTGGGCGAGAGGGCCACCATCAACTG |
| | AAGGCTTCTGGAGGCACCTTCAGCAGCTATGCTA | CAAGTCCAGCCAGAGTGTTTTGTACAGTTCAAAT |
| | TCAGCTGGGTGCGACAGGCCCCTGGACAAGGGC | AACAAGAACTACTTAGCTTGGTACCAGCAGAAAC |
| | TTGAGTGGGTGGGAAGGATCCATCCACGCGACG | CAGGACAGCCTCCTAAGCTGCTCATTTACTGGGC |
| | GTAGCACAGACTACGCACAGAAGTTCCAGGGCA | ATCTACCCGGGCATCCGGGGTCCCTGACCGATTC |
| | GAGTCACGATTACCGCGGACGAATCCACGAGCA | AGTGGCAGCGGGTCTGGGACAGATTTCACTCTCA |
| | CAGCCTACATGGAGCTGAGCAGCCTGAGATCTG | CCATCAGCAGCCTGCAGGCTGAAGATGTGGCAG |
| | AGGACACGGCCGTGTATTACTGTGCGAGAGTTTC | TTTATTACTGTCAGCAATATTATACTACTCCTCCA |
| | CCTGCGCTTCCTAGAGGACTACGGTATGGACGTC | ACCTTCGGCCAAGGGACACGACTGGAGATTAAA |
| | TGGGGCCAAGGGACCACGGTCACCGTCTCCTCA | (SEQ ID NO: 498) |
| | (SEQ ID NO: 451) | |
| Antibody | CAGGTGCAGCTGGTGCAGTCTGGGGCTGAGGTG | GATATTGTGATGACTCAGTCTCCACTCTCCCTGCC |
| 41, 75 | AAGAAGCCTGGGTCCTCGGTGAAGGTCTCCTGC | CGTCACCCCTGGAGAGCCGGCCTCCATCTCCTGC |
| | AAGGCTTCTGGAGGCACCTTCAGCAACTATGCTA | AGGTCTAGTCAGAGCCTCCTGCATTCAAACGGGT |
| | TCAGCTGGGTGCGACAGGCCCCTGGACAAGGGC | ACAACTATTTGGATTGGTACCTGCAGAAGCCAGG |
| | TTGAGTGGATGGGAGGGATCATTCCCATCTTTGG | GCAGTCTCCACAGCTCCTGATCTATGCTGCTTCTA |
| | TTCGACAGCAAGCTACGCACAGAAGTTCCAGGG | GCCTGCAGTCCGGGGTCCCTGACAGGTTCAGTG |
| | CAGAGTCACGATTACCGCGGACGAATCCACGAG | GCAGTGGATCAGGCACAGATTTTACACTGAAAAT |
| | CACAGCCTACATGGAGCTGAGCAGCCTGAGATCT | CAGCAGAGTGGAGGCTGAGGATGTTGGGGTTTA |
| | GAGGACACGGCCGTGTATTACTGTGCGAGAGGT | TTACTGCATGCAAGCTCTACAAACTCCTCTCACTT |
| | GTCGGAGACGACACCCTGACTGATTGGGGCCAA | TCGGCGGAGGGACCAAGGTGGAGATCAAA (SEQ |
| | GGGACCCTGGTCACCGTCTCCTCA (SEQ ID NO: | ID NO: 499) |
| | 452) | |
| Antibody 42 | CAGGTGCAGCTGGTGCAGTCTGGGGCTGAGGTG | GACATCGTGATGACCCAGTCTCCAGACTCCCTGG |
| | AAGAAGCCTGGGGCCTCAGTGAAGGTTTCCTGC | CTGTGTCTCTGGGCGAGAGGGCCACCATCAACTG |
| | AAGGCATCTGGATACACCTTCACCAGCTACCACA | CAAGTCCAGCCAGAGTCTTCTGTATACTTCCAAC |
| | TGCACTGGGTGCGACAGGCCCCTGGACAAGGGC | GATCGCAACTACTTAGCTTGGTACCAGCAGAAAC |
| | TTGAGTGGATGGGATGGATGAACCCGAACAGTG | CAGGACAGCCTCCTAAGCTGCTCATTTACTGGGC |
| | GTAACACAGGGTACGCACAGAAGTTCCAGGGCA | ATCTACCCGGGAATCCGGGGTCCCTGACCGATTC |
| | GAGTCACCATGACCAGGGACACGTCCACGAGCA | AGTGGCAGCGGGTCTGGGACAGATTTCACTCTCA |
| | CAGTCTACATGGAGCTGAGCAGCCTGAGATCTG | CCATCAGCAGCCTGCAGGCTGAAGATGTGGCAG |
| | AGGACACGGCCGTGTATTACTGTGCTAGAGGTG | TTTATTACTGTCAGCAATATTATAGTAGCCCTTAC |
| | GACGCGTAGTGAGGGGGGTGATCAAAGGGTGG | ACGTTCGGCCAAGGGACCAAGGTGGAAATCAAA |
| | TTCGACCCCTGGGGCCAGGGAACCCTGGTCACC | (SEQ ID NO: 500) |
| | GTCTCCTCA (SEQ ID NO: 453) | |
| Antibody 43 | CAGGTTCAGCTGGTGCAGTCTGGAGCTGAGGTG | GACATCCAGATGACCCAGTCTCCATCCTCCCTGTC |
| | AAGAAGCCTGGGGCCTCAGTGAAGGTCTCCTGC | TGCATCTGTAGGAGACAGAGTCACCATCACTTGC |
| | AAGGCTTCTGGTGGAACCTTTACCAGCTATGACA | CGGGCAAGTCAGAGCATTAGCAGCTGGTTAGCC |
| | TCAATTGGGTGCGACAGGCCCCTGGACAAGGGC | TGGTATCAGCAGAAACCAGGGAAAGCCCCTAAG |
| | TTGAGTGGATGGGATGGATCAGCGCATACAATG | CTCCTGATCTATGACGCATCCAATTTGGAGACTG |
| | GTCATACAAACTATGCACAGAATCTCCAGGGCAG | GGGTCCCATCAAGGTTCAGTGGCAGTGGATCTG |
| | AGTCACCATGACCCGCGACACATCCACGAGCACA | GGACAGATTTCACTCTCACCATCAGCAGTCTGCA |
| | GTGTACATGGAGCTGTCGAGCCTGAGATCTGAA | ACCTGAAGATTTTGCAACTTACTACTGTCAACAG |
| | GACACGGCCGTGTATTACTGTGCGAGAGAAGAC | AGTTTCAGTACCCCTCCAACGTTCGGCCAAGGGA |
| | AGTTCTTGGTACTTCGATCTCTGGGGCCGTGGCA | CCAAGGTGGAAATCAAA (SEQ ID NO: 501) |
| | CCCTGGTCACTGTCTCCTCA (SEQ ID NO: 454) | |
| Antibody | CAGGTGCAGCTGGTGCAGTCTGGGGCTGAGGTG | GAAATAGTGATGACGCAGTCTCCAGCCACCCTGT |
| 44, 76, 93 | AAGAAGCCTGGGTCCTCGGTGAAGGTCTCCTGC | CTGTGTCTCCAGGGGAAAGAGCCACCCTCTCCTG |
| | AAGGCTTCTGGAGGCACCTTCAGCAAGATTGCTG | CAGGGCCAGTCAGAGTGTTAGCAGCAACTTAGC |
| | TGAGCTGGGTGCGACAGGCCCCTGGACAAGGGC | CTGGTACCAGCAGAAACCTGGCCAGGCTCCCAG |
| | TTGAGTGGATGGGACTTATCAACCCAAACTCCGG | GCTCCTCATCTATGGTGCATCCACCAGGGCCACT |
| | TGGGACTGACTACGCACAGAAGTTCCAGGGCAG | GGTATCCCAGCCAGGTTCAGTGGCAGTGGGTCT |
| | AGTCACGATTACCGCGGACGAATCCACGAGCAC | GGGACAGAGTTCACTCTCACCATCAGCAGCCTGC |
| | AGCCTACATGGAGCTGAGCAGCCTGAGATCTGA | AGTCTGAAGATTTTGCAGTTTATTACTGTCAGCA |
| | GGACACGGCCGTGTATTACTGTGCGAAGGACTTT | GTATAATACATACCCTATCACGTTCGGCCAAGGG |
| | AGAGGCGGGGATTATTACTATTATGGAATGGAC | ACCAAGGTGGAAATCAAA (SEQ ID NO: 502) |
| | GTATGGGGCCAAGGGACCACGGTCACCGTCTCC | |
| | TCA (SEQ ID NO: 455) | |
| Antibody | CAGGTTCAGCTGGTGCAGTCTGGAGCTGAGGTG | GACATCCAGATGACCCAGTCTCCATCCTCCCTGTC |
| 45, 77, 94, | AAGAAGCCTGGGGCCTCAGTGAAGGTCTCCTGC | TGCATCTGTAGGAGACAGAGTCACCATCACTTGC |
| 106 | AAGGCTTCTGGTTACACCTTTACCAGCTATGGTA | CGGGCAAGTCAGAGCATTAGCAGCTATTTAAATT |
| | TCAGCTGGGTGCGACAGGCCCCTGGACAAGGGC | GGTATCAGCAGAAACCAGGGAAAGCCCCTAAGC |
| | TTGAGTGGATGGGATGGATCAGCGCCTACAATG | TCCTGATCTATGCTGCATCCAGTTTGCAAAGTGG |
| | GTAACACAAACTATGCACAGAAGTTCCAGGGCA | GGTCCCATCAAGGTTCAGTGGCAGTGGATCTGG |
| | GAGTCACCATGACCCGAGACACATCCACGAGCA | GACAGATTTCACTCTCACCATCAGCAGTCTGCAA |
| | CAGTGTACATGGAGCTGAGCAGCCTGAGATCTG | CCTGAAGATTTTGCAACTTACTACTGTCAACAGA |
| | AGGACACGGCCGTGTATTACTGTGCGAGAGGAA | GTTACAGTACCCCTCTCACTTTCGGCGGAGGGAC |
| | CTTACTACGATTATATTTGGGGATCTTACAGGTA | CAAGGTGGAGATCAAA (SEQ ID NO: 503) |
| | CAGCTGGTTTGACTACTGGGGCCAAGGGACCCT | |
| | GGTCACCGTCTCCTCA (SEQ ID NO: 456) | |
| Antibody 46 | CAGGTGCAGCTGGTGCAGTCTGGGGCTGAGGTG | GACATCCAGATGACCCAGTCTCCATCCTCCCTGTC |
| | AAGAAGCCTGGGGCCTCAGTGAAGGTCTCCTGC | TGCATCTGTAGGAGACAGAGTCACCATCACTTGC |
| | AAGGCTTCTGGATACACCTTCTCCAGTTATGCTAT | CAGGCAAGTCAGGACATTAGCAACTATTTAAATT |
| | CTCTTGGGTGCGACAGGCCCCCGGACAAGGGCT | GGTATCAGCAGAAACCAGGGAAAGCCCCTAAGC |
| | TGAGTGGATGGGATGGATGGACCCTAACAGTGG | TCCTGATCTATGCTGCATCCACCTTGCAAACAGG |
| | TAACACAGGCTATGCACAGAAGTTCCAGGGCAG | GGTCCCATCAAGGTTCAGTGGCAGTGGATCTGG |
| | AGTCACCATGACCAGGGATACCTCCACCAGCACA | GACAGATTTCACTCTCACCATCAGCAGTCTGCAA |
| | GTCTACATGGAGCTGAGCAGCCTGAGATCTGAG | CCTGAAGATTTTGCAACTTACTACTGTCAACAGA |
| | GACACGGCCGTGTATTACTGTGCGAGAGAGTCG | GTTACAGTACCCCTTGGACTTTCGGCCCTGGGAC |
| | TCCTACTATTATGATAGCAGCGGCTACTATCCTG | CAAAGTGGATATCAAA (SEQ ID NO: 504) |
| | GATGGTACTTCGATCTCTGGGGCCGTGGCACCCT | |
| | GGTCACTGTCTCCTCA (SEQ ID NO: 457) | |
| Antibody | CAGGTGCAGCTGGTGCAGTCTGGGGCTGAGGTG | GACATCCAGATGACCCAGTCTCCATCTTCTCTTTC |
| 47, 78 | AAGAAGCCTGGGGCCTCAGTGAAGGTCTCCTGC | TGCATCTGTAGGAGACAGAGTCACCATCACTTGT |
| | AAGGCTTCTGGATACACCTTCACCACCTATGGGA | CGGGCGAGTCAGCGCATTGGTAGCTACTTAAACT |
| | TCTCCTGGGTGCGACAGGCCCCAGGACAAGGGC | GGTATCAGCAGAAACCAGGGAAAGCCCCTAAGC |
| | TTGAGTGGATGGGATGGATGGACCCAAACAGTG | TCCTGATCTATGGAGCATCCAATTTGCAAAGTGG |
| | GTTCTACAGGCTATGCACAGAAGTTCCAGGGCA | GGTCCCATCAAGGTTCAGCGGCAGTGGATCTGG |
| | GAGTCACCATGACCAGGGATACCTCCACCAGCAC | GACAGATTTCACTCTCACTATCAGCAGCCTGCAG |
| | AGTGTACATGGAGCTGAGCAGCCTGAGATCTGA | CCTGAAGATTTTGCAACTTACTATTGTCAACAGG |
| | GGACACGGCCGTGTATTACTGTGCGAGAGGCCG | CTAACAGTTTCCCTTTCACTTTCGGCCCTGGGACC |
| | GGGCAACAGCTACTACTACTACTACATGGACGTC | AAAGTGGATATCAAA (SEQ ID NO: 505) |
| | TGGGGCAAAGGGACCACGGTCACCGTCTCCTCA | |
| | (SEQ ID NO: 458) | |
| Antibody | CAGGTGCAGCTGGTGCAGTCTGGGGCTGAGGTG | GACATCCAGATGACCCAGTCTCCATCCTCCCTGTC |
| 48, 79, 95, | AAGAAGCCTGGGGCCTCAGTGAAGGTTTCCTGC | TGCATCTGTAGGAGACAGAGTCACCATCACTTGC |
| 107 | AAGGCATCTGGATACACCTTCACCAGCTACTATA | CGGGCAAGTGAAAGCATTACAAACTGGTTAGCTT |
| | TGCACTGGGTGCGACAGGCCCCTGGACAAGGGC | GGTATCAGCAGAAACCAGGGAAAGCCCCTAAGC |
| | TTGAGTGGATGGGATGGATGAACCCCAATAGTG | TCCTGATCTATAAAGCATCCAGTTTGGAAAGTGG |
| | GTAATACAGGCTACGCACAGAAGTTCCAGGGCA | GGTCCCATCAAGGTTCAGTGGCAGTGGATCTGG |
| | GAGTCACCATGACCAGGGACACGTCCACGAGCA | GACAGATTTCACTCTCACCATCAGCAGTCTGCAA |
| | CAGTCTACATGGAGCTGAGCAGCCTGAGATCTG | CCTGAAGATTTTGCAACTTACTACTGTCAACAGA |
| | AGGACACGGCCGTGTATTACTGTGCTAGAGATCA | GTTACGGTACCCCTTACACGTTCGGCCAAGGGAC |
| | GATGTATAGTTCATACGGCATGGATGTCTGGGG | CAAGGTGGAAATCAAA (SEQ ID NO: 506) |
| | CCAAGGGACAACTGTCACCGTCTCTTCA (SEQ ID | |
| | NO: 459) | |
| Antibody 49 | CAGGTGCAGCTGGTGCAGTCTGGGGCTGAGGTG | GACATCGTGATGACCCAGTCTCCAGACTCCCTGG |
| | AAGAAGCCTGGGTCCTCGGTGAAGGTCTCCTGC | CTGTGTCTCTGGGCGAGAGGGCCACCATCAACTG |
| | AAGGCTTCTGGAGGCACCTTCAGCAGCTATGCTA | CAAGTCCAGCCAGAGTGTTCTCTATTCGTCCAAT |
| | TCAGCTGGGTGCGACAGGCCCCTGGACAAGGGC | AACAAGAACTACTTAGCTTGGTACCAGCAGAAAC |
| | TTGAGTGGATGGGAGTCATCAACCCTTCAGGTG | CAGGACAGCCTCCTAAGCTGCTCATTTACTGGGC |
| | GGTCCACATCATACGCACAGAAGTTCCAGGGCA | ATCTACCCGGGAATCCGGGGTCCCTGACCGATTC |
| | GAGTCACGATTACCGCGGACGAATCCACGAGCA | AGTGGCAGCGGGTCTGGGACAGATTTCACTCTCA |
| | CAGCCTACATGGAGCTGAGCAGCCTGAGATCTG | CCATCAGCAGCCTGCAGGCTGAAGATGTGGCAG |
| | AGGACACGGCCGTGTATTACTGTGCGAGAGAAT | TTTATTACTGTCAGCAATATTATAGTACTCCTTAC |
| | CTAGAGGGTACTATGGGATGGACGTGTGGGGCC | ACGTTCGGCCAAGGGACCAAGGTGGAAATCAAA |
| | AAGGGACCACTGTCACCGTCTCCTCA (SEQ ID | (SEQ ID NO: 507) |
| | NO: 460) | |
| Antibody 50 | CAGGTGCAGCTGGTGCAGTCTGGGGCTGAGGTG | GACATCCAGATGACCCAGTCTCCATCTTCTCTGTC |
| | AAGAAGCCTGGGGCCTCAGTGAAGGTTTCCTGC | TGCATCTGTAGGAGACAGAGTCACCATCACTTGT |
| | AAGGCATCTGGATACACCTTCACCGGCTACTATA | CGGGCGAGTCAGTCAATTAGCAGCTGGTTAGCCT |
| | TGCACTGGGTGCGACAGGCCCCTGGACAAGGGC | GGTATCAGCAGAAACCAGGGAAAGCCCCTAAGC |
| | TTGAGTGGATGGGAACCATCAACCCTAGTGGTG | TCCTGATCTATGATACATCCAACTTGCAAGGTGG |
| | GCAGCACAAGCTACGCACAGAAGTTCCAGGGCA | GGTCCCATCAAGGTTCAGCGGCAGTGGATCTGG |
| | GAGTCACCATGACCAGGGACACGTCCACGAGCA | GACAGATTTCACTCTCACTATCAGCAGCCTGCAG |
| | CAGTCTACATGGAGCTGAGCAGCCTGAGATCTG | CCTGAAGATTTTGCAACTTACTATTGTCAACAGTA |
| | AGGACACGGCCGTGTATTACTGTGTCAAAGGTA | TGATAGTTTCCCTTGGACGTTCGGCCAAGGGACC |
| | ACGGCTACTACGACCTGTGGGGCCAAGGGACCC | AAGGTGGAAATCAAA (SEQ ID NO: 508) |
| | TGGTCACCGTCTCCTCA (SEQ ID NO: 461) | |
| Antibody | CAGGTGCAGCTGGTGCAGTCTGGGGCTGAGGTG | GACATCCAGATGACCCAGTCTCCATCCTCCCTGTC |
| 51, 80, 96, | AAGAAGCCTGGGGCCTCAGTGAAGGTCTCCTGC | TGCATCTGTAGGAGACAGAGTCACCATCACTTGC |
| 108 | AAGGCTTCTGGATACACCTTCACCCGATATGATA | CGGGCAAGTCAGGGCATTAGAAATGATTTAGGC |
| | TCAACTGGGTGCGACAGGCCCCCGGACAAGGGC | TGGTATCAGCAGAAACCAGGGAAAGCCCCTAAG |
| | TTGAGTGGATGGGATGGATGAACCCTAACAGTG | CTCCTGATCTATAGTGCATCCAATTTACAAAGTG |
| | GTAACACAGGCTATGCACAGAAGTTACAGGGCA | GGGTCCCATCAAGGTTCAGCGGCAGTGGATCTG |
| | GAGTCACCATGACCAGGGACACCTCCACAAGCA | GCACAGATTTCACTCTCACCATCAGCAGCCTGCA |
| | CAGTCTACATGGAGCTGAGCAGCCTGAGATCTG | GCCTGAAGATTTTGCAACTTATTACTGTCAGCAA |
| | AGGACACGGCCGTGTATTACTGTGCGAGAGAGC | AGCTACGATGTCCCTCTCACTTTCGGCGGAGGGA |
| | GTGTGGGGTACTGCTCGAGCACCTCGTGTCTTCA | CCAAGGTGGAGATCAAA (SEQ ID NO: 509) |
| | TCCACTGGACTACTGGGGCCAAGGGACCCTGGT | |
| | CACCGTCTCCTCA (SEQ ID NO: 462) | |
| Antibody | CAGGTTCAGCTGGTGCAGTCTGGAGCTGAGGTG | GACATCCAGATGACCCAGTCTCCTTCCAGTCTGTC |
| 52, 81 | AAGAAGCCTGGGGCCTCAGTGAAGGTCTCCTGC | TGCATCTGTAGGAGACAGAGTCACCATCACTTGC |
| | AAGGCTTCTGGTTACACCTTTACCAGCTATGACAT | CGGGCCAGTCAGAGTATTAGTAGCTGGTTGGCC |
| | CAACTGGGTGCGACAGGCCCCTGGACAAGGGCT | TGGTATCAGCAGAAACCAGGGAAAGCCCCTAAG |
| | TGAGTGGATGGGATGGATCAGCGGGTACAATGG | CTCCTGATCTATGATGCCACTACCTTGGAAAGTG |
| | TAACACAAACTATGCACAGAAGTTCCAGGGCAG | GGGTCCCATCAAGGTTCAGCGGCAGTGGATCTG |
| | AGTCACCATGACCAGGGACACATCCACGAGCAC | GGACAGATTTCACTCTCACCATCAGCAGCCTGCA |
| | AGTTTACATGGAGCTGAGCAGCCTGAGATCTGA | GCCTGAAGATTTTGCAACTTATTACTGCCAACAG |
| | GGACACGGCCGTGTATTACTGTGCGAGATCTTTT | TATGACAGTTATCCATGGACGTTCGGCCAAGGGA |
| | GATTGGTTACTGCTACTTGACTACTGGGGCCAAG | CCAAGCTCGAAATCAAA (SEQ ID NO: 510) |
| | GGACCCTGGTCACCGTCTCCTCA (SEQ ID NO: | |
| | 463) | |
| Antibody | CAGGTGCAGCTGGTGCAGTCTGGGGCTGAGGTG | GAAATAGTGATGACGCAGTCTCCAGCCACCCTGT |
| 53, 82, 97, | AAGAAGCCTGGGGCCTCAGTGAAGGTTTCCTGC | CTGTGTCTCCAGGGGAAAGAGCCACCCTCTCCTG |
| 109 | AAGGCATCTGGACACACCTTCAACAATAATTATA | CAGGGCCAGTCAGAGTGTTAGCAAGTATTTAGCC |
| | TTCACTGGGTGCGACAGGCCCCTGGACAAGGGC | TGGTACCAGCAGAAACCTGGCCAGGCTCCCAGG |
| | TTGAGTGGATGGGAATAATCAACCCTAGTGGTG | CTCCTCATCTATGCCATCTCCGCGAGGGCCGCTG |
| | GGAGCACAAGCTACGCACAGAAGTTCCAGGGCA | GTATCCCAGCCAGGTTCAGTGGCAGTGGGTCTG |
| | GAGTCACCATGACCAGGGACACGTCCACGAGCA | GGACAGAGTTCACTCTCACCATCAGCAGCCTGCA |
| | CAGTCTACATGGAGCTGAGCAGCCTGAGATCTG | GTCTGAAGATTTTGCAGTTTATTACTGTCAGCAG |
| | AGGACACGGCCGTGTATTACTGTGCGAGACAAA | TATTATTCCAGTCCTCCTACGTTCGGCCAAGGGA |
| | TACAGGATTACGGATACTACTATTACGGCATGGA | CCAAGGTGGAAATCAAA (SEQ ID NO: 511) |
| | CGTGTGGGGCCAAGGGACCACCGTCACCGTCTC | |
| | CTCA (SEQ ID NO: 464) | |

Accordingly, in some embodiments a nucleic acid sequence encoding for a SIRPγ antibody of the disclosure comprises a variable heavy chain nucleic acid sequence selected from SEQ ID NOS: 418-464, or at least 70% sequence identity thereto. In some embodiments a nucleic acid sequence encoding for a SIRPγ antibody of the disclosure comprises a variable light chain nucleic acid sequence selected from SEQ ID NOS: 465-511, or at least 70% sequence identity thereto. The person of ordinary skill in the art will appreciate that, because of redundancy in the triplet code, multiple nucleic acids may encode the same amino acid sequence. Thus, nucleic acid sequences that are not identical to those set forth in Table 3 can still encode the amino acid sequences set forth in preceding section.

In some embodiments, provided herein is a nucleic acid encoding any of the SIRPγ antibodies disclosed herein. In some embodiments, provided herein is a nucleic acid comprising any one or more of the nucleic acid sequences of Table 3. In some embodiments, the heavy and light chain variable domains of the SIRPγ antibodies disclosed herein are encoded by a nucleic acid comprising any one or more of the nucleic acid sequences of Table 3.

In some embodiments, provided herein is a SIRPγ antibody, wherein the heavy chain variable domain of the antibody is encoded by a nucleic acid sequence comprising the sequence of SEQ ID NO: 418, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or the light chain variable domain of the antibody is encoded by a nucleic acid sequence comprising the sequence of SEQ ID NO: 465, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain of the antibody is encoded by a nucleic acid sequence comprising the sequence of SEQ ID NO: 419, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or the light chain variable domain of the antibody is encoded by a nucleic acid sequence comprising the sequence of SEQ ID NO: 466, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain of the antibody is encoded by a nucleic acid sequence comprising the sequence of SEQ ID NO: 420, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or the light chain variable domain of the antibody is encoded by a nucleic acid sequence comprising the sequence of SEQ ID NO: 467, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain of the antibody is encoded by a nucleic acid sequence comprising the sequence of SEQ ID NO: 421, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or the light chain variable domain of the antibody is encoded by a nucleic acid sequence comprising the sequence of SEQ ID NO: 468, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain of the antibody is encoded by a nucleic acid sequence comprising the sequence of SEQ ID NO: 422, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or the light chain variable domain of the antibody is encoded by a nucleic acid sequence comprising the sequence of SEQ ID NO: 469, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain of the antibody is encoded by a nucleic acid sequence comprising the sequence of SEQ ID NO: 423, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or the light chain variable domain of the antibody is encoded by a nucleic acid sequence comprising the sequence of SEQ ID NO: 470, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain of the antibody is encoded by a nucleic acid sequence comprising the sequence of SEQ ID NO: 424, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or the light chain variable domain of the antibody is encoded by a nucleic acid sequence comprising the sequence of SEQ ID NO: 471, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain of the antibody is encoded by a nucleic acid sequence comprising the sequence of SEQ ID NO: 425, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or the light chain variable domain of the antibody is encoded by a nucleic acid sequence comprising the sequence of SEQ ID NO: 472, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain of the antibody is encoded by a nucleic acid sequence comprising the sequence of SEQ ID NO: 426, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or the light chain variable domain of the antibody is encoded by a nucleic acid sequence comprising the sequence of SEQ ID NO: 473, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain of the antibody is encoded by a nucleic acid sequence comprising the sequence of SEQ ID NO: 427, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or the light chain variable domain of the antibody is encoded by a nucleic acid sequence comprising the sequence of SEQ ID NO: 474, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain of the antibody is encoded by a nucleic acid sequence comprising the sequence of SEQ ID NO: 428, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or the light chain variable domain of the antibody is encoded by a nucleic acid sequence comprising the sequence of SEQ ID NO: 475, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain of the antibody is encoded by a nucleic acid sequence comprising the sequence of SEQ ID NO: 429, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or the light chain variable domain of the antibody is encoded by a nucleic acid sequence comprising the sequence of SEQ ID NO: 476, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain of the antibody is encoded by a nucleic acid sequence comprising the sequence of SEQ ID NO: 430, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or the light chain variable domain of the antibody is encoded by a nucleic acid sequence comprising the sequence of SEQ ID NO: 477, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain of the antibody is encoded by a nucleic acid sequence comprising the sequence of SEQ ID NO: 431, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or the light chain variable domain of the antibody is encoded by a nucleic acid sequence comprising the sequence of SEQ ID NO: 478, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain of the antibody is encoded by a nucleic acid sequence comprising the sequence of SEQ ID NO: 432, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or the light chain variable domain of the antibody is encoded by a nucleic acid sequence comprising the sequence of SEQ ID NO: 479, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain of the antibody is encoded by a nucleic acid sequence comprising the sequence of SEQ ID NO: 433, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or the light chain variable domain of the antibody is encoded by a nucleic acid sequence comprising the sequence of SEQ ID NO: 480, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain of the antibody is encoded by a nucleic acid sequence comprising the sequence of SEQ ID NO: 434, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or the light chain variable domain of the antibody is encoded by a nucleic acid sequence comprising the sequence of SEQ ID NO: 481, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain of the antibody is encoded by a nucleic acid sequence comprising the sequence of SEQ ID NO: 435, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or the light chain variable domain of the antibody is encoded by a nucleic acid sequence comprising the sequence of SEQ ID NO: 482, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain of the antibody is encoded by a nucleic acid sequence comprising the sequence of SEQ ID NO: 436, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or the light chain variable domain of the antibody is encoded by a nucleic acid sequence comprising the sequence of SEQ ID NO: 483, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain of the antibody is encoded by a nucleic acid sequence comprising the sequence of SEQ ID NO: 437, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or the light chain variable domain of the antibody is encoded by a nucleic acid sequence comprising the sequence of SEQ ID NO: 484, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain of the antibody is encoded by a nucleic acid sequence comprising the sequence of SEQ ID NO: 438, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or the light chain variable domain of the antibody is encoded by a nucleic acid sequence comprising the sequence of SEQ ID NO: 485, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain of the antibody is encoded by a nucleic acid sequence comprising the sequence of SEQ ID NO: 439, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or the light chain variable domain of the antibody is encoded by a nucleic acid sequence comprising the sequence of SEQ ID NO: 486, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain of the antibody is encoded by a nucleic acid sequence comprising the sequence of SEQ ID NO: 440, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or the light chain variable domain of the antibody is encoded by a nucleic acid sequence comprising the sequence of SEQ ID NO: 487, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain of the antibody is encoded by a nucleic acid sequence comprising the sequence of SEQ ID NO: 441, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or the light chain variable domain of the antibody is encoded by a nucleic acid sequence comprising the sequence of SEQ ID NO: 488, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain of the antibody is encoded by a nucleic acid sequence comprising the sequence of SEQ ID NO: 442, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or the light chain variable domain of the antibody is encoded by a nucleic acid sequence comprising the sequence of SEQ ID NO: 489, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain of the antibody is encoded by a nucleic acid sequence comprising the sequence of SEQ ID NO: 443, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or the light chain variable domain of the antibody is encoded by a nucleic acid sequence comprising the sequence of SEQ ID NO: 490, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain of the antibody is encoded by a nucleic acid sequence comprising the sequence of SEQ ID NO: 444, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or the light chain variable domain of the antibody is encoded by a nucleic acid sequence comprising the sequence of SEQ ID NO: 491, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain of the antibody is encoded by a nucleic acid sequence comprising the sequence of SEQ ID NO: 445, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or the light chain variable domain of the antibody is encoded by a nucleic acid sequence comprising the sequence of SEQ ID NO: 492, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain of the antibody is encoded by a nucleic acid sequence comprising the sequence of SEQ ID NO: 446, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or the light chain variable domain of the antibody is encoded by a nucleic acid sequence comprising the sequence of SEQ ID NO: 493, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain of the antibody is encoded by a nucleic acid sequence comprising the sequence of SEQ ID NO: 447, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or the light chain variable domain of the antibody is encoded by a nucleic acid sequence comprising the sequence of SEQ ID NO: 494, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain of the antibody is encoded by a nucleic acid sequence comprising the sequence of SEQ ID NO: 448, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or the light chain variable domain of the antibody is encoded by a nucleic acid sequence comprising the sequence of SEQ ID NO: 495, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain of the antibody is encoded by a nucleic acid sequence comprising the sequence of SEQ ID NO: 449, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or the light chain variable domain of the antibody is encoded by a nucleic acid sequence comprising the sequence of SEQ ID NO: 496, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain of the antibody is encoded by a nucleic acid sequence comprising the sequence of SEQ ID NO: 450, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or the light chain variable domain of the antibody is encoded by a nucleic acid sequence comprising the sequence of SEQ ID NO: 497, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain of the antibody is encoded by a nucleic acid sequence comprising the sequence of SEQ ID NO: 451, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or the light chain variable domain of the antibody is encoded by a nucleic acid sequence comprising the sequence of SEQ ID NO: 498, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain of the antibody is encoded by a nucleic acid sequence comprising the sequence of SEQ ID NO: 452, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or the light chain variable domain of the antibody is encoded by a nucleic acid sequence comprising the sequence of SEQ ID NO: 499, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain of the antibody is encoded by a nucleic acid sequence comprising the sequence of SEQ ID NO: 453, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or the light chain variable domain of the antibody is encoded by a nucleic acid sequence comprising the sequence of SEQ ID NO: 500, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain of the antibody is encoded by a nucleic acid sequence comprising the sequence of SEQ ID NO: 454, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or the light chain variable domain of the antibody is encoded by a nucleic acid sequence comprising the sequence of SEQ ID NO: 501, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain of the antibody is encoded by a nucleic acid sequence comprising the sequence of SEQ ID NO: 455, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or the light chain variable domain of the antibody is encoded by a nucleic acid sequence comprising the sequence of SEQ ID NO: 502, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain of the antibody is encoded by a nucleic acid sequence comprising the sequence of SEQ ID NO: 456, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or the light chain variable domain of the antibody is encoded by a nucleic acid sequence comprising the sequence of SEQ ID NO: 503, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain of the antibody is encoded by a nucleic acid sequence comprising the sequence of SEQ ID NO: 457, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or the light chain variable domain of the antibody is encoded by a nucleic acid sequence comprising the sequence of SEQ ID NO: 504, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain of the antibody is encoded by a nucleic acid sequence comprising the sequence of SEQ ID NO: 458, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or the light chain variable domain of the antibody is encoded by a nucleic acid sequence comprising the sequence of SEQ ID NO: 505, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain of the antibody is encoded by a nucleic acid sequence comprising the sequence of SEQ ID NO: 459, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or the light chain variable domain of the antibody is encoded by a nucleic acid sequence comprising the sequence of SEQ ID NO: 506, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain of the antibody is encoded by a nucleic acid sequence comprising the sequence of SEQ ID NO: 460, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or the light chain variable domain of the antibody is encoded by a nucleic acid sequence comprising the sequence of SEQ ID NO: 507, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain of the antibody is encoded by a nucleic acid sequence comprising the sequence of SEQ ID NO: 461, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or the light chain variable domain of the antibody is encoded by a nucleic acid sequence comprising the sequence of SEQ ID NO: 508, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain of the antibody is encoded by a nucleic acid sequence comprising the sequence of SEQ ID NO: 462, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or the light chain variable domain of the antibody is encoded by a nucleic acid sequence comprising the sequence of SEQ ID NO: 509, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain of the antibody is encoded by a nucleic acid sequence comprising the sequence of SEQ ID NO: 463, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or the light chain variable domain of the antibody is encoded by a nucleic acid sequence comprising the sequence of SEQ ID NO: 510, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, provided herein is a SIRPγ antibody, wherein the heavy chain variable domain of the antibody is encoded by a nucleic acid sequence comprising the sequence of SEQ ID NO: 464, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or the light chain variable domain of the antibody is encoded by a nucleic acid sequence comprising the sequence of SEQ ID NO: 511, or a nucleic acid sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

The disclosure also provides vectors comprising any nucleic acid of the disclosure. In some embodiments, the nucleic acid of the vector comprises any one or more of the nucleic acid sequences selected from Table 3. In some embodiments, the vector is an expression vector or an expression construct. In some embodiments, the vector is a mammalian vector. In some embodiments, the vector is a viral vector.

In some embodiments, the SIRPγ antibodies provided herein are produced by culturing a cell under suitable conditions for expressing the SIRPγ antibody, wherein the cell comprises a vector.

### II. Uses of SIRPγ Antibodies

### A. SIRPγ-Expressing Cell Depletion

Provided herein are methods of inducing Fc-mediated cell depletion, the method comprising contacting a SIRPγ-expressing cell with any of the Fc-containing SIRPγ antibodies of the disclosure. The method may be carried out in vitro or in vivo. In some embodiments, the cell depletion involves antibody dependent cellular phagocytosis (ADCP). In some embodiments, the cell depletion involves antibody dependent cellular cytotoxicity (ADCC). In some embodiments, the cell depletion involves complement dependent cytotoxicity (CDC). In some embodiments, the cell depletion involves one or more of ADCP, ADCC, and CDC. One of more Fc substitutions of the disclosure may further modulate the depletion.

In some embodiments, the cells are in a stimulated (activated) state. Without being held to any theory or mechanism, the SIRPγ antibodies of the disclosure can be used to preferentially deplete pathogenic T-cells, activated T-cells, exhausted T-cells, unactivated T-cells (or other cells). In some embodiments, without being held to theory or mechanism, this may be due to the increased expression of SIRPγ on an activated T-cell. In some embodiments, without being held to theory or mechanism, this may be due to the differential expression of specific SIRPγ isoforms on specific T-cell subsets (e.g. isoforms set forth in SEQ ID NOS: 1-3, and 37). T-cell subsets include cells that are in different cell states, e.g. stimulated, exhausted; subsets also include T-cells that express different subsets of markers.

In some embodiments, the SIRPγ-expressing cells are SIRPγ-expressing T-cells. In some embodiments, the SIRPγ-expressing T-cells are in a naïve state. In some embodiments, the SIRPγ-expressing T-cells are in an activated (stimulated) state. In some embodiments, the SIRPγ-expressing T-cells are in an exhausted state. In some embodiments, the SIRPγ-expressing T-cells are in an undifferentiated state. In some embodiments, the T-cell is a cytotoxic T-cell, helper T-cell, a memory T-cell, a regulatory T-cell, a natural killer T-cell, a mucosal associated invariant T-cell, or an alpha beta (αβ) T-cell, or a gamma delta (gd) T-cell. In some embodiments, the T-cell is a naïve, central memory, effector memory, exhausted, or terminal effector memory cell. In some embodiments, the T-cell is a CD4+ T-cell, CD8+ T-cell, CD3+ T-cell, Th1 cell, Th2 cell, Th17 cell or a T follicular helper cell.

In some embodiments, the T-cell is a CD3+ T-cell, CD4+ T-cell, CD8+ T-cell, CD25+ T-cell, CD69+ T-cell, and/or PD1+ T-cell. In some embodiments, the T-cell is a CD4+/CD8+ T-cell. In some embodiments, the T-cell is a CD69+/CD8+ T-cell. In some embodiments, the T-cell is a CD25+/CD8+ T-cell. In some embodiments, the T-cell is a PD1+ T-cell.

Certain SIRPγ antibodies of the disclosure also display preferential binding to and subsequent Fc-mediated depletion in certain cell types. Without being held to any theory or mechanism, the SIRPγ antibodies of the disclosure can be used to preferentially deplete certain cell types (expressing certain markers), or cells in a certain state (stimulated, exhausted). Without being held to theory or mechanism, this may be due to the differential expression of specific SIRPγ isoforms on specific T-cell subsets (e.g. isoforms set forth in SEQ ID NOS: 1-3, and 37).

The preferential binding may be at least 1.5-fold, at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, or even at least 50-fold, as compared to the binding that is observed in a reference cell type or a reference cell state.

The increase in Fc-mediated depletion may be at least 1.5-fold, at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, or even at least 50-fold increase in depletion, as compared to the depletion that is observed in a reference cell type or a reference cell state.

In some embodiments, certain SIRPγ antibodies of the disclosure exhibit preferential binding to and depletion of an activated (stimulated) T-cell, as compared to an unstimulated T-cell.

In some embodiments, certain SIRPγ antibodies of the disclosure exhibit preferential binding to and depletion of a SIRPγ-expressing CD8+ T-cell.

In some embodiments, certain SIRPγ antibodies of the disclosure exhibit preferential binding to and depletion of a SIRPγ-expressing CD4+ T-cell.

In some embodiments, certain SIRPγ antibodies of the disclosure exhibit preferential binding to and depletion of a SIRPγ-expressing CD8+/CD69+ T-cell.

In some embodiments, certain SIRPγ antibodies of the disclosure exhibit preferential binding to and depletion of a SIRPγ-expressing CD8+/CD25+ T-cell.

In some embodiments, certain SIRPγ antibodies of the disclosure exhibit preferential binding to and depletion of a SIRPγ-expressing CD8+ T-cell, when compared to a SIRPγ-expressing CD4+ T-cell.

In some embodiments, certain SIRPγ antibodies of the disclosure exhibit preferential binding to and depletion of a SIRPγ-expressing CD4+ T-cell, when compared to a SIRPγ-expressing CD8+ T-cell.

In some embodiments, certain SIRPγ antibodies of the disclosure exhibit preferential binding to and depletion of a SIRPγ-expressing CD8+/CD69+ T-cell, when compared to a SIRPγ-expressing CD8+/CD69− T-cell.

In some embodiments, certain SIRPγ antibodies of the disclosure exhibit preferential binding to and depletion of a SIRPγ-expressing CD8+/CD25+ T-cell, when compared to a SIRPγ-expressing CD8+/CD25− T-cell.

In some embodiments, certain SIRPγ antibodies of the disclosure exhibit preferential binding to and depletion of a SIRPγ-expressing PD1+ T-cell, compared to a PD1− T-cell.

In some embodiments, exemplary antibodies that exhibit increased binding to and depletion of stimulated T-cells when compared to unstimulated T-cells include an antibody comprising the following sequences, making reference to Table 1 above:
a. the CDR-H1 amino acid sequence of SEQ ID NO: 211; the CDR-H2 amino acid sequence of SEQ ID NO: 247; the CDR-H3 amino acid sequence of SEQ ID NO: 279; the CDR-L1 amino acid sequence of SEQ ID NO: 102; the CDR-L2 amino acid sequence of SEQ ID NO: 140; and the CDR-L3 amino acid sequence of SEQ ID NO: 169.
b. the CDR-H1 amino acid sequence of SEQ ID NO: 216; the CDR-H2 amino acid sequence of SEQ ID NO: 254; the CDR-H3 amino acid sequence of SEQ ID NO: 286; the CDR-L1 amino acid sequence of SEQ ID NO: 108; the CDR-L2 amino acid sequence of SEQ ID NO: 144; and the CDR-L3 amino acid sequence of SEQ ID NO: 176.
c. the CDR-H1 amino acid sequence of SEQ ID NO: 213; the CDR-H2 amino acid sequence of SEQ ID NO: 249; the CDR-H3 amino acid sequence of SEQ ID NO: 290; the CDR-L1 amino acid sequence of SEQ ID NO: 112; the CDR-L2 amino acid sequence of SEQ ID NO: 148; and the CDR-L3 amino acid sequence of SEQ ID NO: 179.
d. the CDR-H1 amino acid sequence of SEQ ID NO: 221; the CDR-H2 amino acid sequence of SEQ ID NO: 257; the CDR-H3 amino acid sequence of SEQ ID NO: 291; the CDR-L1 amino acid sequence of SEQ ID NO: 113; the CDR-L2 amino acid sequence of SEQ ID NO: 143; and the CDR-L3 amino acid sequence of SEQ ID NO: 180.
e. the CDR-H1 amino acid sequence of SEQ ID NO: 222; the CDR-H2 amino acid sequence of SEQ ID NO: 258; the CDR-H3 amino acid sequence of SEQ ID NO: 292; the CDR-L1 amino acid sequence of SEQ ID NO: 106; the CDR-L2 amino acid sequence of SEQ ID NO: 149; and the CDR-L3 amino acid sequence of SEQ ID NO: 181.
f. the CDR-H1 amino acid sequence of SEQ ID NO: 222; the CDR-H2 amino acid sequence of SEQ ID NO: 262; the CDR-H3 amino acid sequence of SEQ ID NO: 297; the CDR-L1 amino acid sequence of SEQ ID NO: 118; the CDR-L2 amino acid sequence of SEQ ID NO: 143; and the CDR-L3 amino acid sequence of SEQ ID NO: 186.
g. the CDR-H1 amino acid sequence of SEQ ID NO: 226; the CDR-H2 amino acid sequence of SEQ ID NO: 263; the CDR-H3 amino acid sequence of SEQ ID NO: 299; the CDR-L1 amino acid sequence of SEQ ID NO: 120; the CDR-L2 amino acid sequence of SEQ ID NO: 140; and the CDR-L3 amino acid sequence of SEQ ID NO: 188.
h. the CDR-H1 amino acid sequence of SEQ ID NO: 216; the CDR-H2 amino acid sequence of SEQ ID NO: 266; the CDR-H3 amino acid sequence of SEQ ID NO: 303; the CDR-L1 amino acid sequence of SEQ ID NO: 124; the CDR-L2 amino acid sequence of SEQ ID NO: 143; and the CDR-L3 amino acid sequence of SEQ ID NO: 191.
i. the CDR-H1 amino acid sequence of SEQ ID NO: 234; the CDR-H2 amino acid sequence of SEQ ID NO: 249; the CDR-H3 amino acid sequence of SEQ ID NO: 308; the CDR-L1 amino acid sequence of SEQ ID NO: 127; the CDR-L2 amino acid sequence of SEQ ID NO: 155; and the CDR-L3 amino acid sequence of SEQ ID NO: 196.
j. the CDR-H1 amino acid sequence of SEQ ID NO: 240; the CDR-H2 amino acid sequence of SEQ ID NO: 250; the CDR-H3 amino acid sequence of SEQ ID NO: 315; the CDR-L1 amino acid sequence of SEQ ID NO: 109; the CDR-L2 amino acid sequence of SEQ ID NO: 143; and the CDR-L3 amino acid sequence of SEQ ID NO: 203.
k. the CDR-H1 amino acid sequence of SEQ ID NO: 216; the CDR-H2 amino acid sequence of SEQ ID NO: 249; the CDR-H3 amino acid sequence of SEQ ID NO: 318; the CDR-L1 amino acid sequence of SEQ ID NO: 135; the CDR-L2 amino acid sequence of SEQ ID NO: 162; and the CDR-L3 amino acid sequence of SEQ ID NO: 189.
l. the CDR-H1 amino acid sequence of SEQ ID NO: 243; the CDR-H2 amino acid sequence of SEQ ID NO: 249; the CDR-H3 amino acid sequence of SEQ ID NO: 321; the CDR-L1 amino acid sequence of SEQ ID NO: 136; the CDR-L2 amino acid sequence of SEQ ID NO: 164; and the CDR-L3 amino acid sequence of SEQ ID NO: 207.
m. the CDR-H1 amino acid sequence of SEQ ID NO: 244; the CDR-H2 amino acid sequence of SEQ ID NO: 256; the CDR-H3 amino acid sequence of SEQ ID NO: 323; the CDR-L1 amino acid sequence of SEQ ID NO: 137; the CDR-L2 amino acid sequence of SEQ ID NO: 166; and the CDR-L3 amino acid sequence of SEQ ID NO: 169.

Making reference to the data in Example 4, Antibodies 83, 84, 85, 86, 88, 89, 90, 92, 94, 95, 96, and 97 exhibit increased binding to stimulated T-cells when compared to unstimulated T-cells. The CDR and VH/VL sequences for these antibodies are provided in Tables 1 and 2.

In some embodiments, an exemplary antibody that preferentially binds to and depletes CD8+ T-cells when compared to CD4+ T-cells, includes an antibody comprising the following sequences, making reference to Table 1 above: the CDR-H1 amino acid sequence of SEQ ID NO: 216; the CDR-H2 amino acid sequence of SEQ ID NO: 254; the CDR-H3 amino acid sequence of SEQ ID NO: 286; the CDR-L1 amino acid sequence of SEQ ID NO: 108; the CDR-L2 amino acid sequence of SEQ ID NO: 144; and the CDR-L3 amino acid sequence of SEQ ID NO: 176.

Making reference to Example 4, Antibody 84 preferentially binds to CD8+ T-cells when compared to CD4+ T-cells. The CDR and VH/VL sequences for this antibody is provided in Tables 1 and 2.

In some embodiments, an exemplary antibody that preferentially binds to and depletes CD69+/CD8+ T-cells and CD25+/CD8+ T-cells when compared to CD69−/CD8+ T-cells and CD25−/CD8+ T-cells includes an antibody comprising the following sequences, making reference to Table 1 above: the CDR-H1 amino acid sequence of SEQ ID NO: 216; the CDR-H2 amino acid sequence of SEQ ID NO: 254; the CDR-H3 amino acid sequence of SEQ ID NO: 286; the CDR-L1 amino acid sequence of SEQ ID NO: 108; the CDR-L2 amino acid sequence of SEQ ID NO: 144; and the CDR-L3 amino acid sequence of SEQ ID NO: 176.

Making reference to the data in Example 4, Antibody 84 preferentially binds to CD69+/CD8+ T-cells and CD25+/CD8+ T-cells when compared to CD69−/CD8+ T-cells and CD25−/CD8+ T-cells. The CDR and VH/VL sequences for this antibody is provided in Tables 1 and 2.

In some embodiments, exemplary antibodies that preferentially bind to and deplete PD1+ T-cells when compared to PD1− T-cells includes antibodies comprising the following sequences, making reference to Table 1 above:
a. the CDR-H1 amino acid sequence of SEQ ID NO: 213; the CDR-H2 amino acid sequence of SEQ ID NO: 249; the CDR-H3 amino acid sequence of SEQ ID NO: 290; the CDR-L1 amino acid sequence of SEQ ID NO: 112; the CDR-L2 amino acid sequence of SEQ ID NO: 148; and the CDR-L3 amino acid sequence of SEQ ID NO: 179;
b. the CDR-H1 amino acid sequence of SEQ ID NO: 222; the CDR-H2 amino acid sequence of SEQ ID NO: 262; the CDR-H3 amino acid sequence of SEQ ID NO: 297; the CDR-L1 amino acid sequence of SEQ ID NO: 118; the CDR-L2 amino acid sequence of SEQ ID NO: 143; and the CDR-L3 amino acid sequence of SEQ ID NO: 186;
c. the CDR-H1 amino acid sequence of SEQ ID NO: 216; the CDR-H2 amino acid sequence of SEQ ID NO: 249; the CDR-H3 amino acid sequence of SEQ ID NO: 318; the CDR-L1 amino acid sequence of SEQ ID NO: 135; the CDR-L2 amino acid sequence of SEQ ID NO: 162; and the CDR-L3 amino acid sequence of SEQ ID NO: 189;
d. the CDR-H1 amino acid sequence of SEQ ID NO: 244; the CDR-H2 amino acid sequence of SEQ ID NO: 256; the CDR-H3 amino acid sequence of SEQ ID NO: 323; the CDR-L1 amino acid sequence of SEQ ID NO: 137; the CDR-L2 amino acid sequence of SEQ ID NO: 166; and the CDR-L3 amino acid sequence of SEQ ID NO: 169.

Making reference to the data in Example 4, Antibodies 85, 89, 95, and 97 preferentially binds to PD1+ T-cells when compared to PD1−/T-cells. The CDR and VH/VL sequences for these antibodies are provided in Tables 1 and 2.

In some embodiments, the SIRPγ-expressing cells are SIRPγ-expressing B-cells. In some embodiments, the SIRPγ-expressing B-cells may be in an activated state and may be preferentially depleted.

In some embodiments, the SIRPγ-expressing cells are NK-cells. In some embodiments, the SIRPγ-expressing NK-cells may be in an activated state and may be preferentially depleted.

In some embodiments, contacting a SIRPγ-expressing cell with a Fc-containing SIRPγ antibody of the disclosure leads to depletion of the SIRPγ-expressing cell by ADCC. In some embodiments, the SIRPγ antibody increases ADCC of a by at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%. The method may be carried out in vitro or in vivo. The SIRPγ-expressing cell may be in an activated state. The SIRPγ-expressing cell may be a T-cell, a B-cell, or a NK-cell, as described above.

In some embodiments, contacting a SIRPγ-expressing cell with a Fc-containing SIRPγ antibody of the disclosure leads to depletion of the SIRPγ-expressing cell by ADCP. In some embodiments, the SIRPγ antibody increases ADCP of a by at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%. The method may be carried out in vitro or in vivo. The SIRPγ-expressing cell may be in an activated state. The SIRPγ-expressing cell may be a T-cell, a B-cell, or a NK-cell, as described above.

In some embodiments, contacting a SIRPγ-expressing cell with a Fc-containing SIRPγ antibody of the disclosure leads to depletion of the SIRPγ-expressing cell by CDC. In some embodiments, the SIRPγ antibody increases CDC of a by at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%. The method may be carried out in vitro or in vivo. The SIRPγ-expressing cell may be in an activated state. The SIRPγ-expressing cell may be a T-cell, a B-cell, or a NK-cell, as described above.

In some embodiments, contacting a SIRPγ-expressing cell with a Fc-containing SIRPγ antibody of the disclosure leads to depletion of the SIRPγ-expressing cell by ADCC and ADCP. In some embodiments, the SIRPγ antibody increases each of ADCC and ADCP of a by at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%. The method may be carried out in vitro or in vivo. The SIRPγ-expressing cell may be in an activated state. The SIRPγ-expressing cell may be a T-cell, a B-cell, or a NK-cell, as described above.

In some embodiments, contacting a SIRPγ-expressing cell with a Fc-containing SIRPγ antibody of the disclosure leads to depletion of the SIRPγ-expressing cell by ADCC and CDC. In some embodiments, the SIRPγ antibody increases each of ADCC and CDC of a by at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%. The method may be carried out in vitro or in vivo. The SIRPγ-expressing cell may be in an activated state. The SIRPγ-expressing cell may be a T-cell, a B-cell, or a NK-cell, as described above.

In some embodiments, contacting a SIRPγ-expressing cell with a Fc-containing SIRPγ antibody of the disclosure leads to depletion of the SIRPγ-expressing cell by ADCP and CDC. In some embodiments, the SIRPγ antibody increases each of ADCP and CDC of a by at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%. The method may be carried out in vitro or in vivo. The SIRPγ-expressing cell may be in an activated state. The SIRPγ-expressing cell may be a T-cell, a B-cell, or a NK-cell, as described above.

In some embodiments, contacting a SIRPγ-expressing cell with a Fc-containing SIRPγ antibody of the disclosure leads to depletion of the SIRPγ-expressing cell by ADCC, ADCP, and CDC. In some embodiments, the SIRPγ antibody increases each of ADCC, ADCP, and CDC of a by at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%. The method may be carried out in vitro or in vivo. The SIRPγ-expressing cell may be in an activated state. The SIRPγ-expressing cell may be a T-cell, a B-cell, or a NK-cell, as described above.

### B. Therapeutic SIRPγ Antibodies

As discussed in the preceding section, provided herein are antibodies that recognize and bind to SIRPγ. In some embodiments the antibody does not disrupt the binding of CD47 to SIRPγ. In some embodiments, the antibody shows increased binding to activated SIRPγ-expressing cells. The antibodies disclosed herein may be used for therapeutics in a subject.

Accordingly, provided herein are methods of treating a disease or condition in a subject in need thereof, the method comprising administering to the subject a therapeutically effective amount of a SIRPγ antibody of the disclosure, or a pharmaceutical composition thereof. In some embodiments, the subject is a mammalian subject. In some embodiments, the mammalian subject is a human subject. In some embodiments, the mammalian subject is a non-human primate, e.g. a cynomolgus monkey. In some embodiments, the mammalian subject is a model organism, e.g. a mouse, whereas treatment effects are modeled. An exemplary model includes a mouse GvHD model.

In some embodiments, the Fc-containing SIRPγ antibodies provided herein are useful for depleting a population of SIRPγ-expressing cells in the subject, for the treatment of a disease or condition. In some embodiments, the disease or condition is associated with overactivation, hyperproliferation, aberrant proliferation, dysfunction and/or dysregulation of SIRPγ-expressing cells. In some embodiments, the SIRPγ-expressing cell is a T-cell, a B-cell, or a NK-cell.

In some embodiments, the SIRPγ-expressing cell is in an activated state. Without being held to any theory or mechanism, the SIRPγ antibodies of the disclosure may be used to preferentially deplete pathogenic, activated T-cells (or other cells) sparing naïve T-cells (or other cells), allowing for the maintenance of immune surveillance.

In some embodiments the SIRPγ-expressing cell expresses one or more isoforms of SIRPγ. In some embodiments, a therapeutically effective amount of the antibody or the pharmaceutical composition is sufficient to deplete a population of SIRPγ-expressing cells in the subject, e.g. by ADCC, ADCP, and/or CDC. In some embodiments, the SIRPγ-expressing cells are tissue resident cells. In other embodiments, the SIRPγ-expressing cells are circulating. In some embodiments, the cell depletion is antibody dose-dependent.

In some embodiments, the Fc-containing SIRPγ antibodies provided herein are useful for the treatment of an autoimmune, inflammatory, or oncological disease or condition. In some embodiments, the Fc-containing SIRPγ antibodies provided herein are useful for the treatment of a disease or condition selected from: Acute and Chronic eosinophilic pneumonia, Acute disseminated encephalomyelitis, Acute Disseminated Encephalomyelitis, Acute Lymphoblastic Leukemia (ALL), Acute myelogenous leukemia (AML), Addison's disease, Adult-onset Still's disease (AOSD), Aplastic anemia, Ataxia Telangiectasia, Atopic dermatitis, Autoimmune Hepatitis, Autoimmune lymphoproliferative syndrome, Axial spondyloarthritis (AxSpA), Birdshot Retinochoroidopathy, Castleman disease, Celiac disease, Chediak-Higashi syndrome, Coronary artery disease/peripheral artery disease, Crohn's Disease, Episodic angioedema with eosinophilia/Gleich syndrome, Giant-Lymphocyte arteritis, Graft failure post-HSCT, Graft vs Host Disease (GvHD), Graves' disease, Hepatosplenic lymphoma, Hypothyroidism, Idiopathic interstitial pneumonias, IgA nephropathy, Inclusion Body Myositis (IBM), Inflammatory bowel disease (IBD), Large granular lymphocytic leukemia, Lymphocyte-variant hypereosinophila, Multiple sclerosis, Myelodysplastic syndromes (MDS), Myocarditis, Neuromyelitis optica spectrum disorders, Paraneoplastic syndromes, Primary biliary cholangitis, Primary sclerosing cholangitis, Rasmussen's Encephalitis, Rheumatoid arthritis (RA), Sarcoidosis, Schmidt syndrome/Autoimmune polyendocrine syndrome type II, Stiff Person Syndrome, Susac syndrome, Sympathetic Opthalmis, Systemic juvenile idiopathic arthritis (sJIA), Systemic Lupus Erythematosus (SLE), T-Lymphocyte mediated rejection of solid organ transplants, T-Cell Prolymphocytic Leukemia (TPLL), Type 1 diabetes, early-onset Type 1 diabetes, Ulcerative colitis, Uveitis, Vitiligo, X-linked Hyper IgM Syndrome, and X-linked lymphoproliferative disease.

In some embodiments, the Fc-containing SIRPγ antibodies provided herein are useful for the treatment of disease or condition in which the disease or condition is driven by CD8+ T-cells (e.g. IBM and early-onset Type 1 diabetes) as it is noted that certain SIRPγ antibodies of the disclosure show preferential subtype binding.

In some embodiments, the Fc-containing SIRPγ antibodies provided herein are useful for the treatment of disease or condition in which the disease or condition is driven by CD4+ T-cells, as it is noted that certain SIRγ antibodies of the disclosure show preferential T-cell subtype binding.

In some embodiments, the Fc-containing SIRPγ antibodies provided herein are useful for the treatment of disease or condition in which the disease or condition is driven by T-cells that are CD8+ and CD4+.

In some embodiments, the Fc-containing SIRPγ antibodies provided herein are useful for the treatment of disease or condition in which the disease or condition is driven by CD3+ T-cells, as it is noted that certain SIRγ antibodies of the disclosure show preferential T-cell subtype binding.

In some embodiments, the Fc-containing SIRPγ antibodies provided herein are useful for the treatment of disease or condition in which the disease or condition is driven by CD25+ T-cells, as it is noted that certain SIRγ antibodies of the disclosure show preferential T-cell subtype binding.

In some embodiments, the Fc-containing SIRPγ antibodies provided herein are useful for the treatment of disease or condition in which the disease or condition is driven by CD69+ T-cells, as it is noted that certain SIRγ antibodies of the disclosure show preferential T-cell subtype binding.

In some embodiments, the Fc-containing SIRPγ antibodies provided herein are useful for the treatment of disease or condition in which the disease or condition is driven by PD1+ T-cells, as it is noted that certain SIRγ antibodies of the disclosure show preferential T-cell subtype binding.

In some embodiments, the Fc-containing SIRPγ antibodies provided herein are useful for the treatment of disease or condition in which the disease or condition is driven by CD4+/CD8+ T-cells, as it is noted that certain SIRγ antibodies of the disclosure show preferential T-cell subtype binding.

In some embodiments, the Fc-containing SIRPγ antibodies provided herein are useful for the treatment of disease or condition in which the disease or condition is driven by CD69+/CD8+ T-cells, as it is noted that certain SIRγ antibodies of the disclosure show preferential T-cell subtype binding.

In some embodiments, the Fc-containing SIRPγ antibodies provided herein are useful for the treatment of disease or condition in which the disease or condition is driven by CD25+/CD8+ T-cells, as it is noted that certain SIRγ antibodies of the disclosure show preferential T-cell subtype binding.

### C. Pharmaceutical Compositions

The disclosure also provides pharmaceutical compositions comprising any one of the SIRPγ antibodies disclosed herein, and optionally a pharmaceutical acceptable excipient or carrier. In some embodiments, the pharmaceutical composition is sterile. The pharmaceutical compositions may be formulated to be compatible with their intended routes of administration. In some embodiments, the pharmaceutical compositions of the disclosure are suitable for administration to a human subject.

### D. Combination Therapies

The administration of any one of the therapeutic SIRPγ antibodies provided herein may be in combination with any other known drugs or treatments for diseases or conditions described above. Exemplary combinations, e.g. for the treatment of an oncology disease, includes one of the SIRPγ antibodies of the disclosure, administered in conjunction with a chemotherapeutic agent, cytotoxic agents, and corticosteroid drugs. Exemplary agents to be administered in combination include, but are not limited to cisplatin, Cladribine (2-CdA), Fludarabine, 6-thioguanine (6-TG), hydroxyurea, prednisone, dexamethasone, methotrexate (MTX), 6-mercaptopurine (6-MP), Azacitidine, and Decitabine.

### E. Administration of Therapeutic SIRPγ Antibodies

The in vivo administration of the therapeutic SIRPγ antibodies described herein may be carried out intravenously, intramuscularly, subcutaneously, topically, orally, transdermally, intraperitoneally, intraorbitally, intrathecally, intraventricularly, intranasally, transmucosally, through implantation, or through inhalation. Intravenous administration may be carried out via injection or infusion. In some embodiments, the SIRPγ antibodies of the disclosure are administered intravenously. In some embodiments, the SIRPγ antibodies of the disclosure are administered intraperitoneally. In some embodiments, the SIRPγ antibodies of the disclosure are administered subcutaneously. Administration of the therapeutic SIRPγ antibodies may be performed with any suitable excipients, carriers, or other agents to provide suitable or improved tolerance, transfer, delivery, and the like.

Exemplary dosages include administration of one of the SIRPγ antibodies of the disclosure at a dose of about 0.5 mg/kg, about 1 mg/kg, about 3 mg/kg, about 5 mg/kg, about 10 mg/kg, about 15 mg/kg, about 20 mg/kg, about 25 mg/kg, about 30 mg/kg, about 40 mg/kg, or about 50 mg/kg.

### F. Diagnostic Antibodies

The antibodies provided herein may also be used for diagnostic purposes. For example, diagnostic antibodies could be used for detecting the presence of a SIRPγ mediated disorder, or for detecting SIRPγ levels in a subject prior to dosing (e.g. as a companion diagnostic).

### III. Kits and Articles of Manufacture

The disclosure also provides a kit or article of manufacture comprising any one of the antibodies disclosed herein, or any pharmaceutical composition disclosed herein. In some embodiments, the kits may further include instructional materials for carrying out any of the methods disclosed herein. In some embodiments, the kits may further include sterile containers or vials for holding the antibodies and/or pharmaceutical compositions disclosed herein. In some embodiments, the kits may further include sterile delivery devices for administering the antibodies and/or pharmaceutical compositions disclosed herein. In some embodiments, an article of manufacture comprises any pharmaceutical composition of the disclosure.

### IV. Exemplary Enumerated Embodiments

Exemplary enumerated embodiments of the disclosure are as follows.

### Set I

Embodiment I-1. An antibody comprising any one of the CDR combinations of Table 1 or any one of the VH/VL combinations of Table 2.

Embodiment I-2. The antibody of embodiment I-1, wherein the antibody is a monoclonal antibody.

Embodiment I-3. The antibody of any one of embodiments I-1 to I-2, wherein the antibody is an antibody fragment.

Embodiment I-4. The antibody of any one of embodiments I-1 to I-3, wherein the antibody is a human antibody.

Embodiment I-5. The antibody of any one of embodiments I-1 to I-3, wherein the antibody is a humanized antibody.

Embodiment I-6. The antibody of any one of embodiments I-1 to I-3, wherein the antibody is a chimeric antibody.

Embodiment I-7. The antibody of any one of embodiments I-1 to I-3, wherein the antibody is a full-length antibody.

Embodiment I-8. The antibody of any one of embodiments I-1 to I-7, wherein the Fc domain is selected from the group consisting of human IgG1, IgG2, IgG3, and IgG4.

Embodiment I-9. The antibody of embodiment I-8, wherein the Fc domain comprises SEQ ID NO: 5 or SEQ ID NO: 6, optionally with one or more Fc amino acid substitutions.

Embodiment I-10. The antibody embodiment I-9, wherein the Fc domain comprises one or more amino acid substitutions relative to SEQ ID NO: 5 or SEQ ID NO: 6 at a position selected from the group consisting of: 215, 221, 222, 228, 234, 235, 236, 239, 240, 241, 243, 244, 245, 247, 250, 252, 254, 256, 262, 263, 264, 265, 266, 267, 268, 269, 270, 292, 296, 297, 298, 299, 300, 305, 313, 324, 325, 326, 327, 328, 329, 330, 332, 333, 334, 345, 396, 428, 430, 433, 434, and 440 wherein the position numbers of the amino acid residues are of the EU numbering scheme.

Embodiment I-11. The antibody of any one of embodiments I-1 to I-10, wherein the binding of the antibody does not disrupt the interaction between CD47 and SIRPγ.

Embodiment I-12. The antibody of any one of embodiments I-1 to I-10, wherein the binding of the antibody disrupts the interaction between CD47 and SIRPγ.

Embodiment I-13. The antibody of any one of embodiments I-1 to I-10, wherein the binding of the antibody stabilizes the interaction between CD47 and SIRPγ.

Embodiment I-14. The antibody of any one of embodiments I-1 to I-13, wherein, the antibody binds human SIRPγ and cynomolgus monkey SIRPγ.

Embodiment I-15. The antibody of any one of embodiments I-1 to I-14, wherein the antibody comprises a binding affinity to SIRPγ lower than about 500 nM.

Embodiment I-16. An Fc-containing antibody that is specific for SIRPγ, wherein the antibody has low or no affinity for binding SIRPα and SIRPβ1, and wherein binding of the antibody to a SIRPγ-expressing cell induces effector-mediated depletion of the SIRPγ-expressing cell.

Embodiment I-17. The antibody of embodiment I-16, wherein the SIRPγ-expressing cell is a T-cell, B-cell, or a NK-cell.

Embodiment I-18. The antibody of embodiment I-17, wherein the SIRPγ-expressing cell is a T-cell.

Embodiment I-19. The antibody of embodiment I-18, wherein the T-cell is a naïve, activated, central memory, effector memory, exhausted, or terminal effector memory cell.

Embodiment I-20. The antibody of embodiment I-18, wherein the T-cell is a cytotoxic T-cell, helper T-cell, a memory T-cell, a regulatory T-cell, a natural killer T-cell, a mucosal associated invariant T-cell, an alpha beta T-cell, or a gamma delta T-cell.

Embodiment I-21. The antibody of embodiment I-18, wherein the T-cell is a CD3+ T-cell, CD4+ T-cell or CD8+ T-cell.

Embodiment I-22. The antibody of embodiment I-18, wherein the T-cell is a Th1 cell, Th2 cell, Th17 cell or T follicular helper cell.

Embodiment I-23. The antibody of embodiment I-17, wherein the SIRPγ-expressing cell is a B-cell.

Embodiment I-24. The antibody of embodiment I-17, wherein the SIRPγ-expressing cell is an NK-cell.

Embodiment I-25. The antibody of any one of embodiments I-1 to I-24, wherein the SIRPγ-expressing cell is activated.

Embodiment I-26. The antibody of any one of embodiments I-1 to I-25, wherein the antibody preferentially depletes activated (stimulated) cells as a result of an increase in surface SIRPγ expression compared to naïve unactivated (unstimulated) cells.

Embodiment I-27. The antibody of any one of embodiments I-1 to I-26, wherein the binding of the antibody does not disrupt the interaction between CD47 and SIRPγ.

Embodiment I-28. The antibody of any one of embodiments I-1 to I-26, wherein the binding of the antibody stabilizes the interaction between CD47 and SIRPγ.

Embodiment I-29. The antibody of any one of embodiments I-1 to I-26, wherein the binding of the antibody disrupts the interaction between CD47 and SIRPγ.

Embodiment I-30. The antibody of any one of embodiments I-1 to I-29, wherein the cell depletion involves antibody dependent cellular phagocytosis (ADCP).

Embodiment I-31. The antibody of any one of embodiments I-1 to I-29, wherein the cell depletion involves antibody dependent cellular cytotoxicity (ADCC).

Embodiment I-32. The antibody of any one of embodiments I-1 to I-29, wherein the cell depletion involves complement dependent cytotoxicity (CDC).

Embodiment I-33. The antibody of any one of embodiments I-1 to I-32, wherein the antibody is a monoclonal antibody.

Embodiment I-34. The antibody of any one of embodiments I-1 to I-33, wherein the antibody is an antibody fragment.

Embodiment I-35. The antibody of any one of embodiments I-1 to I-34, wherein the antibody is a human antibody.

Embodiment I-36. The antibody of any one of embodiments I-1 to I-34, wherein the antibody is a humanized antibody.

Embodiment I-37. The antibody of any one of embodiments I-1 to I-34, wherein the antibody is a chimeric antibody.

Embodiment I-38. The antibody of any one of embodiments I-1 to I-34, wherein the antibody is a full-length antibody.

Embodiment I-39. The antibody of any one of embodiments I-1 to I-38, wherein the Fc domain is selected from the group consisting of human IgG1, IgG2, IgG3, and IgG4.

Embodiment I-40. The antibody of embodiment I-39, wherein the Fc domain comprises SEQ ID NO: 5 or SEQ ID NO: 6, optionally with one or more Fc amino acid substitutions.

Embodiment I-41. The antibody of any one embodiments I-38 to I-40, wherein the Fc domain comprises one or more amino acid substitutions relative to SEQ ID NO: 5 or SEQ ID NO: 6 at a position selected from the group consisting of: 215, 221, 222, 228, 234, 235, 236, 239, 240, 241, 243, 244, 245, 247, 250, 252, 254, 256, 262, 263, 264, 265, 266, 267, 268, 269, 270, 292, 296, 297, 298, 299, 300, 305, 313, 324, 325, 326, 327, 328, 329, 330, 332, 333, 334, 345, 396, 428, 430, 433, 434, and 440 wherein the position numbers of the amino acid residues are of the EU numbering scheme.

Embodiment I-42. The antibody of any one of embodiments I-1 to I-41, wherein, the antibody binds human SIRPγ and cynomolgus monkey SIRPγ.

Embodiment I-43. The antibody comprising of anyone of embodiments I-1 to I-42, wherein the antibody comprises a binding affinity to SIRPγ lower than about 500 nM.

Embodiment I-44. A pharmaceutical composition comprising the antibody of any one of embodiments I-1-43, and optionally a pharmaceutically acceptable carrier.

Embodiment I-45. A nucleic acid encoding for the antibody of any one of embodiments I-1 to I-43.

Embodiment I-46. A vector comprising the nucleic acid embodiment I-45.

Embodiment I-47. A method of inducing the depletion of a population of SIRPγ-expressing cells, the method comprising contacting the population with the antibody of any one of embodiments I-1 to I-43.

Embodiment I-48. The method of embodiment I-47, wherein the SIRPγ-expressing cells are T-cells, B-cells and/or NK-cells.

Embodiment I-49. The method of embodiment I-47, wherein the SIRPγ-expressing cells are T-cells.

Embodiment I-50. The method of embodiment I-49, wherein the T-cells are naïve, central memory, effector memory, exhausted, or terminal effector memory cells.

Embodiment I-51. The method of embodiment I-49, wherein the T-cell is a cytotoxic T-cell, helper T-cell, a memory T-cell, a regulatory T-cell, a natural killer T-cell, a mucosal associated invariant T-cell, an alpha beta T-cell, or a gamma delta T-cell.

Embodiment I-52. The method of embodiment I-49, wherein the T-cells are CD3+ T-cells, CD4+ T-cells or CD8+ T-cells

Embodiment I-53. The method of embodiment I-49, wherein the T-cells are Th1 cells, Th2 cells, Th17 cells or T follicular helper cells.

Embodiment I-54. The method of embodiment I-47, wherein the SIRPγ-expressing cells are B-cells.

Embodiment I-55. The method of embodiment I-47, wherein the SIRPγ-expressing cells are NK-cells.

Embodiment I-56. The method of any one of embodiments I-47 to I-55, wherein, the SIRPγ-expressing cells are activated.

Embodiment I-57. The method of any one of embodiments I-47 to I-56, wherein the method is in vitro.

Embodiment I-58. The method of any one of embodiments I-47 to I-56, wherein the method is in vivo.

Embodiment I-59. The method any one of embodiments I-47 to I-58, wherein the population of SIRPγ-expressing cells comprises tissue-resident cells.

Embodiment I-60. The method any one of embodiments I-47 to I-59, wherein the population of SIRPγ-expressing cells comprises circulating cells.

Embodiment I-61. The method of any one of embodiments I-47 to I-60, wherein the depletion involves one or more of ADCC, ADCP, and CDC.

Embodiment I-62. A method of treating a disease or condition in a subject in need thereof, comprising administering to the subject a therapeutically effective amount of the antibody of any one of embodiments I-1 to I-43 or the pharmaceutical composition of embodiment I-44.

Embodiment I-63. The method of embodiment I-62, wherein the disease or condition involves SIRPγ-expressing cells.

Embodiment I-64. The method of embodiment I-63, wherein the SIRPγ-expressing cell is a T-cell, B-cell, or a NK-cell.

Embodiment I-65. The method of embodiment I-64, wherein the SIRPγ-expressing cell is a T-cell.

Embodiment I-66. The method of embodiment I-65, wherein the T-cell is activated.

Embodiment I-67. The method of any one of embodiments I-64 to I-66, wherein the T-cell is a CD4+ T-cell, CD8+ T-cell, Th1 cell, Th2 cell, Th17 cell or a T follicular helper cell.

Embodiment I-68. The method of any one of embodiments I-64 to I-66, wherein the T-cell is a cytotoxic T-cell, helper T-cell, a memory T-cell, a regulatory T-cell, a natural killer T-cell, a mucosal associated invariant T-cell or a gamma delta T-cell.

Embodiment I-69. The method of embodiment I-65, wherein the T-cell is a naïve, activated, central memory, effector memory, exhausted, or terminal effector memory cell.

Embodiment I-70. The method of embodiment I-64, wherein the SIRPγ-expressing cell is a B-cell.

Embodiment I-71. The method of embodiment I-70, wherein the B-Cell is activated.

Embodiment I-72. The method of embodiment I-64, wherein the SIRPγ-expressing cell is an NK-cell.

Embodiment I-73. The method of embodiment I-72, wherein the NK-cell is activated.

Embodiment I-74. The method of embodiments I-62 to I-73, wherein the disease or condition comprises an autoimmune, oncology, or inflammatory disorder.

Embodiment I-75. The method of embodiments I-64 to I-69, wherein the disease or condition comprises a T-cell-mediated autoimmune disease, T-cell-mediated inflammatory disease, or T-cell-mediated oncology disease.

Embodiment I-76. The method of embodiment I-74, wherein the disease or condition is selected from: Acute and Chronic eosinophilic pneumonia, Acute disseminated encephalomyelitis, Acute Disseminated Encephalomyelitis, Acute Lymphoblastic Leukemia (ALL), Acute myelogenous leukemia (AML), Addison's disease, Adult-onset Still's disease (AOSD), Aplastic anemia, Ataxia Telangiectasia, Atopic dermatitis, Autoimmune lymphoproliferative syndrome, Axial spondyloarthritis (AxSpA), Birdshot Retinochoroidopathy, Castleman disease, Celiac disease, Chediak-Higashi syndrome, Coronary artery disease/peripheral artery disease, Crohn's Disease, Episodic angioedema with eosinophilia/Gleich syndrome, Giant-Lymphocyte arteritis, Graft failure post-HSCT, Graft vs Host Disease (GvHD), Graves' disease, Hepatosplenic lymphoma, Hypothyroidism, Idiopathic interstitial pneumonias, IgA nephropathy, Inclusion Body Myositis (IBM), Inflammatory bowel disease (IBD), Large granular lymphocytic leukemia, Lymphocyte-variant hypereosinophila, Multiple sclerosis, Myelodysplastic syndromes (MDS), Myocarditis, Neuromyelitis optica spectrum disorders, Paraneoplastic syndromes, Primary biliary cholangitis, Primary sclerosing cholangitis, Rasmussen's Encephalitis, Rheumatoid arthritis (RA), Sarcoidosis, Schmidt syndrome/Autoimmune polyendocrine syndrome type II, Stiff Person Syndrome, Susac syndrome, Sympathetic Opthalmis, Systemic juvenile idiopathic arthritis (sJIA), Systemic Lupus Erythematosus (SLE), T-Lymphocyte mediated rejection of solid organ transplants, T-Cell Prolymphocytic Leukemia (TPLL), Type 1 diabetes, Ulcerative colitis, Uveitis, Vitiligo, X-linked Hyper IgM Syndrome, Autoimmune Hepatitis, and X-linked lymphoproliferative disease.

Embodiment I-77. The method of any one of embodiments I-62 to I-76, wherein the subject is human.

Embodiment I-78. A cell expressing SIRPγ, wherein the cell bound is to an antibody of any one of embodiments I-1 to I-43, wherein the antibody is bound to the SIRPγ.

Embodiment I-79. A kit or article of manufacture comprising an antibody of any one of embodiments I-1 to I-43, or the pharmaceutical composition of embodiment I-44.

Embodiment I-80. Use of the antibody of any one of embodiments I-1 to I-43, or the pharmaceutical composition of embodiment I-44 for the treatment of a disease or disorder in a subject in need thereof.

Embodiment I-81. Us of the antibody of any one of embodiments I-1 to I-43, or the pharmaceutical composition of embodiment I-44 for the manufacture of a medicament for the treatment of a disease or disorder in a subject in need thereof.

### Set II:

Embodiment II-1. An Fc-containing antibody that is specific for SIRPγ, wherein the antibody has low or no affinity for binding SIRPα and SIRPβ1, and wherein binding of the antibody to a SIRPγ-expressing cell induces effector-mediated depletion of the SIRPγ-expressing cell.

Embodiment II-2. The antibody of embodiment II-1, wherein the SIRPγ-expressing cell is a T-cell, B-cell, or a NK-cell.

Embodiment II-3. The antibody of embodiment II-2, wherein the SIRPγ-expressing cell is a T-cell.

Embodiment II-4. The antibody of embodiment II-2, wherein the SIRPγ-expressing cell is a stimulated (activated) T-cell.

Embodiment II-5. The antibody of embodiment II-2, wherein the SIRPγ-expressing cell is an exhausted T-cell.

Embodiment II-6. The antibody of embodiment II-3, wherein the T-cell is a naïve, activated, central memory, effector memory, or terminal effector memory cell.

Embodiment II-7. The antibody of embodiment II-3, wherein the T-cell is a cytotoxic T-cell, helper T-cell, a memory T-cell, a regulatory T-cell, a natural killer T-cell, a mucosal associated invariant T-cell, an alpha beta T-cell, or a gamma delta T-cell.

Embodiment II-8. The antibody of embodiment II-3, wherein the T-cell is a Th1 cell, Th2 cell, Th17 cell or T follicular helper cell.

Embodiment II-9. The antibody of embodiment II-3, wherein the T-cell is a CD3+ T-cell, CD4+ T-cell, CD8+ T-cell, CD25+ T-cell, CD69+ T-cell, and/or PD1+ T-cell.

Embodiment II-10. The antibody of embodiment II-3, wherein the T-cell is a CD4+/CD8+ T-cell.

Embodiment II-11. The antibody of embodiment II-3, wherein the T-cell is a CD69+/CD8+ T-cell.

Embodiment II-12. The antibody of embodiment II-3, wherein the T-cell is a CD25+/CD8+ T-cell.

Embodiment II-13. The antibody of embodiment II-3, wherein the T-cell is a PD1+ T-cell.

Embodiment II-14. The antibody of embodiment II-3, wherein the binding of the antibody to a SIRPγ-expressing cell is preferential for a stimulated T-cell, as compared to an unstimulated T-cell.

Embodiment II-15. The antibody of embodiment II-3, wherein the binding of the antibody to a SIRPγ-expressing cell induces preferential effector-mediated depletion of a stimulated T-cell.

Embodiment II-16. The antibody of embodiment II-3, wherein the binding of the antibody to a SIRPγ-expressing cell induces preferential effector-mediated depletion of an exhausted T-cell.

Embodiment II-17. The antibody of embodiment II-9, wherein binding of the antibody to a SIRPγ-expressing cell induces preferential effector-mediated depletion of a SIRPγ-expressing CD8+ T-cell.

Embodiment II-18. The antibody of embodiment II-9, wherein binding of the antibody to a SIRPγ-expressing cell induces preferential effector-mediated depletion of a SIRPγ-expressing CD4+ T-cell.

Embodiment II-19. The antibody of embodiment II-9, wherein binding of the antibody to a SIRPγ-expressing cell induces preferential effector-mediated depletion of a SIRPγ-expressing CD8+/CD69+ T-cell.

Embodiment II-20. The antibody of embodiment II-9, wherein binding of the antibody to a SIRPγ-expressing cell induces preferential effector-mediated depletion of a SIRPγ-expressing CD8+/CD25+ T-cell.

Embodiment II-21. The antibody of embodiment II-9, wherein binding of the antibody to a SIRPγ-expressing cell induces preferential effector-mediated depletion of a SIRPγ-expressing CD8+ T-cell, when compared to a SIRPγ-expressing CD4+ T-cell.

Embodiment II-22. The antibody of embodiment II-9, wherein binding of the antibody to a SIRPγ-expressing cell induces preferential effector-mediated depletion of a SIRPγ-expressing CD4+ T-cell, when compared to a SIRPγ-expressing CD8+ T-cell.

Embodiment II-23. The antibody of embodiment II-9, wherein binding of the antibody to a SIRPγ-expressing cell induces preferential effector-mediated depletion of a SIRPγ-expressing CD8+/CD69+ T-cell, when compared to a SIRPγ-expressing CD8+/CD69− T-cell.

Embodiment II-24. The antibody of embodiment II-9, wherein binding of the antibody to a SIRPγ-expressing cell induces preferential effector-mediated depletion of a SIRPγ-expressing CD8+/CD25+ T-cell, when compared to a SIRPγ-expressing CD8+/CD25− T-cell.

Embodiment II-25. The antibody of embodiment II-9, wherein binding of the antibody to a SIRPγ-expressing cell induces preferential effector-mediated depletion of a SIRPγ-expressing PD1+ T-cell, when compared to a SIRPγ-expressing PD1− T-cell.

Embodiment II-26. The antibody of embodiment II-2, wherein the SIRPγ-expressing cell is a B-cell.

Embodiment II-27. The antibody of embodiment II-2, wherein the SIRPγ-expressing cell is an NK-cell.

Embodiment II-28. The antibody of any one of embodiments II-1 to II-27, wherein the SIRPγ-expressing cell is stimulated (activated).

Embodiment II-29. The antibody of any one of embodiments II-1 to II-27, wherein the SIRPγ-expressing cell is exhausted.

Embodiment II-30. The antibody of any one of embodiments II-1 to II-29, wherein the antibody preferentially depletes activated (stimulated) cells as a result of an increase in surface SIRPγ expression compared to naïve unactivated (unstimulated) cells.

Embodiment II-31. The antibody of any one of embodiments II-1 to II-30, wherein the cell depletion involves antibody dependent cellular phagocytosis (ADCP).

Embodiment II-32. The antibody of any one of embodiments II-1 to II-30, wherein the cell depletion involves antibody dependent cellular cytotoxicity (ADCC).

Embodiment II-33. The antibody of any one of embodiments II-1 to II-30, wherein the cell depletion involves complement dependent cytotoxicity (CDC).

Embodiment II-34. The antibody of any one of embodiments II-1 to II-33, wherein the antibody comprises the CDR amino acid sequences of any one of the combinations of Table 1, as presented in embodiment II-46.

Embodiment II-35. The antibody of any one of embodiments II-1 to II-34, wherein the antibody comprises the VH and VL amino acid sequences of any one of the combinations of Table 2, or comprises a sequence having at least 70% sequence identity thereto, as presented in embodiment II-47.

Embodiment II-36. A SIRPγ antibody that has low or no affinity for binding SIRPα and SIRPβ1, and wherein the antibody exhibits preferential binding to an activated (stimulated) T-cell, as compared to an unstimulated T-cell.

Embodiment II-37. A SIRPγ antibody that has low or no affinity for binding SIRPα and SIRPβ1, and wherein the antibody exhibits preferential binding to a CD8+ T-cell.

Embodiment II-38. A SIRPγ antibody that has low or no affinity for binding SIRPα and SIRPβ1, and wherein the antibody exhibits preferential binding to a CD4+ T-cell.

Embodiment II-39. A SIRPγ antibody that has low or no affinity for binding SIRPα and SIRPβ1, and wherein the antibody exhibits preferential binding to a CD8+/CD69+ T-cell.

Embodiment II-40. A SIRPγ antibody that has low or no affinity for binding SIRPα and SIRPβ1, and wherein the antibody exhibits preferential binding to a CD8+/CD25+ T-cell.

Embodiment II-41. A SIRPγ antibody that has low or no affinity for binding SIRPα and SIRPβ1, and wherein the antibody exhibits preferential binding to a CD8+ T-cell, when compared to a SIRPγ-expressing CD4+ T-cell.

Embodiment II-42. A SIRPγ antibody that has low or no affinity for binding SIRPα and SIRPβ1, and wherein the antibody exhibits preferential binding to a CD4+ T-cell, when compared to a SIRPγ-expressing CD8+ T-cell.

Embodiment II-43. A SIRPγ antibody that has low or no affinity for binding SIRPα and SIRPβ1, and wherein the antibody exhibits preferential binding to a CD8+/CD69+ T-cell, when compared to a SIRPγ-expressing CD8+/CD69− T-cell.

Embodiment II-44. A SIRPγ antibody that has low or no affinity for binding SIRPα and SIRPβ1, and wherein the antibody exhibits preferential binding to a CD8+/CD25+ T-cell, when compared to a SIRPγ-expressing CD8+/CD25− T-cell.

Embodiment II-45. A SIRPγ antibody that has low or no affinity for binding SIRPα and SIRPβ1, and wherein the antibody exhibits preferential binding to a PD1+ T-cell, when compared to a SIRPγ-expressing PD1− T-cell.

Embodiment II-46. A SIRPγ antibody that has low or no affinity for binding SIRPα and SIRPβ1, and wherein the antibody comprises the amino acid sequences of any one of the forty-seven CDR combinations of Table 1.

Embodiment II-47. A SIRPγ antibody that has low or no affinity for binding SIRPα and SIRPβ1, and wherein the antibody comprises the amino acid sequences of any one of the forty-seven VH/VL combinations of Table 1, or a sequence having at least 70% identity thereto.

Embodiment II-48. The antibody of any one of embodiments II-1 to II-47, wherein the antibody is an antibody fragment.

Embodiment II-49. The antibody of any one of embodiments II-1 to II-48, wherein the antibody is a human antibody.

Embodiment II-50. The antibody of any one of embodiments II-1 to II-48, wherein the antibody is a humanized antibody.

Embodiment II-51. The antibody of any one of embodiments II-1 to II-48, wherein the antibody is a chimeric antibody.

Embodiment II-52. The antibody of any one of embodiments II-1 to II-48, wherein the antibody is a full-length antibody.

Embodiment II-53. The antibody of any one of embodiments II-1 to II-52, wherein the antibody comprises a Fc domain, and the Fc domain is selected from the group consisting of human IgG1, IgG2, IgG3, and IgG4 heavy chain sequence.

Embodiment II-54. The antibody of embodiment II-53, wherein the Fc domain is from the heavy chain IgG amino acid sequences of SEQ ID NO: 5 or SEQ ID NO: 28, optionally with one or more Fc amino acid substitutions.

Embodiment II-55. The antibody of embodiment II-54, wherein the Fc domain is from the heavy chain IgG amino acid sequences of any one of SEQ ID NOS: 5-36.

Embodiment II-56. The antibody embodiment II-54, wherein the heavy chain Fc domain comprises one or more amino acid substitutions relative to SEQ ID NO: 5 or SEQ ID NO: 28 at a position selected from the group consisting of: 215, 221, 222, 228, 234, 235, 236, 239, 240, 241, 243, 244, 245, 247, 250, 252, 254, 256, 262, 263, 264, 265, 266, 267, 268, 269, 270, 292, 296, 297, 298, 299, 300, 305, 313, 324, 325, 326, 327, 328, 329, 330, 332, 333, 334, 345, 396, 428, 430, 433, 434, and 440 wherein the position numbers of the amino acid residues are of the EU numbering scheme.

Embodiment II-57. The antibody of any one of embodiments II-1 to II-56, wherein the antibody comprises a light chain constant region that is from the amino acid sequences of any one of SEQ ID NOS: 38-42.

Embodiment II-58. The antibody of any one of embodiments II-1 to II-57, wherein the binding of the antibody does not disrupt the interaction between CD47 and SIRPγ.

Embodiment II-59. The antibody of any one of embodiments II-1 to II-57, wherein the binding of the antibody disrupts the interaction between CD47 and SIRPγ.

Embodiment II-60. The antibody of any one of embodiments II-1 to II-57, wherein the binding of the antibody stabilizes or promotes the interaction between CD47 and SIRPγ.

Embodiment II-61. The antibody of any one of embodiments II-1 to II-57, wherein, the antibody binds human SIRPγ and cynomolgus monkey SIRPγ.

Embodiment II-62. The antibody comprising of anyone of embodiments II-1 to II-57, wherein the antibody comprises a binding affinity to SIRPγ lower than about 500 nM.

Embodiment II-63. A pharmaceutical composition comprising the antibody of any one of embodiments II-1 to II-62, and optionally a pharmaceutically acceptable carrier.

Embodiment II-64. A nucleic acid encoding for the antibody of any one of embodiments II-1 to II-62.

Embodiment II-65. A vector comprising the nucleic acid embodiment II-64.

Embodiment II-66. A method of inducing the preferential depletion of a population of SIRPγ-expressing cells, the method comprising contacting the population with the antibody of any one of embodiments II-1 to II-62.

Embodiment II-67. The method of embodiment II-66, wherein the SIRPγ-expressing cells are T-cells, B-cells and/or NK-cells.

Embodiment II-68. The method of embodiment II-66, wherein the SIRPγ-expressing cells are T-cells.

Embodiment II-69. The method of embodiment II-68, wherein the T-cells are activated (stimulated).

Embodiment II-70. The method of embodiment II-68, wherein the T-cells are activated (stimulated), and the depletion is preferential for activated (stimulated) T-cells.

Embodiment II-71. The method of embodiment II-68, wherein the T-cells are exhausted.

Embodiment II-72. The method of embodiment II-68, wherein the T-cells are exhausted, and the depletion is preferential for exhausted T-cells.

Embodiment II-73. The method of embodiment II-68, wherein the T-cells are naïve, central memory, effector memory, exhausted, or terminal effector memory cells.

Embodiment II-74. The method of embodiment II-68, wherein the T-cells are cytotoxic T-cells, helper T-cells, memory T-cells, regulatory T-cells, natural killer T-cells, mucosal associated invariant T-cells, alpha beta T-cells, or gamma delta T-cells.

Embodiment II-75. The method of embodiment II-68, wherein the T-cells are Th1 cells, Th2 cells, Th17 cells or T follicular helper cells

Embodiment II-76. The method of embodiment II-68, wherein the T-cells are a CD3+ T-cell, CD4+ T-cell, CD8+ T-cell, CD25+ T-cell, CD69+ T-cell, and/or PD1+ T-cell.

Embodiment II-77. The method of embodiment II-76, wherein the T-cells are CD4+/CD8+ T-cells.

Embodiment II-78. The method of embodiment II-76, wherein the T-cells are CD69+/CD8+ T-cells.

Embodiment II-79. The method of embodiment II-76, wherein the T-cells are CD25+/CD8+ T-cells.

Embodiment II-80. The method of embodiment II-76, wherein the T-cells are PD1+ T-cells.

Embodiment II-81. The method of embodiment II-76, wherein the depletion is preferential for stimulated T-cells, as compared to unstimulated T-cells.

Embodiment II-82. The method of embodiment II-76, wherein the depletion is preferential for SIRPγ-expressing CD8+ T-cells.

Embodiment II-83. The method of embodiment II-76, wherein the depletion is preferential for SIRPγ-expressing CD4+ T-cells.

Embodiment II-84. The method of embodiment II-76, wherein the depletion is preferential for SIRPγ-expressing CD8+/CD69+ T-cells.

Embodiment II-85. The method of embodiment II-76, wherein the depletion is preferential for SIRPγ-expressing CD8+/CD25+ T-cells.

Embodiment II-86. The method of embodiment II-76, wherein the depletion is preferential for SIRPγ-expressing CD8+ T-cells, when compared to SIRPγ-expressing CD4+ T-cells.

Embodiment II-87. The method of embodiment II-76, wherein the depletion is preferential for SIRPγ-expressing CD4+ T-cells, when compared to SIRPγ-expressing CD8+ T-cells.

Embodiment II-88. The method of embodiment II-76, wherein the depletion is preferential for SIRPγ-expressing CD8+/CD69+ T-cells, when compared to SIRPγ-expressing CD8+/CD69− T-cells.

Embodiment II-89. The method of embodiment II-76, wherein the depletion is preferential for SIRPγ-expressing CD8+/CD25+ T-cells, when compared to SIRPγ-expressing CD8+/CD25− T-cells.

Embodiment II-90. The method of embodiment II-66, wherein the SIRPγ-expressing cells are B-cells.

Embodiment II-91. The method of embodiment II-66, wherein the SIRPγ-expressing cells are NK-cells.

Embodiment II-92. The method of any one of embodiments II-66 to II-91, wherein, the SIRPγ-expressing cells are activated.

Embodiment II-93. The method of any one of embodiments II-66 to II-92, wherein the method is in vitro.

Embodiment II-94. The method of any one of embodiments II-66 to II-92, wherein the method is in vivo.

Embodiment II-95. The method any one of embodiments II-66 to II-94, wherein the population of SIRPγ-expressing cells comprises tissue-resident cells.

Embodiment II-96. The method any one of embodiments II-66 to II-95, wherein the population of SIRPγ-expressing cells comprises circulating cells.

Embodiment II-97. The method of any one of embodiments II-66 to II-96, wherein the depletion involves one or more of ADCC, ADCP, and CDC.

Embodiment II-98. A method of treating a disease or condition in a subject in need thereof, comprising administering to the subject a therapeutically effective amount of the antibody of any one of embodiments II-1 to II-62 or the pharmaceutical composition of embodiment II-63.

Embodiment II-99. The method of embodiment II-98, wherein the disease or condition involves SIRPγ-expressing cells.

Embodiment II-100. The method of embodiment II-99, wherein the SIRPγ-expressing cell is a T-cell, B-cell, or a NK-cell.

Embodiment II-101. The method of embodiment II-100, wherein the SIRPγ-expressing cell is a T-cell.

Embodiment II-102. The method of embodiment II-101, wherein the T-cell is stimulated (activated).

Embodiment II-103. The method of embodiment II-101, wherein the T-cell is exhausted.

Embodiment II-104. The method of any one of embodiments II-100 to II-102, wherein the T-cell is a cytotoxic T-cell, helper T-cell, a memory T-cell, a regulatory T-cell, a natural killer T-cell, a mucosal associated invariant T-cell or a gamma delta T-cell.

Embodiment II-105. The method of any one of embodiments II-100 to II-102, wherein the T-cell is a naïve, central memory, effector memory, or terminal effector memory cell.

Embodiment II-106. The method of any one of embodiments II-100 to II-102, wherein the T-cell is a CD3+ T-cell, CD4+ T-cell, CD8+ T-cell, CD25+ T-cell, CD69+ T-cell, and/or PD1+ T-cell.

Embodiment II-107. The method of embodiment II-106, wherein the T-cell is a CD4+/CD8+ T-cell.

Embodiment II-108. The method of embodiment II-106, wherein the T-cell is a CD69+/CD8+ T-cell.

Embodiment II-109. The method of embodiment II-106, wherein the T-cell is a CD25+/CD8+ T-cell.

Embodiment II-110. The method of embodiment II-106, wherein the T-cell is a PD1+ T-cell.

Embodiment II-111. The method of embodiment II-106, wherein the binding of the antibody is preferential for an stimulated T-cell, as compared to an unstimulated T-cell.

Embodiment II-112. The method of embodiment II-106, wherein binding of the antibody is preferential for a SIRPγ-expressing CD8+ T-cell.

Embodiment II-113. The method of embodiment II-106, wherein binding of the antibody is preferential for a SIRPγ-expressing CD4+ T-cell.

Embodiment II-114. The method of embodiment II-106, wherein binding of the antibody to a SIRPγ-expressing cell induces preferential effector-mediated depletion of a SIRPγ-expressing CD8+/CD69+ T-cell.

Embodiment II-115. The method of embodiment II-106, wherein binding of the antibody is preferential for a SIRPγ-expressing CD8+/CD25+ T-cell.

Embodiment II-116. The method of embodiment II-106, wherein binding of the antibody is preferential for a SIRPγ-expressing CD8+ T-cell, when compared to a SIRPγ-expressing CD4+ T-cell.

Embodiment II-117. The method of embodiment II-106, wherein binding of the antibody is preferential for a SIRPγ-expressing CD4+ T-cell, when compared to a SIRPγ-expressing CD8+ T-cell.

Embodiment II-118. The method of embodiment II-106, wherein binding of the antibody is preferential for a SIRPγ-expressing CD8+/CD69+ T-cell, when compared to a SIRPγ-expressing CD8+/CD69− T-cell.

Embodiment II-119. The method of embodiment II-106, wherein binding of the antibody is preferential for a SIRPγ-expressing CD8+/CD25+ T-cell, when compared to a SIRPγ-expressing CD8+/CD25− T-cell.

Embodiment II-120. The method of embodiment II-100, wherein the SIRPγ-expressing cell is a B-cell.

Embodiment II-121. The method of embodiment II-120, wherein the B-Cell is activated.

Embodiment II-122. The method of embodiment II-100, wherein the SIRPγ-expressing cell is an NK-cell.

Embodiment II-123. The method of embodiment II-122, wherein the NK-cell is activated.

Embodiment II-124. The method of any one of embodiments II-98 to 11-123, wherein the disease or condition comprises an autoimmune, oncology, or inflammatory disorder.

Embodiment II-125. The method of embodiment II-124, wherein the disease or condition comprises a T-cell-mediated autoimmune disease, T-cell-mediated inflammatory disease, or T-cell-mediated oncology disease.

Embodiment II-126. The method of any one of embodiments II-98 to 11-123, wherein the disease or condition is driven by CD8+ T-cells.

Embodiment II-127. The method of any one of embodiments II-98 to 11-123, wherein the disease or condition is driven by CD4+ T-cells.

Embodiment II-128. The method of any one of embodiments II-98 to 11-123, wherein the disease or condition is driven by CD8+ T-cells and CD4+ T-cells.

Embodiment II-129. The method of any one of embodiments II-98 to 11-125, wherein the disease or condition is selected from: Acute and Chronic eosinophilic pneumonia, Acute disseminated encephalomyelitis, Acute Disseminated Encephalomyelitis, Acute Lymphoblastic Leukemia (ALL), Acute myelogenous leukemia (AML), Addison's disease, Adult-onset Still's disease (AOSD), Aplastic anemia, Ataxia Telangiectasia, Atopic dermatitis, Autoimmune lymphoproliferative syndrome, Axial spondyloarthritis (AxSpA), Birdshot Retinochoroidopathy, Castleman disease, Celiac disease, Chediak-Higashi syndrome, Coronary artery disease/peripheral artery disease, Crohn's Disease, Episodic angioedema with eosinophilia/Gleich syndrome, Giant-Lymphocyte arteritis, Graft failure post-HSCT, Graft vs Host Disease (GvHD), Graves' disease, Hepatosplenic lymphoma, Hypothyroidism, Idiopathic interstitial pneumonias, IgA nephropathy, Inclusion Body Myositis (IBM), Inflammatory bowel disease (IBD), Large granular lymphocytic leukemia, Lymphocyte-variant hypereosinophila, Multiple sclerosis, Myelodysplastic syndromes (MDS), Myocarditis, Neuromyelitis optica spectrum disorders, Paraneoplastic syndromes, Primary biliary cholangitis, Primary sclerosing cholangitis, Rasmussen's Encephalitis, Rheumatoid arthritis (RA), Sarcoidosis, Schmidt syndrome/Autoimmune polyendocrine syndrome type II, Stiff Person Syndrome, Susac syndrome, Sympathetic Opthalmis, Systemic juvenile idiopathic arthritis (sJIA), Systemic Lupus Erythematosus (SLE), T-Lymphocyte mediated rejection of solid organ transplants, T-Cell Prolymphocytic Leukemia (TPLL), Type 1 diabetes, Ulcerative colitis, Uveitis, Vitiligo, X-linked Hyper IgM Syndrome, Autoimmune Hepatitis, and X-linked lymphoproliferative disease.

Embodiment II-130. The method of any one of embodiments II-98 to II-129, wherein the subject is human.

Embodiment II-131. A cell expressing SIRPγ, wherein the cell bound is to an antibody of any one of embodiments II-1 to II-62, wherein the antibody is bound to the SIRPγ.

Embodiment II-132. A kit or article of manufacture comprising an antibody of any one of embodiments II-1 to II-62, or the pharmaceutical composition of embodiment II-63.

Embodiment II-133. Use of the antibody of any one of embodiments II-1 to II-62, or the pharmaceutical composition of embodiment II-63 for the treatment of a disease or disorder in a subject in need thereof.

Embodiment II-134. Us of the antibody of any one of embodiments II-1 to II-62, or the pharmaceutical composition of embodiment II-63 for the manufacture of a medicament for the treatment of a disease or disorder in a subject in need thereof.

### EXAMPLES

### Example 1: Hybridoma Library Screens for Identification of Anti-Human SIRPγ Antibodies

Anti-human SIRPγ (Anti-hSIRPγ) monoclonal antibodies (referred to in these examples simply as SIRPγ antibodies) were identified from a transgenic mouse model. Harbour Mice® H2L2 strain were immunized with the extracellular domain of human SIRPγ (hSIRPγ). Using standard techniques, hybridoma libraries (two libraries) were generated from the splenocytes of immunized animals. Anti-hSIRPγ antibody-producing clones were identified by flow cytometric analyses of hSIRPγ-expressing cells exposed to antibody-containing supernatants of individual clones. Anti-hSIRPγ-specific clones were picked with counter screens against human SIRPα-expressing cells and human SIRPβ1-expressing cells via flow cytometry and confirmed with SIRP protein binding via ELISA. All clones resulted from this campaign have a human variable region and rodent constant regions. Select clones were reformatted to fully human antibodies, that is they were reformatted to have a human variable region, and a human constant region. It is noted that the human constant region could comprise any one of the Fc regions provided in this disclosure, e.g. those of SEQ ID NOS: 5-36, canonical, modified or otherwise.

### Example 2: Phage Antibody Library Screens for Identification of Anti-Human SIRPγ Antibodies

Using a separate approach, additional SIRPγ antibodies were identified from a fully human phage display library. The library was first enriched for human SIRPγ binding on magnetic beads. Multiple sub-libraries were made with deselection of SIRPα and SIRPβ1 binders and/or positive selection cynomolgus monkey SIRPγ cross reactivity. The resulting specific single clone library was generated and sequenced. All clones resulted from this campaign are single-chain fragment variable (scFv) antibodies as crude periplasmic extracts (PPE). Select clones were reformatted to fully human antibodies to include various Fcs of the disclosure (e.g. Fc that increases effector function, Fc that decreases effector function, and the like), that is they were reformatted to have a human variable region and a human constant region. It is noted that the human constant region could comprise any one of the Fc regions provided in this disclosure, e.g. those of SEQ ID NOS: 5-36, canonical, modified or otherwise.

### Example 3: Binding of SIRPγ Antibodies to SIRP Proteins

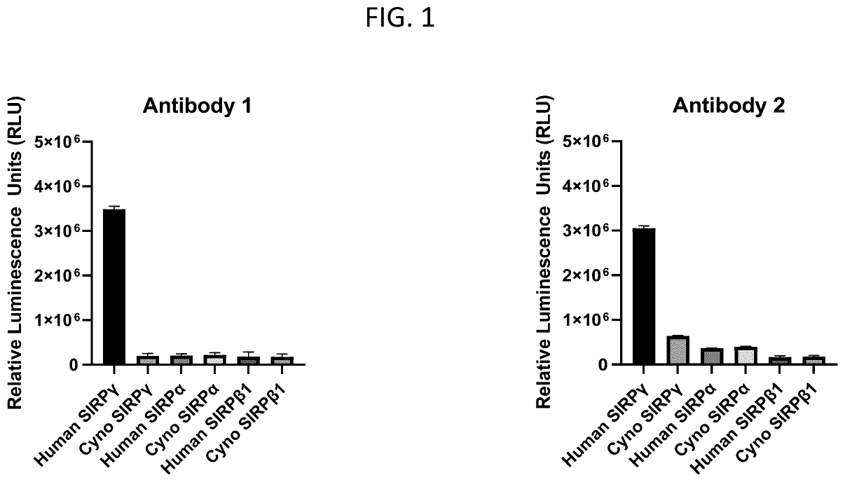

Selected hybridoma supernatants of Example 1 were tested for binding to human SIRPγ, cynomolgus monkey SIRPγ, human SIRPα V1, cynomolgus monkey SIRPα, human SIRPβ1, and cynomolgus monkey SIRPβ1 by enzyme-linked immunosorbent assay (ELISA). Briefly, 2 μg/mL of the extracellular domain of SIRP protein was coated onto high protein-binding plates and blocked. Undiluted supernatants were added to coated plates. The antibodies were detected by an anti-rat antibody and a chemiluminescent substrate. FIG. 1 shows the results of binding of Antibodies 1 and 2 to the various SIRP proteins. The data depict the relative luminescence units read by a plate-reader capable of detecting chemiluminescence.

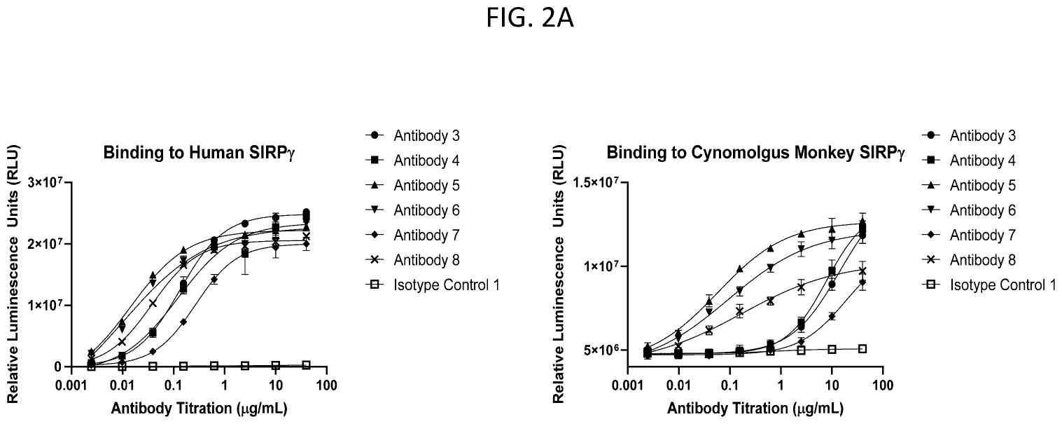

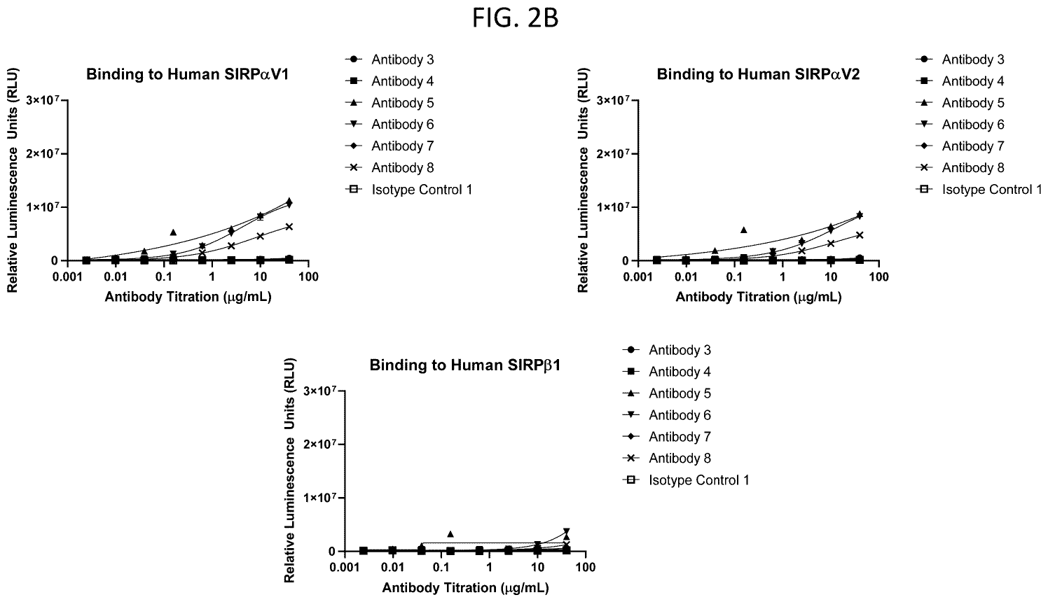

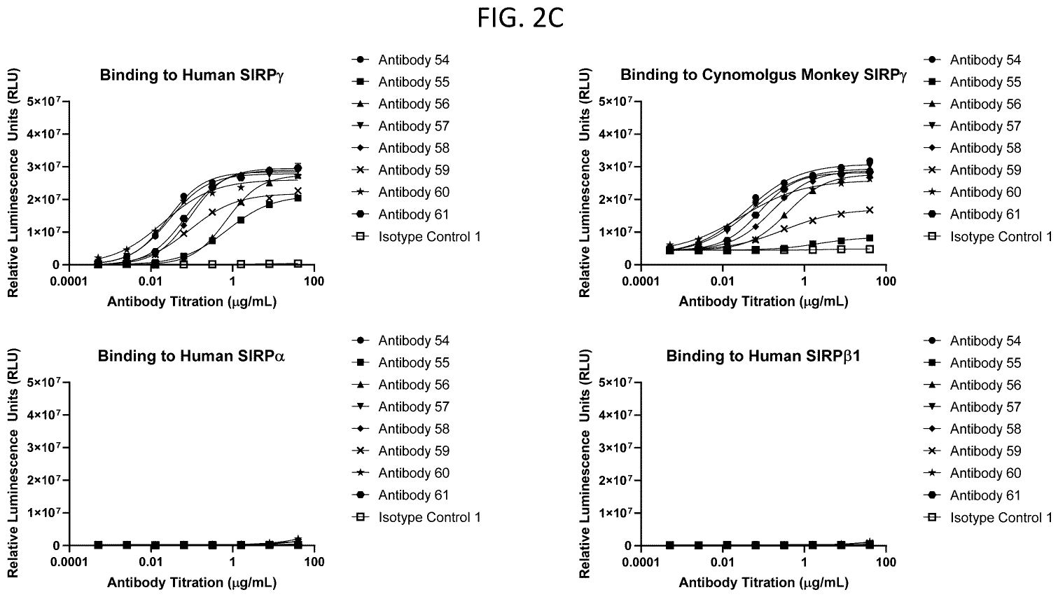

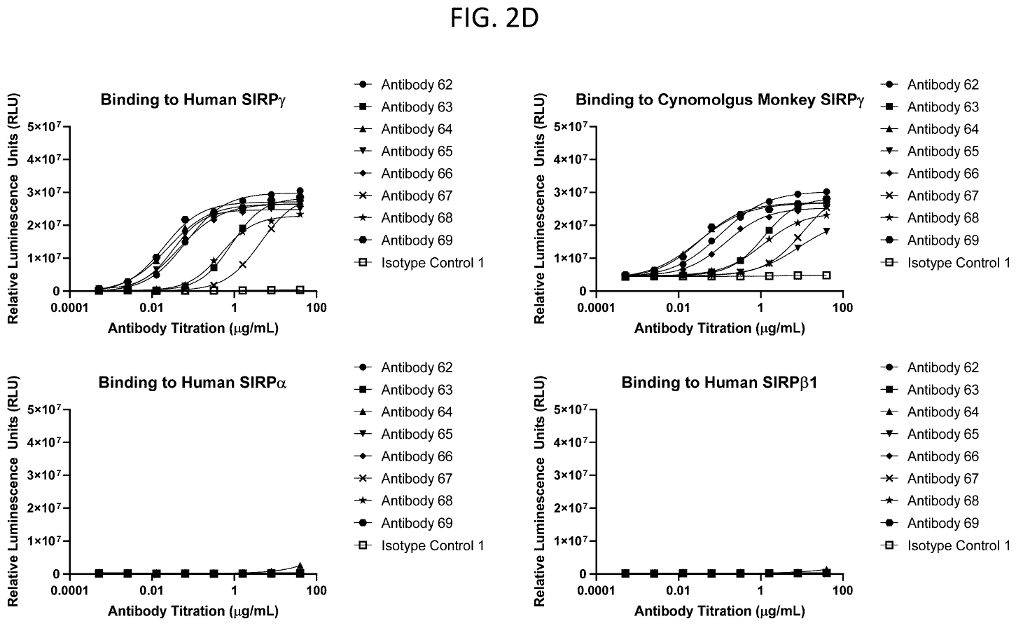

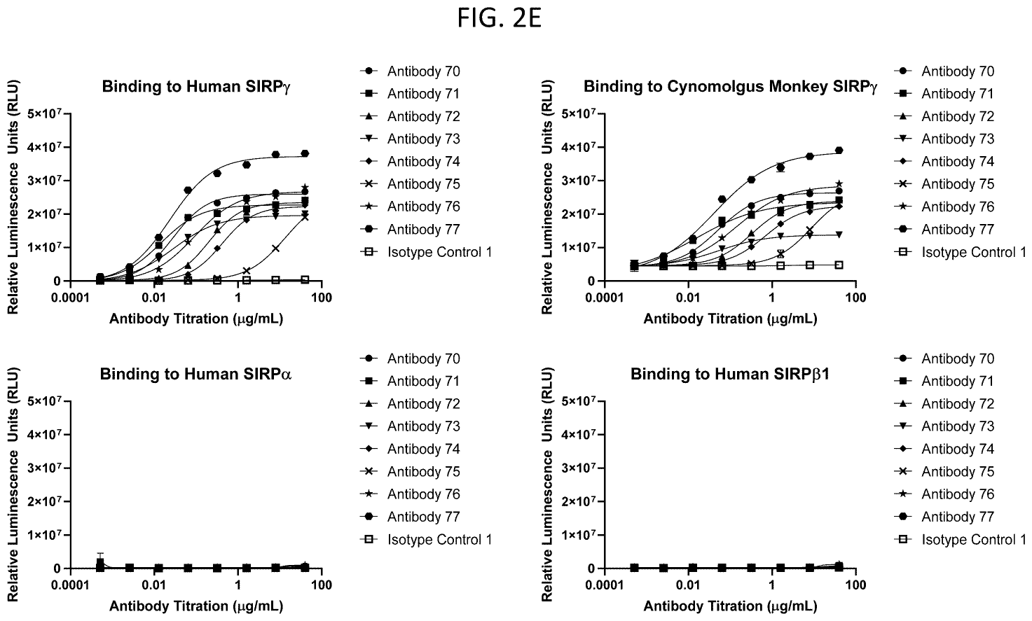

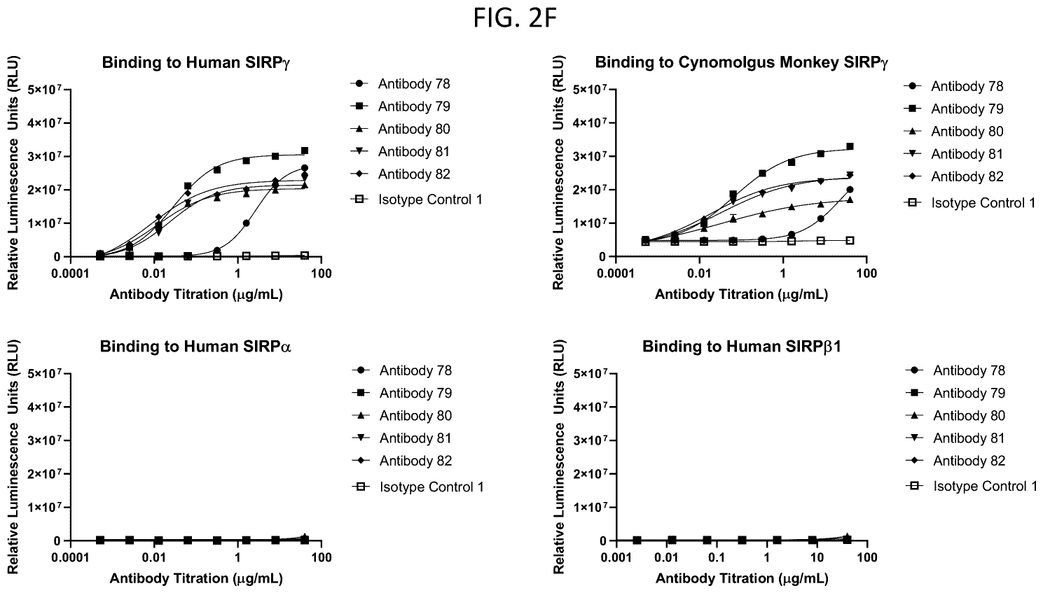

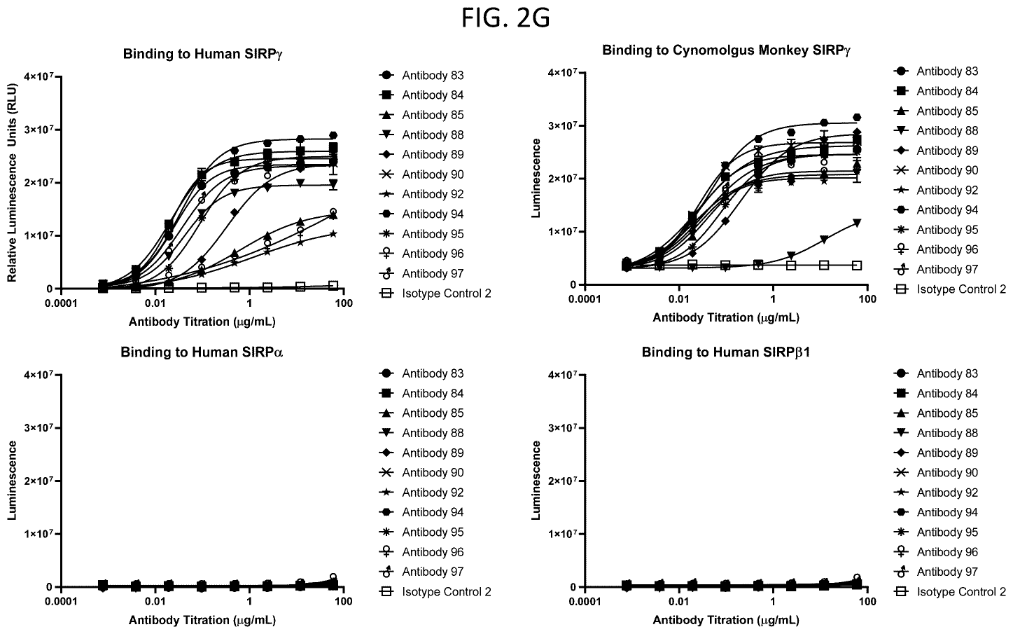

FIGS. 2A-2G show binding curves of SIRPγ antibodies and human Fc isotype controls to human SIRPγ, cynomolgus monkey SIRPγ, human SIRPα V1, human SIRPα V2, and human SIRPβ1 by ELISA. Select SIRPγ antibodies from Example 1 and Example 2 were fully made human as noted above. Isotype control 1 was an unrelated human IgG1 antibody with irrelevant CDRs (non-SIRPγ-binding) containing the same amino acid substitutions in the Fc region as some of the selected SIRPγ antibodies for increased FcγR binding. Isotype control 2 was an unrelated human IgG1 antibody with irrelevant CDRs with no modifications in the Fc region. DNA was transiently transfected into Expi293F cells for 5 days. Antibodies were purified by Protein A from cell supernatants and analyzed in a titration via ELISA as previously described in FIG. 1 using an anti-human IgG antibody as the detection antibody.

Select antibodies from Example 1 of the disclosure were tested for their affinities to human SIRPγ, cynomolgus monkey SIRPγ, human SIRPαV1, human SIRPαV2, and human SIRPβ1 using a biolayer interferometry (BLI) Octet system (Pall ForteBio). Each antibody was immobilized on a biosensor tip by an anti-human IgG capture (AHC). SIRP-His monomer protein at multiple concentrations (starting at 300 nM diluted 1:2 for 7 total concentrations) were exposed to the biosensor to measure on-rate kinetics of SIRPγ antibodies binding to SIRP-His protein. The biosensors were then exposed to wash buffer to measure off-rate kinetics. The resulting kinetic data were analyzed and fitted using a 1:1 binding model. Affinities were calculated as a global KD and is presented in Table 4 below. The table shows the KD (in M) of binding of selected antibodies to monomeric human SIRPγ, cynomolgus monkey SIRPγ, human SIRPαV1, human SIRPαV2, and human SIRPβ1 as assayed by ForteBio Octet.

The enriched library of scFv antibodies from Example 2 were tested for binding to human SIRPγ using the Octet system. Unique binders were further tested for binding to human SIRPαV1, human SIRPαV2, and human SIRPβ1. Briefly, biotinylated anti-V5 was immobilized onto streptavidin biosensors. Each V5-tagged SIRPγ scFv antibody as crude PPE was captured by the anti-V5 antibody. SIRP-His monomer protein at 500 nM was exposed to the biosensor for binding to SIRP scFv. The biosensors were then exposed to wash buffer to measure off-rate kinetics. The resulting association and dissociation kinetics (kdis) was analyzed. Sensorgrams were visually inspected, and antibodies were categorized into strong, medium, weak, hetero or non-binders and the category is presented in Table 5 below. Similarly, cross-reactivity to cynomolgus monkey SIRPγ, cynomolgus monkey SIRPα, and cynomolgus monkey SIRPβ1 were tested. Antibodies were categorized into binders and non-binders and presented in Table 6 below. Making reference to Table 5, Strong kdis<0.001 per second; medium kdis>0.001/second but <0.01 per second; weak kdis>0.01 per second. Heterogeneous (hetero) kinetics indicated baseline drift and binding category could not be determined. Making reference to Table 5 and 6, Binder has analyte response level ≥0.02 nm at 115 seconds during association. Non-binder has analyte response level <0.02 nm at 115 seconds during association.

Select antibodies from Example 2 were tested for their affinities to human SIRPγ and cynomolgus monkey SIRPγ using a biolayer interferometry (BLI) Octet system (Pall ForteBio). Antibodies were coated onto anti-human Fc capture (AHC) biosensors. SIRP-His monomer protein or bivalent SIRP protein with mouse Fc at four concentrations (200 nM, 100 nM, 50 nM, 25 nM) were exposed to the biosensor to measure on-rate kinetics of SIRP antibodies binding to SIRP protein. The biosensors were then exposed to wash buffer to measure off-rate kinetics. The resulting kinetic data were analyzed and fitted using a global 1:2 binding model. The KD affinities for each antibody are presented in Table 7 below.

TABLE 4
Affinities (K_{D}) of Exemplary Antibodies to Various SIRP Proteins

| Antibody | Human | Cyno | Human | Human SIRPα | Human |
|---|---|---|---|---|---|
| No. | SIRPγ (M) | SIRPγ (M) | SIRPα V1 (M) | V2 (M) | SIRPβ1 (M) |
| 7 | 2.96E−07 | 2.20E−06 | Association | Association not | Association not |
| | | | not measurable | measurable | measurable |
| 8 | 2.07E−09 | 8.01E−09 | Association | Association not | Association not |
| | | | not measurable | measurable | measurable |

TABLE 5
Human SIRP Binding Category for Unique scFv Clones

| Antibody | Human | Human | Human | Human |
|---|---|---|---|---|
| No. | SIRPγ | SIRPα V1 | SIRPα V2 | SIRPβ1 |
| 9 | Medium | Non-binder | Non-binder | Non-binder |
| 10 | Medium | Non-binder | Non-binder | Non-binder |
| 11 | Medium | Weak | Non-binder | Non-binder |
| 12 | Medium | Non-binder | Non-binder | Non-binder |
| 13 | Medium | Non-binder | Non-binder | Non-binder |
| 14 | Medium | Non-binder | Non-binder | Non-binder |
| 15 | Medium | Non-binder | Non-binder | Non-binder |
| 16 | hetero | Non-binder | Non-binder | Non-binder |
| 17 | hetero | Non-binder | Non-binder | Weak |
| 18 | Medium | Non-binder | Non-binder | Non-binder |
| 19 | Strong | Non-binder | Non-binder | Non-binder |
| 20 | Strong | Non-binder | Non-binder | Non-binder |
| 21 | Medium | Non-binder | Non-binder | Non-binder |
| 22 | Strong | Non-binder | Non-binder | Strong |
| 23 | Medium | Non-binder | Non-binder | Non-binder |
| 24 | Weak | Non-binder | Non-binder | Non-binder |
| 25 | Medium | Non-binder | Non-binder | Non-binder |
| 26 | Medium | Non-binder | Non-binder | Non-binder |
| 27 | Medium | Non-binder | Non-binder | Non-binder |
| 28 | Medium | Weak | Medium | Strong |
| 29 | Medium | Non-binder | Non-binder | Non-binder |
| 30 | Medium | Non-binder | Non-binder | Non-binder |
| 31 | Medium | Weak | Weak | Non-binder |
| 32 | Strong | Non-binder | Non-binder | Non-binder |
| 33 | Medium | Non-binder | Non-binder | Non-binder |
| 34 | Medium | Non-binder | Non-binder | Non-binder |
| 35 | Medium | Non-binder | Non-binder | Non-binder |
| 36 | Strong | Non-binder | Non-binder | Non-binder |
| 37 | Medium | Weak | Non-binder | Non-binder |
| 38 | Medium | Non-binder | Non-binder | Non-binder |
| 39 | Medium | Non-binder | Non-binder | Non-binder |
| 40 | Medium | Non-binder | Non-binder | Non-binder |
| 41 | Weak | Non-binder | Non-binder | Non-binder |
| 42 | Weak | Non-binder | Non-binder | Non-binder |
| 43 | hetero | Non-binder | Non-binder | Non-binder |
| 44 | Weak | Non-binder | Non-binder | Non-binder |
| 45 | Weak | Non-binder | Non-binder | Non-binder |
| 46 | Weak | Non-binder | Non-binder | Non-binder |
| 47 | Weak | Non-binder | Non-binder | Non-binder |
| 48 | Medium | Non-binder | Non-binder | Non-binder |
| 49 | Weak | Non-binder | Non-binder | Non-binder |
| 50 | Weak | Non-binder | Weak | Non-binder |
| 51 | Medium | Non-binder | Non-binder | Non-binder |
| 52 | Weak | Non-binder | Non-binder | Non-binder |
| 53 | Weak | Non-binder | Non-binder | Non-binder |

TABLE 6
Cynomolgus Monkey SIRP Binding
Category for Unique scFv Clones

| Antibody No. | Cyno SIRPγ | Cyno SIRPα | Cyno SIRPβ1 |
|---|---|---|---|
| 9 | Binder | Non-binder | Non-binder |
| 10 | Binder | Non-binder | Non-binder |
| 11 | Binder | Binder | Binder |
| 12 | Binder | Binder | Non-binder |
| 13 | Binder | Non-binder | Binder |
| 14 | Binder | Binder | Non-binder |
| 15 | Binder | Non-binder | Non-binder |
| 16 | Binder | Non-binder | Non-binder |
| 17 | Binder | Non-binder | Non-binder |
| 18 | Binder | Non-binder | Non-binder |
| 19 | Binder | Non-binder | Non-binder |
| 20 | Binder | Non-binder | Non-binder |
| 21 | Binder | Non-binder | Non-binder |
| 22 | Binder | Binder | Binder |
| 23 | Binder | Non-binder | Non-binder |
| 24 | Binder | Non-binder | Non-binder |
| 25 | Binder | Non-binder | Non-binder |
| 26 | Binder | Non-binder | Non-binder |
| 27 | Binder | Non-binder | Non-binder |
| 28 | Binder | Non-binder | Non-binder |
| 29 | Binder | Non-binder | Non-binder |
| 30 | Binder | Non-binder | Non-binder |
| 31 | Binder | Non-binder | Non-binder |
| 32 | Binder | Non-binder | Non-binder |
| 33 | Binder | Non-binder | Non-binder |
| 34 | Binder | Non-binder | Non-binder |
| 35 | Binder | Non-binder | Non-binder |
| 36 | Binder | Non-binder | Non-binder |
| 37 | Binder | Binder | Binder |
| 38 | Binder | Non-binder | Non-binder |
| 39 | Binder | Non-binder | Non-binder |
| 40 | Binder | Non-binder | Non-binder |
| 41 | Binder | Non-binder | Non-binder |
| 42 | Binder | Non-binder | Non-binder |
| 43 | Binder | Non-binder | Non-binder |
| 44 | Binder | Non-binder | Non-binder |
| 45 | Binder | Non-binder | Non-binder |
| 46 | Binder | Binder | Non-binder |
| 47 | Binder | Non-binder | Non-binder |
| 48 | Binder | Non-binder | Non-binder |
| 49 | Binder | Non-binder | Non-binder |
| 50 | Binder | Non-binder | Non-binder |
| 51 | Binder | Non-binder | Non-binder |
| 52 | Binder | Non-binder | Non-binder |
| 53 | Binder | Non-binder | Non-binder |

TABLE 7
Affinities (K_{D}) of SIRP Antibodies with human Fc to
Human SIRPγ and cynomolgus monkey SIRPγ

| Antibody | Human SIRPγ- | Cyno SIRPγ- | Human SIRPγ- |
|---|---|---|---|
| No. | His (M) | His (M) | mFc (M) |
| 83 | 1.74E−07 | 1.17E−08 | 5.32E−08 |
| 84 | N/A | 3.41E−08 | 2.05E−06 |
| 85 | 9.69E−08 | 9.61E−08 | 3.56E−08 |
| 86 | 5.61E−08 | 4.82E−08 | 3.92E−09 |
| 88 | 1.36E−07 | 6.78E−08 | 5.06E−08 |
| 89 | 2.97E−07 | 2.62E−08 | 1.32E−06 |
| 90 | 1.75E−07 | 6.92E−08 | 8.76E−08 |
| 92 | 1.23E−08 | 2.96E−06 | 9.03E−09 |
| 94 | 3.01E−08 | 4.26E−08 | 8.02E−08 |
| 95 | 1.93E−07 | 4.03E−08 | 1.28E−06 |
| 96 | 7.03E−08 | 2.11E−07 | 2.15E−08 |
| 97 | 3.62E−07 | 1.63E−08 | 4.84E−08 |

*N/A = Not applicable, software unable to fit due to low binding

### Example 4: Binding of SIRPγ Antibodies to Cells In Vitro Via Flow Cytometry

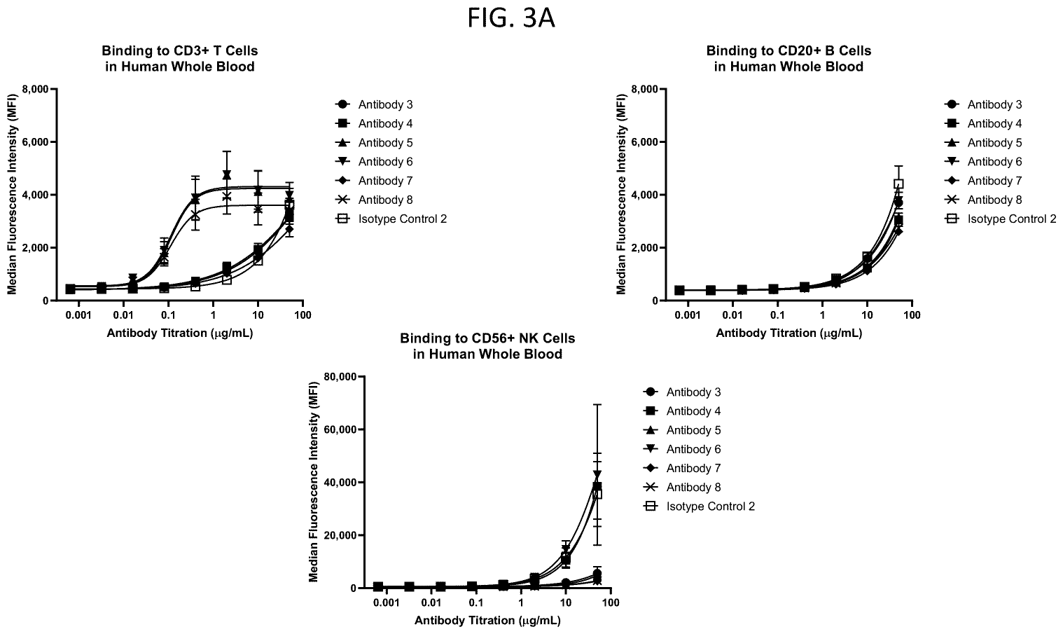

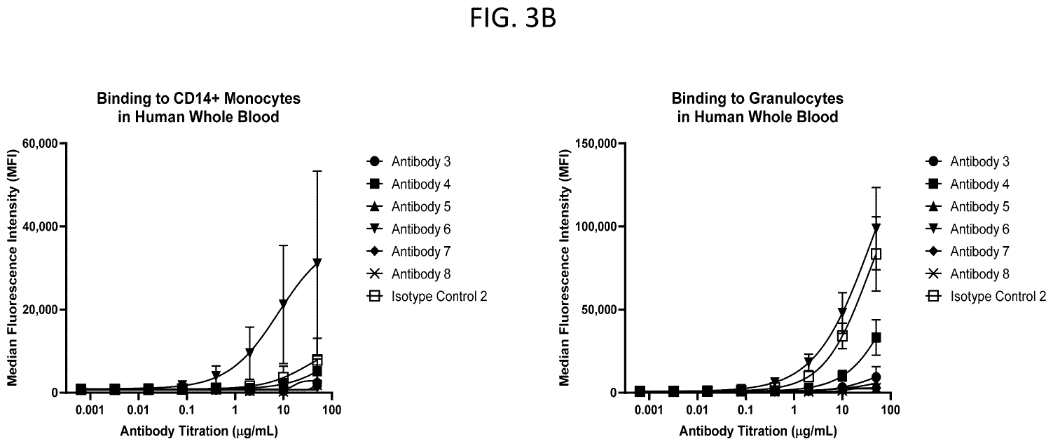

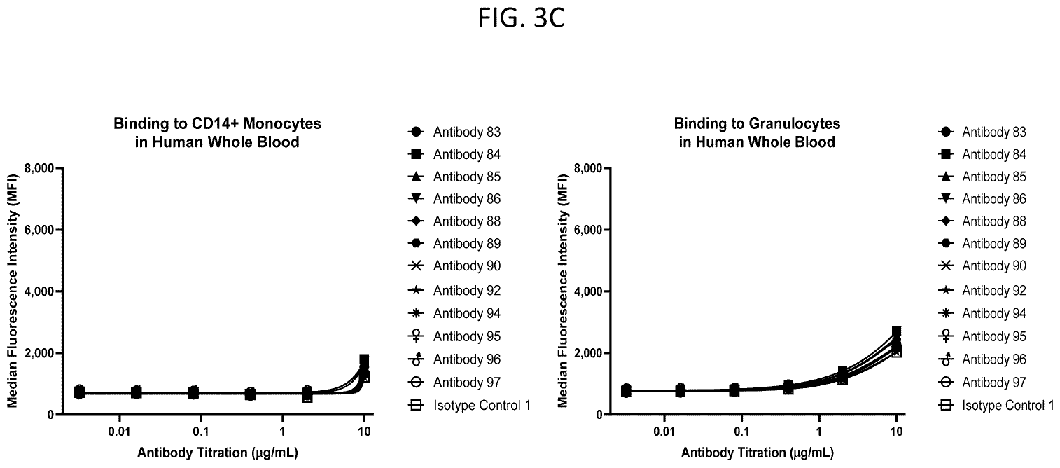

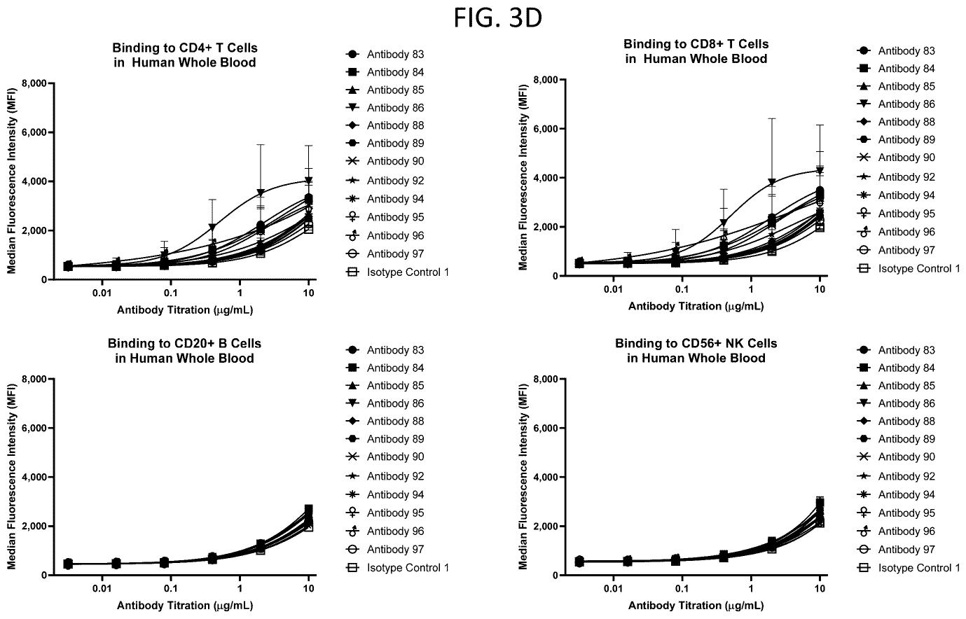

Selected antibodies and an isotype control were tested for binding to human T-cells, B-cells, NK cells, monocytes, and granulocytes. FIGS. 3A-3D show the results of binding studies performed with exemplary antibodies to T-cells, B-cells, NK cells, monocytes, and granulocytes in human whole blood compared to an isotype control. Isotype control 1 was an unrelated human IgG1 antibody with an irrelevant CDR. Isotype control 2 was the same as isotype control 1 but contained substitutions in the Fc region (to increase affinity for FcγR) as some of the selected SIRPγ antibodies. Up to 50 μg/mL fluorescent dye-conjugated antibodies or isotype controls were incubated with whole blood from three normal donors. T-cells were identified as the CD14−, CD20−, SSClow, and CD3+ population or further identified to be CD4+ or CD8+ populations. B-cells were identified as the CD14−, SSClow, and CD20+ population. NK cells were identified as CD14−, CD20−, CD3−, and CD56+. Monocytes were identified as the SSClow and CD14+ population. Granulocytes were identified as the CD14−, CD20−, CD3−, SSChigh population. Graphs depict the median fluorescence intensity (MFI) of each population.

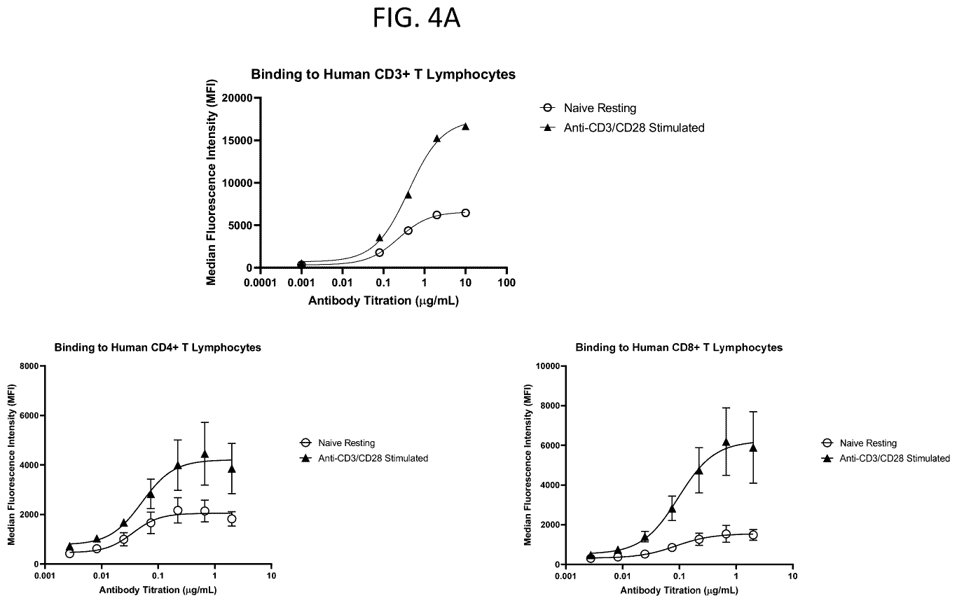

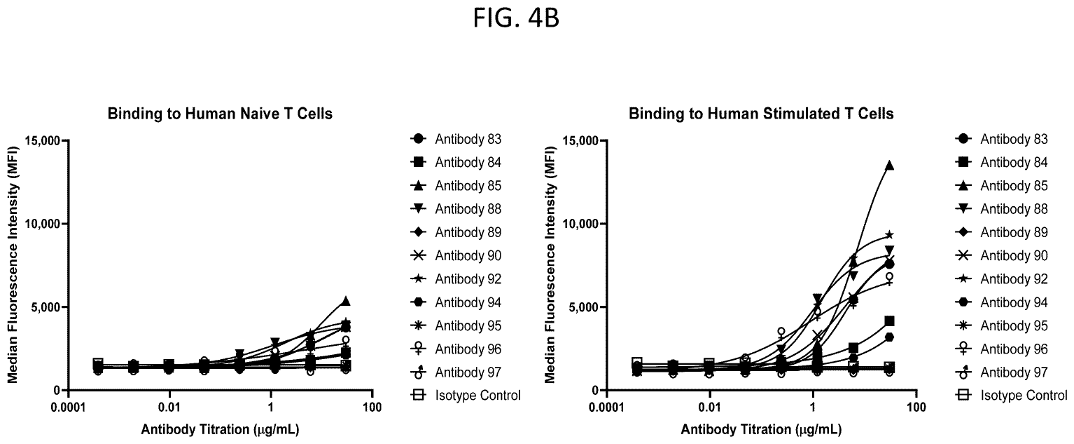

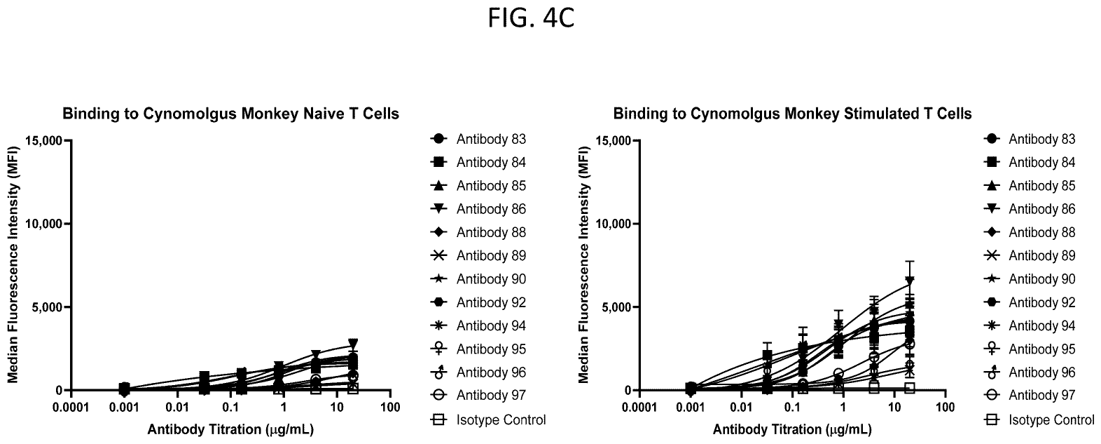

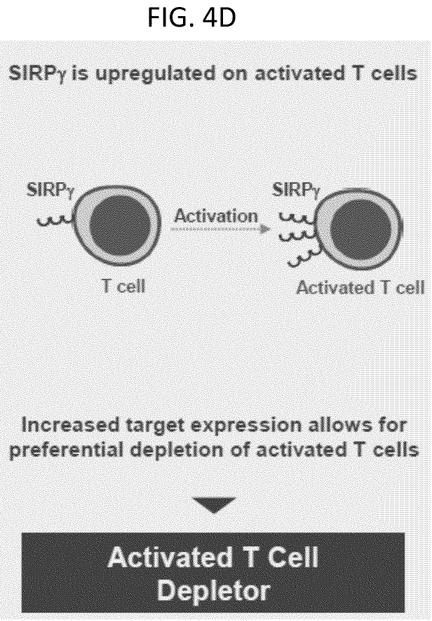

A commercially available mouse monoclonal antibody against human SIRPγ (clone OX-119 from ThermoFisher Scientific, catalogue number MA5-28215) was tested for binding to naïve T-cells and to stimulated T-cells via flow cytometry. To stimulate or activate T-cells, anti human CD3 and anti-human CD28 were incubated with naïve resting CD3+, CD4+, or CD8+ T-cells from individual healthy human donors or cynomolgus monkeys for 7 days in the presence of IL-2. A titration of the commercially available antibody was added to the stimulated cells and to naïve T-cells from the same donors. FIG. 4A shows the binding curves of the mouse monoclonal antibody to the stimulated CD3+, CD4+, and CD8+ T-cells compared to the naïve T-cells. The antibody exhibited increased binding to activated (stimulated) T-cells, indicating an increase in expression of SIRPγ on activated T-cells. Similarly, select antibodies from Example 2 were assessed for binding to naïve and stimulated human and cynomolgus monkey T-cells. FIGS. 4B-4C show the binding curves of the human monoclonal antibodies to the stimulated human and cynomolgus monkey CD3+ T-cells compared to the naïve T-cells.

Selected antibodies were tested for binding to several subpopulations of unstimulated T-cells and of stimulated T-cells, via flow cytometry. To stimulate or activate T-cells, anti human CD3 and anti-human CD28-coated beads were incubated with CD3+ T-cells from individual healthy human donors in the presence of IL-2. Activation beads were removed after 3 days. The cells were stained for multiple T-cell subpopulation markers including but not limited to CD3, CD4, CD8, CD69, CD25, and PD-1 at multiple time points for 16 days. A saturating 20 μg/mL of selected SIRPγ antibody was used. Antibodies 83, 84, 85, 86, 88, 89, 90, 92, 94, 95, 96, and 97 exhibited increased binding to stimulated T-cells when compared to unstimulated T-cells. Antibody 84 preferentially bound to CD8+ T-cells when compared to CD4+ T-cells. Antibody 84 preferentially bound to CD69+/CD8+ T-cells and CD25+/CD8+ T-cells when compared to CD69−/CD8+ T-cells and CD25−/CD8+ T-cells. Antibodies 85, 89, 95, and 97 preferentially bound to PD1+ T-cells when compared to PD1− T-cells.

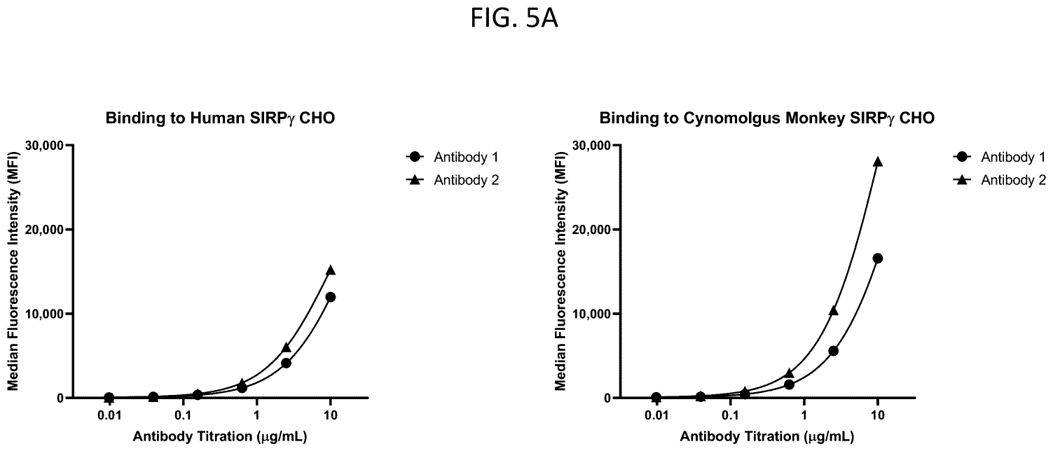

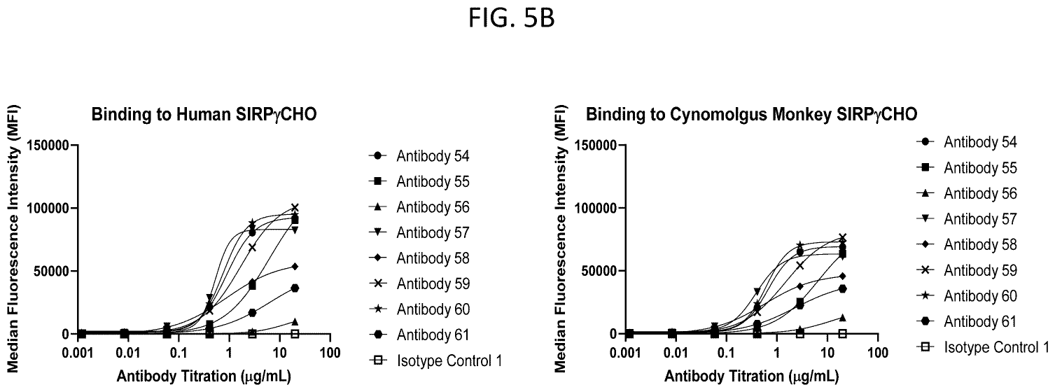

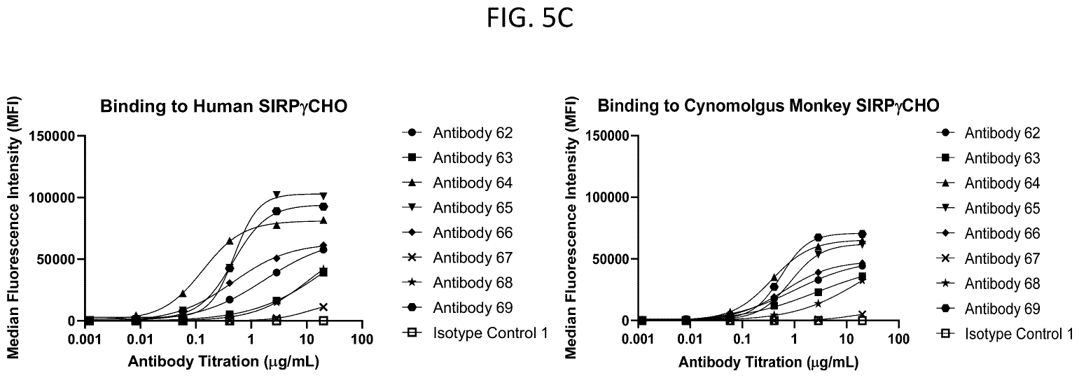

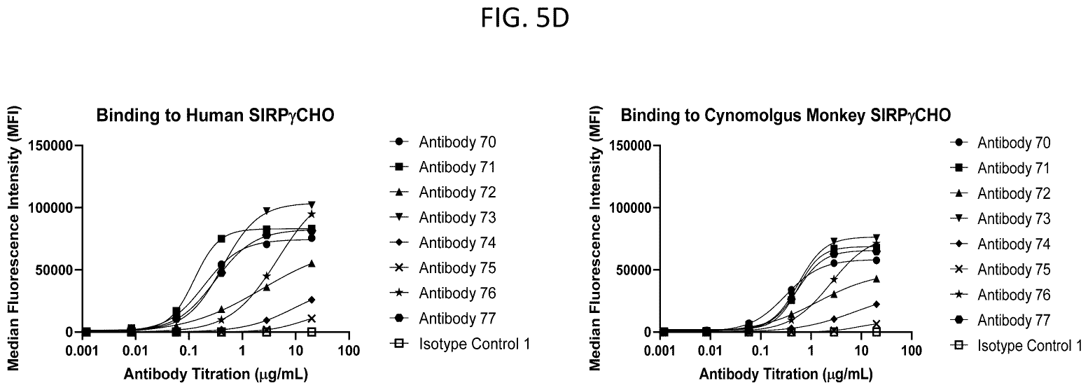

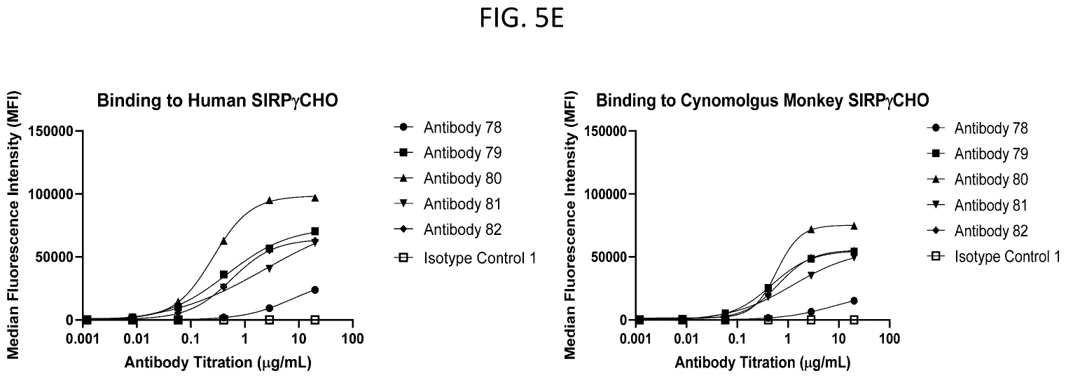

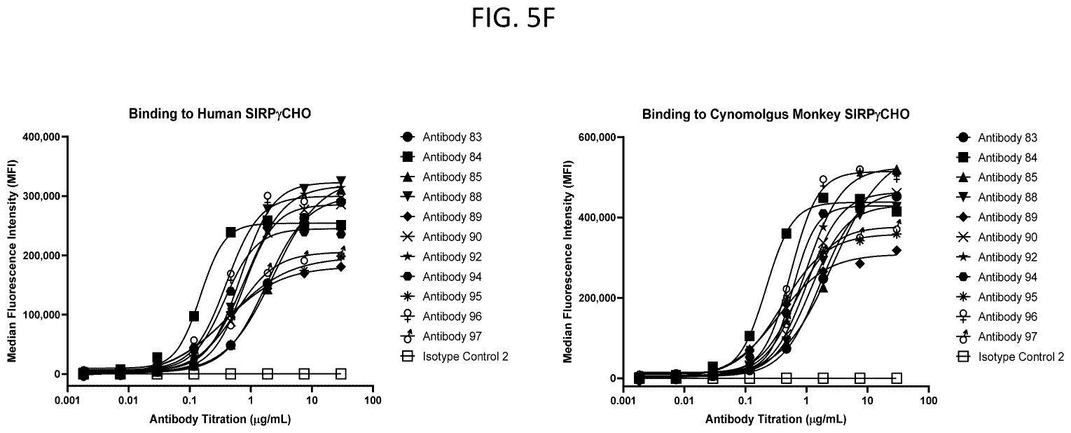

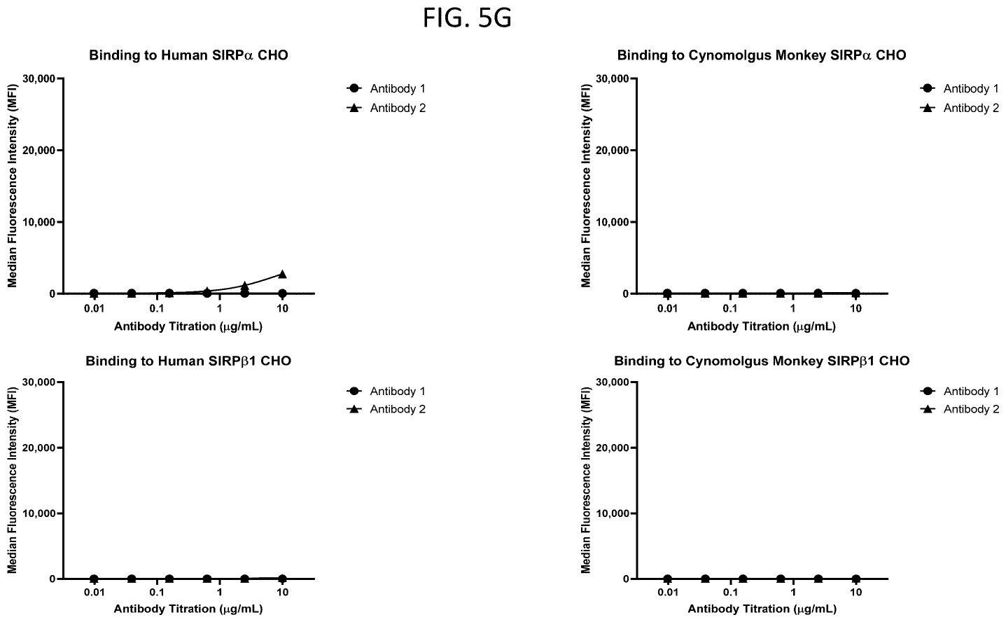

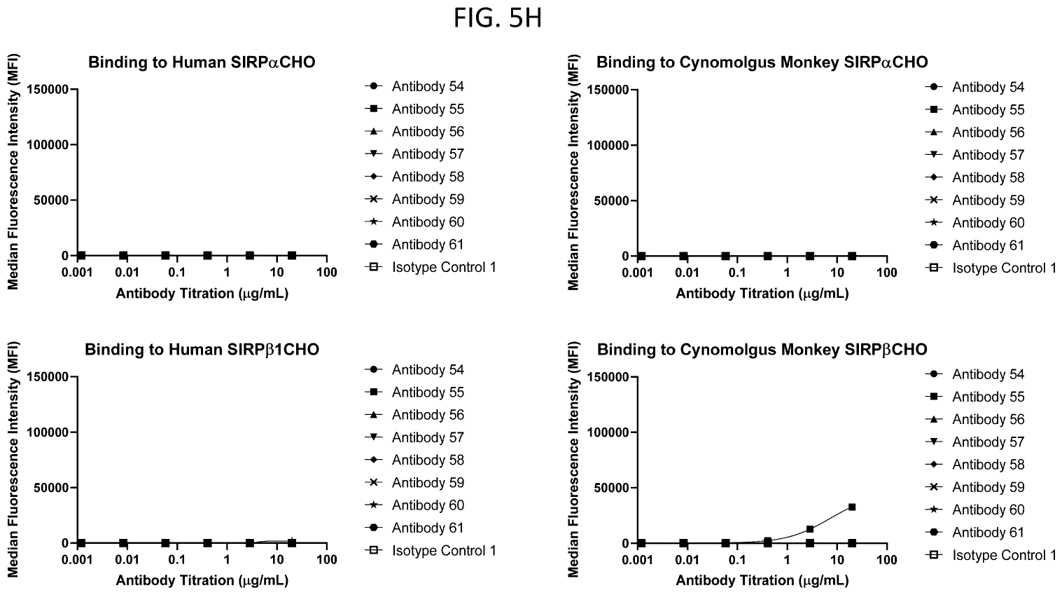

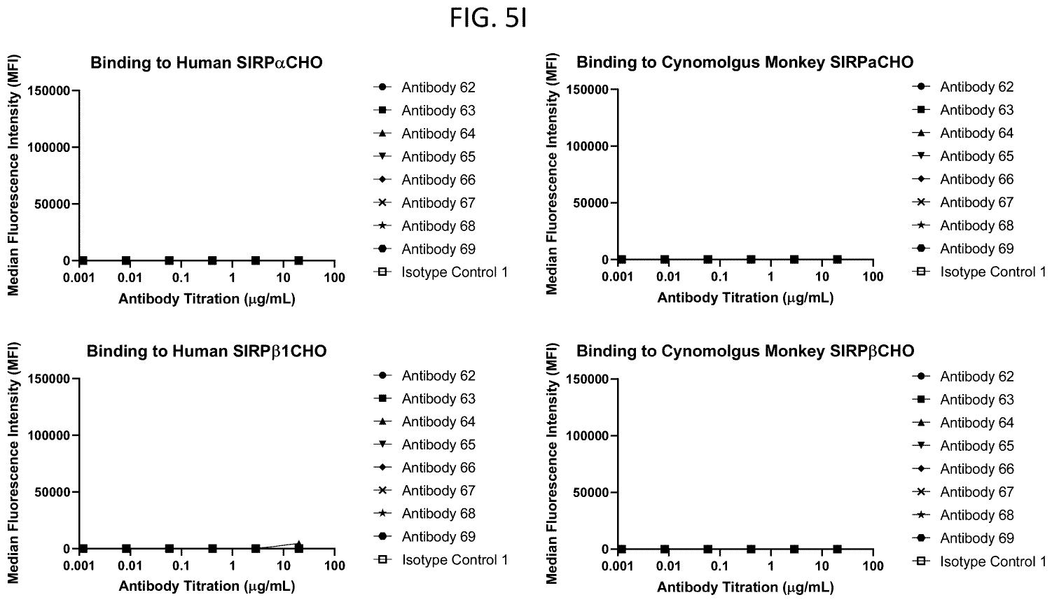

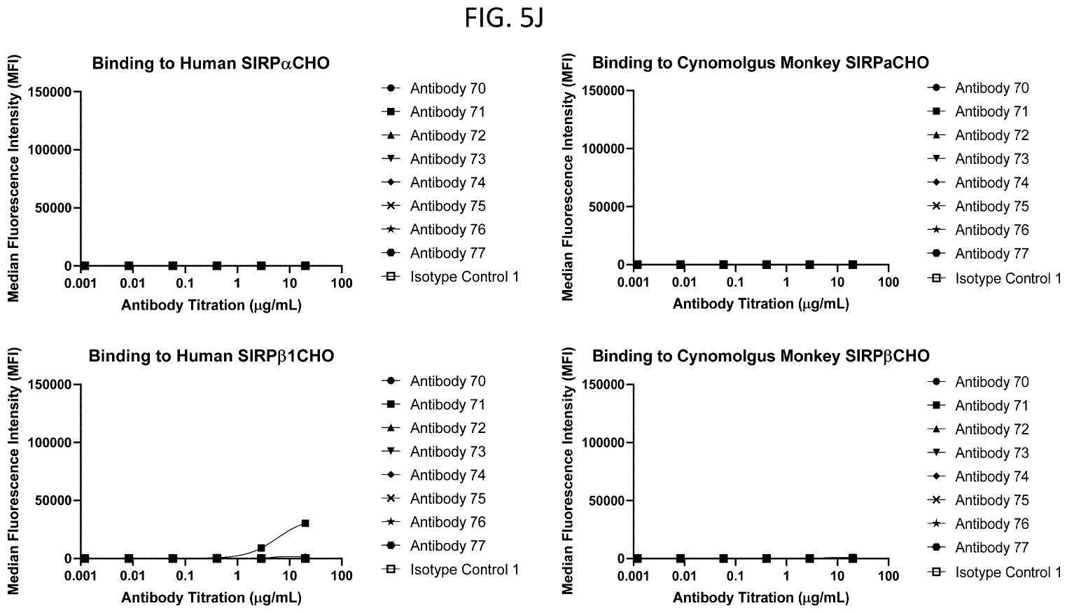

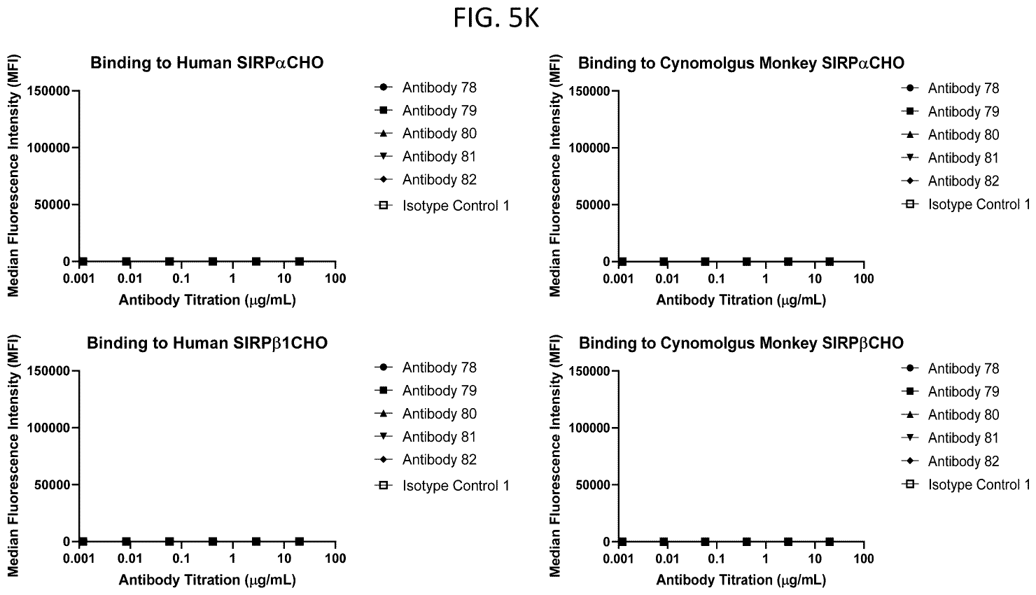

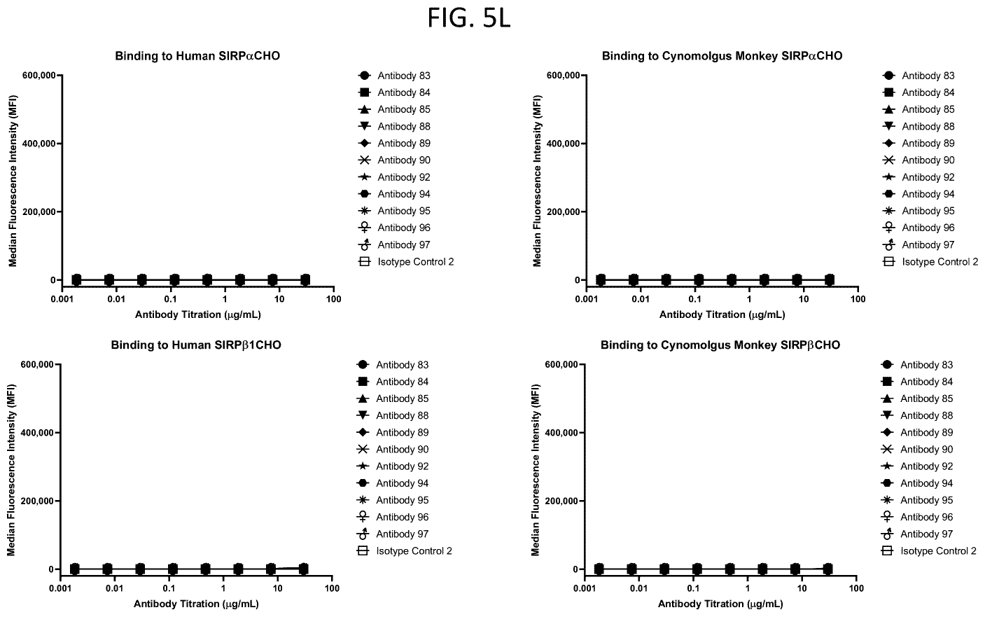

Selected antibodies were tested for binding to stably transfected human SIRPγ, SIRPαV1, or SIRPβ1 (co-transfected with DAP12) Chinese hamster ovary (CHO) cells via flow cytometry. A titration of select antibodies was added to the cells and detected using a fluorescently labelled secondary anti-rat IgG antibody or anti-human IgG antibody. Graphs depict the median fluorescence intensity (MFI) at each concentration. FIGS. 5A and 5G show the binding curves of select antibodies to human SIRPγ, cynomolgus monkey SIRPγ, human SIRPα, cynomolgus monkey SIRPα, human SIRPβ1, or cynomolgus monkey SIRPβl-expressing CHO cells detected using an anti-rat IgG antibody. FIGS. 5B-5F and 5H-5L show the binding curves of select antibodies to human SIRPγ, cynomolgus monkey SIRPγ, human SIRPα, cynomolgus monkey SIRPα, human SIRPβ1, or cynomolgus monkey SIRPβ1-expressing CHO cells detected using an anti-human IgG antibody. Isotype control 1 was an unrelated human IgG1 antibody with irrelevant CDRs (non-SIRPg-binding) containing the same amino acid substitutions in the Fc region as some of the selected SIRPγ antibodies for increased FcγR binding. Isotype control 2 was the same as isotype control 1 but contained no mutations in the Fc region. For select antibodies, each myc-tagged SIRPγ scFv antibody as crude PPE was incubated with each of the SIRP-expressing cell lines stated above along with the parental CHO-K1 cell line. Table 8 below shows MFI fold change values when compared to the parental non-transfected CHO cells for the scFv antibodies using the 6 SIRP-expressing cell lines and detected using anti-myc antibody. Fold change value <2.0 is considered a non-binder. The SIRPγ antibodies exhibited in FIGS. 5A-5L and in Table 8 show preferential binding to human and cynomolgus monkey SIRPγ.

TABLE 8
MFI Fold Change Values Over Binding
to Parental CHO Cells for scFv Clones

| | Human | Cyno | Human | Cyno | Human | Cyno |
|---|---|---|---|---|---|---|
| Antibody | SIRPγ | SIRPγ | SIRPα V1 | SIRPα | SIRPβ1 | SIRβ1 |
| No. | CHO | CHO | CHO | CHO | CHO | CHO |
| 9 | 10.36 | 6.87 | 1.14 | 1.87 | 0.99 | 1.34 |
| 10 | 38.26 | 23.12 | 1.62 | 1.87 | 1.09 | 1.37 |
| 11 | 3.98 | 6.18 | 1.01 | 1.89 | 0.87 | 1.41 |
| 12 | 10.67 | 5.94 | 1.18 | 1.93 | 1.26 | 1.36 |
| 13 | 7.06 | 5.22 | 1.02 | 1.88 | 0.89 | 1.74 |
| 14 | 19.59 | 17.96 | 1.15 | 1.93 | 1.09 | 1.38 |
| 15 | 17.03 | 3.71 | 1.06 | 1.91 | 0.91 | 1.39 |
| 16 | 33.03 | 16.32 | 1.04 | 1.92 | 0.86 | 1.38 |
| 17 | 28.27 | 30.06 | 1.20 | 1.98 | 1.46 | 1.42 |
| 18 | 25.16 | 16.16 | 1.04 | 1.96 | 0.90 | 1.38 |
| 19 | 43.29 | 36.65 | 1.04 | 2.01 | 0.93 | 1.40 |
| 20 | 8.87 | 10.49 | 1.05 | 1.76 | 0.89 | 1.29 |
| 21 | 51.35 | 96.14 | 1.14 | 1.74 | 0.99 | 1.27 |
| 22 | 3.17 | 8.24 | 1.01 | 1.65 | 0.84 | 1.20 |
| 23 | 21.63 | 16.47 | 0.99 | 1.72 | 0.97 | 1.40 |
| 24 | 11.10 | 9.85 | 1.07 | 1.82 | 1.01 | 1.28 |
| 25 | 19.46 | 10.35 | 1.17 | 1.24 | 1.33 | 1.03 |
| 26 | 28.39 | 40.02 | 1.08 | 1.30 | 1.23 | 1.05 |
| 27 | 23.25 | 5.81 | 1.18 | 1.32 | 1.32 | 1.06 |
| 28 | 46.46 | 24.02 | 1.46 | 1.29 | 1.26 | 1.06 |
| 29 | 11.44 | 13.01 | 1.16 | 1.26 | 1.33 | 1.11 |
| 30 | 5.94 | 3.76 | 1.54 | 1.28 | 1.57 | 1.05 |
| 31 | 18.05 | 21.31 | 1.20 | 1.29 | 1.25 | 1.02 |
| 32 | 10.24 | 9.63 | 1.10 | 1.23 | 1.28 | 1.02 |
| 33 | 20.00 | 8.02 | 1.19 | 1.25 | 1.74 | 1.01 |
| 34 | 18.71 | 11.96 | 1.15 | 1.27 | 1.31 | 1.04 |
| 35 | 18.28 | 12.83 | 1.13 | 1.32 | 1.32 | 1.08 |
| 36 | 42.44 | 37.70 | 1.17 | 1.34 | 1.34 | 1.05 |
| 37 | 31.06 | 15.49 | 1.61 | 1.61 | 1.55 | 1.29 |
| 38 | 14.81 | 16.68 | 1.13 | 1.16 | 1.31 | 1.37 |
| 39 | 16.36 | 39.55 | 1.09 | 1.15 | 1.24 | 1.39 |
| 40 | 15.43 | 15.33 | 1.16 | 1.24 | 1.40 | 1.46 |
| 41 | 25.48 | 36.06 | 1.09 | 1.42 | 1.16 | 1.53 |
| 42 | 15.90 | 14.49 | 1.00 | 1.44 | 1.05 | 1.54 |
| 43 | 41.24 | 22.74 | 1.06 | 1.49 | 1.07 | 1.58 |
| 44 | 16.02 | 26.70 | 1.00 | 1.50 | 1.06 | 1.58 |
| 45 | 24.26 | 18.19 | 1.63 | 1.46 | 1.55 | 1.53 |
| 46 | 13.07 | 16.01 | 1.01 | 1.49 | 1.09 | 1.53 |
| 47 | 17.33 | 14.17 | 1.03 | 1.43 | 1.13 | 1.53 |
| 48 | 12.08 | 9.25 | 1.00 | 1.51 | 1.08 | 1.53 |
| 49 | 18.63 | 18.34 | 1.14 | 1.49 | 1.21 | 1.52 |
| 50 | 12.84 | 30.83 | 1.03 | 1.53 | 1.19 | 1.60 |
| 51 | 20.02 | 18.83 | 1.02 | 1.51 | 1.16 | 1.61 |
| 52 | 14.72 | 16.33 | 1.00 | 1.52 | 1.12 | 1.63 |
| 53 | 5.97 | 6.94 | 1.05 | 1.57 | 1.07 | 1.60 |

### Example 5: Effect of SIRPγ Antibodies on ADCC

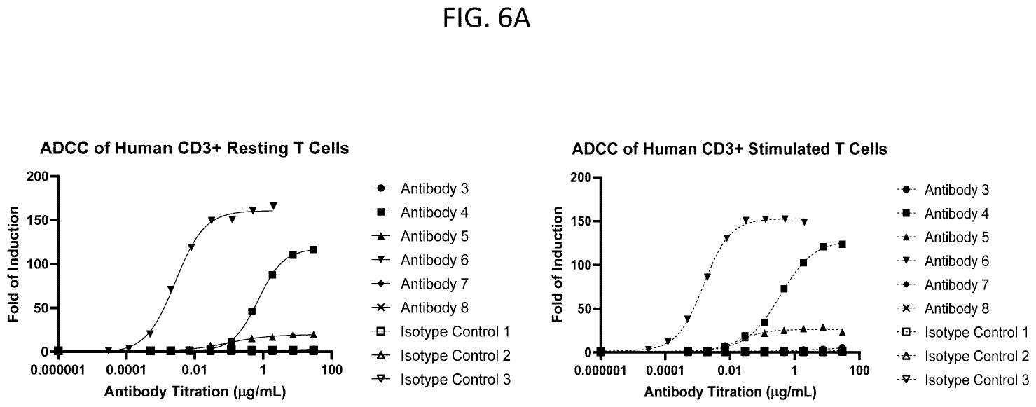

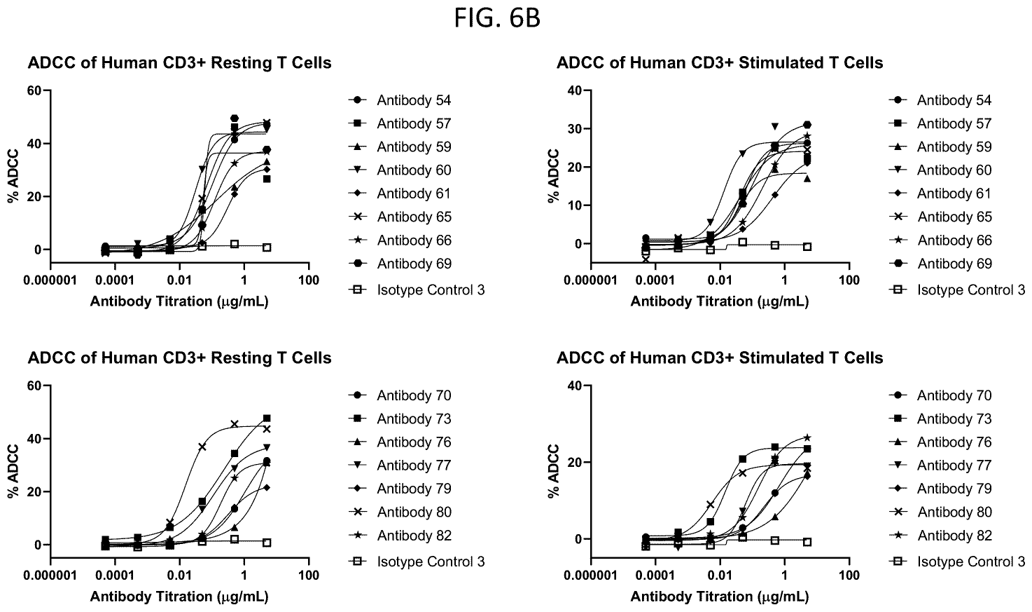

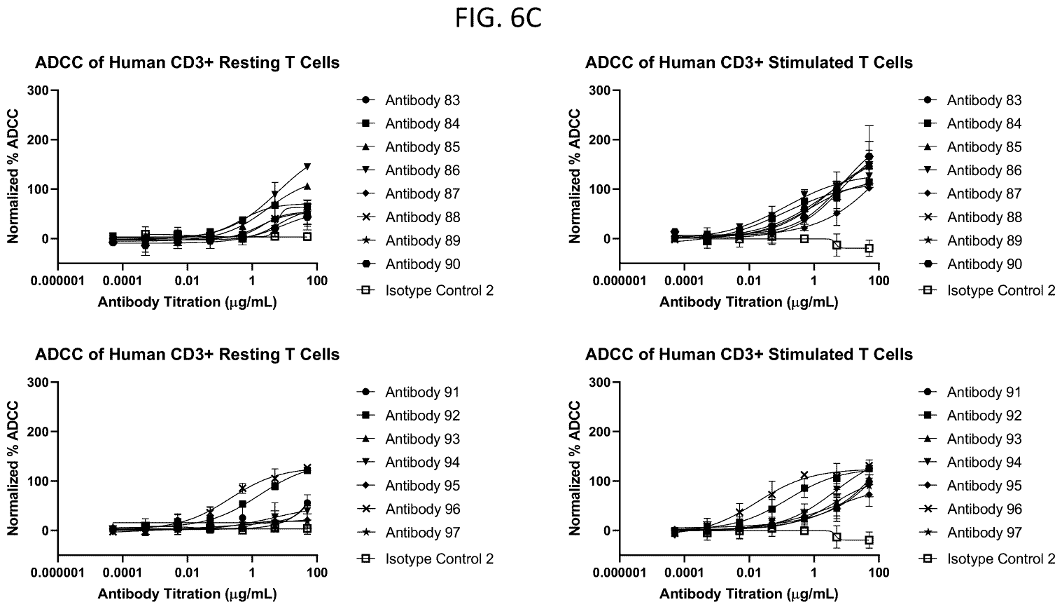

Antibody-dependent cellular cytotoxicity (ADCC) induced by selected SIRPγ antibodies on primary human resting and activated T-cells were evaluated. SIRPγ expressing-human primary resting T-cells (target) cells were incubated with a reporter cell line (effector) expressing FcγRIIIa at an effector-cell to target-cell ratio of 8:1 for 6 hours at 37° C. Following FcγRIIIa engagement with the Fc region of a relevant SIRPγ antibody bound to a target cell, these effector cells would transduce intracellular signals resulting in NFAT-mediated luciferase activity. This activity was read as luminescence signal on a plate reader and would be proportional to FcγRIIIa engagement. To activate T-cells, anti-CD3 and anti-CD28 were preincubated with resting T-cells for 7 days. The same protocol as above was then followed to measure FcγRIIIa engagement. FIGS. 6A-6C shows the luminescence fold of ADCC induction of a titration of selected antibodies over no antibody condition for human CD3+ T-cells. The results are presented as compared to isotype controls. Isotype control 1 was an unrelated human IgG4 antibody with an irrelevant CDR. Isotype control 2 was an unrelated human IgG1 antibody with an irrelevant CDR. Isotype control 3 was the same as isotype control 2 but contained the same high affinity substitutions (to increase affinity for FcγR) in the Fc region as some of the selected SIRPγ antibodies. Table 9 below shows the half maximal effective concentration (EC50) of selected antibodies when a 4-parameter logistic non-linear regression curve fit is applied.

ADCC induced by selected SIRP antibodies on primary human T-cells (target) were evaluated using primary NK cells as effector cells. The target cells were loaded with CellTracker™ Green, washed, and exposed to SIRP antibodies at various concentrations. Then, the target cells were incubated human NK (effector) cells at an effector-cell to target-cell ratio of 2:1 for 4 hours at 37° C. Samples were stained with Zombie Violet dye and analyzed via flow cytometry. Activated T-cells were generated using the same stimulation protocol as above. % ADCC was calculated as the percent of cells positive for Zombie Violet dye with respect to total cells positive for CellTracker™ Green. The graphs in FIGS. 6B-6C depict the average of three target cell donors normalized to an internal positive control antibody. The ADCC effect is antibody-dose dependent. The results are presented as compared to isotype controls.

TABLE 9
EC₅₀ Values of Antibodies in ADCC of CD3+ T-cells

| | Resting T-cells EC₅₀ | Stimulated T-cells EC₅₀ |
|---|---|---|
| Antibody No. | (μg/mL) | (μg/mL) |
| 3 | NC | NC |
| 4 | 0.7162 | 0.355 |
| 5 | 0.09366 | 0.01818 |
| 6 | 0.0027 | 0.00153 |
| 7 | NC | NC |
| 8 | NC | NC |
| Isotype Control 1 | NC | NC |
| Isotype Control 2 | NC | NC |
| Isotype Control 3 | NC | NC |

NC = not calculable

### Example 6: Effect of SIRPγ Antibodies on ADCP

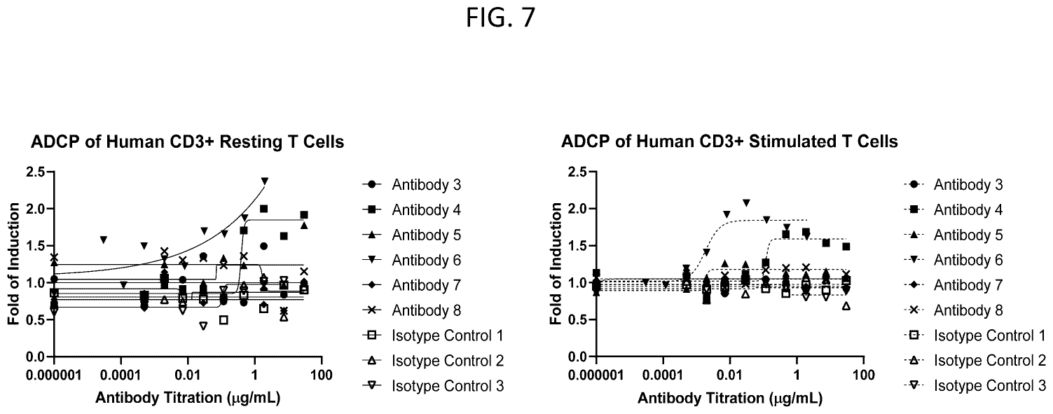

The antibody-dependent cellular phagocytosis (ADCP) induced by selected SIRPγ antibodies on primary human resting and activated T-cells were evaluated in the same way as Example 5 using a reporter cell line (effector) expressing FcγRIIa at an effector-cell to target-cell ratio of 4:1. FIG. 7 shows the luminescence fold of ADCP induction of a titration of selected antibodies over no antibody condition for human CD3+ T-cells. The results are presented as compared to isotype controls. Isotype control 1 was an unrelated IgG4 antibody with an irrelevant CDR. Isotype control 2 was an unrelated IgG1 antibody with an irrelevant CDR. Isotype control 3 was the same as isotype control 2 but contained the same high affinity substitutions (to increase affinity for FcγR) in the Fc region as some of the selected SIRPγ antibodies.

### Example 7: Determination of SIRPγ Antibody Competition with CD47 for Binding to SIRPγ

ELISA analyses were performed to assess whether selected SIRPγ antibodies of the disclosure compete with CD47-Fc for binding to hSIRPγ. To carry out the competition experiment, the extracellular binding domain of SIRPγ was coated onto a high protein-binding plate and blocked. A titration of each SIRPγ antibody was incubated on the plate for 1 hour. Biotinylated CD47-Fc at a concentration of 10 μg/mL was next added and allowed to equilibrate for 1 hour. Following a wash, streptavidin-HRP was added. The plate was washed again and developed using a chemiluminescent substrate. The plate was read on a plate reader to assess luminescence. Isotype control 1 was an unrelated human IgG1 antibody with irrelevant CDRs containing the same amino acid substitutions in the Fc region as some of the selected SIRPγ antibodies and was used as a negative control. Isotype control 2 was the same as isotype control 1 but contained no mutations in the Fc region. A known blocker antibody was used as a positive control. FIGS. 8A-8B shows a subset of antibodies with no effect on CD47/SIRPγ binding (Antibodies 5, 6, 8, 59, 73, 80, 85, 92, and 96), a second set promoted CD47/SIRPγ binding (Antibodies 3, 4, and 7), while the rest inhibited CD47/SIRPγ binding.

### Example 8: Effect of SIRPγ Antibodies on T-Cell Proliferation

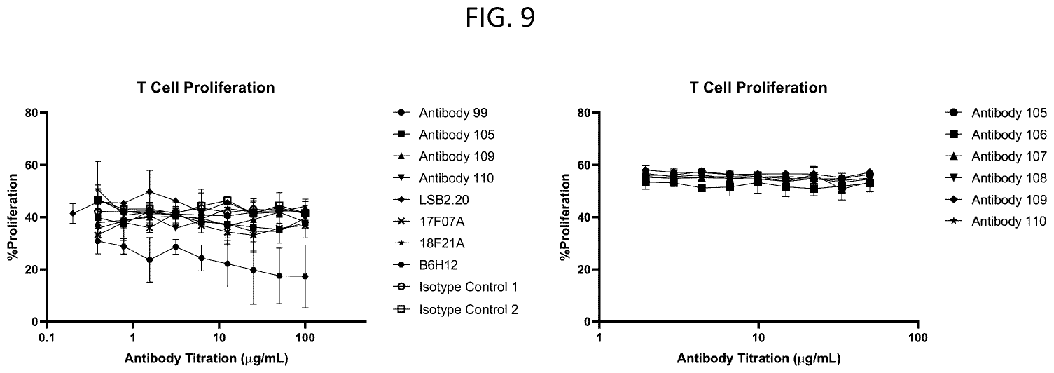

The effect of select SIRPγ antibodies on T-cell proliferation was assessed via one-way mixed lymphocyte reaction (MLR). Monocytes were isolated from PBMCs of a healthy individual and differentiated into dendritic cells (moDC). CD3+ T-cells were isolated from a different healthy individual and labeled with a dye used to track cell proliferation (CFSE). The moDCs and T-cells were cocultured with a titration of select antibodies, up to 100 μg/mL, for 7 days. The cells were then analyzed via flow cytometry. Percent proliferation (% Proliferation) is calculated as the % CD3+ T-cells that had a loss in CFSE dye signal, indicating proliferation. FIG. 9 depicts % Proliferation with respect to antibody concentration. In addition to select SIRPγ antibodies generated from Example 1 and Example 2, a human SIRPγ antibody with mouse Fc (clone LSB2.20), a human CD47 antibody with mouse Fc (clone B6H12), and two human SIRPα antibodies (clones 18F21A and 17F07A) were tested. Results are compared to isotype control antibodies. Isotype control 1 was a human IgG1 antibody with an unrelated CDR containing the same amino acid substitutions in the Fc region to reduce FcγR binding as some select SIRPγ antibodies. Isotype control 2 was a mouse IgG1 antibody with an unrelated CDR. Only the CD47 antibody demonstrated inhibition of T-cell proliferation.

### Example 9: Effect of SIRPγ Antibodies on In Vivo Depletion of Selected Cell Types

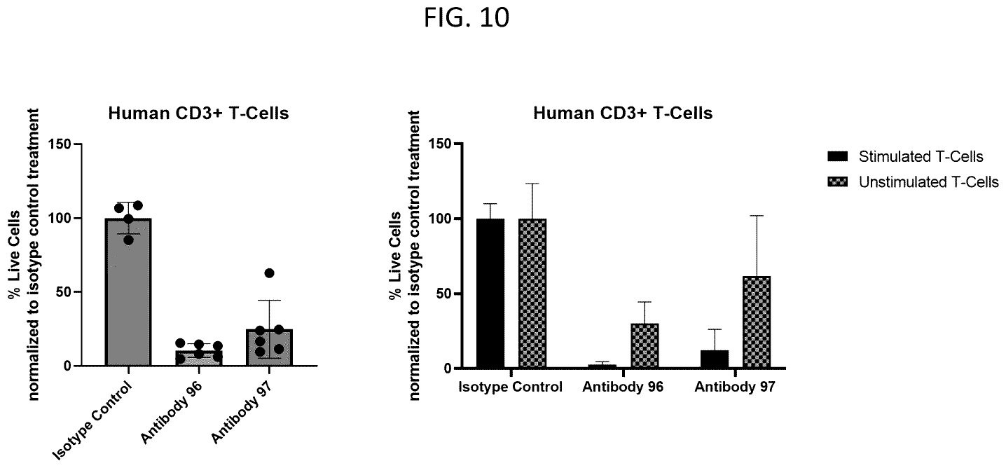

The effect of select SIRPγ antibodies on the depletion of CD3+ T cells was evaluated in vivo using 8-10 week old, female NSG-Tg (Hu-IL15) mice. Mice were administered select SIRPγ antibodies or isotype control, intraperitoneally at a dose of 30 mg/kg, 30 minutes prior to transfer of human PBMC and stimulated T-cells. T-cells were stimulated by adding human anti-CD3 and anti-CD28 antibody-coated beads to isolated CD3+ human T-cells for 5-7 days in the presence of IL-2. Stimulation beads were removed at day 3. Stimulated T-cells were dyed with CellTracker™ prior to injection. Mice were injected with 1.00E+07 human PBMCs and 5.00E+06 stimulated T-cells intravenously via single tail vein injection. Mice were euthanized 3 days post cell transfer. Whole blood was obtained via cardiac puncture and collected in EDTA blood collection tubes. Whole blood was lysed with RBC lysis buffer, washed, and blocked with both human and mouse Fc blocker. Cells were washed again and resuspended in an antibody cocktail mix diluted to suitable concentrations. Staining cocktail mix included anti-human CD45, anti-mouse CD45, anti-human CD3, and live/dead cell marker. Cells were analyzed via flow cytometry. FIG. 10 depicts % live human CD3+ T-cells detected (left panel). Additional analysis separated the detection of stimulated and unstimulated human CD3+ T-cells (right panel). Data in FIG. 10 (left panel) are normalized to number of live human CD3+ T cells detected in mice treated with isotype control. The selected SIRPγ antibodies induced depletion of human CD3+ T-cells when compared to isotype control treatment. Further analyses of CellTracker™ dyed cells (i.e.—Stimulated cells) show that stimulated cells were preferentially depleted compared to unstimulated cells (FIG. 10, right panel).

1.-49. (canceled)

50. A SIRPγ-specific antibody:
(a) comprising the three VL CDRs of amino acid sequences of SEQ ID NO: 100, SEQ ID NO: 138, SEQ ID NO: 167; and/or comprising the three VH CDRs of amino acid sequences of SEQ ID NO: 209, SEQ ID NO: 245, and SEQ ID NO: 277;
(b) comprising the three VL CDRs of amino acid sequences of SEQ ID NO: 101, SEQ ID NO: 139, SEQ ID NO: 168; and/or comprising the three VH CDRs of amino acid sequences of SEQ ID NO: 210, SEQ ID NO: 246, and SEQ ID NO: 278;
(c) comprising the three VL CDRs of amino acid sequences of SEQ ID NO: 102, SEQ ID NO: 140, SEQ ID NO: 169; and/or comprising the three VH CDRs of amino acid sequences of SEQ ID NO: 211, SEQ ID NO: 247, and SEQ ID NO: 279;
(d) comprising the three VL CDRs of amino acid sequences of SEQ ID NO: 103, SEQ ID NO: 141, SEQ ID NO: 170; and/or comprising the three VH CDRs of amino acid sequences of SEQ ID NO: 212, SEQ ID NO: 248, and SEQ ID NO: 280;
(e) comprising the three VL CDRs of amino acid sequences of SEQ ID NO: 104, SEQ ID NO: 141, SEQ ID NO: 171; and/or comprising the three VH CDRs of amino acid sequences of SEQ ID NO: 213, SEQ ID NO: 249, and SEQ ID NO: 281;
(f) comprising the three VL CDRs of amino acid sequences of SEQ ID NO: 105, SEQ ID NO: 142, SEQ ID NO: 172; and/or comprising the three VH CDRs of amino acid sequences of SEQ ID NO: 214, SEQ ID NO: 250, and SEQ ID NO: 282;
(g) comprising the three VL CDRs of amino acid sequences of SEQ ID NO: 106, SEQ ID NO: 143, SEQ ID NO: 173; and/or comprising the three VH CDRs of amino acid sequences of SEQ ID NO: 215, SEQ ID NO: 251, and SEQ ID NO: 283;
(h) comprising the three VL CDRs of amino acid sequences of SEQ ID NO: 106, SEQ ID NO: 144, SEQ ID NO: 174; and/or comprising the three VH CDRs of amino acid sequences of SEQ ID NO: 216, SEQ ID NO: 252, and SEQ ID NO: 284;
(i) comprising the three VL CDRs of amino acid sequences of SEQ ID NO: 107, SEQ ID NO: 141, SEQ ID NO: 175; and/or comprising the three VH CDRs of amino acid sequences of SEQ ID NO: 217, SEQ ID NO: 253, and SEQ ID NO: 285;
(j) comprising the three VL CDRs of amino acid sequences of SEQ ID NO: 108, SEQ ID NO: 144, SEQ ID NO: 176; and/or comprising the three VH CDRs of amino acid sequences of SEQ ID NO: 216, SEQ ID NO: 254, and SEQ ID NO: 286;
(k) comprising the three VL CDRs of amino acid sequences of SEQ ID NO: 109, SEQ ID NO: 145, SEQ ID NO: 171; and/or comprising the three VH CDRs of amino acid sequences of SEQ ID NO: 218, SEQ ID NO: 255, and SEQ ID NO: 287;
(l) comprising the three VL CDRs of amino acid sequences of SEQ ID NO: 110, SEQ ID NO: 146, SEQ ID NO: 177; and/or comprising the three VH CDRs of amino acid sequences of SEQ ID NO: 219, SEQ ID NO: 249, and SEQ ID NO: 288;
(m) comprising the three VL CDRs of amino acid sequences of SEQ ID NO: 111, SEQ ID NO: 147, SEQ ID NO: 178; and/or comprising the three VH CDRs of amino acid sequences of SEQ ID NO: 220, SEQ ID NO: 256, and SEQ ID NO: 289;
(n) comprising the three VL CDRs of amino acid sequences of SEQ ID NO: 112, SEQ ID NO: 148, SEQ ID NO: 179; and/or comprising the three VH CDRs of amino acid sequences of SEQ ID NO: 213, SEQ ID NO: 249, and SEQ ID NO: 290;
(o) comprising the three VL CDRs of amino acid sequences of SEQ ID NO: 113, SEQ ID NO: 143, SEQ ID NO: 180; and/or comprising the three VH CDRs of amino acid sequences of SEQ ID NO: 221, SEQ ID NO: 257, and SEQ ID NO: 291;
(p) comprising the three VL CDRs of amino acid sequences of SEQ ID NO: 106, SEQ ID NO: 149, SEQ ID NO: 181; and/or comprising the three VH CDRs of amino acid sequences of SEQ ID NO: 222, SEQ ID NO: 258, and SEQ ID NO: 292;
(q) comprising the three VL CDRs of amino acid sequences of SEQ ID NO: 114, SEQ ID NO: 150, SEQ ID NO: 182; and/or comprising the three VH CDRs of amino acid sequences of SEQ ID NO: 223, SEQ ID NO: 250, and SEQ ID NO: 293;
(r) comprising the three VL CDRs of amino acid sequences of SEQ ID NO: 115, SEQ ID NO: 151, SEQ ID NO: 183; and/or comprising the three VH CDRs of amino acid sequences of SEQ ID NO: 219, SEQ ID NO: 259, and SEQ ID NO: 294;
(s) comprising the three VL CDRs of amino acid sequences of SEQ ID NO: 116, SEQ ID NO: 152, SEQ ID NO: 184; and/or comprising the three VH CDRs of amino acid sequences of SEQ ID NO: 224, SEQ ID NO: 260, and SEQ ID NO: 295;
(t) comprising the three VL CDRs of amino acid sequences of SEQ ID NO: 117, SEQ ID NO: 153, SEQ ID NO: 185; and/or comprising the three VH CDRs of amino acid sequences of SEQ ID NO: 216, SEQ ID NO: 261, and SEQ ID NO: 296;
(u) comprising the three VL CDRs of amino acid sequences of SEQ ID NO: 118, SEQ ID NO: 143, SEQ ID NO: 186; and/or comprising the three VH CDRs of amino acid sequences of SEQ ID NO: 222, SEQ ID NO: 262, and SEQ ID NO: 297;
(v) comprising the three VL CDRs of amino acid sequences of SEQ ID NO: 119, SEQ ID NO: 154, SEQ ID NO: 187; and/or comprising the three VH CDRs of amino acid sequences of SEQ ID NO: 225, SEQ ID NO: 250, and SEQ ID NO: 298;
(w) comprising the three VL CDRs of amino acid sequences of SEQ ID NO: 120, SEQ ID NO: 140, SEQ ID NO: 188; and/or comprising the three VH CDRs of amino acid sequences of SEQ ID NO: 226, SEQ ID NO: 263, and SEQ ID NO: 299;
(x) comprising the three VL CDRs of amino acid sequences of SEQ ID NO: 121, SEQ ID NO: 141, SEQ ID NO: 189; and/or comprising the three VH CDRs of amino acid sequences of SEQ ID NO: 227, SEQ ID NO: 264, and SEQ ID NO: 300;
(y) comprising the three VL CDRs of amino acid sequences of SEQ ID NO: 122, SEQ ID NO: 155, SEQ ID NO: 190; and/or comprising the three VH CDRs of amino acid sequences of SEQ ID NO: 228, SEQ ID NO: 249, and SEQ ID NO: 301;
(z) comprising the three VL CDRs of amino acid sequences of SEQ ID NO: 123, SEQ ID NO: 143, SEQ ID NO: 186; and/or comprising the three VH CDRs of amino acid sequences of SEQ ID NO: 229, SEQ ID NO: 265, and SEQ ID NO: 302;
(aa) comprising the three VL CDRs of amino acid sequences of SEQ ID NO: 124, SEQ ID NO: 143, SEQ ID NO: 191; and/or comprising the three VH CDRs of amino acid sequences of SEQ ID NO: 216, SEQ ID NO: 266, and SEQ ID NO: 303;
(ab) comprising the three VL CDRs of amino acid sequences of SEQ ID NO: 106, SEQ ID NO: 156, SEQ ID NO: 192; and/or comprising the three VH CDRs of amino acid sequences of SEQ ID NO: 230, SEQ ID NO: 249, and SEQ ID NO: 304;
(ac) comprising the three VL CDRs of amino acid sequences of SEQ ID NO: 106, SEQ ID NO: 145, SEQ ID NO: 193; and/or comprising the three VH CDRs of amino acid sequences of SEQ ID NO: 231, SEQ ID NO: 267, and SEQ ID NO: 305;
(ad) comprising the three VL CDRs of amino acid sequences of SEQ ID NO: 125, SEQ ID NO: 143, SEQ ID NO: 194; and/or comprising the three VH CDRs of amino acid sequences of SEQ ID NO: 232, SEQ ID NO: 268, and SEQ ID NO: 306;
(ae) comprising the three VL CDRs of amino acid sequences of SEQ ID NO: 126, SEQ ID NO: 157, SEQ ID NO: 195; and/or comprising the three VH CDRs of amino acid sequences of SEQ ID NO: 233, SEQ ID NO: 269, and SEQ ID NO: 307;
(af) comprising the three VL CDRs of amino acid sequences of SEQ ID NO: 127, SEQ ID NO: 155, SEQ ID NO: 196; and/or comprising the three VH CDRs of amino acid sequences of SEQ ID NO: 234, SEQ ID NO: 249, and SEQ ID NO: 308;
(ag) comprising the three VL CDRs of amino acid sequences of SEQ ID NO: 128, SEQ ID NO: 158, SEQ ID NO: 197; and/or comprising the three VH CDRs of amino acid sequences of SEQ ID NO: 235, SEQ ID NO: 270, and SEQ ID NO: 309;
(ah) comprising the three VL CDRs of amino acid sequences of SEQ ID NO: 129, SEQ ID NO: 159, SEQ ID NO: 198; and/or comprising the three VH CDRs of amino acid sequences of SEQ ID NO: 226, SEQ ID NO: 271, and SEQ ID NO: 310;
(ai) comprising the three VL CDRs of amino acid sequences of SEQ ID NO: 106, SEQ ID NO: 143, SEQ ID NO: 199; and/or comprising the three VH CDRs of amino acid sequences of SEQ ID NO: 236, SEQ ID NO: 272, and SEQ ID NO: 311;
(aj) comprising the three VL CDRs of amino acid sequences of SEQ ID NO: 130, SEQ ID NO: 155, SEQ ID NO: 200; and/or comprising the three VH CDRs of amino acid sequences of SEQ ID NO: 237, SEQ ID NO: 249, and SEQ ID NO: 312;
(ak) comprising the three VL CDRs of amino acid sequences of SEQ ID NO: 131, SEQ ID NO: 141, SEQ ID NO: 201; and/or comprising the three VH CDRs of amino acid sequences of SEQ ID NO: 238, SEQ ID NO: 264, and SEQ ID NO: 313;
(al) comprising the three VL CDRs of amino acid sequences of SEQ ID NO: 132, SEQ ID NO: 140, SEQ ID NO: 202; and/or comprising the three VH CDRs of amino acid sequences of SEQ ID NO: 239, SEQ ID NO: 273, and SEQ ID NO: 314;
(am) comprising the three VL CDRs of amino acid sequences of SEQ ID NO: 109, SEQ ID NO: 143, SEQ ID NO: 203; and/or comprising the three VH CDRs of amino acid sequences of SEQ ID NO: 240, SEQ ID NO: 250, and SEQ ID NO: 315;
(an) comprising the three VL CDRs of amino acid sequences of SEQ ID NO: 133, SEQ ID NO: 160, SEQ ID NO: 191; and/or comprising the three VH CDRs of amino acid sequences of SEQ ID NO: 241, SEQ ID NO: 274, and SEQ ID NO: 316;
(ao) comprising the three VL CDRs of amino acid sequences of SEQ ID NO: 134, SEQ ID NO: 161, SEQ ID NO: 204; and/or comprising the three VH CDRs of amino acid sequences of SEQ ID NO: 242, SEQ ID NO: 275, and SEQ ID NO: 317;
(ap) comprising the three VL CDRs of amino acid sequences of SEQ ID NO: 135, SEQ ID NO: 162, SEQ ID NO: 189; and/or comprising the three VH CDRs of amino acid sequences of SEQ ID NO: 216, SEQ ID NO: 249, and SEQ ID NO: 318;
(aq) comprising the three VL CDRs of amino acid sequences of SEQ ID NO: 129, SEQ ID NO: 155, SEQ ID NO: 205; and/or comprising the three VH CDRs of amino acid sequences of SEQ ID NO: 226, SEQ ID NO: 276, and SEQ ID NO: 319;
(ar) comprising the three VL CDRs of amino acid sequences of SEQ ID NO: 131, SEQ ID NO: 163, SEQ ID NO: 206; and/or comprising the three VH CDRs of amino acid sequences of SEQ ID NO: 219, SEQ ID NO: 267, and SEQ ID NO: 320;
(as) comprising the three VL CDRs of amino acid sequences of SEQ ID NO: 136, SEQ ID NO: 164, SEQ ID NO: 207; and/or comprising the three VH CDRs of amino acid sequences of SEQ ID NO: 243, SEQ ID NO: 249, and SEQ ID NO: 321;
(at) comprising the three VL CDRs of amino acid sequences of SEQ ID NO: 131, SEQ ID NO: 165, SEQ ID NO: 208; and/or comprising the three VH CDRs of amino acid sequences of SEQ ID NO: 213, SEQ ID NO: 269, and SEQ ID NO: 322; or
(au) comprising the three VL CDRs of amino acid sequences of SEQ ID NO: 137, SEQ ID NO: 166, SEQ ID NO: 169; and/or comprising the three VH CDRs of amino acid sequences of SEQ ID NO: 244, SEQ ID NO: 256, and SEQ ID NO: 323.

51. The antibody of claim 50, wherein:
(a) the VH domain of the antibody comprises the amino acid sequence of SEQ ID NO: 324 or an amino acid sequence with at least 70% sequence identity thereto; and/or the VL of the antibody comprises the amino acid sequence of SEQ ID NO: 371, or an amino acid sequence with at least 70% sequence identity thereto;
(b) the VH domain of the antibody comprises the amino acid sequence of SEQ ID NO: 325 or an amino acid sequence with at least 70% sequence identity thereto; and/or the VL of the antibody comprises the amino acid sequence of SEQ ID NO: 372, or an amino acid sequence with at least 70% sequence identity thereto;
(c) the VH domain of the antibody comprises the amino acid sequence of SEQ ID NO: 326 or an amino acid sequence with at least 70% sequence identity thereto; and/or the VL of the antibody comprises the amino acid sequence of SEQ ID NO: 373, or an amino acid sequence with at least 70% sequence identity thereto;
(d) the VH domain of the antibody comprises the amino acid sequence of SEQ ID NO: 327 or an amino acid sequence with at least 70% sequence identity thereto; and/or the VL of the antibody comprises the amino acid sequence of SEQ ID NO: 374, or an amino acid sequence with at least 70% sequence identity thereto;
(e) the VH domain of the antibody comprises the amino acid sequence of SEQ ID NO: 328 or an amino acid sequence with at least 70% sequence identity thereto; and/or the VL of the antibody comprises the amino acid sequence of SEQ ID NO: 375, or an amino acid sequence with at least 70% sequence identity thereto;
(f) the VH domain of the antibody comprises the amino acid sequence of SEQ ID NO: 329 or an amino acid sequence with at least 70% sequence identity thereto; and/or the VL of the antibody comprises the amino acid sequence of SEQ ID NO: 376, or an amino acid sequence with at least 70% sequence identity thereto;
(g) the VH domain of the antibody comprises the amino acid sequence of SEQ ID NO: 330 or an amino acid sequence with at least 70% sequence identity thereto; and/or the VL of the antibody comprises the amino acid sequence of SEQ ID NO: 377, or an amino acid sequence with at least 70% sequence identity thereto;
(h) the VH domain of the antibody comprises the amino acid sequence of SEQ ID NO: 331 or an amino acid sequence with at least 70% sequence identity thereto; and/or the VL of the antibody comprises the amino acid sequence of SEQ ID NO: 378, or an amino acid sequence with at least 70% sequence identity thereto;
(i) the VH domain of the antibody comprises the amino acid sequence of SEQ ID NO: 332 or an amino acid sequence with at least 70% sequence identity thereto; and/or the VL of the antibody comprises the amino acid sequence of SEQ ID NO: 379, or an amino acid sequence with at least 70% sequence identity thereto;
(j) the VH domain of the antibody comprises the amino acid sequence of SEQ ID NO: 333 or an amino acid sequence with at least 70% sequence identity thereto; and/or the VL of the antibody comprises the amino acid sequence of SEQ ID NO: 380, or an amino acid sequence with at least 70% sequence identity thereto;
(k) the VH domain of the antibody comprises the amino acid sequence of SEQ ID NO: 334 or an amino acid sequence with at least 70% sequence identity thereto; and/or the VL of the antibody comprises the amino acid sequence of SEQ ID NO: 381, or an amino acid sequence with at least 70% sequence identity thereto;
(l) the VH domain of the antibody comprises the amino acid sequence of SEQ ID NO: 335 or an amino acid sequence with at least 70% sequence identity thereto; and/or the VL of the antibody comprises the amino acid sequence of SEQ ID NO: 382, or an amino acid sequence with at least 70% sequence identity thereto;
(m) the VH domain of the antibody comprises the amino acid sequence of SEQ ID NO: 336 or an amino acid sequence with at least 70% sequence identity thereto; and/or the VL of the antibody comprises the amino acid sequence of SEQ ID NO: 383, or an amino acid sequence with at least 70% sequence identity thereto;
(n) the VH domain of the antibody comprises the amino acid sequence of SEQ ID NO: 337 or an amino acid sequence with at least 70% sequence identity thereto; and/or the VL of the antibody comprises the amino acid sequence of SEQ ID NO: 384, or an amino acid sequence with at least 70% sequence identity thereto;
(o) the VH domain of the antibody comprises the amino acid sequence of SEQ ID NO: 338 or an amino acid sequence with at least 70% sequence identity thereto; and/or the VL of the antibody comprises the amino acid sequence of SEQ ID NO: 385, or an amino acid sequence with at least 70% sequence identity thereto;
(p) the VH domain of the antibody comprises the amino acid sequence of SEQ ID NO: 339 or an amino acid sequence with at least 70% sequence identity thereto; and/or the VL of the antibody comprises the amino acid sequence of SEQ ID NO: 386, or an amino acid sequence with at least 70% sequence identity thereto;
(q) the VH domain of the antibody comprises the amino acid sequence of SEQ ID NO: 340 or an amino acid sequence with at least 70% sequence identity thereto; and/or the VL of the antibody comprises the amino acid sequence of SEQ ID NO: 387, or an amino acid sequence with at least 70% sequence identity thereto;
(r) the VH domain of the antibody comprises the amino acid sequence of SEQ ID NO: 341 or an amino acid sequence with at least 70% sequence identity thereto; and/or the VL of the antibody comprises the amino acid sequence of SEQ ID NO: 388, or an amino acid sequence with at least 70% sequence identity thereto;
(s) the VH domain of the antibody comprises the amino acid sequence of SEQ ID NO: 342 or an amino acid sequence with at least 70% sequence identity thereto; and/or the VL of the antibody comprises the amino acid sequence of SEQ ID NO: 389, or an amino acid sequence with at least 70% sequence identity thereto;
(t) the VH domain of the antibody comprises the amino acid sequence of SEQ ID NO: 343 or an amino acid sequence with at least 70% sequence identity thereto; and/or the VL of the antibody comprises the amino acid sequence of SEQ ID NO: 390, or an amino acid sequence with at least 70% sequence identity thereto;
(u) the VH domain of the antibody comprises the amino acid sequence of SEQ ID NO: 344 or an amino acid sequence with at least 70% sequence identity thereto; and/or the VL of the antibody comprises the amino acid sequence of SEQ ID NO: 391, or an amino acid sequence with at least 70% sequence identity thereto;
(v) the VH domain of the antibody comprises the amino acid sequence of SEQ ID NO: 345 or an amino acid sequence with at least 70% sequence identity thereto; and/or the VL of the antibody comprises the amino acid sequence of SEQ ID NO: 392, or an amino acid sequence with at least 70% sequence identity thereto;
(w) the VH domain of the antibody comprises the amino acid sequence of SEQ ID NO: 346 or an amino acid sequence with at least 70% sequence identity thereto; and/or the VL of the antibody comprises the amino acid sequence of SEQ ID NO: 393, or an amino acid sequence with at least 70% sequence identity thereto;
(x) the VH domain of the antibody comprises the amino acid sequence of SEQ ID NO: 347 or an amino acid sequence with at least 70% sequence identity thereto; and/or the VL of the antibody comprises the amino acid sequence of SEQ ID NO: 394, or an amino acid sequence with at least 70% sequence identity thereto;
(y) the VH domain of the antibody comprises the amino acid sequence of SEQ ID NO: 348 or an amino acid sequence with at least 70% sequence identity thereto; and/or the VL of the antibody comprises the amino acid sequence of SEQ ID NO: 395, or an amino acid sequence with at least 70% sequence identity thereto;
(z) the VH domain of the antibody comprises the amino acid sequence of SEQ ID NO: 349 or an amino acid sequence with at least 70% sequence identity thereto; and/or the VL of the antibody comprises the amino acid sequence of SEQ ID NO: 396, or an amino acid sequence with at least 70% sequence identity thereto;
(aa) the VH domain of the antibody comprises the amino acid sequence of SEQ ID NO: 350 or an amino acid sequence with at least 70% sequence identity thereto; and/or the VL of the antibody comprises the amino acid sequence of SEQ ID NO: 397, or an amino acid sequence with at least 70% sequence identity thereto;
(ab) the VH domain of the antibody comprises the amino acid sequence of SEQ ID NO: 351 or an amino acid sequence with at least 70% sequence identity thereto; and/or the VL of the antibody comprises the amino acid sequence of SEQ ID NO: 398, or an amino acid sequence with at least 70% sequence identity thereto;
(ac) the VH domain of the antibody comprises the amino acid sequence of SEQ ID NO: 352 or an amino acid sequence with at least 70% sequence identity thereto; and/or the VL of the antibody comprises the amino acid sequence of SEQ ID NO: 399, or an amino acid sequence with at least 70% sequence identity thereto;
(ad) the VH domain of the antibody comprises the amino acid sequence of SEQ ID NO: 353 or an amino acid sequence with at least 70% sequence identity thereto; and/or the VL of the antibody comprises the amino acid sequence of SEQ ID NO: 400, or an amino acid sequence with at least 70% sequence identity thereto;
(ae) the VH domain of the antibody comprises the amino acid sequence of SEQ ID NO: 354 or an amino acid sequence with at least 70% sequence identity thereto; and/or the VL of the antibody comprises the amino acid sequence of SEQ ID NO: 401, or an amino acid sequence with at least 70% sequence identity thereto;
(af) the VH domain of the antibody comprises the amino acid sequence of SEQ ID NO: 355 or an amino acid sequence with at least 70% sequence identity thereto; and/or the VL of the antibody comprises the amino acid sequence of SEQ ID NO: 402, or an amino acid sequence with at least 70% sequence identity thereto;
(ag) the VH domain of the antibody comprises the amino acid sequence of SEQ ID NO: 356 or an amino acid sequence with at least 70% sequence identity thereto; and/or the VL of the antibody comprises the amino acid sequence of SEQ ID NO: 403, or an amino acid sequence with at least 70% sequence identity thereto;
(ah) the VH domain of the antibody comprises the amino acid sequence of SEQ ID NO: 357 or an amino acid sequence with at least 70% sequence identity thereto; and/or the VL of the antibody comprises the amino acid sequence of SEQ ID NO: 404, or an amino acid sequence with at least 70% sequence identity thereto;
(ai) the VH domain of the antibody comprises the amino acid sequence of SEQ ID NO: 358 or an amino acid sequence with at least 70% sequence identity thereto; and/or the VL of the antibody comprises the amino acid sequence of SEQ ID NO: 405, or an amino acid sequence with at least 70% sequence identity thereto;
(aj) the VH domain of the antibody comprises the amino acid sequence of SEQ ID NO: 359 or an amino acid sequence with at least 70% sequence identity thereto; and/or the VL of the antibody comprises the amino acid sequence of SEQ ID NO: 406, or an amino acid sequence with at least 70% sequence identity thereto;
(ak) the VH domain of the antibody comprises the amino acid sequence of SEQ ID NO: 360 or an amino acid sequence with at least 70% sequence identity thereto; and/or the VL of the antibody comprises the amino acid sequence of SEQ ID NO: 407, or an amino acid sequence with at least 70% sequence identity thereto;
(al) the VH domain of the antibody comprises the amino acid sequence of SEQ ID NO: 361 or an amino acid sequence with at least 70% sequence identity thereto; and/or the VL of the antibody comprises the amino acid sequence of SEQ ID NO: 408, or an amino acid sequence with at least 70% sequence identity thereto;
(am) the VH domain of the antibody comprises the amino acid sequence of SEQ ID NO: 362 or an amino acid sequence with at least 70% sequence identity thereto; and/or the VL of the antibody comprises the amino acid sequence of SEQ ID NO: 409, or an amino acid sequence with at least 70% sequence identity thereto;
(an) the VH domain of the antibody comprises the amino acid sequence of SEQ ID NO: 363 or an amino acid sequence with at least 70% sequence identity thereto; and/or the VL of the antibody comprises the amino acid sequence of SEQ ID NO: 410, or an amino acid sequence with at least 70% sequence identity thereto;
(ao) the VH domain of the antibody comprises the amino acid sequence of SEQ ID NO: 364 or an amino acid sequence with at least 70% sequence identity thereto; and/or the VL of the antibody comprises the amino acid sequence of SEQ ID NO: 411, or an amino acid sequence with at least 70% sequence identity thereto;
(ap) the VH domain of the antibody comprises the amino acid sequence of SEQ ID NO: 365 or an amino acid sequence with at least 70% sequence identity thereto; and/or the VL of the antibody comprises the amino acid sequence of SEQ ID NO: 412, or an amino acid sequence with at least 70% sequence identity thereto;
(aq) the VH domain of the antibody comprises the amino acid sequence of SEQ ID NO: 366 or an amino acid sequence with at least 70% sequence identity thereto; and/or the VL of the antibody comprises the amino acid sequence of SEQ ID NO: 413, or an amino acid sequence with at least 70% sequence identity thereto;
(ar) the VH domain of the antibody comprises the amino acid sequence of SEQ ID NO: 367 or an amino acid sequence with at least 70% sequence identity thereto; and/or the VL of the antibody comprises the amino acid sequence of SEQ ID NO: 414, or an amino acid sequence with at least 70% sequence identity thereto;
(as) the VH domain of the antibody comprises the amino acid sequence of SEQ ID NO: 368 or an amino acid sequence with at least 70% sequence identity thereto; and/or the VL of the antibody comprises the amino acid sequence of SEQ ID NO: 415, or an amino acid sequence with at least 70% sequence identity thereto;
(at) the VH domain of the antibody comprises the amino acid sequence of SEQ ID NO: 369 or an amino acid sequence with at least 70% sequence identity thereto; and/or the VL of the antibody comprises the amino acid sequence of SEQ ID NO: 416, or an amino acid sequence with at least 70% sequence identity thereto; or
(au) the VH domain of the antibody comprises the amino acid sequence of SEQ ID NO: 370 or an amino acid sequence with at least 70% sequence identity thereto; and/or the VL of the antibody comprises the amino acid sequence of SEQ ID NO: 417, or an amino acid sequence with at least 70% sequence identity thereto.

52. The antibody of claim 50, wherein the antibody is an antibody fragment.

53.-56. (canceled)

57. The antibody of claim 50, wherein the antibody comprises a Fc domain, and the Fc domain is selected from the group consisting of human IgG1, IgG2, IgG3, and IgG4 heavy chain sequence.

58. The antibody of claim 57, wherein the Fc domain is from the heavy chain IgG amino acid sequences of SEQ ID NO: 5 or SEQ ID NO: 28, optionally with one or more Fc amino acid substitutions.

59. The antibody of claim 58, wherein the Fc domain is from the heavy chain IgG amino acid sequences of any one of SEQ ID NOS: 5-36.

60. The antibody claim 58, wherein the heavy chain Fc domain comprises one or more amino acid substitutions relative to SEQ ID NO: 5 or SEQ ID NO: 28 at a position selected from the group consisting of: 215, 221, 222, 228, 234, 235, 236, 239, 240, 241, 243, 244, 245, 247, 250, 252, 254, 256, 262, 263, 264, 265, 266, 267, 268, 269, 270, 292, 296, 297, 298, 299, 300, 305, 313, 324, 325, 326, 327, 328, 329, 330, 332, 333, 334, 345, 396, 428, 430, 433, 434, and 440 wherein the position numbers of the amino acid residues are of the EU numbering scheme.

61. The antibody of claim 50, wherein the antibody comprises a light chain constant region that is from the amino acid sequences of any one of SEQ ID NOS: 38-42.

62. The antibody of claim 50, wherein the binding of the antibody does not disrupt the interaction between CD47 and SIRPγ.

63. The antibody of claim 50, wherein the binding of the antibody disrupts the interaction between CD47 and SIRPγ.

64. The antibody of claim 50, wherein the binding of the antibody stabilizes or promotes the interaction between CD47 and SIRPγ.

65.-66. (canceled)

67. A pharmaceutical composition comprising the antibody of claim 50, and optionally a pharmaceutically acceptable carrier.

68. A nucleic acid encoding for the antibody of claim 51.

69. A vector comprising the nucleic acid claim 68.

70. A method of inducing the preferential depletion of a population of SIRPγ-expressing cells, the method comprising contacting the population with the antibody of claim 50.

71. The method of claim 70, wherein the SIRPγ-expressing cells are T-cells, B-cells and/or NK-cells.

72. (canceled)

73. The method of claim 71, wherein the T-cells are activated (stimulated) or exhausted.

74. The method of claim 71, wherein the T-cells are activated (stimulated), and the depletion is preferential for activated (stimulated) T-cells.

75. (canceled)

76. The method of claim 71, wherein the T-cells are exhausted, and the depletion is preferential for exhausted T-cells.

77. The method of claim 71, wherein the T-cells are naïve, central memory, effector memory, exhausted, or terminal effector memory cells.

78. The method of claim 71, wherein the T-cells are cytotoxic T-cells, helper T-cells, memory T-cells, regulatory T-cells, natural killer T-cells, mucosal associated invariant T-cells, alpha beta T-cells, or gamma delta T-cells.

79. The method of claim 71, wherein the T-cells are Th1 cells, Th2 cells, Th17 cells or T follicular helper cells

80. The method of claim 71, wherein the T-cells are a CD3+ T-cell, CD4+ T-cell, CD8+ T-cell, CD25+ T-cell, CD69+ T-cell, and/or PD1+ T-cell.

81.-95. (canceled)

96. The method of claim 70, wherein, the SIRPγ-expressing cells are activated.

97. The method of claim 70, wherein the method is in vitro or in vivo.

98. (canceled)

99. The method of claim 70, wherein the population of SIRPγ-expressing cells comprises tissue-resident cells.

100. The method of claim 70, wherein the population of SIRPγ-expressing cells comprises circulating cells.

101. The method of claim 70, wherein the depletion involves one or more of ADCC, ADCP, and CDC.

102. A method of treating a disease or condition in a subject in need thereof, comprising administering to the subject a therapeutically effective amount of the pharmaceutical composition of claim 67.

103. The method of claim 102, wherein the disease or condition involves SIRPγ-expressing cells.

104. The method of claim 103, wherein the SIRPγ-expressing cell is a T-cell, B-cell, or a NK-cell.

105. (canceled)

106. The method of claim 103, wherein the T-cell is stimulated (activated) or exhausted.

107. (canceled)

108. The method of claim 104, wherein the T-cell is a cytotoxic T-cell, helper T-cell, a memory T-cell, a regulatory T-cell, a natural killer T-cell, a mucosal associated invariant T-cell or a gamma delta T-cell.

109. The method of claim 104, wherein the T-cell is a naïve, central memory, effector memory, or terminal effector memory cell.

110. The method of claim 104, wherein the T-cell is a CD3+ T-cell, CD4+ T-cell, CD8+ T-cell, CD25+ T-cell, CD69+ T-cell, and/or PD1+ T-cell.

111.-124. (canceled)

125. The method of claim 104, wherein the B-Cell is activated.

126. (canceled)

127. The method of claim 104, wherein the NK-cell is activated.

128. The method of claim 102, wherein the disease or condition comprises an autoimmune, oncology, or inflammatory disorder.

129. The method of claim 128, wherein the disease or condition comprises a T-cell-mediated autoimmune disease, T-cell-mediated inflammatory disease, or T-cell-mediated oncology disease.

130.-131. (canceled)

132. The method of claim 102, wherein the disease or condition is driven by CD8+ T-cells and/or CD4+ T-cells.

133. The method of claim 102, wherein the disease or condition is selected from: Acute and Chronic eosinophilic pneumonia, Acute disseminated encephalomyelitis, Acute Disseminated Encephalomyelitis, Acute Lymphoblastic Leukemia (ALL), Acute myelogenous leukemia (AML), Addison's disease, Adult-onset Still's disease (AOSD), Aplastic anemia, Ataxia Telangiectasia, Atopic dermatitis, Autoimmune lymphoproliferative syndrome, Axial spondyloarthritis (AxSpA), Birdshot Retinochoroidopathy, Castleman disease, Celiac disease, Chediak-Higashi syndrome, Coronary artery disease/peripheral artery disease, Crohn's Disease, Episodic angioedema with eosinophilia/Gleich syndrome, Giant-Lymphocyte arteritis, Graft failure post-HSCT, Graft vs Host Disease (GvHD), Graves' disease, Hepatosplenic lymphoma, Hypothyroidism, Idiopathic interstitial pneumonias, IgA nephropathy, Inclusion Body Myositis (IBM), Inflammatory bowel disease (IBD), Large granular lymphocytic leukemia, Lymphocyte-variant hypereosinophila, Multiple sclerosis, Myelodysplastic syndromes (MDS), Myocarditis, Neuromyelitis optica spectrum disorders, Paraneoplastic syndromes, Primary biliary cholangitis, Primary sclerosing cholangitis, Rasmussen's Encephalitis, Rheumatoid arthritis (RA), Sarcoidosis, Schmidt syndrome/Autoimmune polyendocrine syndrome type II, Stiff Person Syndrome, Susac syndrome, Sympathetic Opthalmis, Systemic juvenile idiopathic arthritis (sJIA), Systemic Lupus Erythematosus (SLE), T-Lymphocyte mediated rejection of solid organ transplants, T-Cell Prolymphocytic Leukemia (TPLL), Type 1 diabetes, Ulcerative colitis, Uveitis, Vitiligo, X-linked Hyper IgM Syndrome, Autoimmune Hepatitis, and X-linked lymphoproliferative disease.

134. The method of claim 102, wherein the subject is human.

135.-138. (canceled)
